(19) United States
(12) Patent Application Publication
(10) Pub. No.: US 2023/0140483 A1
(43) Pub. Date: May 4, 2023

(54) COMPOUND FOR ORGANIC OPTOELECTRONIC DEVICE, COMPOSITION FOR ORGANIC OPTOELECTRONIC DEVICE, ORGANIC OPTOELECTRONIC DEVICE, AND DISPLAY DEVICE

(71) Applicant: SAMSUNG SDI CO., LTD., Yongin-si (KR)

(72) Inventors: Jiyun KWON, Suwon-si (KR); Dong Min KANG, Suwon-si (KR); Junmo PARK, Suwon-si (KR); Hansol SEO, Suwon-si (KR); Byoungkwan LEE, Suwon-si (KR); Jonghoon KO, Suwon-si (KR); Namheon LEE, Suwon-si (KR); Mijin LEE, Suwon-si (KR); Kipo JANG, Suwon-si (KR); Sung-Hyun JUNG, Suwon-si (KR); Ho Kuk JUNG, Suwon-si (KR)

(21) Appl. No.: 17/952,410

(22) Filed: Sep. 26, 2022

(30) Foreign Application Priority Data
Sep. 28, 2021 (KR) 10-2021-0128112

(51) Int. Cl. H01L 51/00; C07D 405/04; C07D 209/80

(52) U.S. Cl. CPC H01L 51/0067; C07D 405/04; C07D 209/80; H01L 51/0073; H01L 51/006; H01L 51/0061; H01L 51/0072; H01L 51/5016

(57) ABSTRACT

Provided are a compound for an organic optoelectronic device represented by Chemical Formula 1, a composition for an organic optoelectronic device including the same, an organic optoelectronic device, and a display device. The content of Chemical Formula 1 is as defined in the specification.

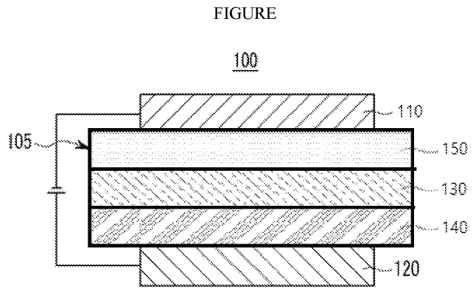

### CROSS-REFERENCE TO RELATED APPLICATION

This application claims priority to and the benefit of Korean Patent Application No. 10-2021-0128112 filed in the Korean Intellectual Property Office on Sep. 28, 2021, the entire contents of which are incorporated herein by reference.

### BACKGROUND

### 1. Field

A compound for an organic optoelectronic device, a composition for an organic optoelectronic device, an organic optoelectronic device, and a display device are disclosed.

### 2. Description of the Related Art

An organic optoelectronic device (organic optoelectronic diode) is a device that converts electrical energy into photoenergy, and vice versa.

An organic optoelectronic device may be classified as follows in accordance with its driving principles. One is a photoelectric device where excitons generated by photoenergy are separated into electrons and holes and the electrons and holes are transferred to different electrodes respectively and electrical energy is generated, and the other is a light emitting device to generate photoenergy from electrical energy by supplying a voltage or a current to electrodes.

Examples of the organic optoelectronic device include an organic photoelectric device, an organic light emitting diode, an organic solar cell, and an organic photo conductor drum.

Among them, the organic light emitting diode (OLED) has recently drawn attention due to an increase in demand for flat panel displays. The organic light emitting diode converts electrical energy into light, and the performance of organic light emitting diode is greatly influenced by the organic materials disposed between electrodes.

### SUMMARY

An embodiment is directed to a compound for an organic optoelectronic device represented by Chemical Formula 1.

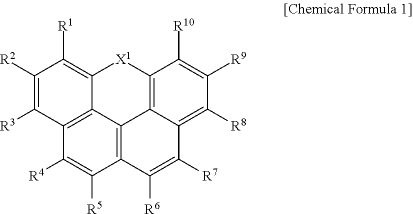

In Chemical Formula 1,

X¹ is O or S,

R¹ to R¹⁰ are each independently hydrogen, deuterium, a halogen, a hydroxy group, a cyano group, a nitro group, a substituted or unsubstituted C1 to C30 alkyl group, a substituted or unsubstituted C6 to C30 aryl group, or a substituted or unsubstituted C2 to C30 heterocyclic group, and

at least one of R¹ to R⁵ is represented by Chemical Formula a.

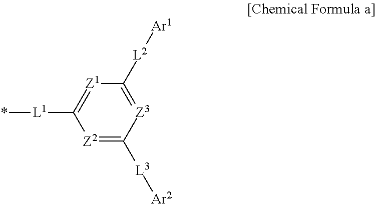

In Chemical Formula a,

Z¹ to Z³ are each independently N or CR^{a},

at least two of Z¹ to Z³ are N,

L¹ to L³ are each independently a single bond or a substituted or unsubstituted C6 to C30 arylene group,

Ar¹ and Ar² are each independently a substituted or unsubstituted C6 to C30 aryl group or a substituted or unsubstituted C2 to C30 heterocyclic group,

R^{a} is hydrogen, deuterium, a halogen, a hydroxy group, a cyano group, a nitro group, a substituted or unsubstituted C1 to C20 alkyl group, or a substituted or unsubstituted C6 to C20 aryl group.

An embodiment is directed to a composition for an organic optoelectronic device that includes a first compound and a second compound.

The first compound may be the aforementioned compound for the organic optoelectronic device, and the second compound may be represented by Chemical Formula 2.

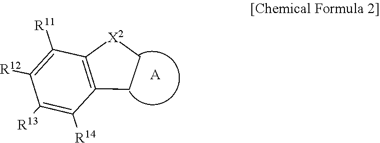

In Chemical Formula 2,

X² is O, S, NR^{b}, CR^{c}R^{d} or SiR^{e}R^{f},

R^{b}, R^{c}, R^{d}, R^{e}, R^{f}, and R¹¹ to R¹⁴ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and

ring A is any one selected from the rings of Group II.

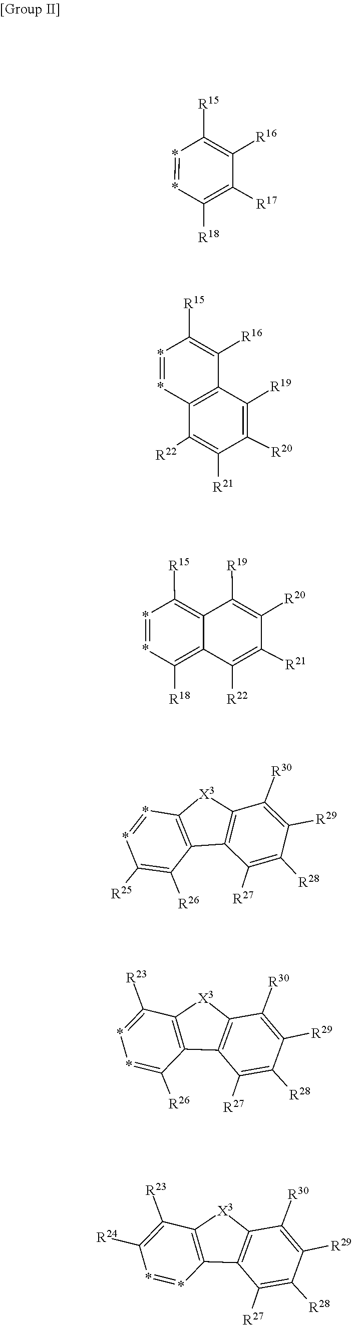

In Group II,

* is a linking point,

X³ is O, S, NR^{g}, CR^{h}Rⁱ, or SiR^{j}R^{k},

R^{g}, R^{h}, Rⁱ, R^{j}, R^{k}, and R¹⁵ to R³⁰ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and

at least one of R¹¹ to R³⁰ is a group represented by Chemical Formula b.

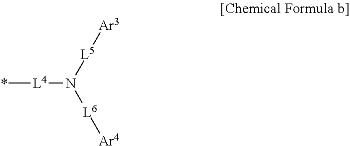

In Chemical Formula b,

L⁴ to L⁶ are each independently a single bond, or a substituted or unsubstituted C6 to C30 arylene group,

Ar³ and Ar⁴ are each independently a substituted or unsubstituted amine group, a substituted or unsubstituted C6 to C30 aryl group, or a substituted or unsubstituted C2 to C30 heterocyclic group, and

* is a linking point.

An embodiment is directed to an organic optoelectronic device that includes an anode and a cathode facing each other, at least one organic layer disposed between the anode and the cathode, wherein the organic layer includes the compound for the organic optoelectronic device or composition for the organic optoelectronic device.

An embodiment is directed to a display device including the organic optoelectronic device.

### BRIEF DESCRIPTION OF THE DRAWINGS

Features will become apparent to those of skill in the art by describing in detail exemplary embodiments with reference to the attached drawings in which:

The FIGURE is a cross-sectional view illustrating an organic light emitting diode according to an example embodiment.

### DETAILED DESCRIPTION

Example embodiments will now be described more fully hereinafter with reference to the accompanying drawing; however, they may be embodied in different forms and should not be construed as limited to the embodiments set forth herein. Rather, these embodiments are provided so that this disclosure will be thorough and complete, and will fully convey exemplary implementations to those skilled in the art.

In the drawing FIGURE, the dimensions of layers and regions may be exaggerated for clarity of illustration. Like reference numerals refer to like elements throughout.

As used herein, when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example, “substituted” refers to replacement of at least one hydrogen of a substituent or a compound by deuterium, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, or a cyano group. In addition, in specific examples, “substituted” refers to replacement of at least one hydrogen of a substituent or a compound by deuterium, a C1 to C20 alkyl group, a C6 to C30 aryl group, or a cyano group. In addition, in specific examples, “substituted” refers to replacement of at least one hydrogen of a substituent or a compound by deuterium, a C1 to C5 alkyl group, a C6 to C18 aryl group, or a cyano group. In addition, in specific examples, “substituted” refers to replacement of at least one hydrogen of a substituent or a compound by deuterium, a cyano group, a methyl group, an ethyl group, a propyl group, a butyl group, a phenyl group, a biphenyl group, a terphenyl group, or a naphthyl group.

As used herein, “unsubstituted” refers to non-replacement of a hydrogen atom by another substituent and remaining of the hydrogen atom.

As used herein, “hydrogen substitution (—H)” may include “deuterium substitution (-D)” or “tritium substitution (-T).”

As used herein, when a definition is not otherwise provided, “hetero” refers to one including one to three heteroatoms selected from N, O, S, P, and Si, and remaining carbons in one functional group.

As used herein, “aryl group” refers to a group including at least one hydrocarbon aromatic moiety, and may include a group in which all elements of the hydrocarbon aromatic moiety have p-orbitals which form conjugation, for example, a phenyl group, a naphthyl group, and the like, a group in which two or more hydrocarbon aromatic moieties may be linked by a sigma bond, for example, a biphenyl group, a terphenyl group, a quarterphenyl group, and the like, and a group in which two or more hydrocarbon aromatic moieties are fused directly or indirectly to provide a non-aromatic fused ring, for example, a fluorenyl group, and the like.

The aryl group may include a monocyclic, polycyclic or fused ring polycyclic (i.e., rings sharing adjacent pairs of carbon atoms) functional group.

As used herein, “heterocyclic group” is a generic concept of a heteroaryl group, and may include at least one heteroatom selected from N, O, S, P, and Si instead of carbon (C) in a cyclic compound such as an aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” refers to an aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzthiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted carbazolyl group, a substituted or unsubstituted dibenzofuranyl group, a substituted or unsubstituted dibenzothiophenyl group, a substituted or unsubstituted benzonaphthofuranyl group, a substituted or unsubstituted benzonaphthothiophenyl group a substituted or unsubstituted dibenzosilolyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to the highest occupied molecular orbital (HOMO) level.

In addition, electron characteristics refer to an ability to accept an electron when an electric field is applied and that electron formed in the cathode may be easily injected into the light emitting layer and transported in the light emitting layer due to conductive characteristics according to the lowest unoccupied molecular orbital (LUMO) level.

Hereinafter, a compound for an organic optoelectronic device according to an example embodiment is described.

The compound for an organic optoelectronic device according to an example embodiment is represented by Chemical Formula 1.

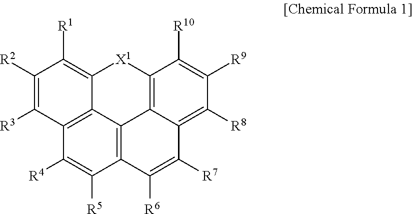

In Chemical Formula 1,

X¹ is O or S,

R¹ to R¹⁰ are each independently hydrogen, deuterium, a halogen, a hydroxy group, a cyano group, a nitro group, a substituted or unsubstituted C1 to C30 alkyl group, a substituted or unsubstituted C6 to C30 aryl group, or a substituted or unsubstituted C2 to C30 heterocyclic group, and

at least one of R¹ to R⁵ is represented by Chemical Formula a.

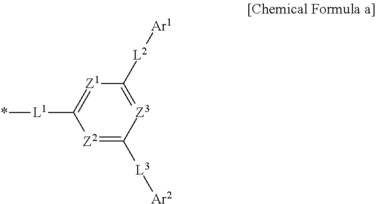

In Chemical Formula a,

Z¹ to Z³ are each independently N or CR^{a},

at least two of Z¹ to Z³ are N,

L¹ to L³ are each independently a single bond or a substituted or unsubstituted C6 to C30 arylene group,

Ar¹ and Ar² are each independently a substituted or unsubstituted C6 to C30 aryl group or a substituted or unsubstituted C2 to C30 heterocyclic group,

R^{a} is hydrogen, deuterium, a halogen, a hydroxy group, a cyano group, a nitro group, a substituted or unsubstituted C1 to C20 alkyl group, or a substituted or unsubstituted C6 to C20 aryl group.

The compound represented by Chemical Formula 1 has a structure in which a fused xanthene core is substituted with pyrimidine or triazine.

Since the structure including the fused xanthene core has high charge mobility and a large current on/off ratio, an organic light emitting diode to which it is applied may realize high efficiency, low voltage, and long life-span characteristics.

In particular, as substituted with pyrimidine or triazine, high electron mobility may be obtained, thereby lowering a driving voltage.

In addition, since the polycyclic fused structure contains —O— (or —S—) bridges, a glass transition temperature (Tg) may be increased compared with structures without such bridges, and thus it has an advantageous effect in terms of processability and stability.

Chemical Formula 1 may be represented by any one of Chemical Formula 1-1 to Chemical Formula 1-5, depending on the substitution position of pyrimidine or triazine.

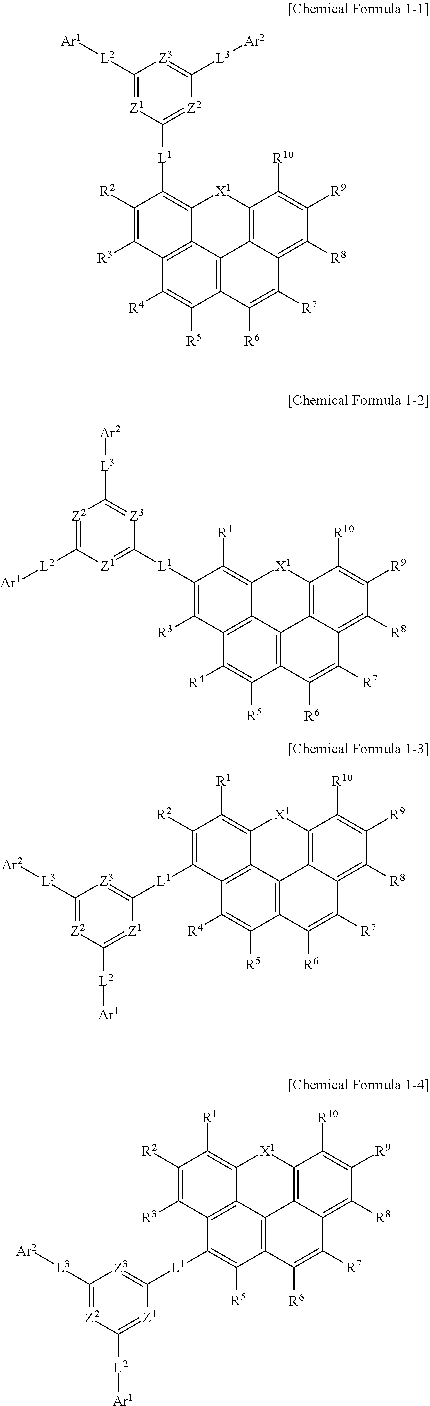

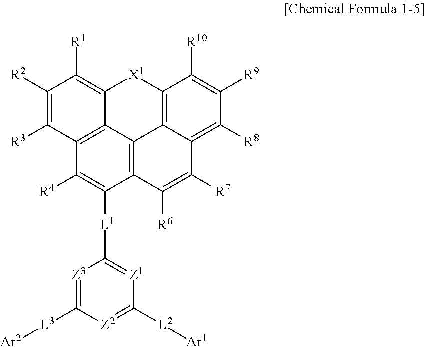

In Chemical Formula 1-1 to Chemical Formula 1-5, X¹, R¹ to R¹⁰, L¹ to L³, Z¹ to Z³, Ar¹, and Ar² are the same as described above.

In an example embodiment, the compound for the organic optoelectronic device according to an example embodiment may be represented by Chemical Formula 1-1 or Chemical Formula 1-2.

In an example embodiment, L¹ to L³ may each independently be a single bond, a substituted or unsubstituted phenylene group, a substituted or unsubstituted biphenylene group, a substituted or unsubstituted naphthylene group, or a substituted or unsubstituted phenanthrenylene group.

In an example embodiment, Ar¹ and Ar² may each independently be a substituted or unsubstituted phenyl group, a substituted or unsubstituted biphenyl group, a substituted or unsubstituted naphthyl group, a substituted or unsubstituted phenanthrenyl group, a substituted or unsubstituted fluorenyl group, a substituted or unsubstituted chrysenyl group, a substituted or unsubstituted carbazolyl group, a substituted or unsubstituted dibenzofuranyl group, a substituted or unsubstituted dibenzothiophenyl group, a substituted or unsubstituted dibenzosilolyl group, a substituted or unsubstituted benzonaphthofuranyl group, a substituted or unsubstituted benzonaphthothiophenyl group, a substituted or unsubstituted dinaphthofuranyl group, a substituted or unsubstituted dinaphthothiophenyl group, a substituted or unsubstituted benzophenanthrofuranyl group, or a substituted or unsubstituted benzophenanthrothiophenyl group.

In an example embodiment, Ar¹ and Ar² may each independently be a substituted or unsubstituted phenyl group, a substituted or unsubstituted naphthyl group, a substituted or unsubstituted carbazolyl group, a substituted or unsubstituted dibenzofuranyl group, a substituted or It may be an unsubstituted dibenzothiophenyl group, or a substituted or unsubstituted benzonaphthofuranyl group.

In an example embodiment, *-L²-Ar¹ and *-L³-Ar² may be each independently selected from the substituents of Group I.

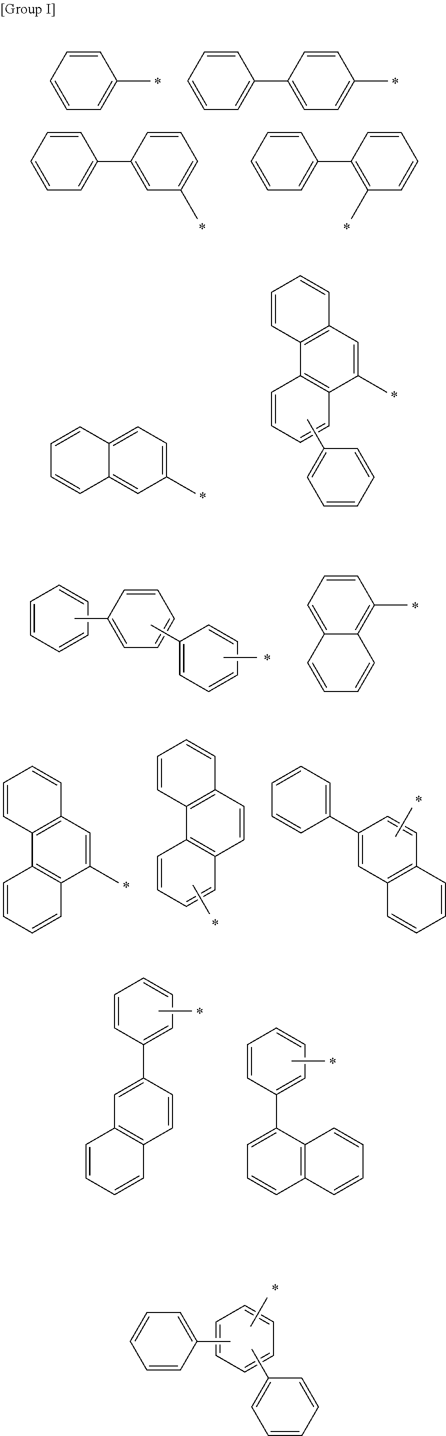

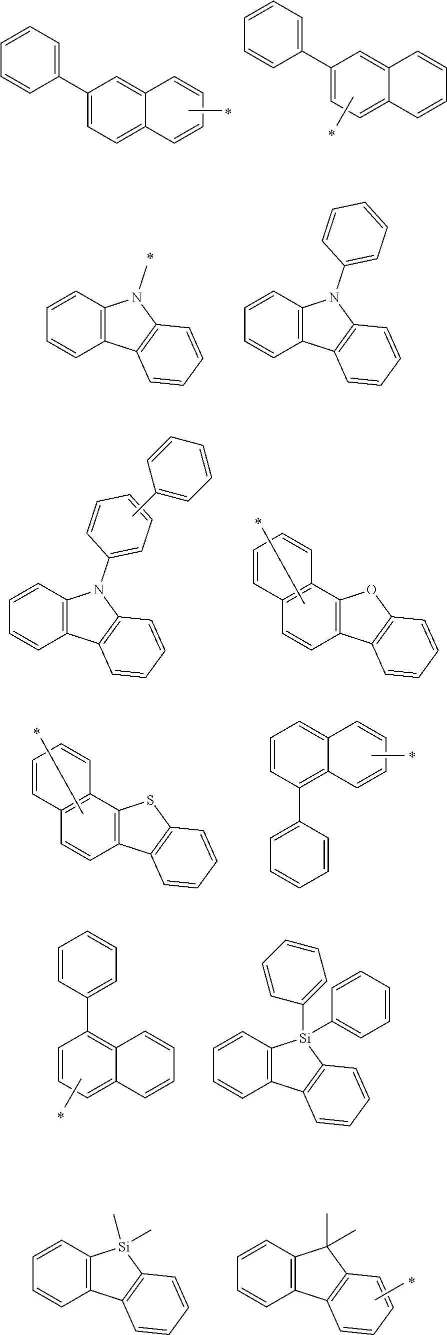

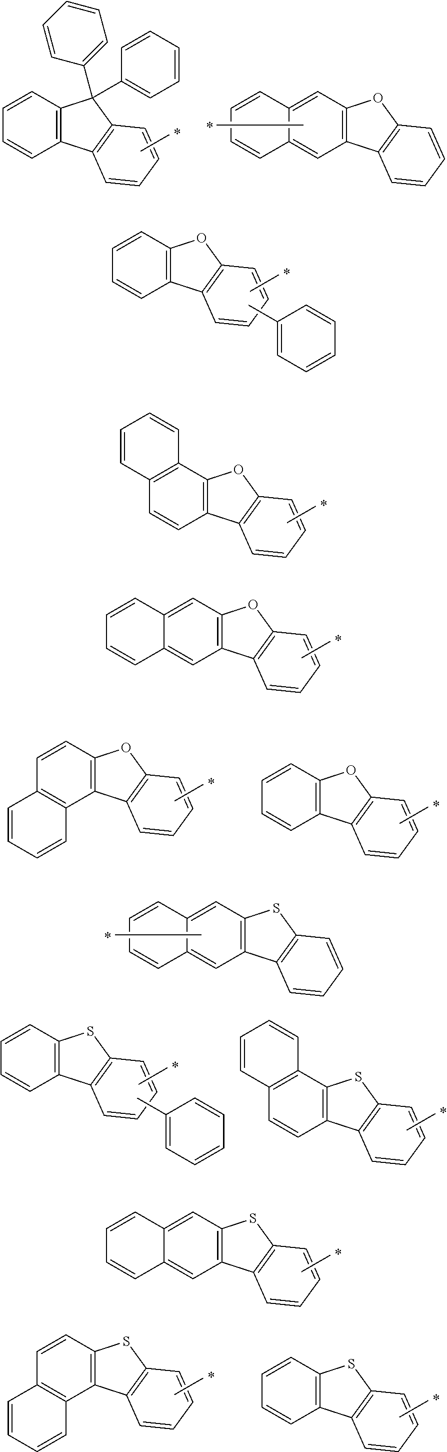

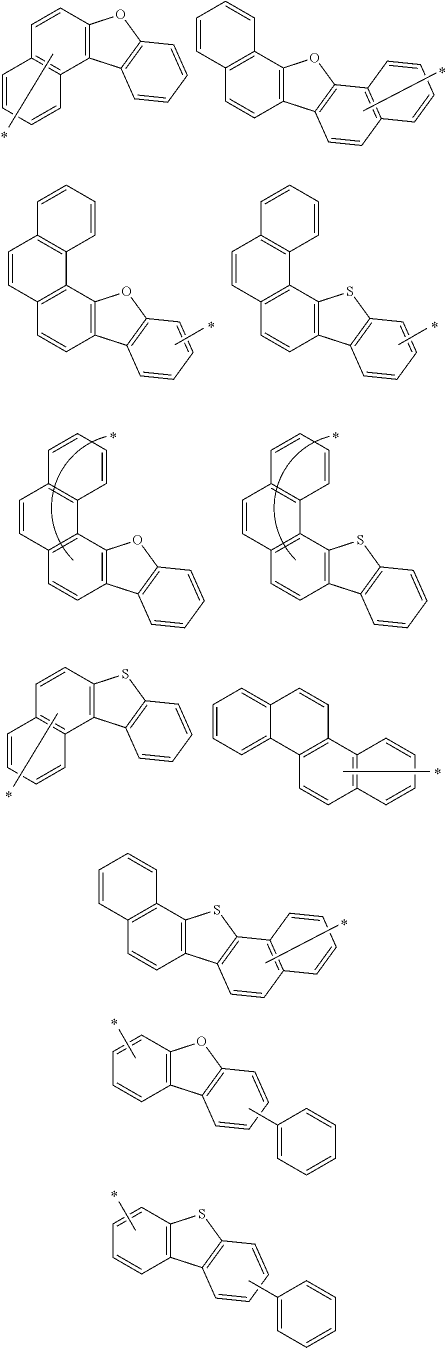

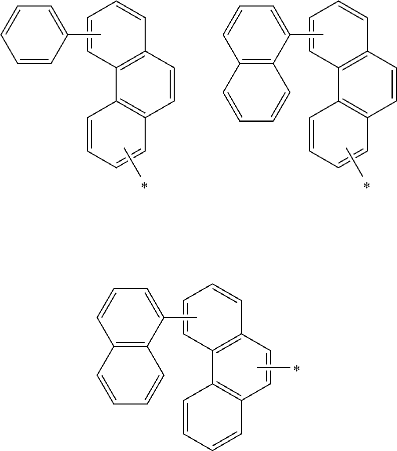

In Group I, * is a linking point.

The substituents listed in Group I may be in an unsubstituted form or a substituted form having an additional substituent, and in the case of a substituted form, it may be substituted with at least one selected from the substituents of the definition of “substituted” above.

In an example embodiment, the compound for the organic optoelectronic device represented by Chemical Formula 1 may be one selected from the compounds of Group 1.

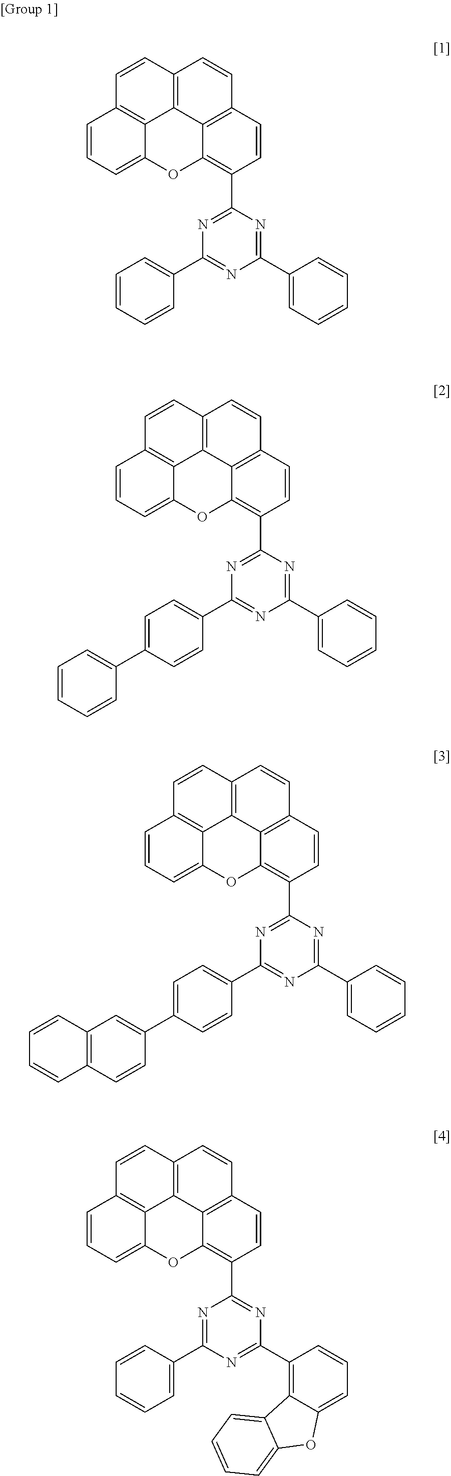 [SMILES: c1ccc(-c2nc(-c3ccccc3)nc(-c3ccc4ccc5ccc6cccc7c6c5c4c3O7)n2)cc1 ; c1ccc(-c2ccc(-c3nc(-c4ccccc4)nc(-c4ccc5ccc6ccc7cccc8c7c6c5c4O8)n3)cc2)cc1 ; c1ccc(-c2nc(-c3ccc(-c4ccc5ccccc5c4)cc3)nc(-c3ccc4ccc5ccc6cccc7c6c5c4c3O7)n2)cc1 ; c1ccc(-c2nc(-c3ccc4ccc5ccc6cccc7c6c5c4c3O7)nc(-c3cccc4oc5ccccc5c34)n2)cc1]

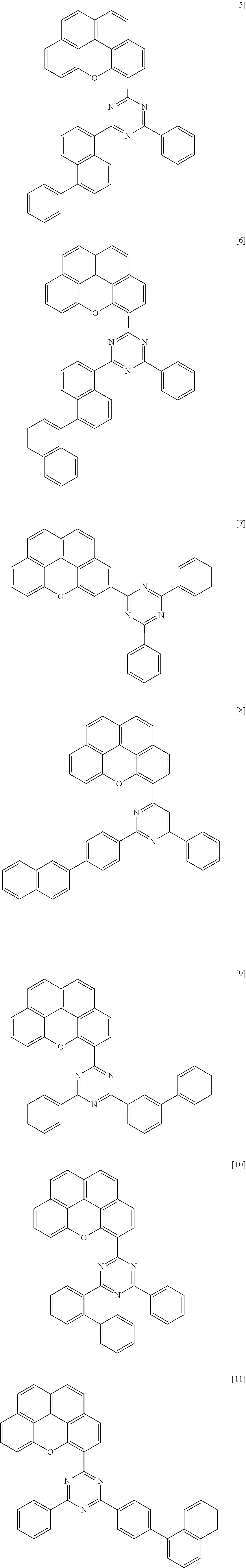

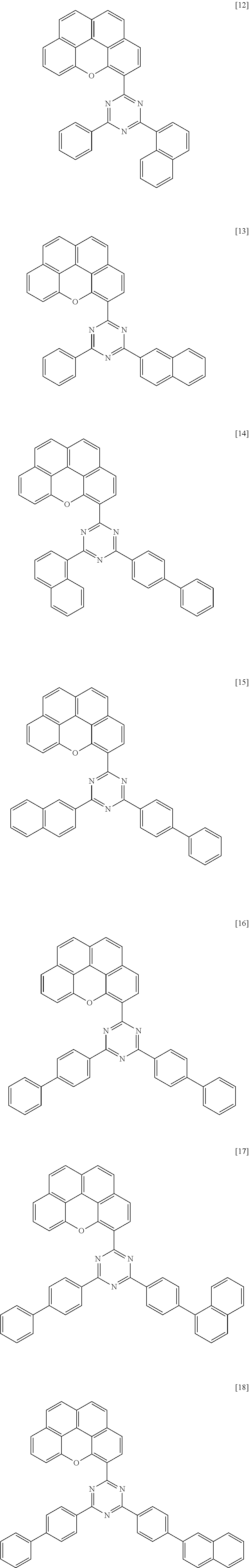

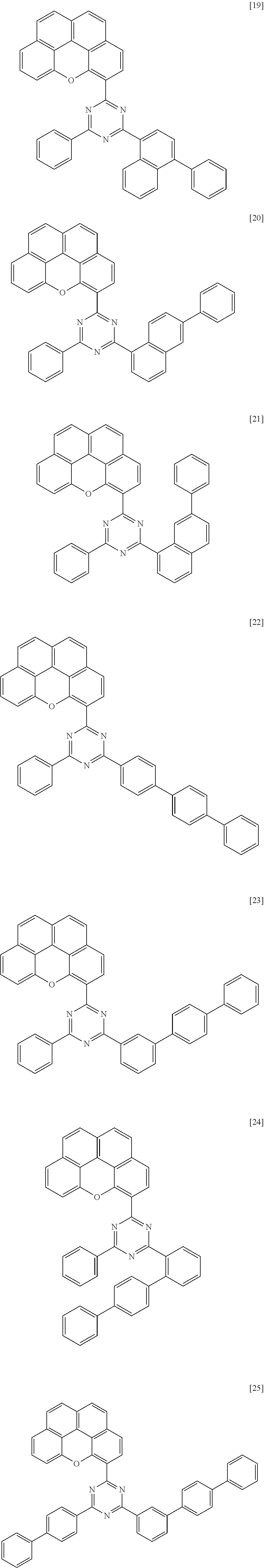

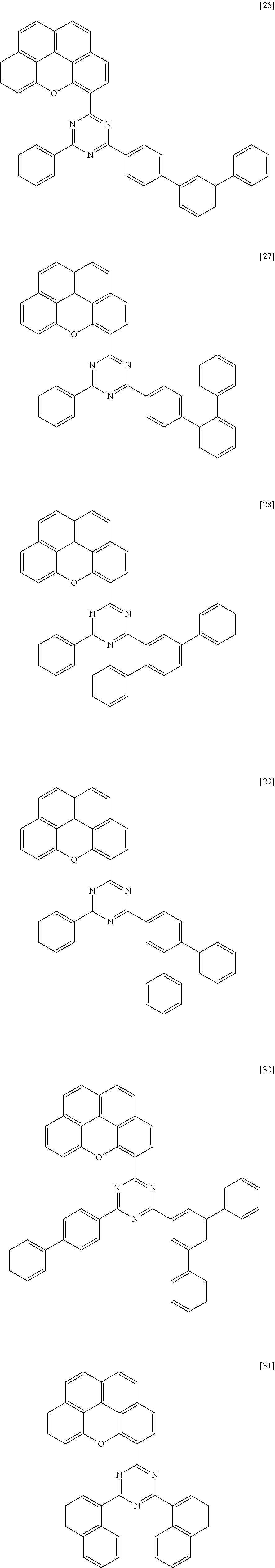

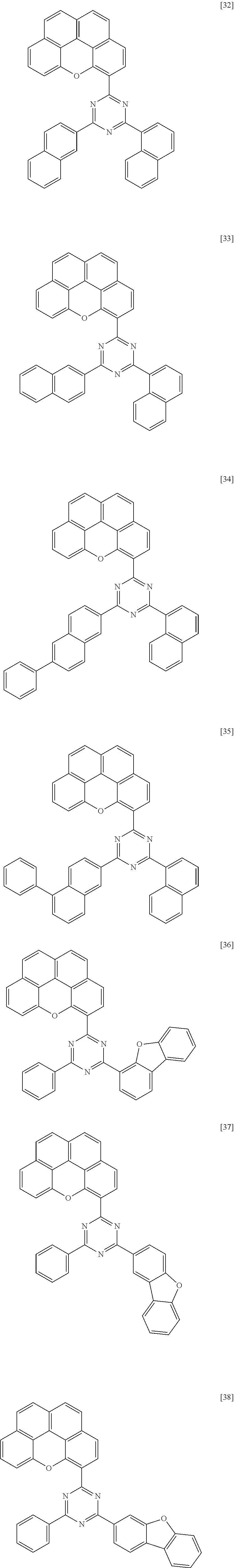

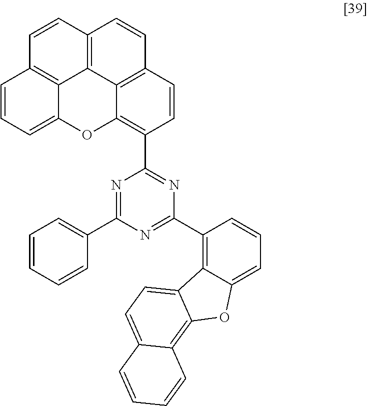 [SMILES: c1ccc(-c2nc(-c3ccc4ccc5ccc6cccc7c6c5c4c3O7)nc(-c3cccc4oc5c6ccccc6ccc5c34)n2)cc1]

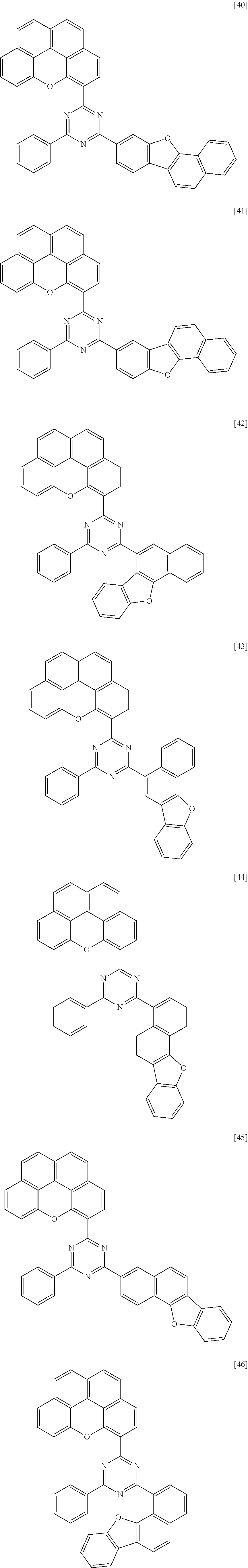

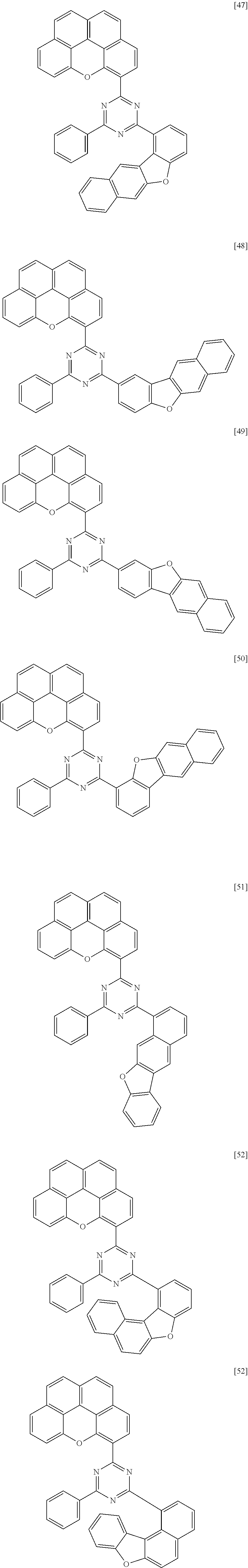

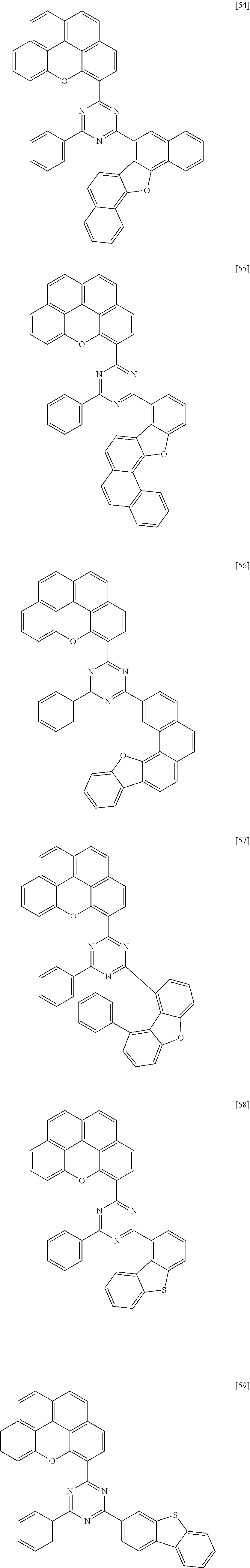

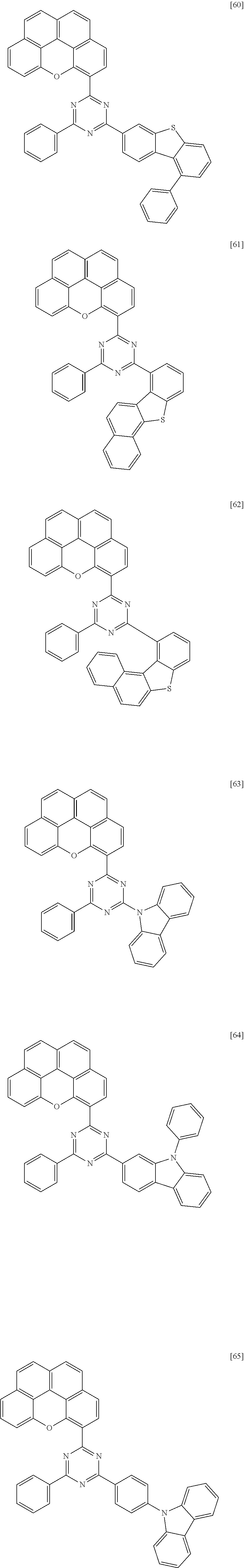 [SMILES: c1ccc(-c2nc(-c3ccc4c(c3)sc3cccc(-c5ccccc5)c34)nc(-c3ccc4ccc5ccc6cccc7c6c5c4c3O7)n2)cc1 ; c1ccc(-c2nc(-c3ccc4ccc5ccc6cccc7c6c5c4c3O7)nc(-c3cccc4sc5c6ccccc6ccc5c34)n2)cc1 ; c1ccc(-c2nc(-c3ccc4ccc5ccc6cccc7c6c5c4c3O7)nc(-c3cccc4sc5ccc6ccccc6c5c34)n2)cc1 ; c1ccc(-c2nc(-c3ccc4ccc5ccc6cccc7c6c5c4c3O7)nc(-n3c4ccccc4c4ccccc43)n2)cc1 ; c1ccc(-c2nc(-c3ccc4c5ccccc5n(-c5ccccc5)c4c3)nc(-c3ccc4ccc5ccc6cccc7c6c5c4c3O7)n2)cc1 ; c1ccc(-c2nc(-c3ccc(-n4c5ccccc5c5ccccc54)cc3)nc(-c3ccc4ccc5ccc6cccc7c6c5c4c3O7)n2)cc1]

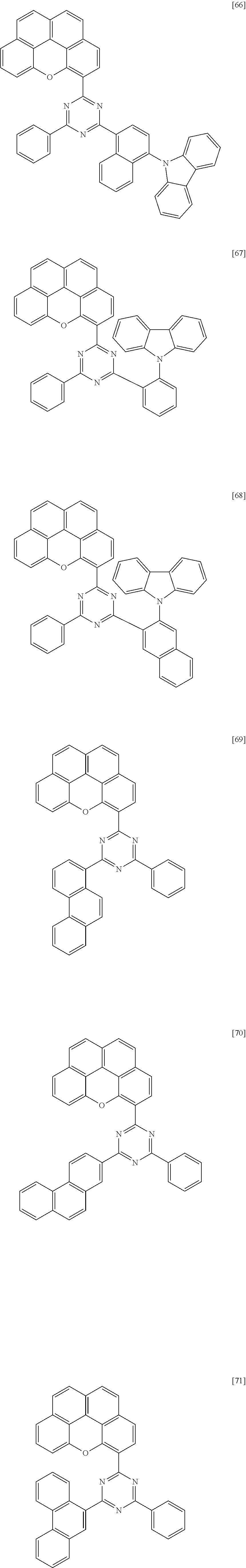 [SMILES: c1ccc(-c2nc(-c3ccc4ccc5ccc6cccc7c6c5c4c3O7)nc(-c3ccc(-n4c5ccccc5c5ccccc54)c4ccccc34)n2)cc1 ; c1ccc(-c2nc(-c3ccccc3-n3c4ccccc4c4ccccc43)nc(-c3ccc4ccc5ccc6cccc7c6c5c4c3O7)n2)cc1 ; c1ccc(-c2nc(-c3cc4ccccc4cc3-n3c4ccccc4c4ccccc43)nc(-c3ccc4ccc5ccc6cccc7c6c5c4c3O7)n2)cc1 ; c1ccc(-c2nc(-c3ccc4ccc5ccc6cccc7c6c5c4c3O7)nc(-c3cccc4c3ccc3ccccc34)n2)cc1 ; c1ccc(-c2nc(-c3ccc4c(ccc5ccccc54)c3)nc(-c3ccc4ccc5ccc6cccc7c6c5c4c3O7)n2)cc1 ; c1ccc(-c2nc(-c3ccc4ccc5ccc6cccc7c6c5c4c3O7)nc(-c3cc4ccccc4c4ccccc34)n2)cc1]

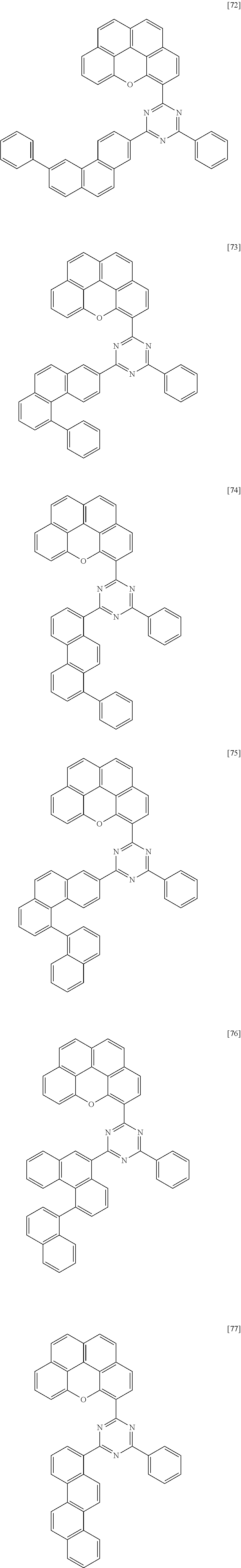

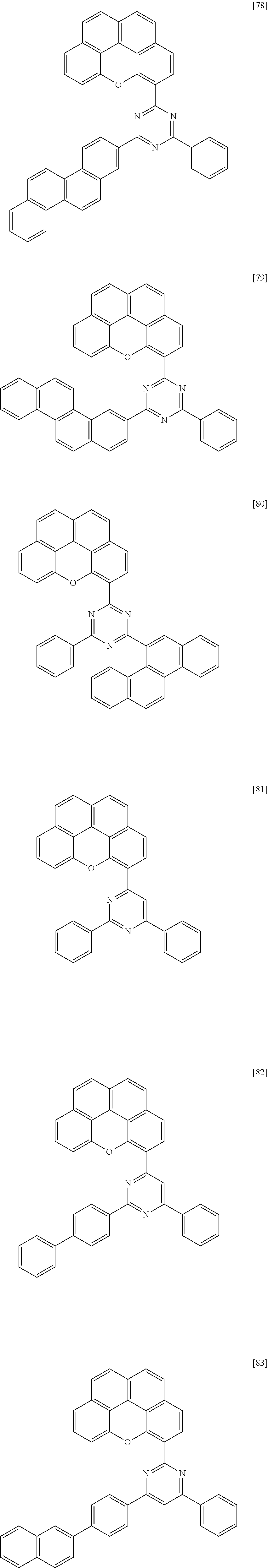 [SMILES: c1ccc(-c2nc(-c3ccc4c(ccc5c6ccccc6ccc45)c3)nc(-c3ccc4ccc5ccc6cccc7c6c5c4c3O7)n2)cc1 ; c1ccc(-c2nc(-c3ccc4ccc5c6ccccc6ccc5c4c3)nc(-c3ccc4ccc5ccc6cccc7c6c5c4c3O7)n2)cc1 ; c1ccc(-c2nc(-c3ccc4ccc5ccc6cccc7c6c5c4c3O7)nc(-c3cc4ccccc4c4ccc5ccccc5c34)n2)cc1 ; c1ccc(-c2cc(-c3ccc4ccc5ccc6cccc7c6c5c4c3O7)nc(-c3ccccc3)n2)cc1 ; c1ccc(-c2ccc(-c3nc(-c4ccccc4)cc(-c4ccc5ccc6ccc7cccc8c7c6c5c4O8)n3)cc2)cc1 ; c1ccc(-c2cc(-c3ccc(-c4ccc5ccccc5c4)cc3)nc(-c3ccc4ccc5ccc6cccc7c6c5c4c3O7)n2)cc1]

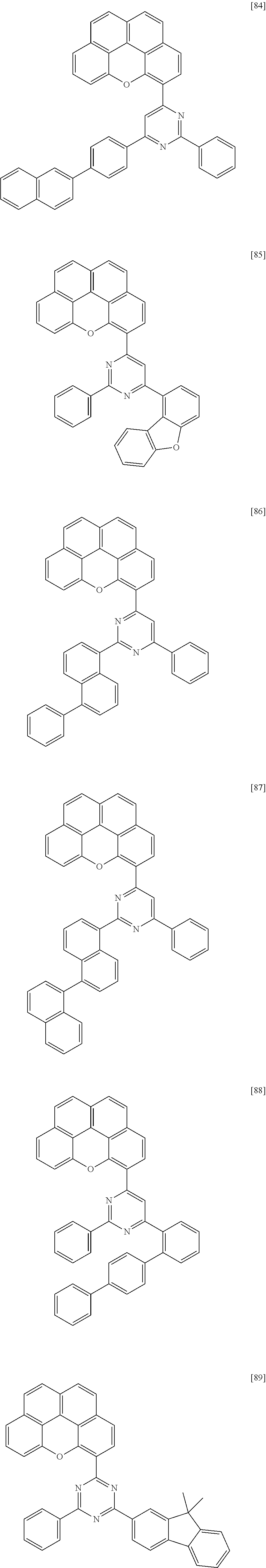 [SMILES: c1ccc(-c2nc(-c3ccc(-c4ccc5ccccc5c4)cc3)cc(-c3ccc4ccc5ccc6cccc7c6c5c4c3O7)n2)cc1 ; c1ccc(-c2nc(-c3ccc4ccc5ccc6cccc7c6c5c4c3O7)cc(-c3cccc4oc5ccccc5c34)n2)cc1 ; c1ccc(-c2cc(-c3ccc4ccc5ccc6cccc7c6c5c4c3O7)nc(-c3cccc4c(-c5ccccc5)cccc34)n2)cc1 ; c1ccc(-c2cc(-c3ccc4ccc5ccc6cccc7c6c5c4c3O7)nc(-c3cccc4c(-c5cccc6ccccc56)cccc34)n2)cc1 ; c1ccc(-c2ccc(-c3ccccc3-c3cc(-c4ccc5ccc6ccc7cccc8c7c6c5c4O8)nc(-c4ccccc4)n3)cc2)cc1 ; CC1(C)c2ccccc2-c2ccc(-c3nc(-c4ccccc4)nc(-c4ccc5ccc6ccc7cccc8c7c6c5c4O8)n3)cc21]

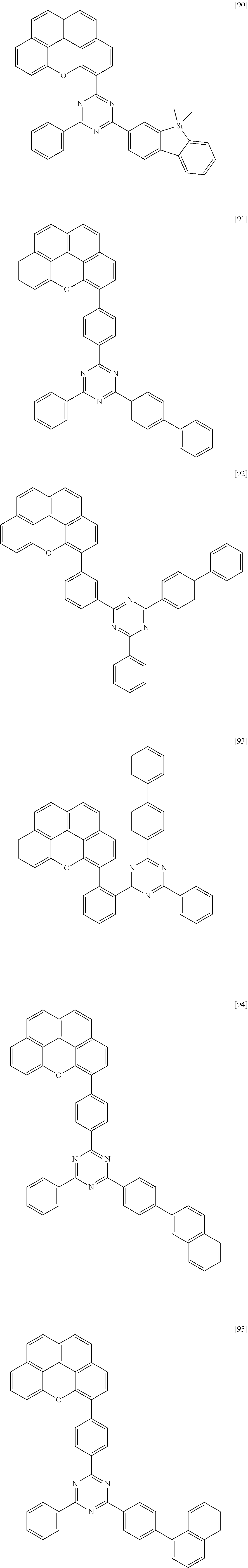

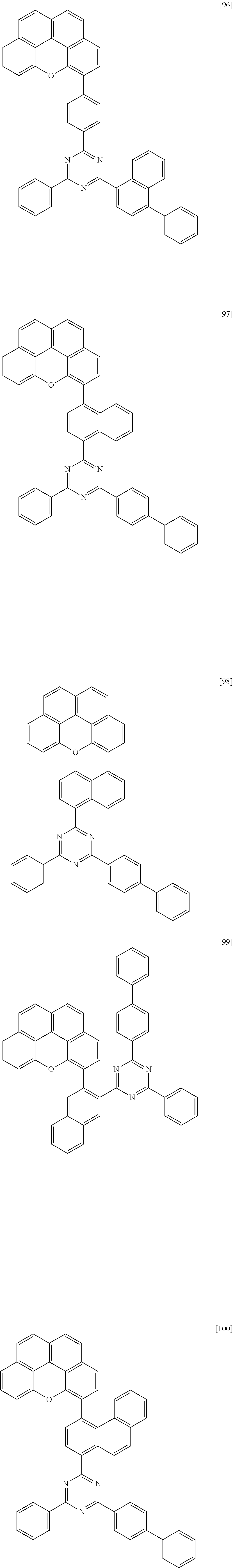 [SMILES: c1ccc(-c2nc(-c3ccc(-c4ccc5ccc6ccc7cccc8c7c6c5c4O8)cc3)nc(-c3ccc(-c4ccccc4)c4ccccc34)n2)cc1 ; c1ccc(-c2ccc(-c3nc(-c4ccccc4)nc(-c4ccc(-c5ccc6ccc7ccc8cccc9c8c7c6c5O9)c5ccccc45)n3)cc2)cc1 ; c1ccc(-c2ccc(-c3nc(-c4ccccc4)nc(-c4cccc5c(-c6ccc7ccc8ccc9cccc%10c9c8c7c6O%10)cccc45)n3)cc2)cc1 ; c1ccc(-c2ccc(-c3nc(-c4ccccc4)nc(-c4cc5ccccc5cc4-c4ccc5ccc6ccc7cccc8c7c6c5c4O8)n3)cc2)cc1 ; c1ccc(-c2ccc(-c3nc(-c4ccccc4)nc(-c4ccc(-c5ccc6ccc7ccc8cccc9c8c7c6c5O9)c5c4ccc4ccccc45)n3)cc2)cc1]

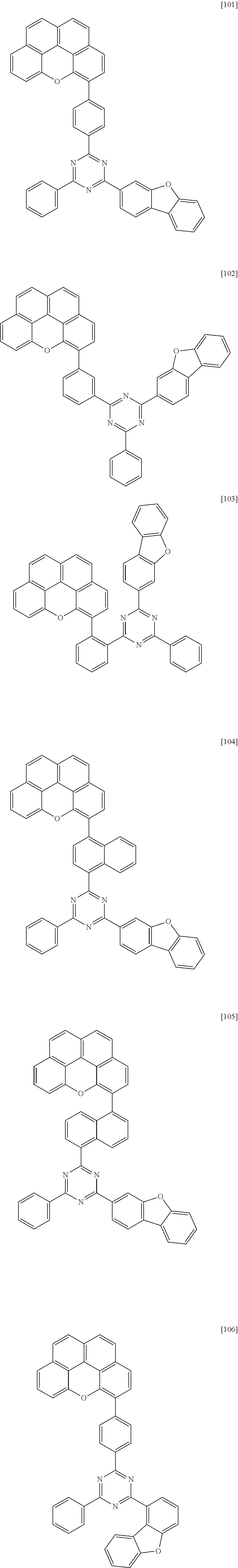

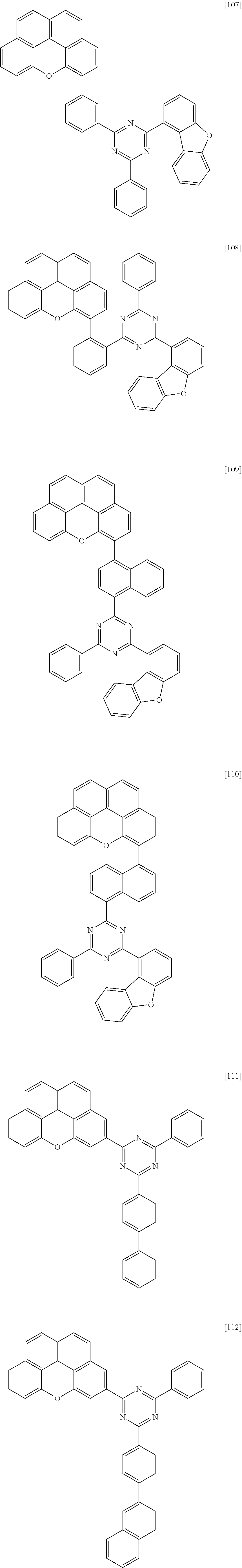

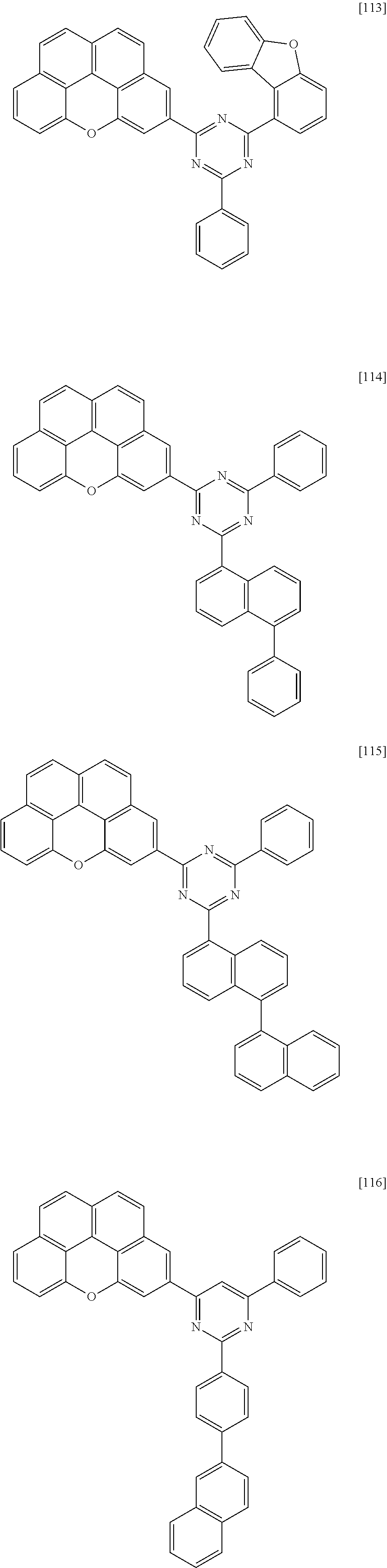 [SMILES: c1ccc(-c2nc(-c3cc4c5c(ccc6ccc7cccc(c7c65)O4)c3)nc(-c3cccc4oc5ccccc5c34)n2)cc1 ; c1ccc(-c2nc(-c3cc4c5c(ccc6ccc7cccc(c7c65)O4)c3)nc(-c3cccc4c(-c5ccccc5)cccc34)n2)cc1 ; c1ccc(-c2nc(-c3cc4c5c(ccc6ccc7cccc(c7c65)O4)c3)nc(-c3cccc4c(-c5cccc6ccccc56)cccc34)n2)cc1 ; c1ccc(-c2cc(-c3cc4c5c(ccc6ccc7cccc(c7c65)O4)c3)nc(-c3ccc(-c4ccc5ccccc5c4)cc3)n2)cc1]

A composition for an organic optoelectronic device according to another example embodiment includes a first compound and a second compound. The first compound may be the aforementioned compound for the organic optoelectronic device, and the second compound may be represented by Chemical Formula 2.

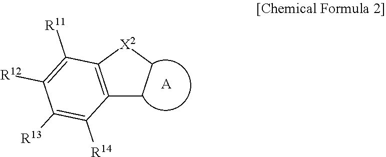

In Chemical Formula 2,

X² is O, S, NR^{b}, CR^{c}R^{d}, or SiR^{e}R^{f},

R^{b}, R^{c}, R^{d}, R^{e}, R^{f}, and R¹¹ to R¹⁴ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and

ring A is any one selected from the rings of Group II.

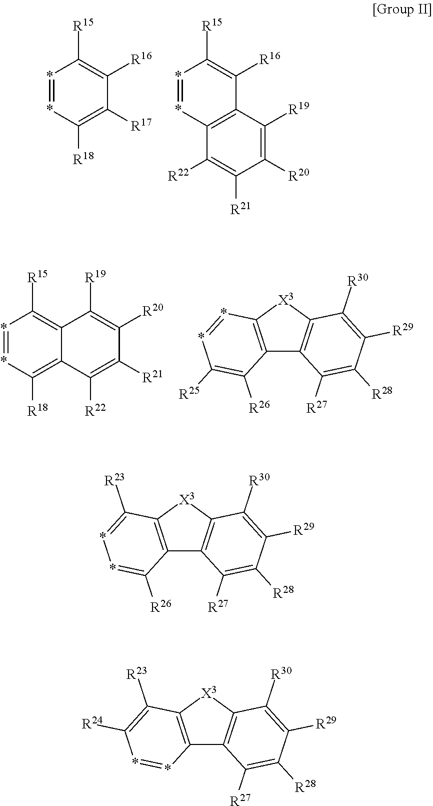

In Group II,

* is a linking point,

X³ is O, S, NR^{g}, CR^{h}Rᵢ, or SiR^{j}R^{k},

R^{g}, R^{h}, Rⁱ, R^{j}, R^{k}, and R¹⁵, to R³⁰ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and

at least one of R¹¹ to R³⁰ is a group represented by Chemical Formula b.

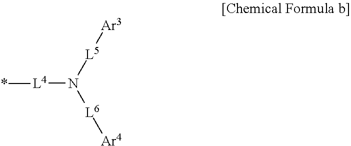

In Chemical Formula b,

L⁴ to L⁶ are each independently a single bond, or a substituted or unsubstituted C6 to C30 arylene group,

Ar³ and Ar⁴ are each independently a substituted or unsubstituted amine group, a substituted or unsubstituted C6 to C30 aryl group, or a substituted or unsubstituted C2 to C30 heterocyclic group, and

* is a linking point.

The second compound may have a structure in which carbazole/fused carbazole/fused dibenzofuran/fused dibenzothiophene/fused dibenzosilole is substituted with amine, and may be represented by any one of Chemical Formula 2-I to Chemical Formula 2-IX, for example, depending on a type and a fusion position of an additional benzene ring.

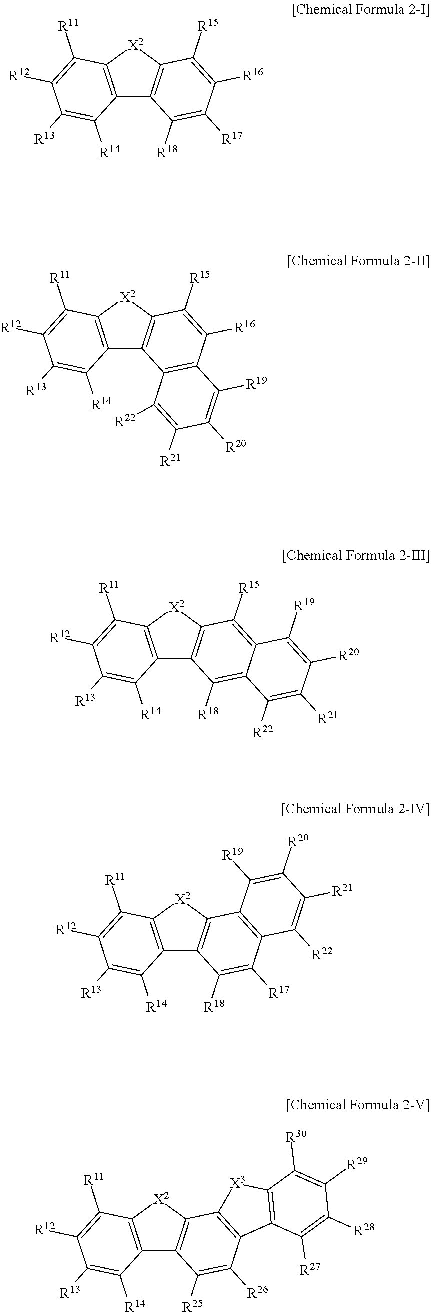

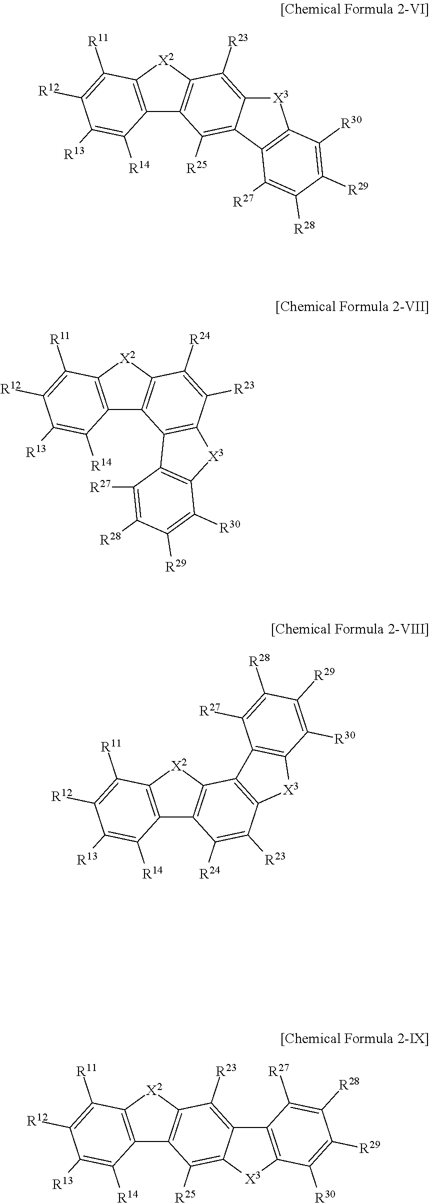

In Chemical Formula 2-I to Chemical Formula 2-IX, X², X³, and R¹¹ to R³⁰ are the same as described above.

In addition, the second compound may be represented by any one of Chemical Formula 2-IA to Chemical Formula 2-IXA, Chemical Formula 2-IB to Chemical Formula 2-IXB, and Chemical Formula 2-IC to Chemical Formula 2-IIIC, depending on substitution direction of an amine group.

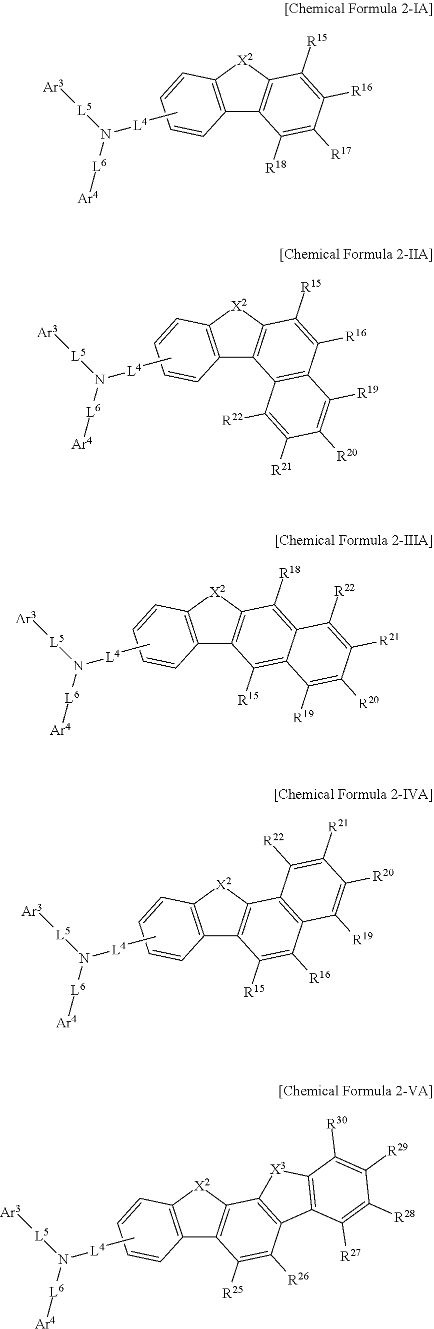

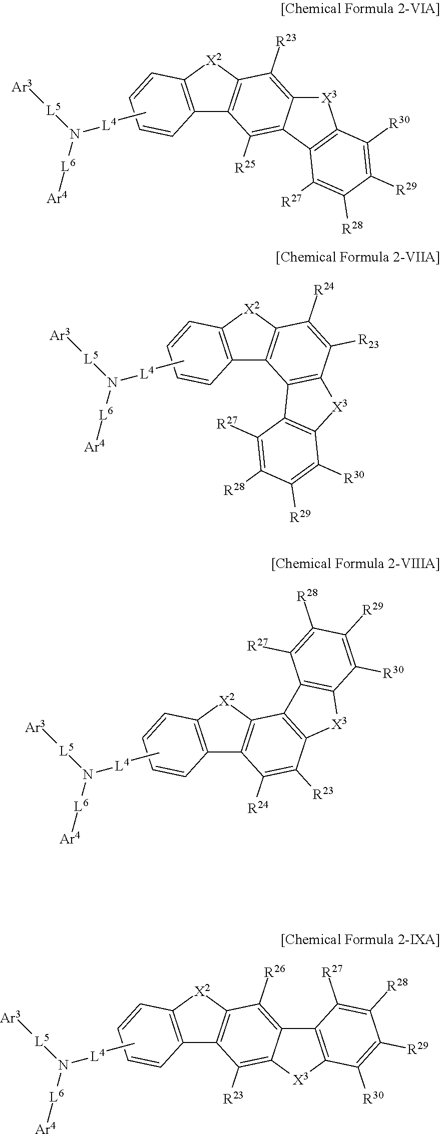

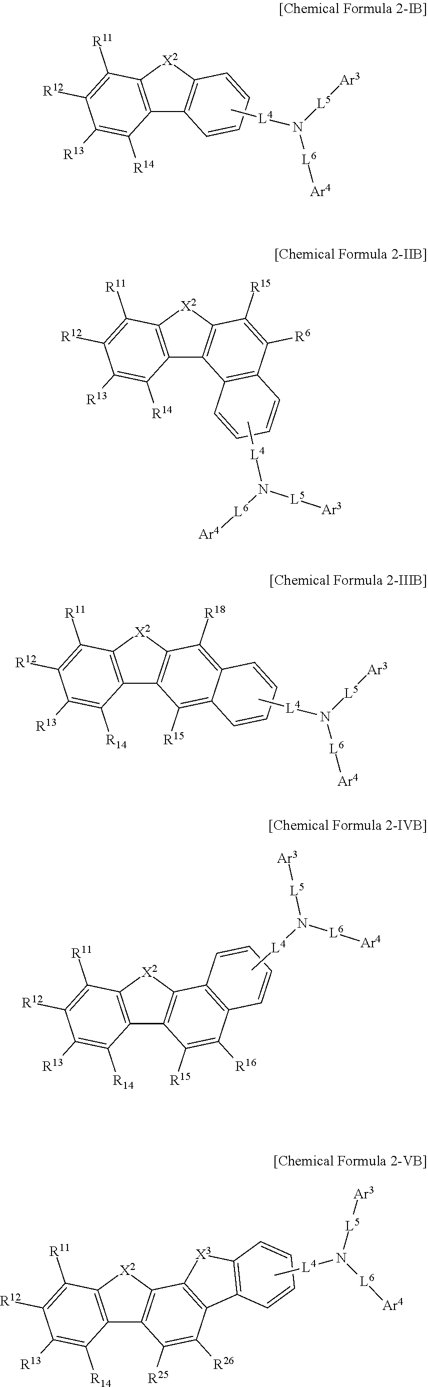

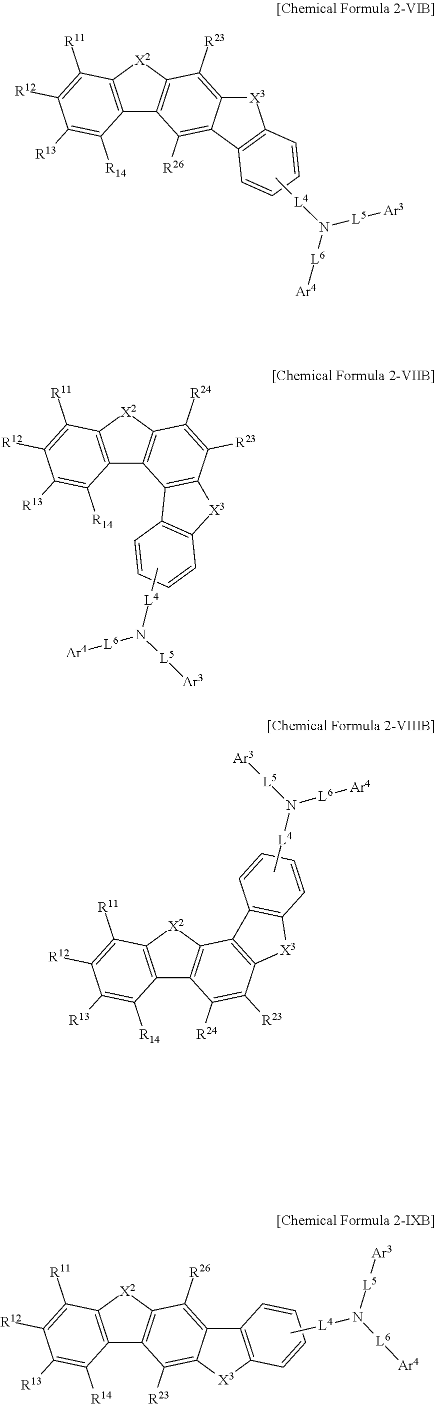

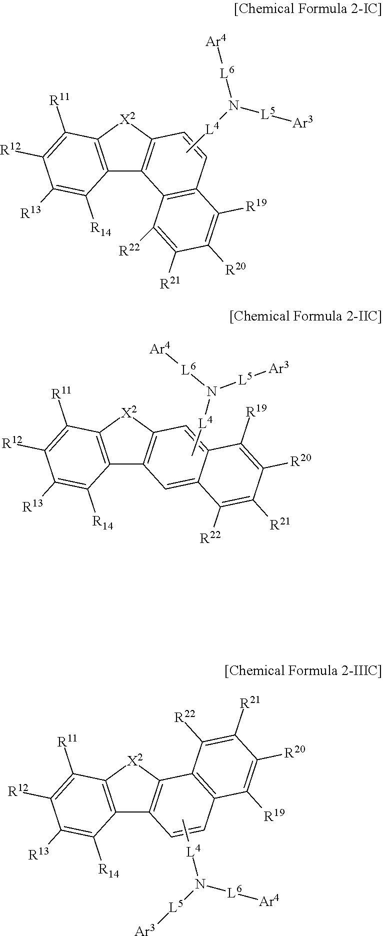

In Chemical Formula 2-IA to Chemical Formula 2-IXA, Chemical Formula 2-TB to Chemical Formula 2-IXB, and Chemical Formula 2-IC to Chemical Formula 2-IIIC,

X², X³, L⁴ to L⁶, Ar³, and Ar⁴ are the same as described above, and

R¹¹ to R³⁰ are each independently hydrogen, deuterium, a substituted or unsubstituted C1 to C30 alkyl group, or a substituted or unsubstituted C6 to C30 aryl group.

The second compound according to an example embodiment may be represented by Chemical Formula 2-IVB or Chemical Formula 2-VIIIB.

For example, X² of Chemical Formula 2-IVB may be NR^{b}.

For example, in Chemical Formula 2-VIIIB, X² may be O or S, and X³ may be CR^{h}Rⁱ or SiR^{j}R^{k}.

Herein, R^{b}, R^{h}, Rⁱ, R^{j}, and R^{k} may each independently be a substituted or unsubstituted C1 to C10 alkyl group, or a substituted or unsubstituted C6 to C20 aryl group.

The second compound according to a specific embodiment may be represented by Chemical Formula 2-IVB-2 or Chemical Formula 2-VIIIB-2.

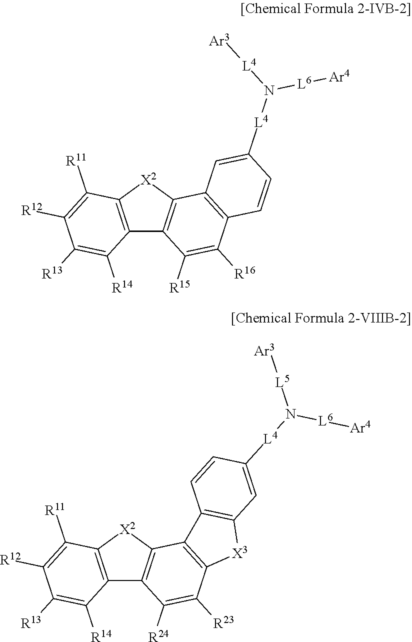

In Chemical Formula 2-IVB-2 and Chemical Formula 2-VIIIB-2,

L⁴ to L⁶ are each independently a single bond or a substituted or unsubstituted phenylene group,

Ar³ and Ar⁴ are each independently a substituted or unsubstituted phenyl group, a substituted or unsubstituted biphenyl group, or a substituted or unsubstituted naphthyl group,

X² is NR, O, or S,

X³ is CR^{h}Rⁱ or SiR^{j}R^{k},

R^{b}, R^{h}, Rⁱ, R^{j}, and R^{k} are each independently a substituted or unsubstituted C1 to C30 alkyl group, or a substituted or unsubstituted C6 to C30 aryl group, and

R¹¹ to R¹⁴, R¹⁵, R¹⁶, R²³, and R²⁴ are each independently hydrogen, deuterium, a substituted or unsubstituted C1 to C30 alkyl group or a substituted or unsubstituted C6 to C30 aryl group.

For example, L⁵ and L⁶ may each independently be a single bond, a substituted or unsubstituted phenylene group, or a substituted or unsubstituted biphenylene group.

For example, Ar³ and Ar⁴ may each independently be a substituted or unsubstituted phenyl group, a substituted or unsubstituted biphenyl group, a substituted or unsubstituted terphenyl group, a substituted or unsubstituted naphthyl group, a substituted or unsubstituted phenanthrenyl group, a substituted or unsubstituted triphenylene group, a substituted or unsubstituted fluorenyl group, a substituted or unsubstituted carbazolyl group, a substituted or unsubstituted dibenzofuranyl group, a substituted or unsubstituted dibenzothiophenyl group, a substituted or unsubstituted benzofuranofluorenyl group, or a substituted or unsubstituted benzothiophenefluorenyl group.

For example, the second compound may be one selected from the compounds of Group 2, but is not limited thereto.

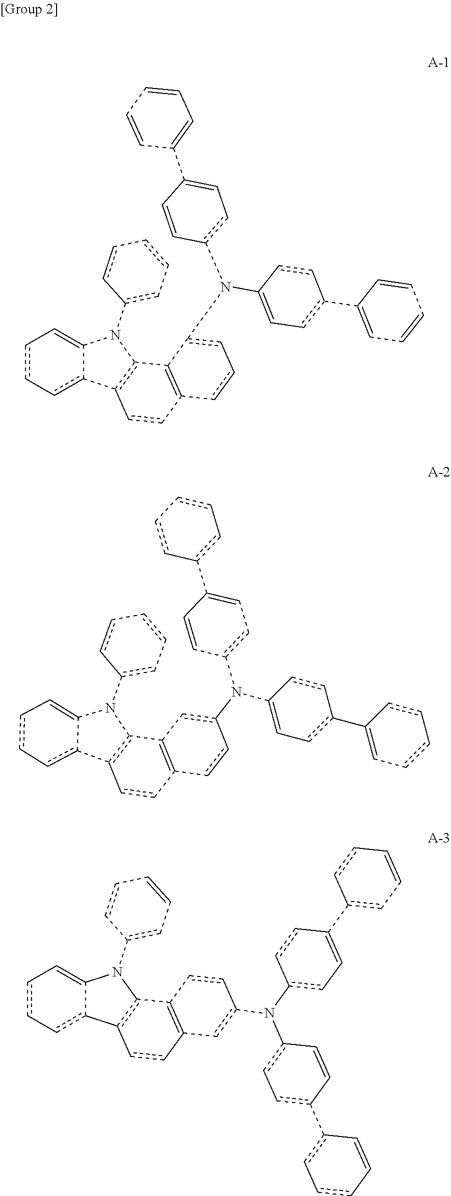

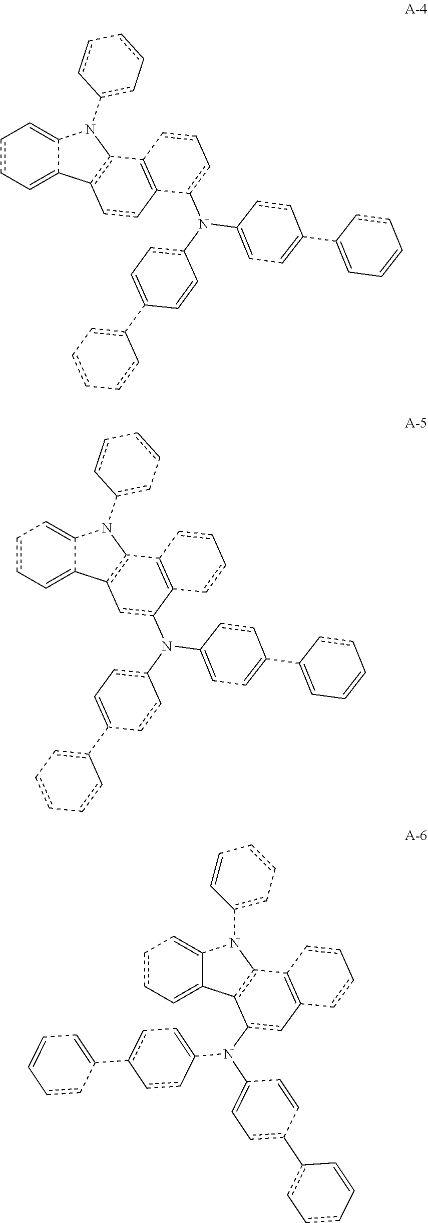

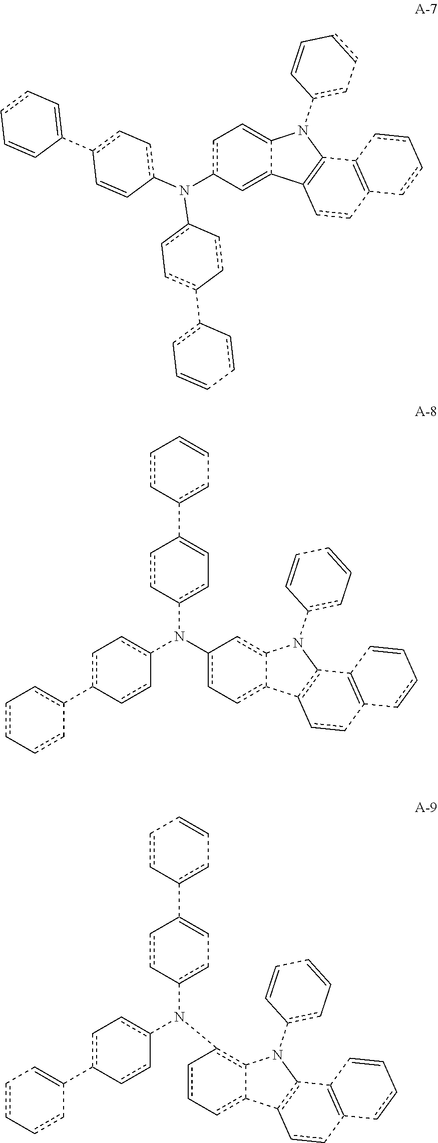

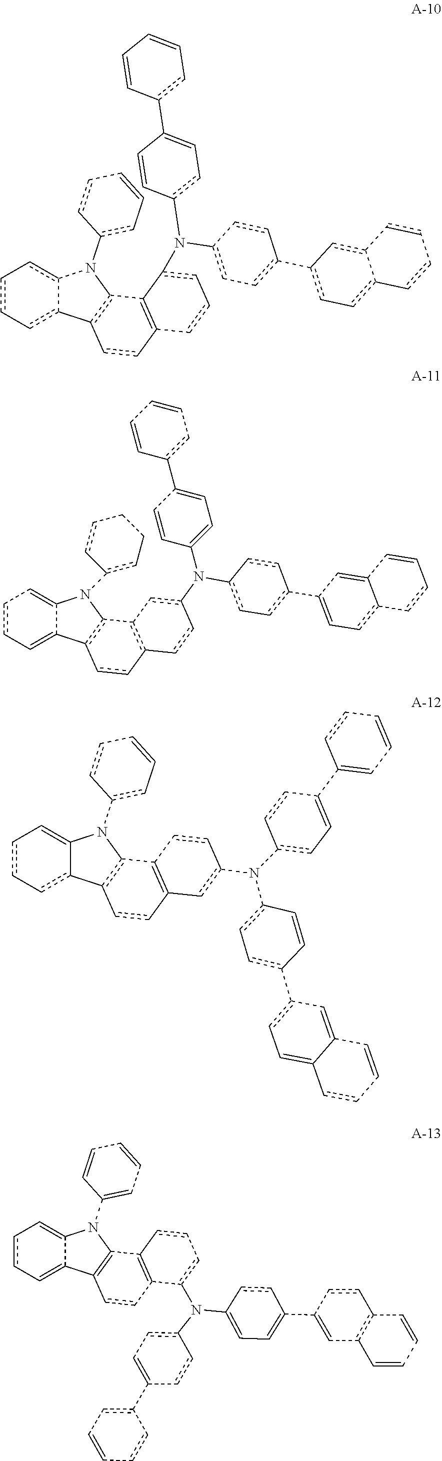

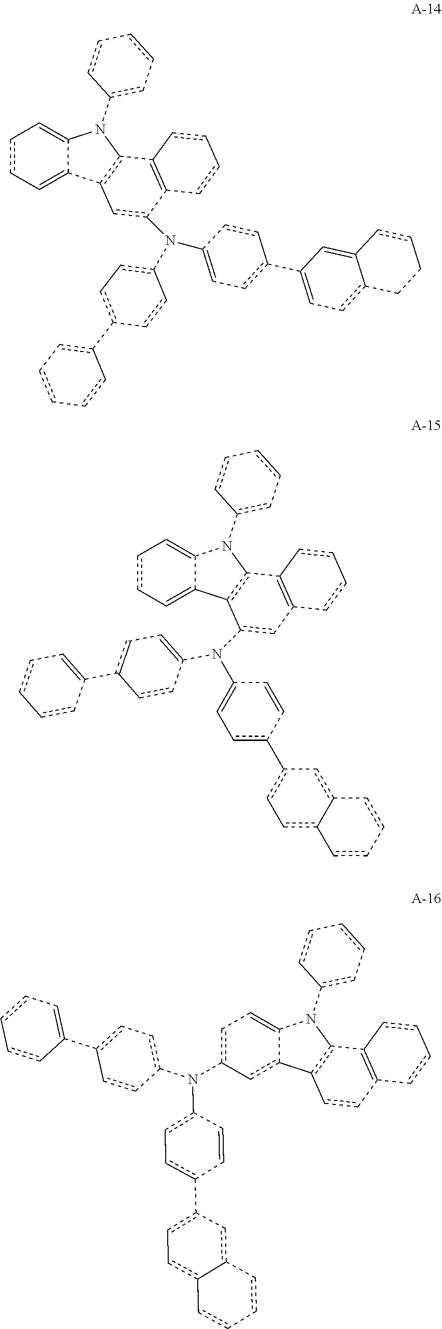

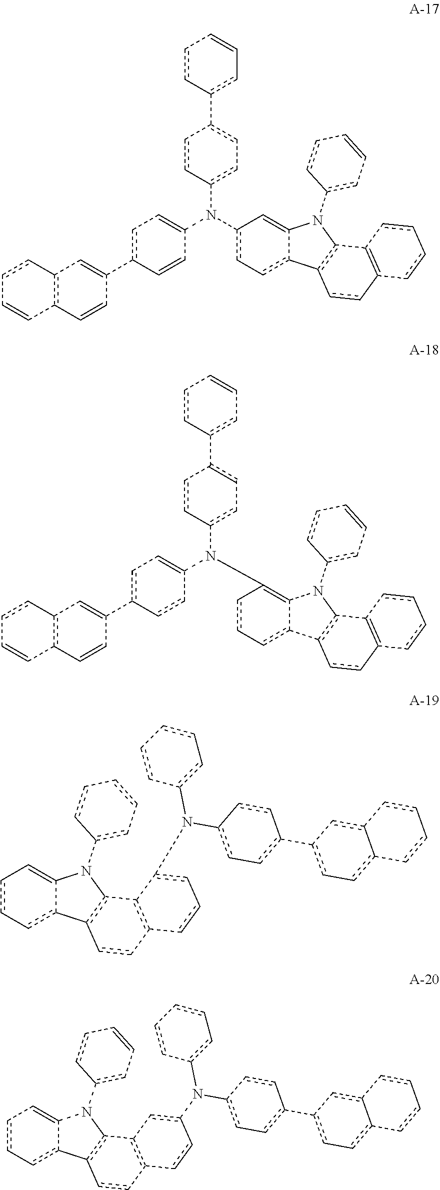

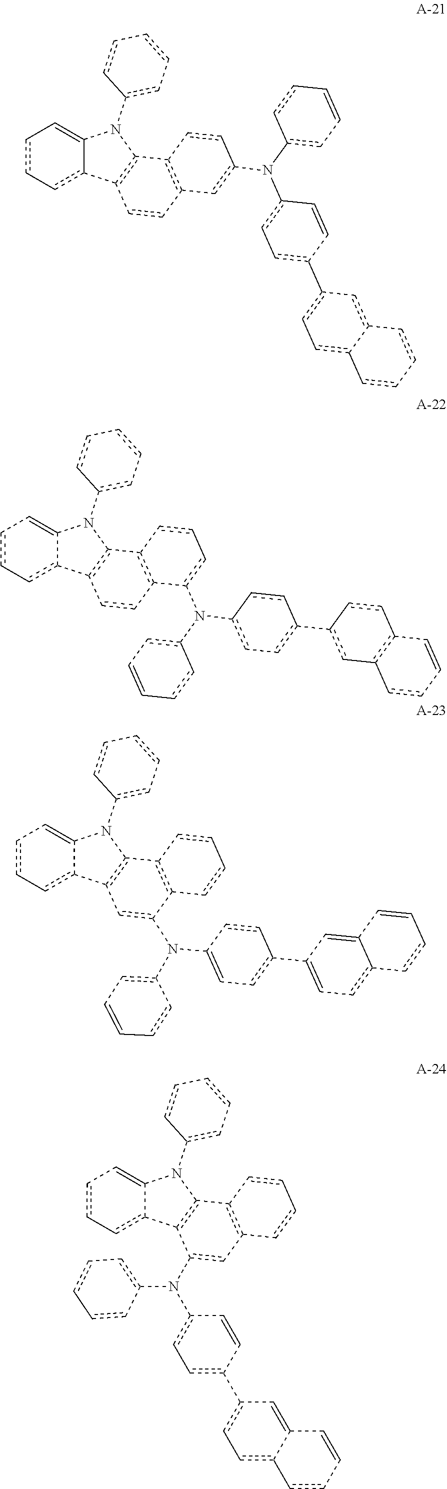

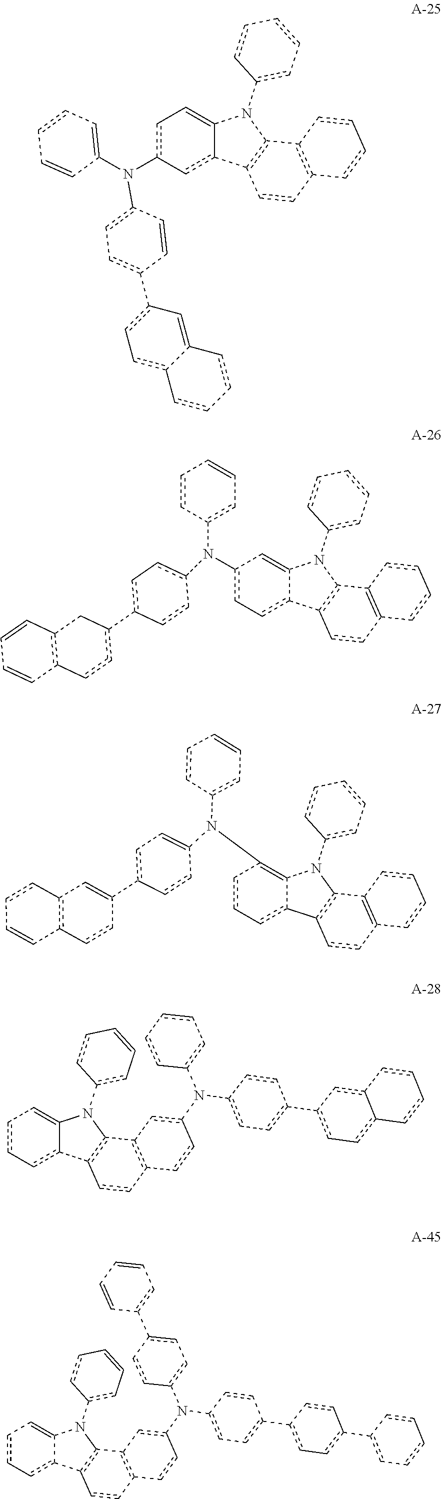

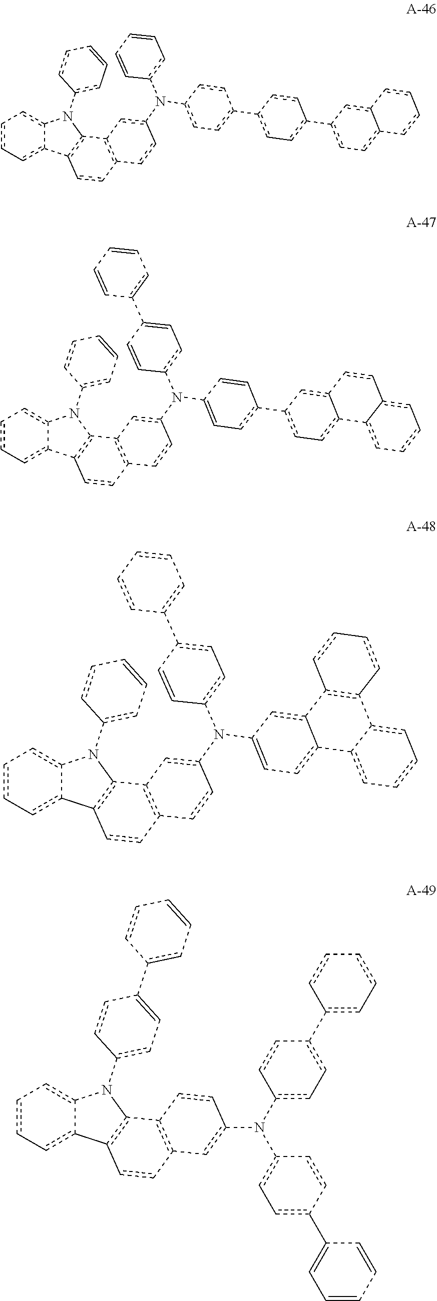

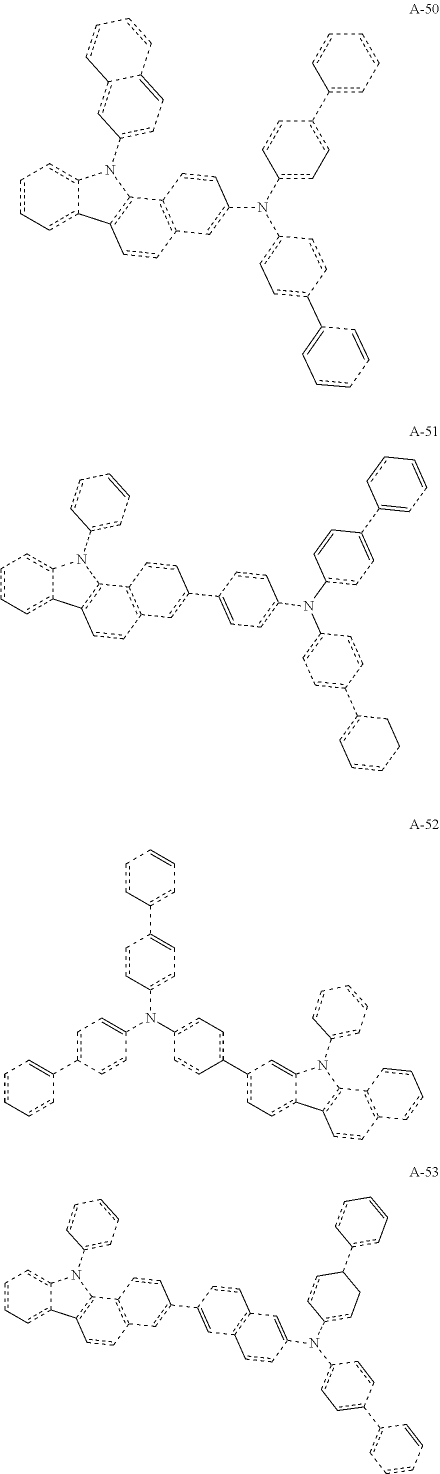

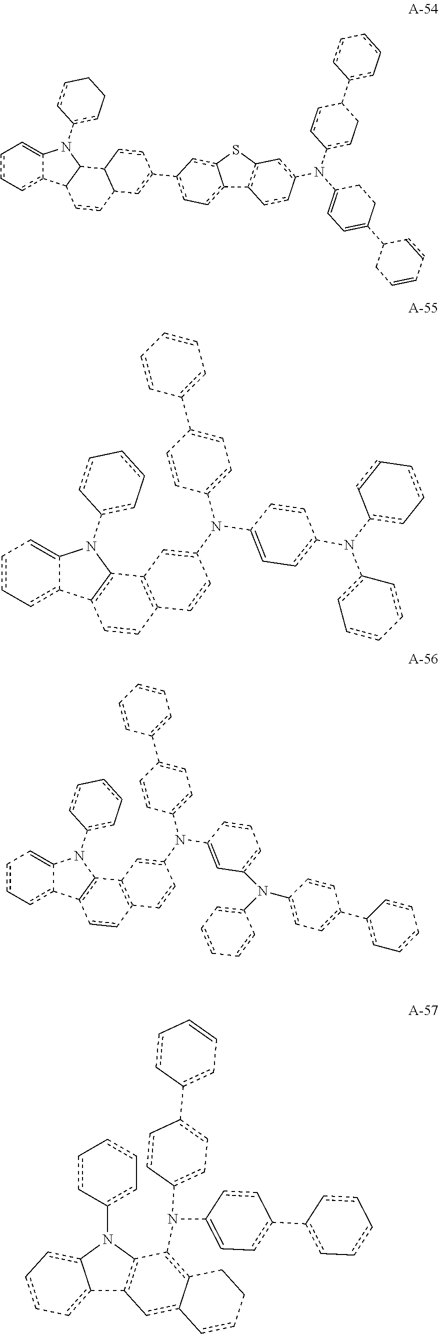

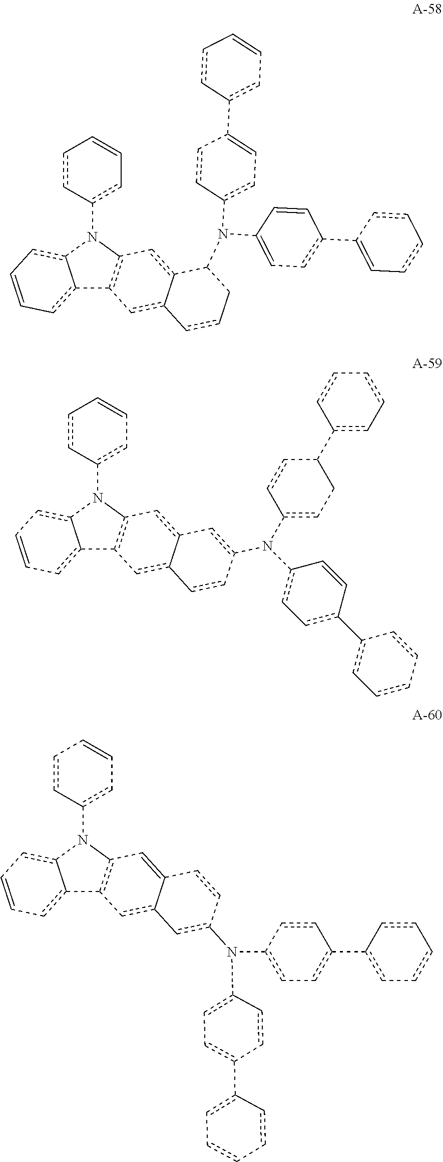

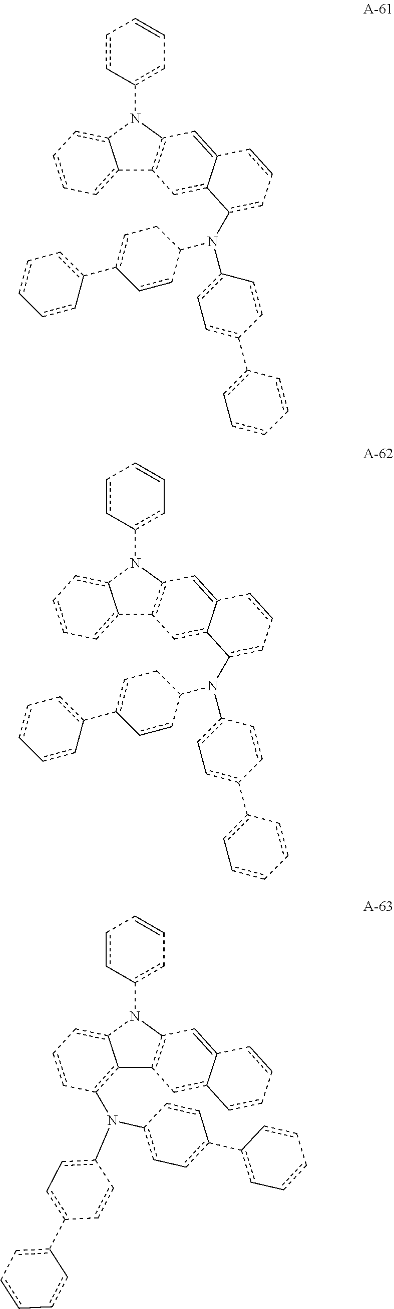

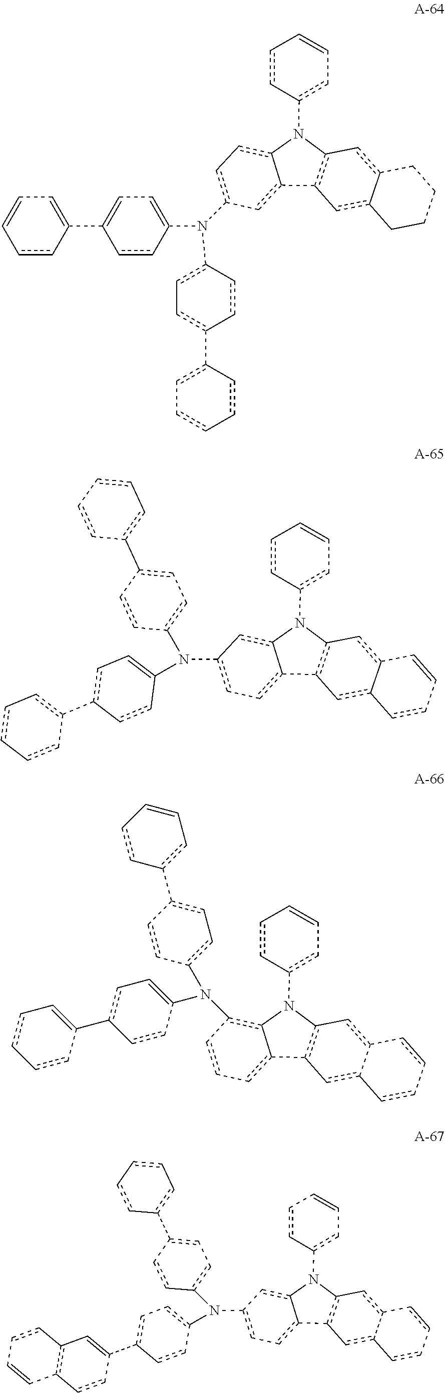

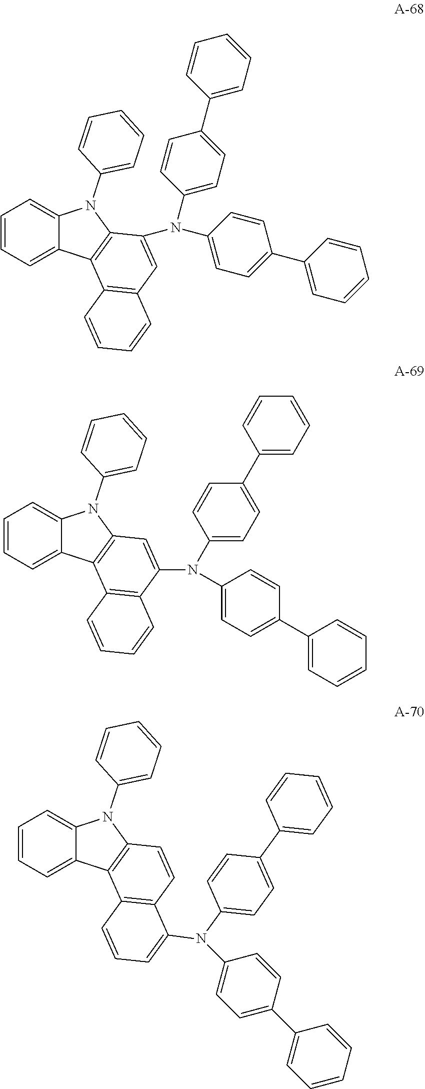 [SMILES: c1ccc(-c2ccc(N(c3ccc(-c4ccccc4)cc3)c3cc4ccccc4c4c5ccccc5n(-c5ccccc5)c34)cc2)cc1 ; c1ccc(-c2ccc(N(c3ccc(-c4ccccc4)cc3)c3cc4c(c5ccccc35)c3ccccc3n4-c3ccccc3)cc2)cc1 ; c1ccc(-c2ccc(N(c3ccc(-c4ccccc4)cc3)c3cccc4c3ccc3c4c4ccccc4n3-c3ccccc3)cc2)cc1]

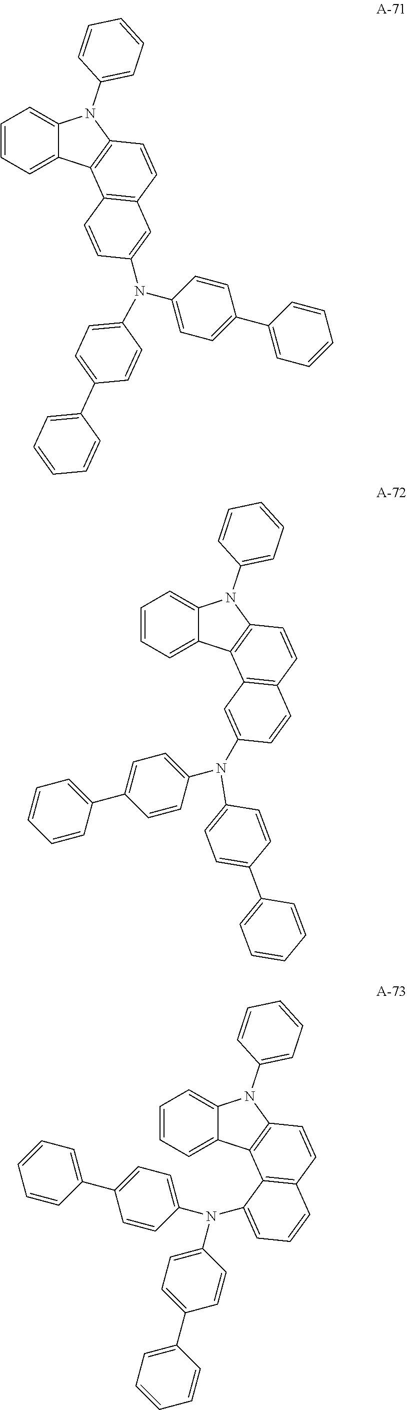

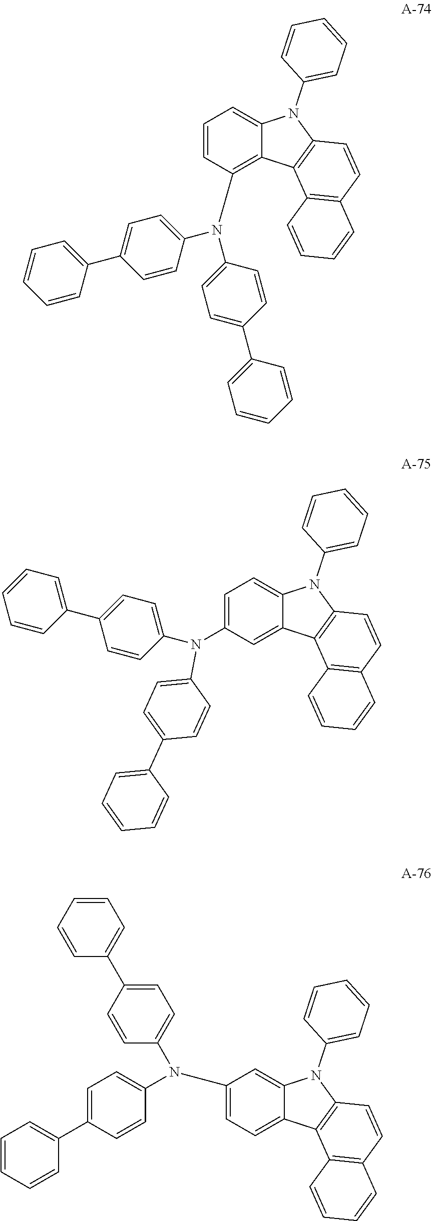 [SMILES: c1ccc(-c2ccc(N(c3ccc(-c4ccccc4)cc3)c3cccc4c3c3c5ccccc5ccc3n4-c3ccccc3)cc2)cc1 ; c1ccc(-c2ccc(N(c3ccc(-c4ccccc4)cc3)c3ccc4c(c3)c3c5ccccc5ccc3n4-c3ccccc3)cc2)cc1 ; c1ccc(-c2ccc(N(c3ccc(-c4ccccc4)cc3)c3ccc4c5c6ccccc6ccc5n(-c5ccccc5)c4c3)cc2)cc1]

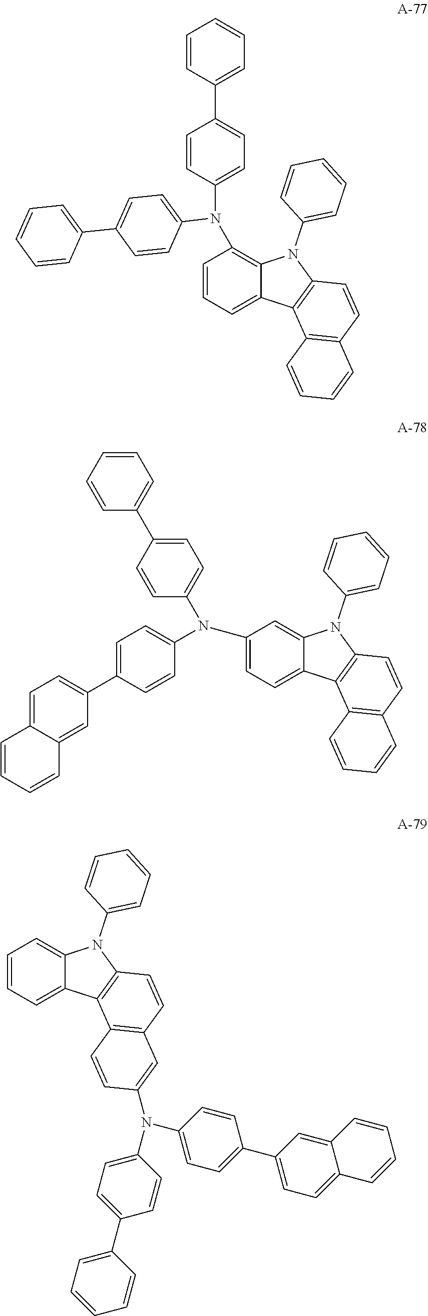 [SMILES: c1ccc(-c2ccc(N(c3ccc(-c4ccccc4)cc3)c3cccc4c5c6ccccc6ccc5n(-c5ccccc5)c34)cc2)cc1 ; c1ccc(-c2ccc(N(c3ccc(-c4ccc5ccccc5c4)cc3)c3ccc4c5c6ccccc6ccc5n(-c5ccccc5)c4c3)cc2)cc1 ; c1ccc(-c2ccc(N(c3ccc(-c4ccc5ccccc5c4)cc3)c3ccc4c(ccc5c4c4ccccc4n5-c4ccccc4)c3)cc2)cc1]

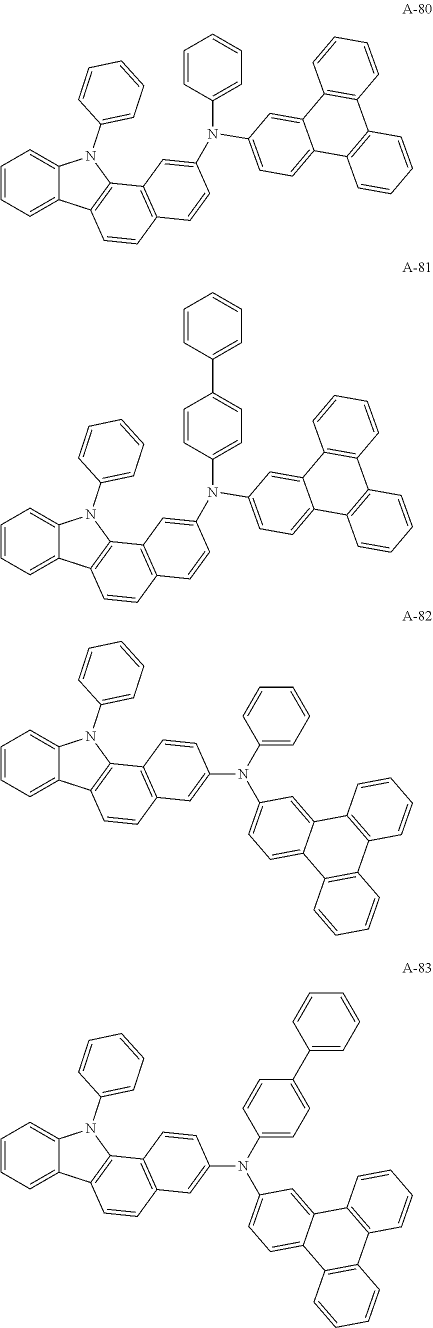

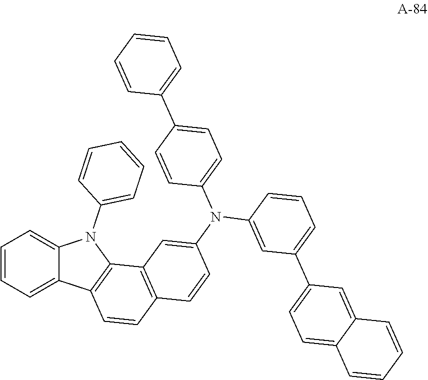 [SMILES: c1ccc(-c2ccc(N(c3cccc(-c4ccc5ccccc5c4)c3)c3ccc4ccc5c6ccccc6n(-c6ccccc6)c5c4c3)cc2)cc1]

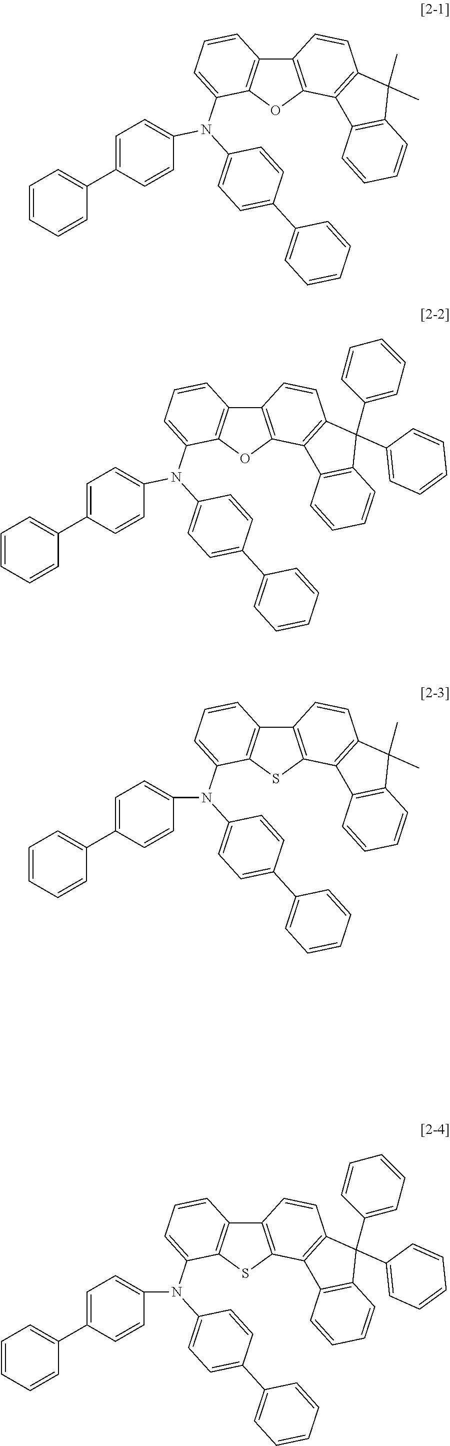 [SMILES: CC1(C)c2ccccc2-c2c1ccc1c2oc2c(N(c3ccc(-c4ccccc4)cc3)c3ccc(-c4ccccc4)cc3)cccc21 ; c1ccc(-c2ccc(N(c3ccc(-c4ccccc4)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4ccccc4)cc3)cccc21 ; c1ccc(-c2ccc(N(c3ccc(-c4ccccc4)cc3)c3cccc4c3sc3c5c(ccc34)C(c3ccccc3)(c3ccccc3)c3ccccc3-5)cc2)cc1]

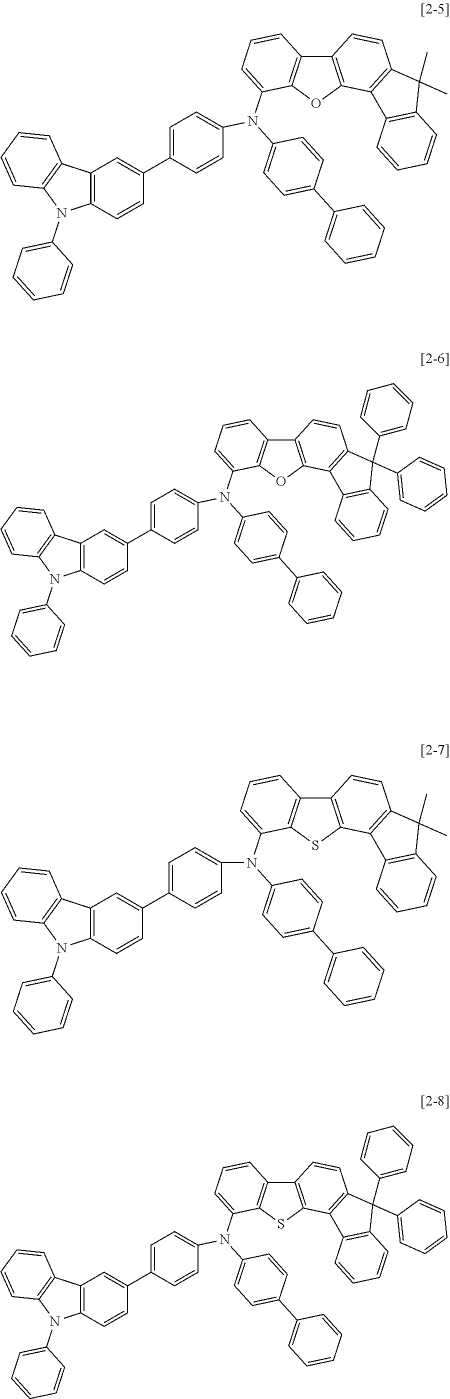 [SMILES: CC1(C)c2ccccc2-c2c1ccc1c2oc2c(N(c3ccc(-c4ccccc4)cc3)c3ccc(-c4ccc5c(c4)c4ccccc4n5-c4ccccc4)cc3)cccc21 ; c1ccc(-c2ccc(N(c3ccc(-c4ccc5c(c4)c4ccccc4n5-c4ccccc4)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4ccc5c(c4)c4ccccc4n5-c4ccccc4)cc3)cccc21 ; c1ccc(-c2ccc(N(c3ccc(-c4ccc5c(c4)c4ccccc4n5-c4ccccc4)cc3)c3cccc4c3sc3c5c(ccc34)C(c3ccccc3)(c3ccccc3)c3ccccc3-5)cc2)cc1]

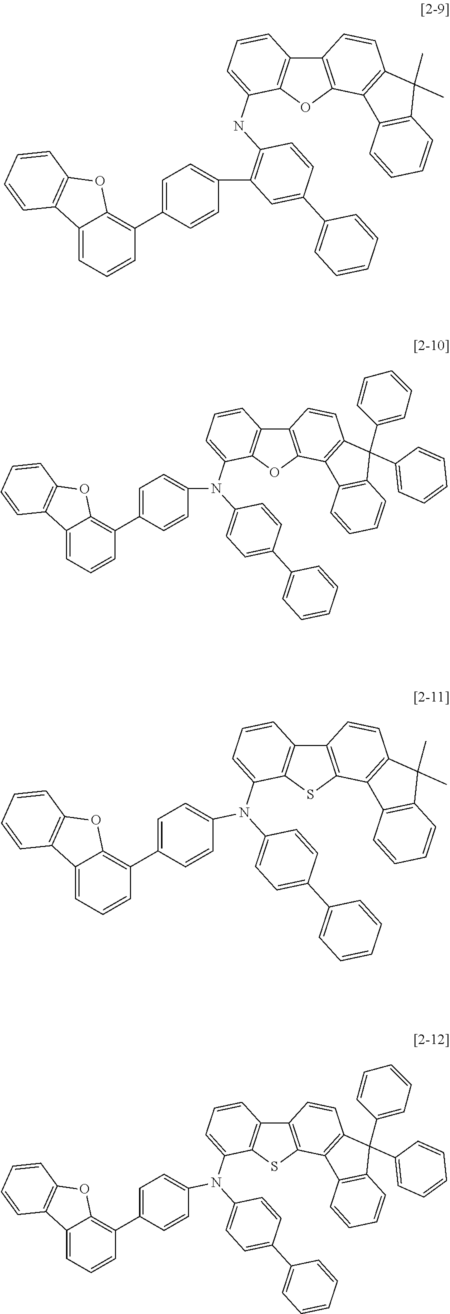 [SMILES: CC1(C)c2ccccc2-c2c1ccc1c2oc2c(Nc3ccc(-c4ccccc4)cc3-c3ccc(-c4cccc5c4oc4ccccc45)cc3)cccc21 ; c1ccc(-c2ccc(N(c3ccc(-c4cccc5c4oc4ccccc45)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4cccc5c4oc4ccccc45)cc3)cccc21 ; c1ccc(-c2ccc(N(c3ccc(-c4cccc5c4oc4ccccc45)cc3)c3cccc4c3sc3c5c(ccc34)C(c3ccccc3)(c3ccccc3)c3ccccc3-5)cc2)cc1]

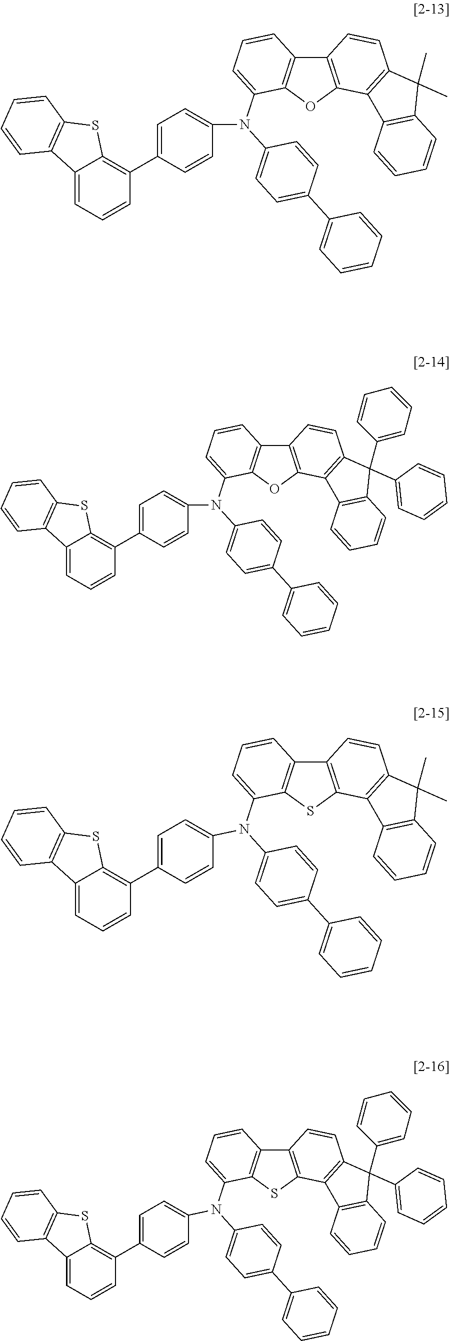 [SMILES: CC1(C)c2ccccc2-c2c1ccc1c2oc2c(N(c3ccc(-c4ccccc4)cc3)c3ccc(-c4cccc5c4sc4ccccc45)cc3)cccc21 ; c1ccc(-c2ccc(N(c3ccc(-c4cccc5c4sc4ccccc45)cc3)c3cccc4c3oc3c5c(ccc34)C(c3ccccc3)(c3ccccc3)c3ccccc3-5)cc2)cc1 ; CC1(C)c2ccccc2-c2c1ccc1c2sc2c(N(c3ccc(-c4ccccc4)cc3)c3ccc(-c4cccc5c4sc4ccccc45)cc3)cccc21 ; c1ccc(-c2ccc(N(c3ccc(-c4cccc5c4sc4ccccc45)cc3)c3cccc4c3sc3c5c(ccc34)C(c3ccccc3)(c3ccccc3)c3ccccc3-5)cc2)cc1]

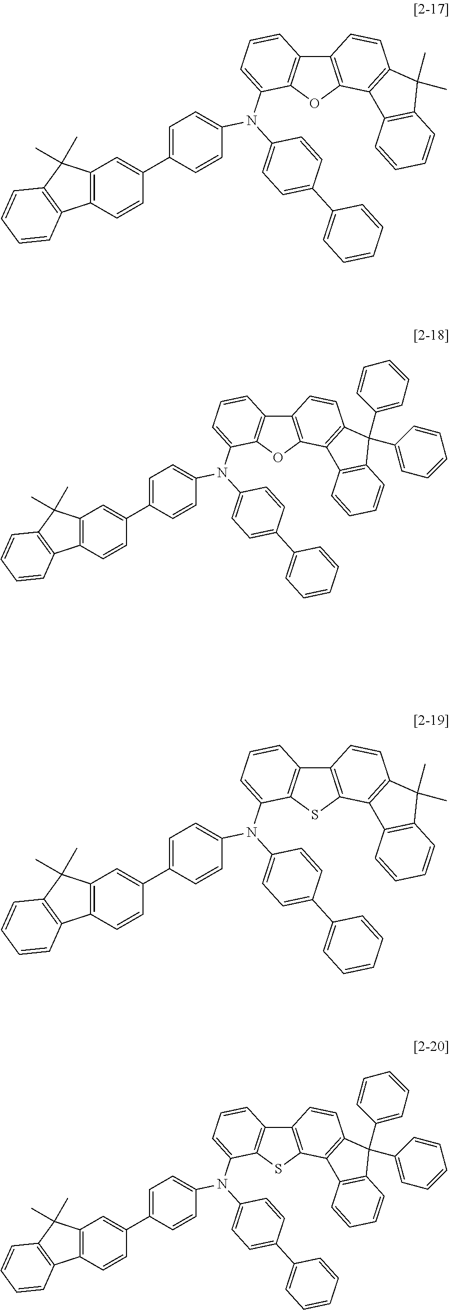 [SMILES: CC1(C)c2ccccc2-c2ccc(-c3ccc(N(c4ccc(-c5ccccc5)cc4)c4cccc5c4oc4c6c(ccc45)C(C)(C)c4ccccc4-6)cc3)cc21 ; CC1(C)c2ccccc2-c2ccc(-c3ccc(N(c4ccc(-c5ccccc5)cc4)c4cccc5c4oc4c6c(ccc45)C(c4ccccc4)(c4ccccc4)c4ccccc4-6)cc3)cc21 ; CC1(C)c2ccccc2-c2ccc(-c3ccc(N(c4ccc(-c5ccccc5)cc4)c4cccc5c4sc4c6c(ccc45)C(C)(C)c4ccccc4-6)cc3)cc21 ; CC1(C)c2ccccc2-c2ccc(-c3ccc(N(c4ccc(-c5ccccc5)cc4)c4cccc5c4sc4c6c(ccc45)C(c4ccccc4)(c4ccccc4)c4ccccc4-6)cc3)cc21]

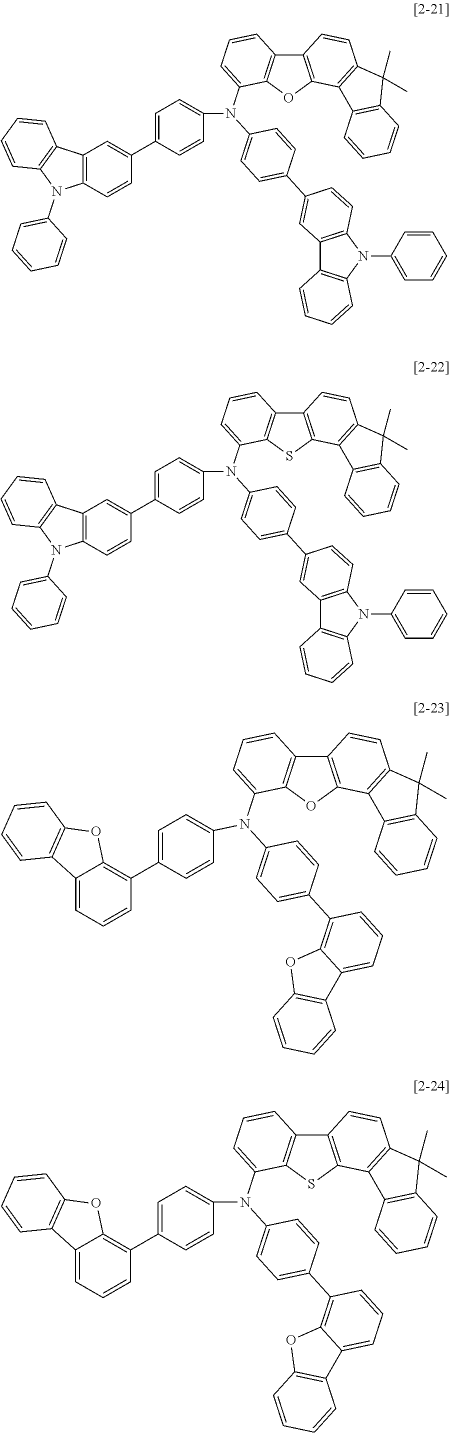 [SMILES: CC1(C)c2ccccc2-c2c1ccc1c2oc2c(N(c3ccc(-c4ccc5c(c4)c4ccccc4n5-c4ccccc4)cc3)c3ccc(-c4ccc5c(c4)c4ccccc4n5-c4ccccc4)cc3)cccc21 ; CC1(C)c2ccccc2-c2c1ccc1c2sc2c(N(c3ccc(-c4ccc5c(c4)c4ccccc4n5-c4ccccc4)cc3)c3ccc(-c4ccc5c(c4)c4ccccc4n5-c4ccccc4)cc3)cccc21 ; CC1(C)c2ccccc2-c2c1ccc1c2oc2c(N(c3ccc(-c4cccc5c4oc4ccccc45)cc3)c3ccc(-c4cccc5c4oc4ccccc45)cc3)cccc21 ; CC1(C)c2ccccc2-c2c1ccc1c2sc2c(N(c3ccc(-c4cccc5c4oc4ccccc45)cc3)c3ccc(-c4cccc5c4oc4ccccc45)cc3)cccc21]

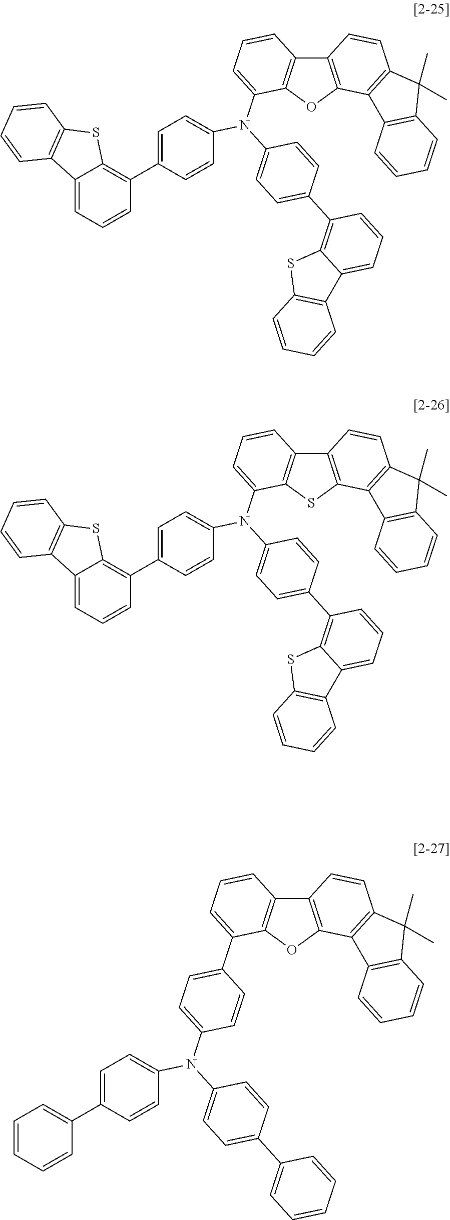 [SMILES: CC1(C)c2ccccc2-c2c1ccc1c2oc2c(N(c3ccc(-c4cccc5c4sc4ccccc45)cc3)c3ccc(-c4cccc5c4sc4ccccc45)cc3)cccc21 ; CC1(C)c2ccccc2-c2c1ccc1c2sc2c(N(c3ccc(-c4cccc5c4sc4ccccc45)cc3)c3ccc(-c4cccc5c4sc4ccccc45)cc3)cccc21 ; CC1(C)c2ccccc2-c2c1ccc1c2oc2c(-c3ccc(N(c4ccc(-c5ccccc5)cc4)c4ccc(-c5ccccc5)cc4)cc3)cccc21]

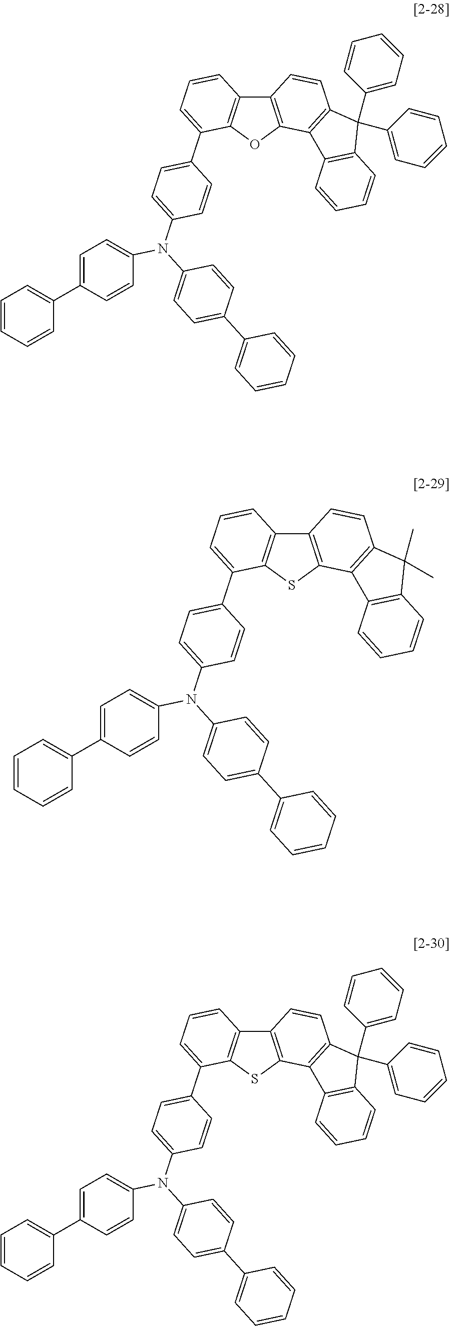 [SMILES: c1ccc(-c2ccc(N(c3ccc(-c4ccccc4)cc3)c3ccc(-c4cccc5c4oc4c6c(ccc45)C(c4ccccc4)(c4ccccc4)c4ccccc4-6)cc3)cc2)cc1 ; CC1(C)c2ccccc2-c2c1ccc1c2sc2c(-c3ccc(N(c4ccc(-c5ccccc5)cc4)c4ccc(-c5ccccc5)cc4)cc3)cccc21 ; c1ccc(-c2ccc(N(c3ccc(-c4ccccc4)cc3)c3ccc(-c4cccc5c4sc4c6c(ccc45)C(c4ccccc4)(c4ccccc4)c4ccccc4-6)cc3)cc2)cc1]

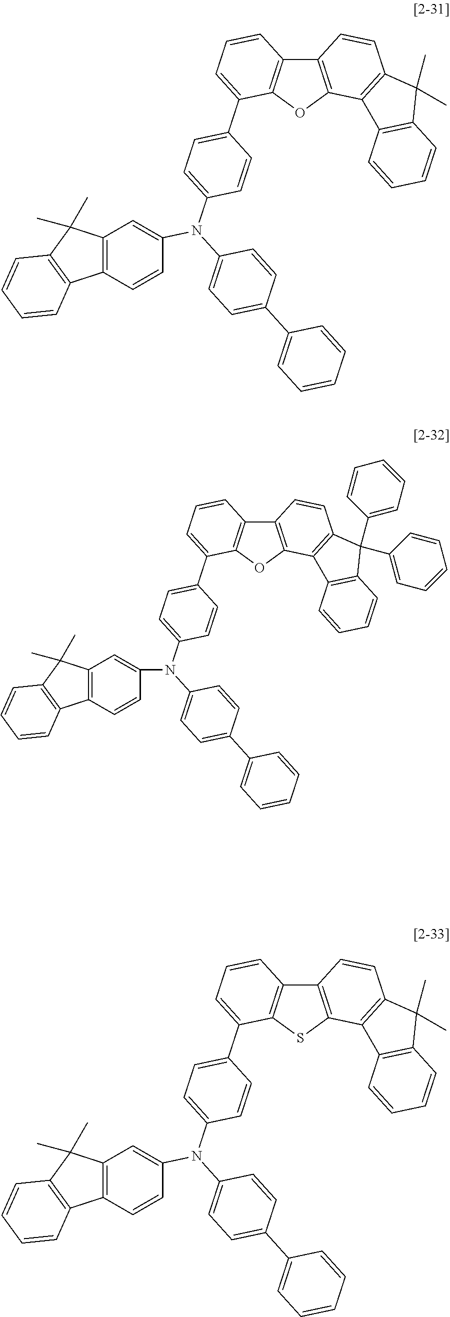 [SMILES: CC1(C)c2ccccc2-c2ccc(N(c3ccc(-c4ccccc4)cc3)c3ccc(-c4cccc5c4oc4c6c(ccc45)C(C)(C)c4ccccc4-6)cc3)cc21 ; CC1(C)c2ccccc2-c2ccc(N(c3ccc(-c4ccccc4)cc3)c3ccc(-c4cccc5c4oc4c6c(ccc45)C(c4ccccc4)(c4ccccc4)c4ccccc4-6)cc3)cc21 ; CC1(C)c2ccccc2-c2ccc(N(c3ccc(-c4ccccc4)cc3)c3ccc(-c4cccc5c4sc4c6c(ccc45)C(C)(C)c4ccccc4-6)cc3)cc21]

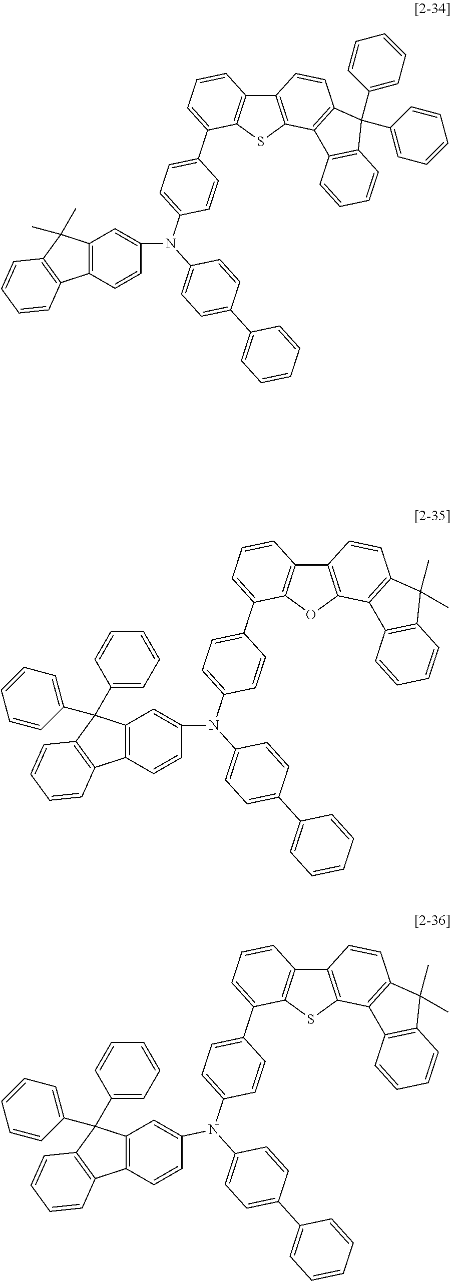 [SMILES: CC1(C)c2ccccc2-c2ccc(N(c3ccc(-c4ccccc4)cc3)c3ccc(-c4cccc5c4sc4c6c(ccc45)C(c4ccccc4)(c4ccccc4)c4ccccc4-6)cc3)cc21 ; CC1(C)c2ccccc2-c2c1ccc1c2oc2c(-c3ccc(N(c4ccc(-c5ccccc5)cc4)c4ccc5c(c4)C(c4ccccc4)(c4ccccc4)c4ccccc4-5)cc3)cccc21 ; CC1(C)c2ccccc2-c2c1ccc1c2sc2c(-c3ccc(N(c4ccc(-c5ccccc5)cc4)c4ccc5c(c4)C(c4ccccc4)(c4ccccc4)c4ccccc4-5)cc3)cccc21]

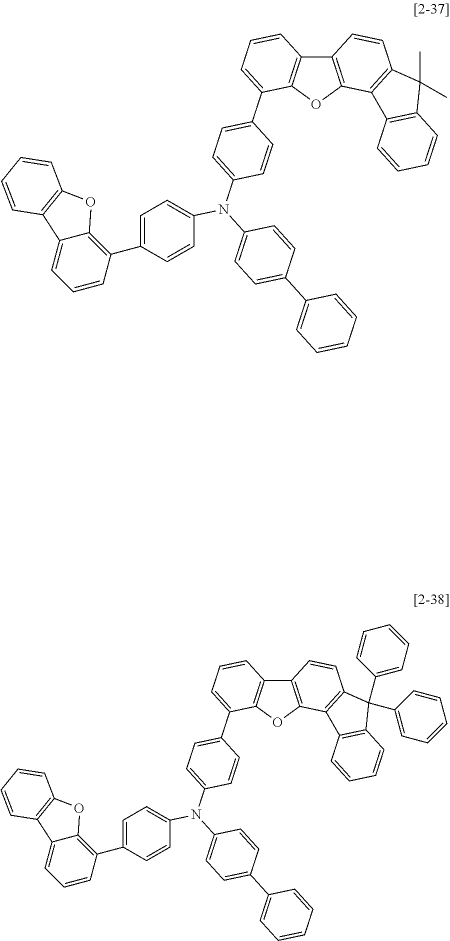 [SMILES: CC1(C)c2ccccc2-c2c1ccc1c2oc2c(-c3ccc(N(c4ccc(-c5ccccc5)cc4)c4ccc(-c5cccc6c5oc5ccccc56)cc4)cc3)cccc21 ; c1ccc(-c2ccc(N(c3ccc(-c4cccc5c4oc4ccccc45)cc3)c3ccc(-c4cccc5c4oc4c6c(ccc45)C(c4ccccc4)(c4ccccc4)c4ccccc4-6)cc3)cc2)cc1]

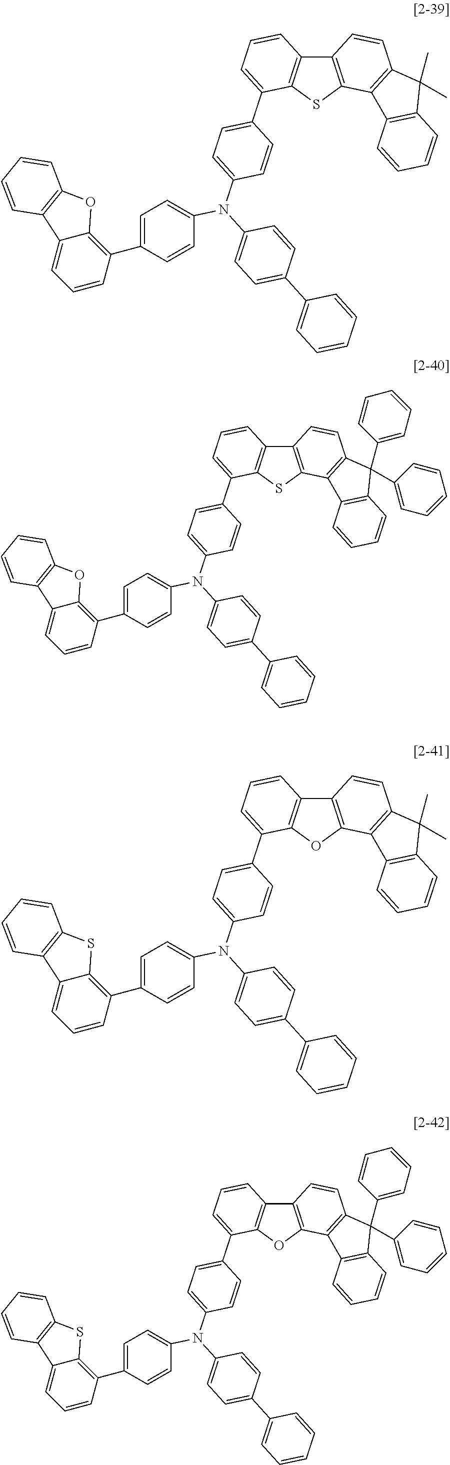 [SMILES: CC1(C)c2ccccc2-c2c1ccc1c2sc2c(-c3ccc(N(c4ccc(-c5ccccc5)cc4)c4ccc(-c5cccc6c5oc5ccccc56)cc4)cc3)cccc21 ; c1ccc(-c2ccc(N(c3ccc(-c4cccc5c4oc4ccccc45)cc3)c3ccc(-c4cccc5c4sc4c6c(ccc45)C(c4ccccc4)(c4ccccc4)c4ccccc4-6)cc3)cc2)cc1 ; CC1(C)c2ccccc2-c2c1ccc1c2oc2c(-c3ccc(N(c4ccc(-c5ccccc5)cc4)c4ccc(-c5cccc6c5sc5ccccc56)cc4)cc3)cccc21 ; c1ccc(-c2ccc(N(c3ccc(-c4cccc5c4oc4c6c(ccc45)C(c4ccccc4)(c4ccccc4)c4ccccc4-6)cc3)c3ccc(-c4cccc5c4sc4ccccc45)cc3)cc2)cc1]

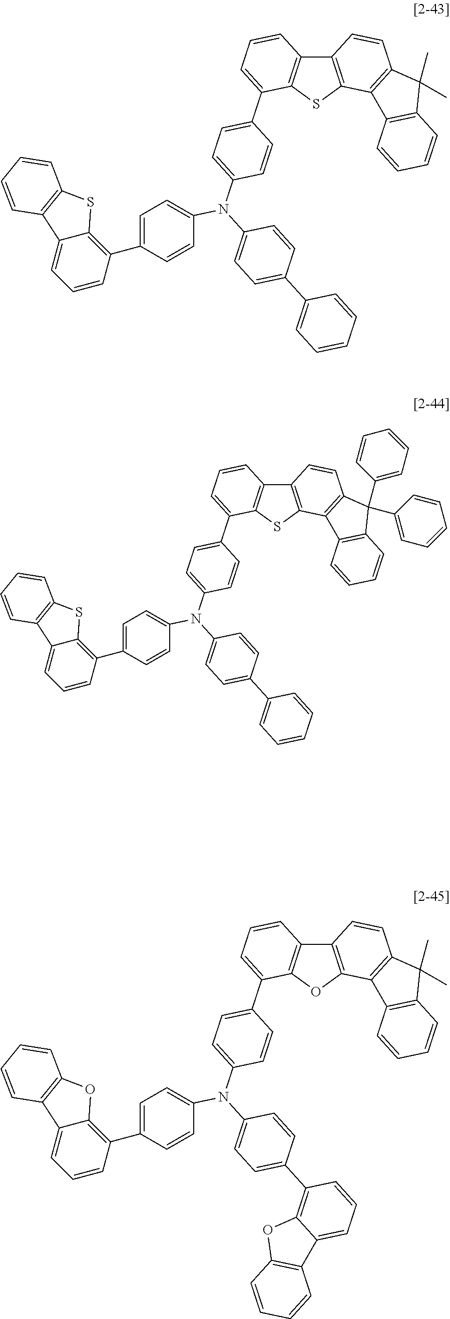 [SMILES: CC1(C)c2ccccc2-c2c1ccc1c2sc2c(-c3ccc(N(c4ccc(-c5ccccc5)cc4)c4ccc(-c5cccc6c5sc5ccccc56)cc4)cc3)cccc21 ; c1ccc(-c2ccc(N(c3ccc(-c4cccc5c4sc4ccccc45)cc3)c3ccc(-c4cccc5c4sc4c6c(ccc45)C(c4ccccc4)(c4ccccc4)c4ccccc4-6)cc3)cc2)cc1 ; CC1(C)c2ccccc2-c2c1ccc1c2oc2c(-c3ccc(N(c4ccc(-c5cccc6c5oc5ccccc56)cc4)c4ccc(-c5cccc6c5oc5ccccc56)cc4)cc3)cccc21]

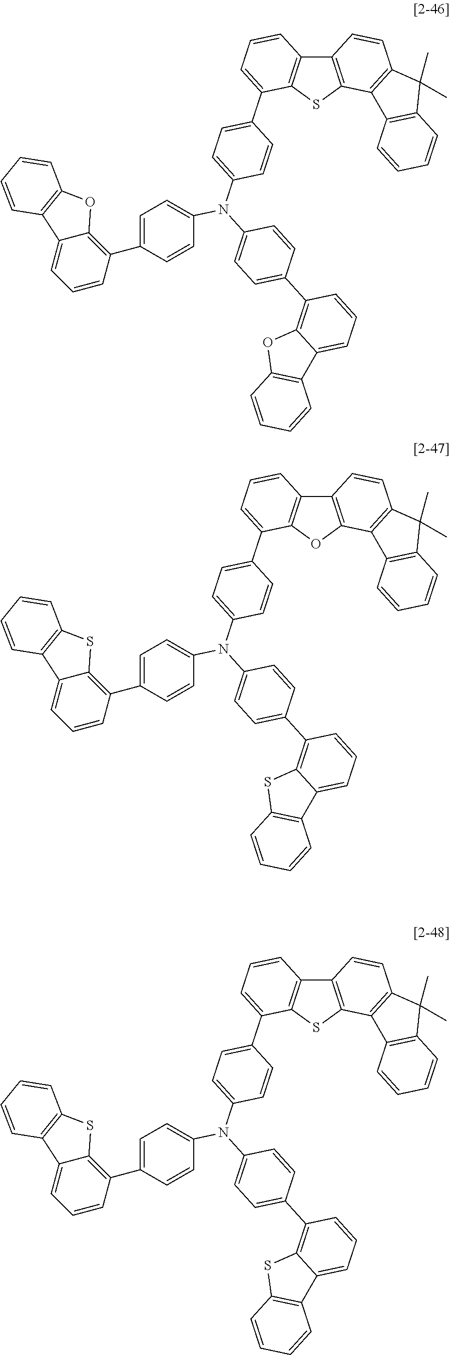 [SMILES: CC1(C)c2ccccc2-c2c1ccc1c2sc2c(-c3ccc(N(c4ccc(-c5cccc6c5oc5ccccc56)cc4)c4ccc(-c5cccc6c5oc5ccccc56)cc4)cc3)cccc21 ; CC1(C)c2ccccc2-c2c1ccc1c2oc2c(-c3ccc(N(c4ccc(-c5cccc6c5sc5ccccc56)cc4)c4ccc(-c5cccc6c5sc5ccccc56)cc4)cc3)cccc21 ; CC1(C)c2ccccc2-c2c1ccc1c2sc2c(-c3ccc(N(c4ccc(-c5cccc6c5sc5ccccc56)cc4)c4ccc(-c5cccc6c5sc5ccccc56)cc4)cc3)cccc21]

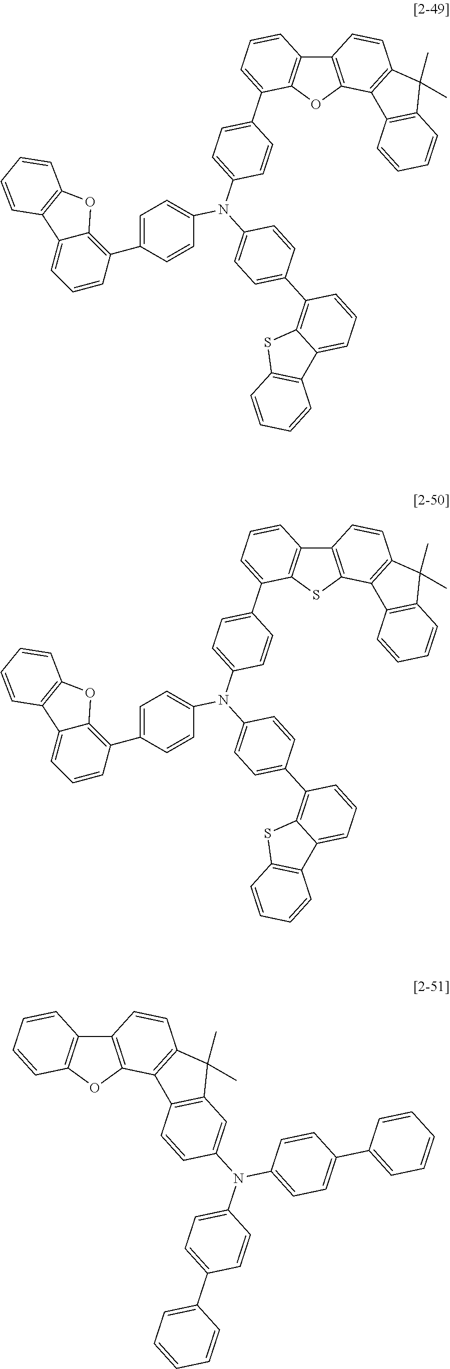 [SMILES: CC1(C)c2ccccc2-c2c1ccc1c2oc2c(-c3ccc(N(c4ccc(-c5cccc6c5oc5ccccc56)cc4)c4ccc(-c5cccc6c5sc5ccccc56)cc4)cc3)cccc21 ; CC1(C)c2ccccc2-c2c1ccc1c2sc2c(-c3ccc(N(c4ccc(-c5cccc6c5oc5ccccc56)cc4)c4ccc(-c5cccc6c5sc5ccccc56)cc4)cc3)cccc21 ; CC1(C)c2cc(N(c3ccc(-c4ccccc4)cc3)c3ccc(-c4ccccc4)cc3)ccc2-c2c1ccc1c2oc2ccccc21]

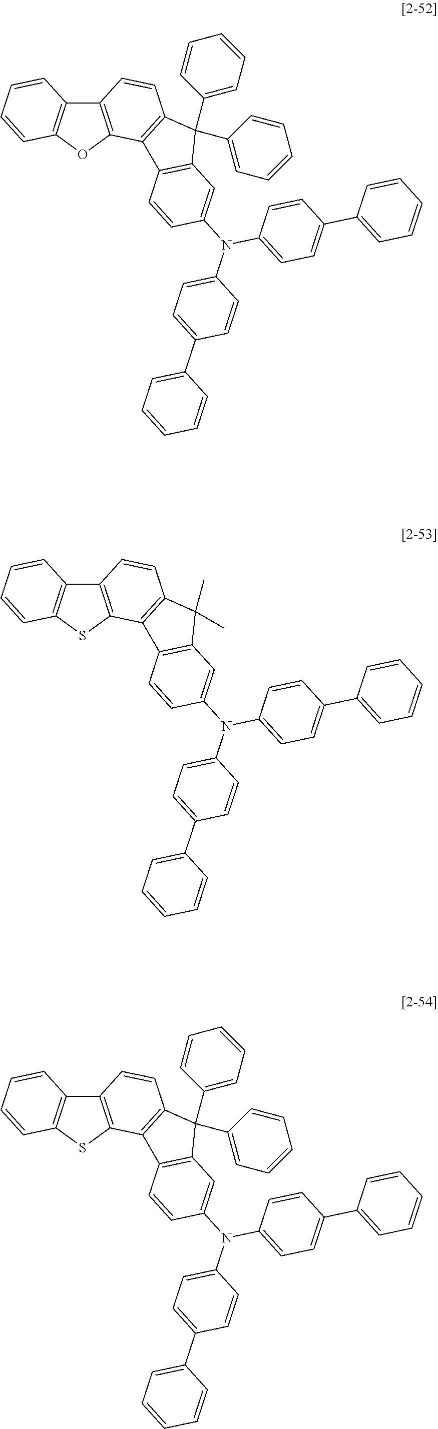 [SMILES: c1ccc(-c2ccc(N(c3ccc(-c4ccccc4)cc3)c3ccc4c(c3)C(c3ccccc3)(c3ccccc3)c3ccc5c(oc6ccccc65)c3-4)cc2)cc1 ; CC1(C)c2cc(N(c3ccc(-c4ccccc4)cc3)c3ccc(-c4ccccc4)cc3)ccc2-c2c1ccc1c2sc2ccccc21 ; c1ccc(-c2ccc(N(c3ccc(-c4ccccc4)cc3)c3ccc4c(c3)C(c3ccccc3)(c3ccccc3)c3ccc5c(sc6ccccc65)c3-4)cc2)cc1]

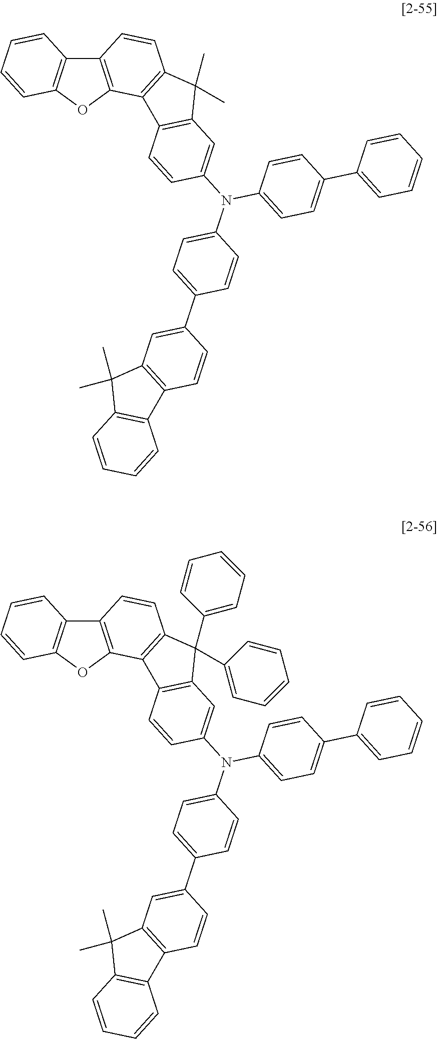 [SMILES: CC1(C)c2ccccc2-c2ccc(-c3ccc(N(c4ccc(-c5ccccc5)cc4)c4ccc5c(c4)C(C)(C)c4ccc6c(oc7ccccc76)c4-5)cc3)cc21 ; CC1(C)c2ccccc2-c2ccc(-c3ccc(N(c4ccc(-c5ccccc5)cc4)c4ccc5c(c4)C(c4ccccc4)(c4ccccc4)c4ccc6c(oc7ccccc76)c4-5)cc3)cc21]

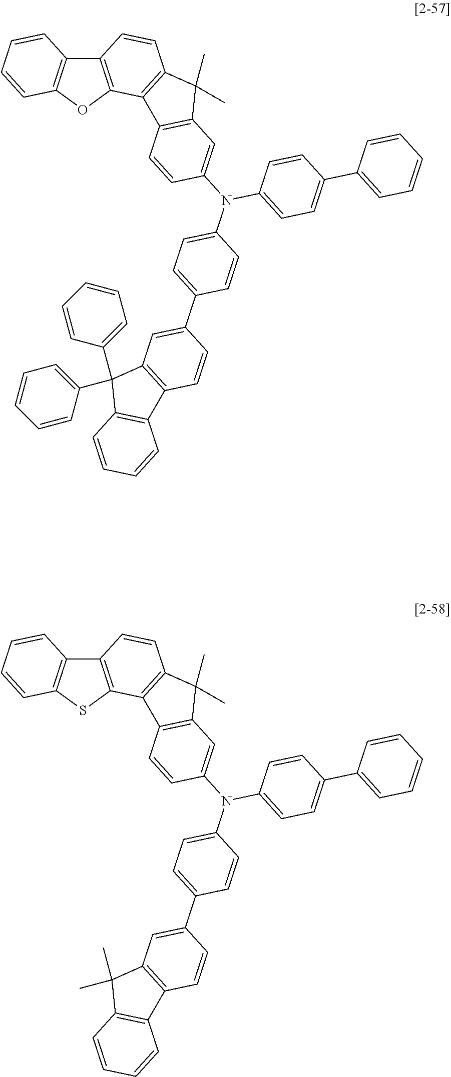 [SMILES: CC1(C)c2cc(N(c3ccc(-c4ccccc4)cc3)c3ccc(-c4ccc5c(c4)C(c4ccccc4)(c4ccccc4)c4ccccc4-5)cc3)ccc2-c2c1ccc1c2oc2ccccc21 ; CC1(C)c2ccccc2-c2ccc(-c3ccc(N(c4ccc(-c5ccccc5)cc4)c4ccc5c(c4)C(C)(C)c4ccc6c(sc7ccccc76)c4-5)cc3)cc21]

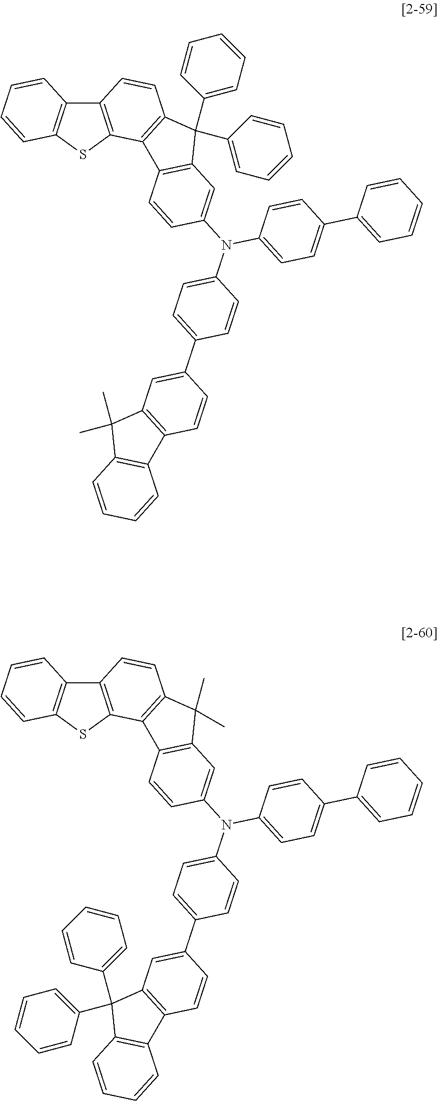 [SMILES: CC1(C)c2ccccc2-c2ccc(-c3ccc(N(c4ccc(-c5ccccc5)cc4)c4ccc5c(c4)C(c4ccccc4)(c4ccccc4)c4ccc6c(sc7ccccc76)c4-5)cc3)cc21 ; CC1(C)c2cc(N(c3ccc(-c4ccccc4)cc3)c3ccc(-c4ccc5c(c4)C(c4ccccc4)(c4ccccc4)c4ccccc4-5)cc3)ccc2-c2c1ccc1c2sc2ccccc21]

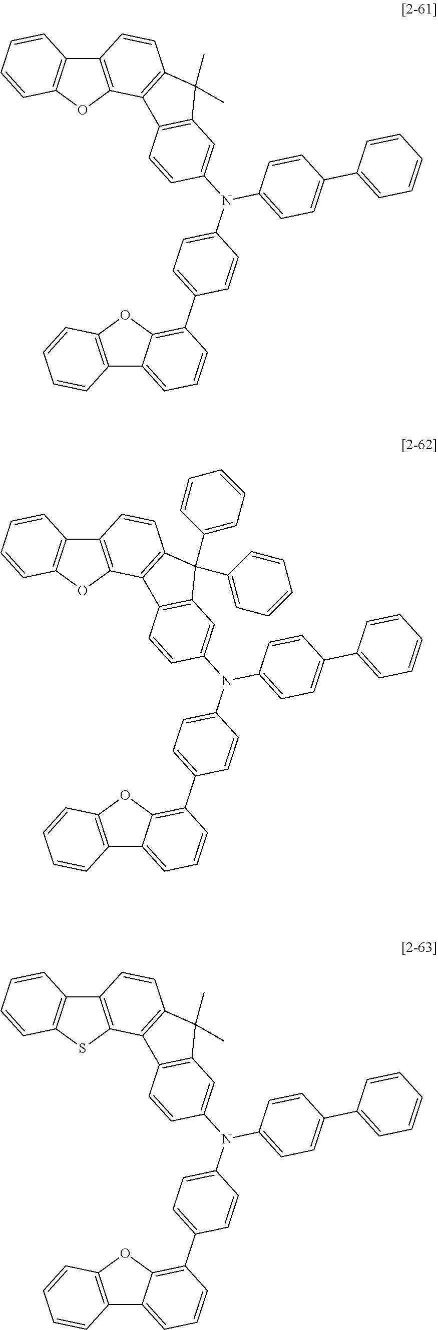 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccc(-c4cccc5c4oc4ccccc45)cc3)c3ccc4c(c3)C(c3ccccc3)(c3ccccc3)c3ccc5c(oc6ccccc65)c3-4)cc2)cc1 ; CC1(C)c2cc(N(c3ccc(-c4ccccc4)cc3)c3ccc(-c4cccc5c4oc4ccccc45)cc3)ccc2-c2c1ccc1c2sc2ccccc21]

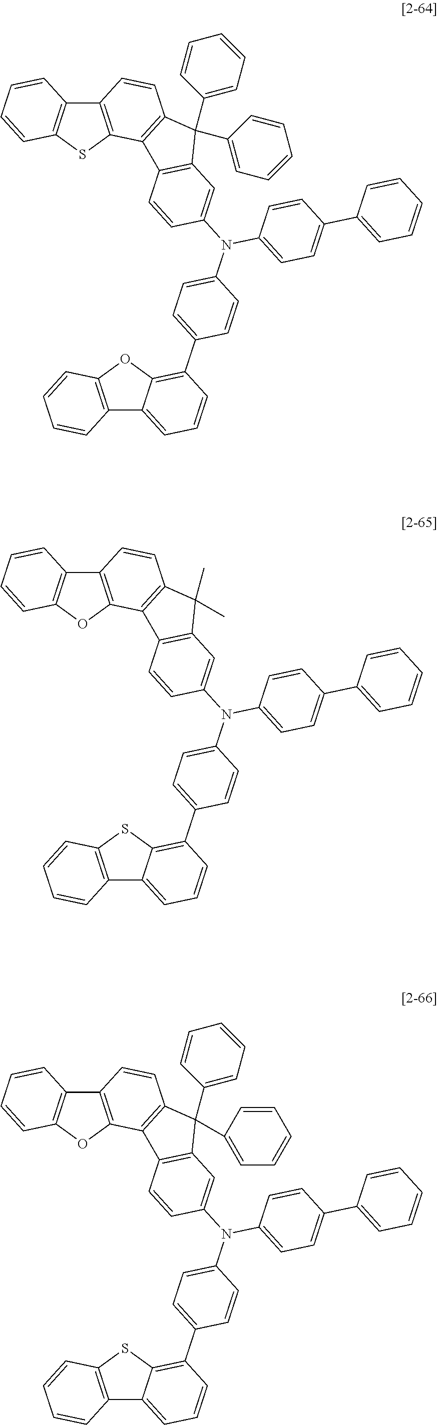 [SMILES: c1ccc(-c2ccc(N(c3ccc(-c4cccc5c4oc4ccccc45)cc3)c3ccc4c(c3)C(c3ccccc3)(c3ccccc3)c3ccc5c(sc6ccccc65)c3-4)cc2)cc1 ; CC1(C)c2cc(N(c3ccc(-c4ccccc4)cc3)c3ccc(-c4cccc5c4sc4ccccc45)cc3)ccc2-c2c1ccc1c2oc2ccccc21 ; c1ccc(-c2ccc(N(c3ccc(-c4cccc5c4sc4ccccc45)cc3)c3ccc4c(c3)C(c3ccccc3)(c3ccccc3)c3ccc5c(oc6ccccc65)c3-4)cc2)cc1]

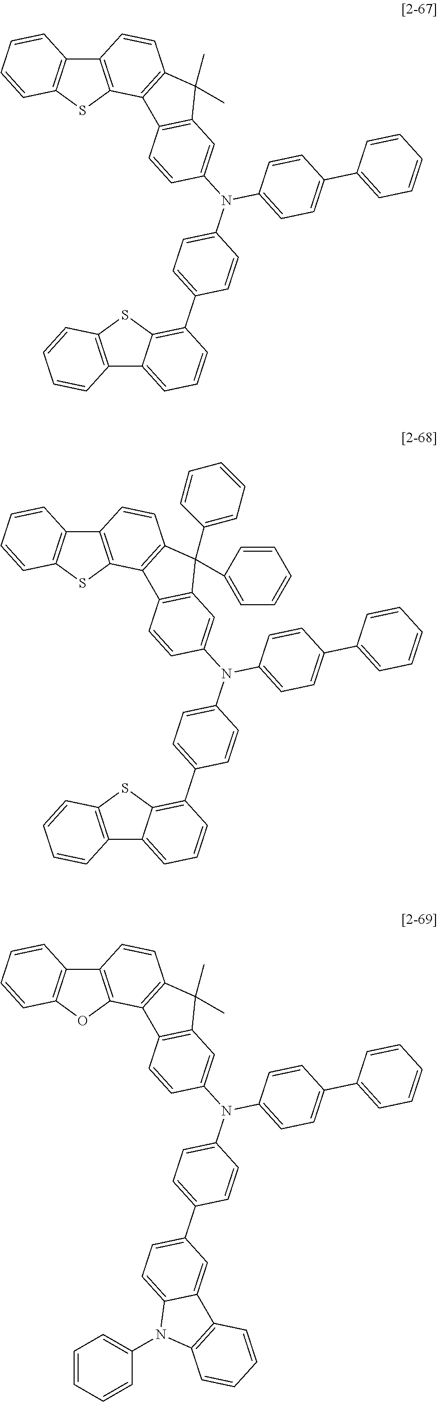 [SMILES: CC1(C)c2cc(N(c3ccc(-c4ccccc4)cc3)c3ccc(-c4cccc5c4sc4ccccc45)cc3)ccc2-c2c1ccc1c2sc2ccccc21 ; c1ccc(-c2ccc(N(c3ccc(-c4cccc5c4sc4ccccc45)cc3)c3ccc4c(c3)C(c3ccccc3)(c3ccccc3)c3ccc5c(sc6ccccc65)c3-4)cc2)cc1 ; CC1(C)c2cc(N(c3ccc(-c4ccccc4)cc3)c3ccc(-c4ccc5c(c4)c4ccccc4n5-c4ccccc4)cc3)ccc2-c2c1ccc1c2oc2ccccc21]

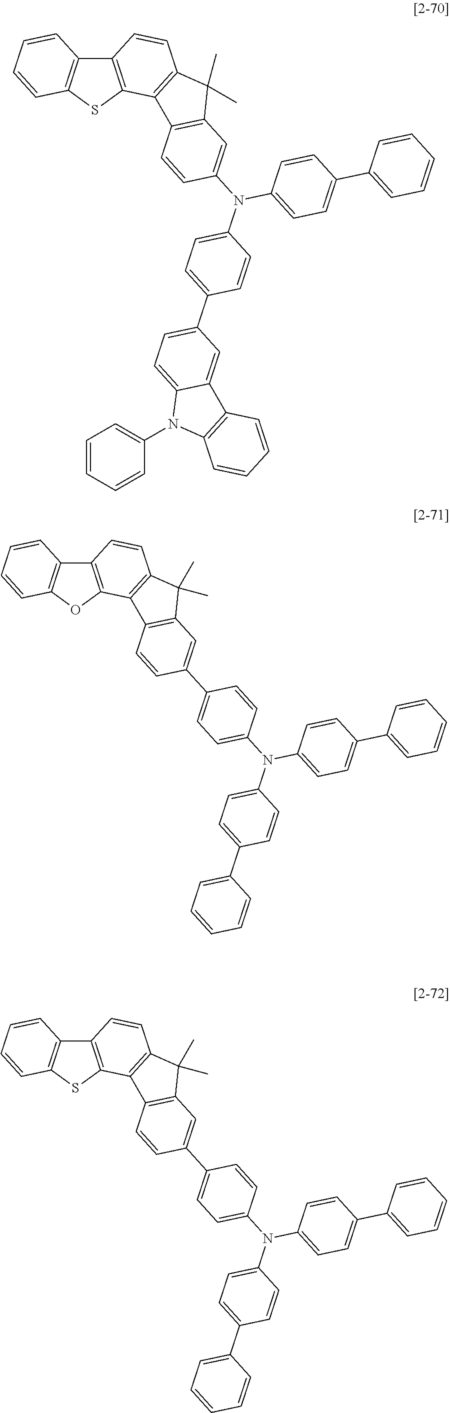 [SMILES: CC1(C)c2cc(N(c3ccc(-c4ccccc4)cc3)c3ccc(-c4ccc5c(c4)c4ccccc4n5-c4ccccc4)cc3)ccc2-c2c1ccc1c2sc2ccccc21 ; CC1(C)c2cc(-c3ccc(N(c4ccc(-c5ccccc5)cc4)c4ccc(-c5ccccc5)cc4)cc3)ccc2-c2c1ccc1c2oc2ccccc21 ; CC1(C)c2cc(-c3ccc(N(c4ccc(-c5ccccc5)cc4)c4ccc(-c5ccccc5)cc4)cc3)ccc2-c2c1ccc1c2sc2ccccc21]

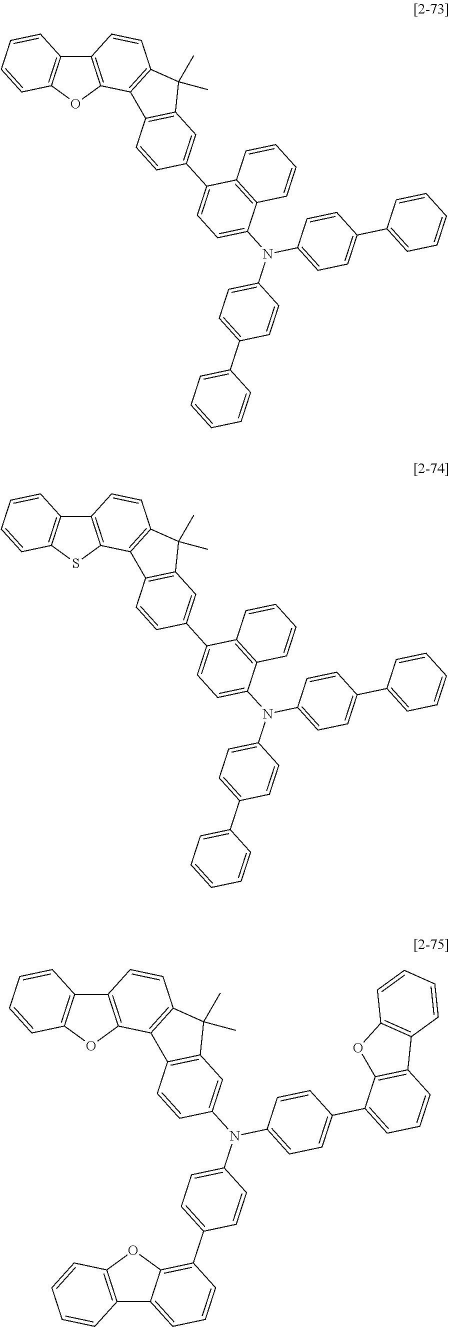 [SMILES: CC1(C)c2cc(-c3ccc(N(c4ccc(-c5ccccc5)cc4)c4ccc(-c5ccccc5)cc4)c4ccccc34)ccc2-c2c1ccc1c2oc2ccccc21 ; CC1(C)c2cc(-c3ccc(N(c4ccc(-c5ccccc5)cc4)c4ccc(-c5ccccc5)cc4)c4ccccc34)ccc2-c2c1ccc1c2sc2ccccc21 ; CC1(C)c2cc(N(c3ccc(-c4cccc5c4oc4ccccc45)cc3)c3ccc(-c4cccc5c4oc4ccccc45)cc3)ccc2-c2c1ccc1c2oc2ccccc21]

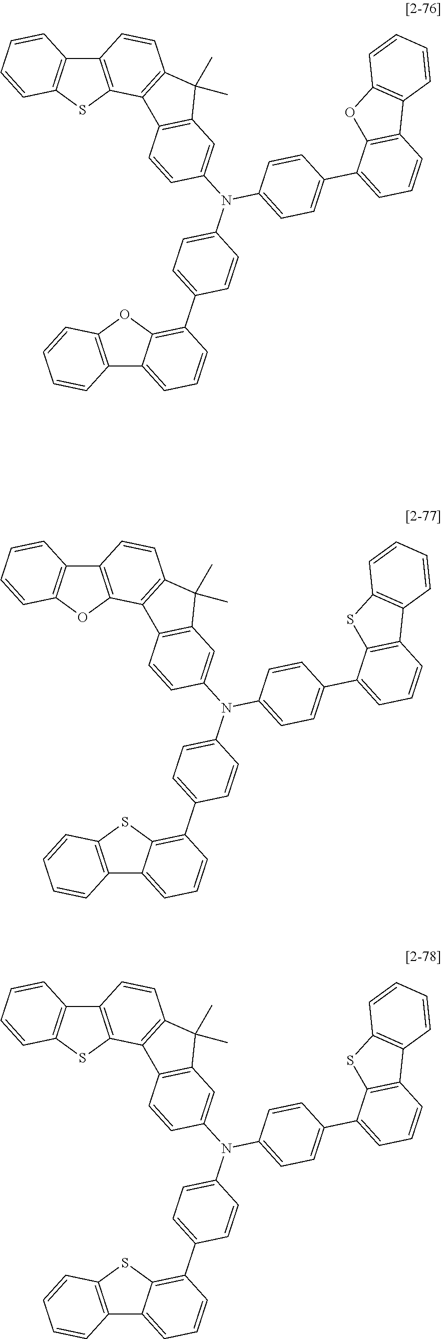 [SMILES: CC1(C)c2cc(N(c3ccc(-c4cccc5c4oc4ccccc45)cc3)c3ccc(-c4cccc5c4oc4ccccc45)cc3)ccc2-c2c1ccc1c2sc2ccccc21 ; CC1(C)c2cc(N(c3ccc(-c4cccc5c4sc4ccccc45)cc3)c3ccc(-c4cccc5c4sc4ccccc45)cc3)ccc2-c2c1ccc1c2oc2ccccc21 ; CC1(C)c2cc(N(c3ccc(-c4cccc5c4sc4ccccc45)cc3)c3ccc(-c4cccc5c4sc4ccccc45)cc3)ccc2-c2c1ccc1c2sc2ccccc21]

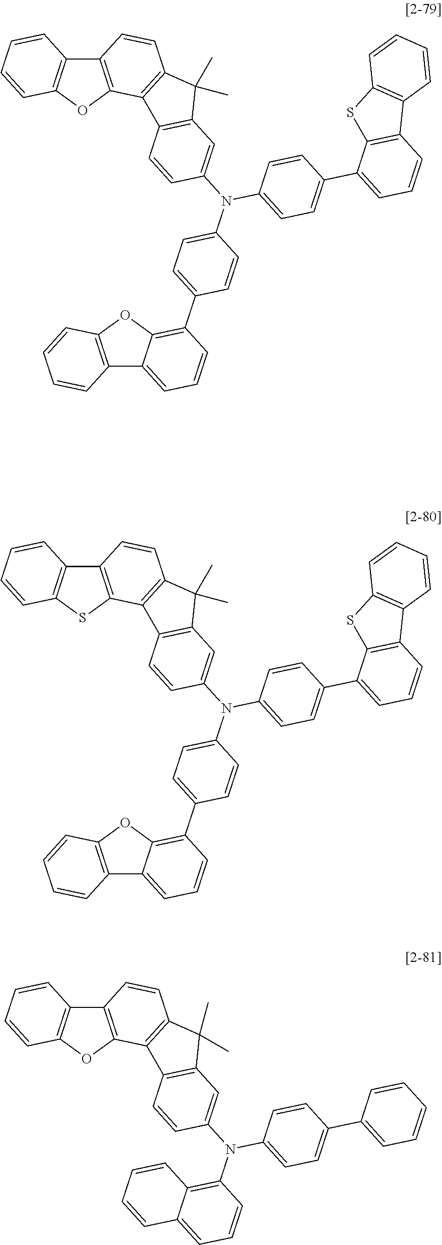 [SMILES: CC1(C)c2cc(N(c3ccc(-c4cccc5c4oc4ccccc45)cc3)c3ccc(-c4cccc5c4sc4ccccc45)cc3)ccc2-c2c1ccc1c2oc2ccccc21 ; CC1(C)c2cc(N(c3ccc(-c4cccc5c4oc4ccccc45)cc3)c3ccc(-c4cccc5c4sc4ccccc45)cc3)ccc2-c2c1ccc1c2sc2ccccc21 ; CC1(C)c2cc(N(c3ccc(-c4ccccc4)cc3)c3cccc4ccccc34)ccc2-c2c1ccc1c2oc2ccccc21]

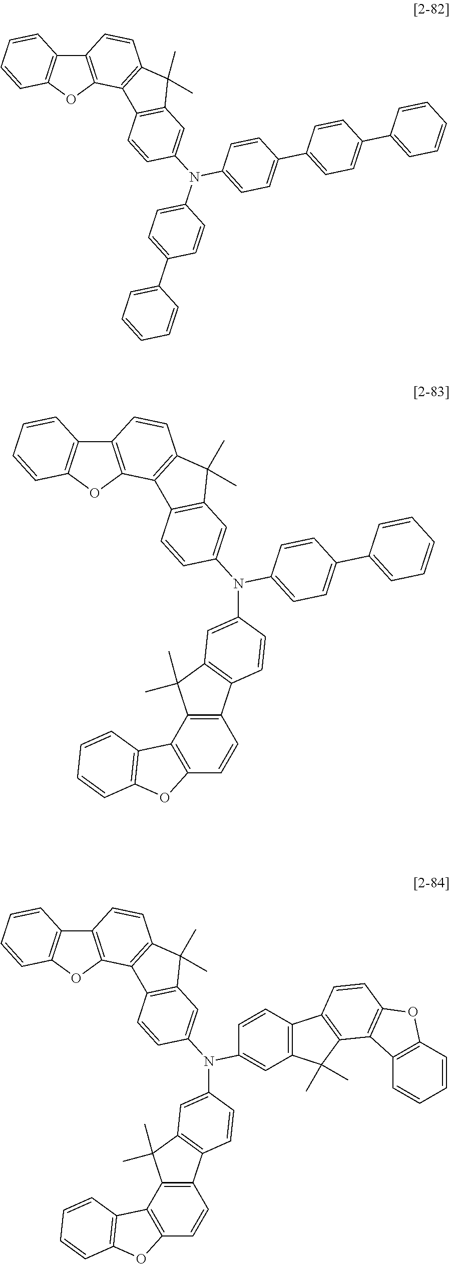 [SMILES: CC1(C)c2cc(N(c3ccc(-c4ccccc4)cc3)c3ccc(-c4ccc(-c5ccccc5)cc4)cc3)ccc2-c2c1ccc1c2oc2ccccc21 ; CC1(C)c2cc(N(c3ccc(-c4ccccc4)cc3)c3ccc4c(c3)C(C)(C)c3c-4ccc4oc5ccccc5c34)ccc2-c2c1ccc1c2oc2ccccc21 ; CC1(C)c2cc(N(c3ccc4c(c3)C(C)(C)c3c-4ccc4oc5ccccc5c34)c3ccc4c(c3)C(C)(C)c3c-4ccc4oc5ccccc5c34)ccc2-c2c1ccc1c2oc2ccccc21]

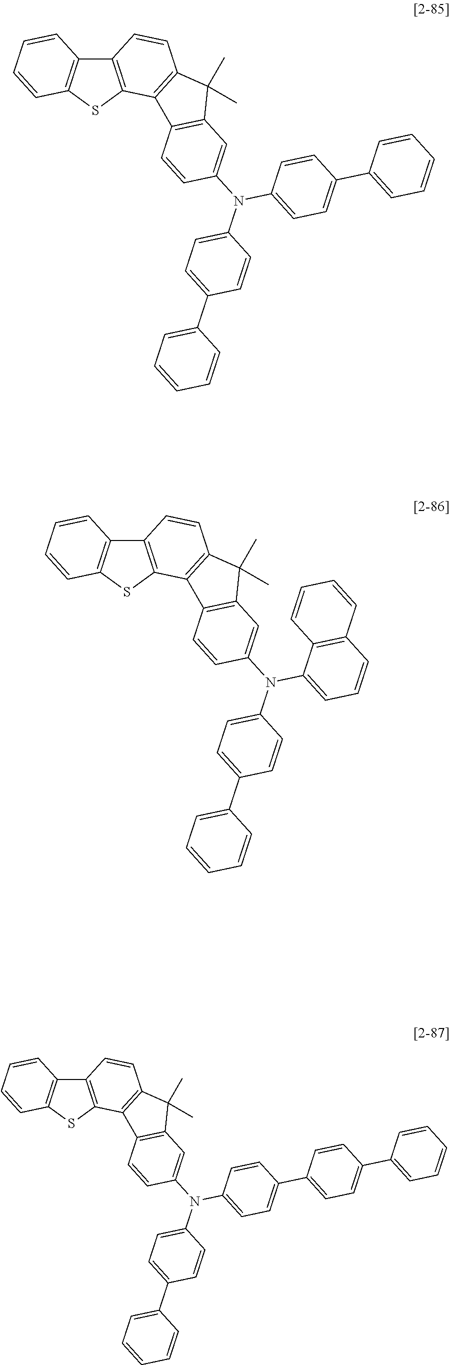 [SMILES: CC1(C)c2cc(N(c3ccc(-c4ccccc4)cc3)c3ccc(-c4ccccc4)cc3)ccc2-c2c1ccc1c2sc2ccccc21 ; CC1(C)c2cc(N(c3ccc(-c4ccccc4)cc3)c3cccc4ccccc34)ccc2-c2c1ccc1c2sc2ccccc21 ; CC1(C)c2cc(N(c3ccc(-c4ccccc4)cc3)c3ccc(-c4ccc(-c5ccccc5)cc4)cc3)ccc2-c2c1ccc1c2sc2ccccc21]

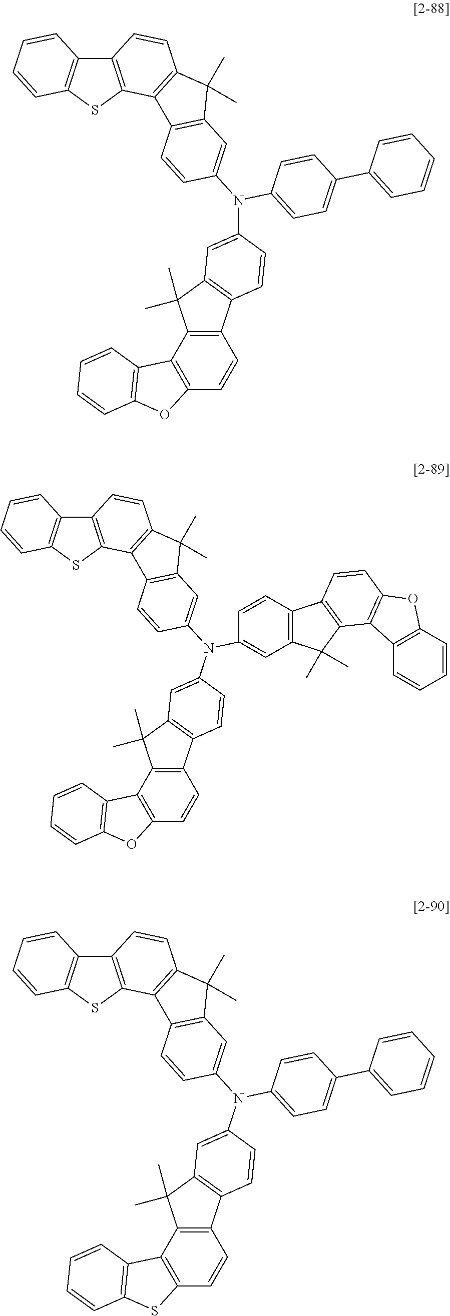 [SMILES: CC1(C)c2cc(N(c3ccc(-c4ccccc4)cc3)c3ccc4c(c3)C(C)(C)c3c-4ccc4oc5ccccc5c34)ccc2-c2c1ccc1c2sc2ccccc21 ; CC1(C)c2cc(N(c3ccc4c(c3)C(C)(C)c3c-4ccc4oc5ccccc5c34)c3ccc4c(c3)C(C)(C)c3c-4ccc4oc5ccccc5c34)ccc2-c2c1ccc1c2sc2ccccc21 ; CC1(C)c2cc(N(c3ccc(-c4ccccc4)cc3)c3ccc4c(c3)C(C)(C)c3c-4ccc4sc5ccccc5c34)ccc2-c2c1ccc1c2sc2ccccc21]

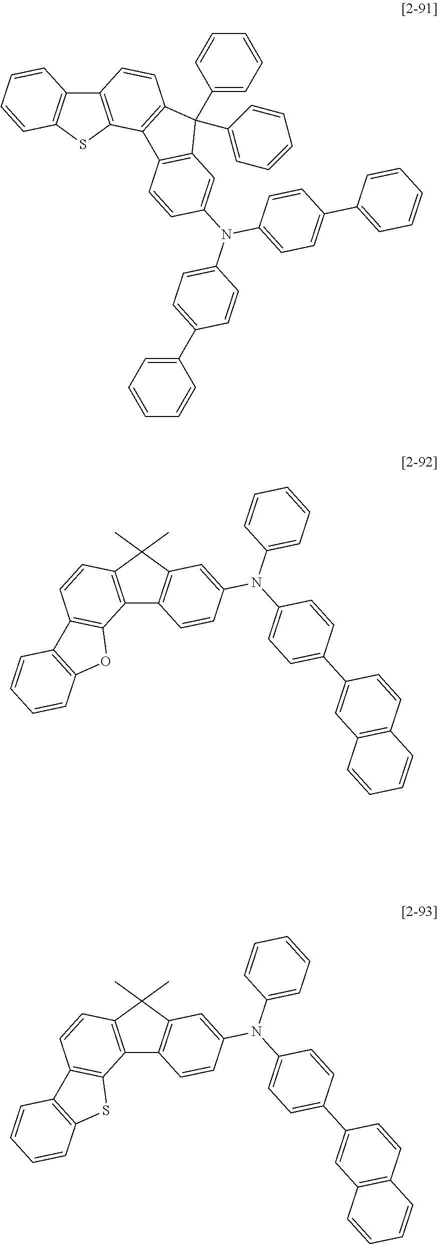 [SMILES: c1ccc(-c2ccc(N(c3ccc(-c4ccccc4)cc3)c3ccc4c(c3)C(c3ccccc3)(c3ccccc3)c3ccc5c(sc6ccccc65)c3-4)cc2)cc1 ; CC1(C)c2cc(N(c3ccccc3)c3ccc(-c4ccc5ccccc5c4)cc3)ccc2-c2c1ccc1c2oc2ccccc21 ; CC1(C)c2cc(N(c3ccccc3)c3ccc(-c4ccc5ccccc5c4)cc3)ccc2-c2c1ccc1c2sc2ccccc21]

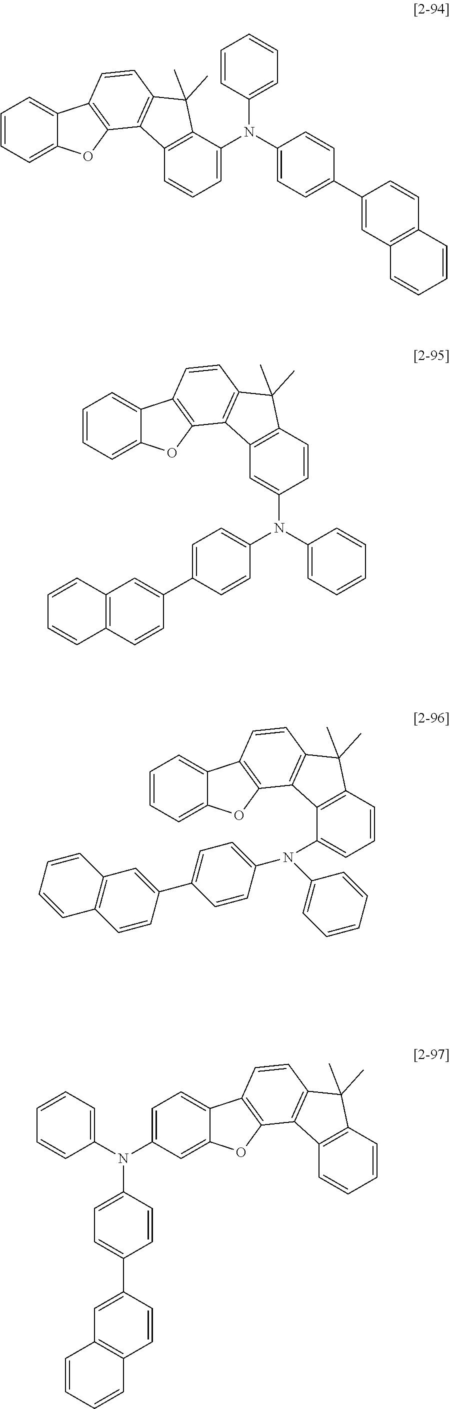 [SMILES: CC1(C)c2ccc3c(oc4ccccc43)c2-c2cccc(N(c3ccccc3)c3ccc(-c4ccc5ccccc5c4)cc3)c21 ; CC1(C)c2ccc(N(c3ccccc3)c3ccc(-c4ccc5ccccc5c4)cc3)cc2-c2c1ccc1c2oc2ccccc21 ; CC1(C)c2cccc(N(c3ccccc3)c3ccc(-c4ccc5ccccc5c4)cc3)c2-c2c1ccc1c2oc2ccccc21 ; CC1(C)c2ccccc2-c2c1ccc1c2oc2cc(N(c3ccccc3)c3ccc(-c4ccc5ccccc5c4)cc3)ccc21]

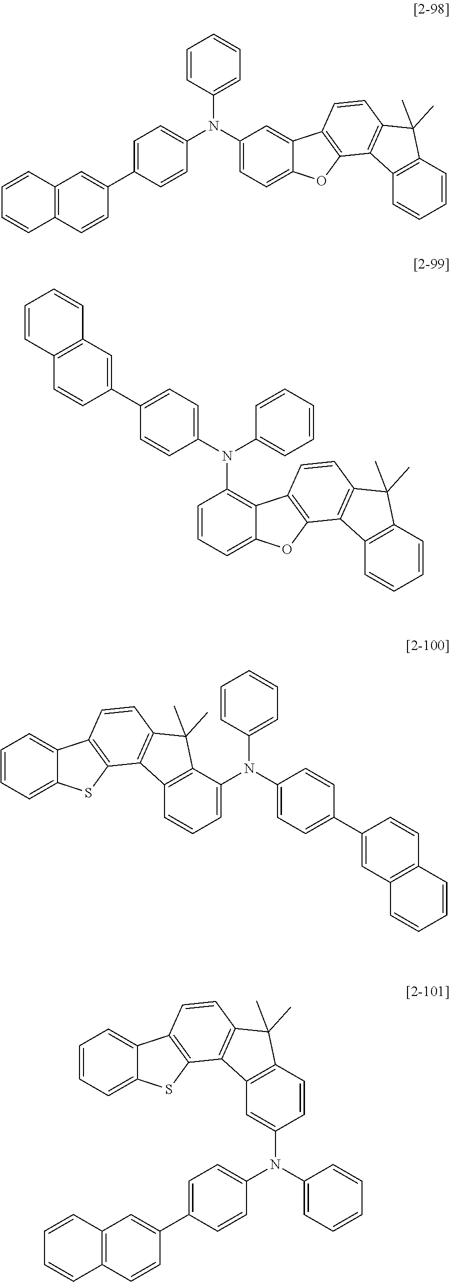 [SMILES: CC1(C)c2ccccc2-c2c1ccc1c2oc2ccc(N(c3ccccc3)c3ccc(-c4ccc5ccccc5c4)cc3)cc21 ; CC1(C)c2ccccc2-c2c1ccc1c2oc2cccc(N(c3ccccc3)c3ccc(-c4ccc5ccccc5c4)cc3)c21 ; CC1(C)c2ccc3c(sc4ccccc43)c2-c2cccc(N(c3ccccc3)c3ccc(-c4ccc5ccccc5c4)cc3)c21 ; CC1(C)c2ccc(N(c3ccccc3)c3ccc(-c4ccc5ccccc5c4)cc3)cc2-c2c1ccc1c2sc2ccccc21]

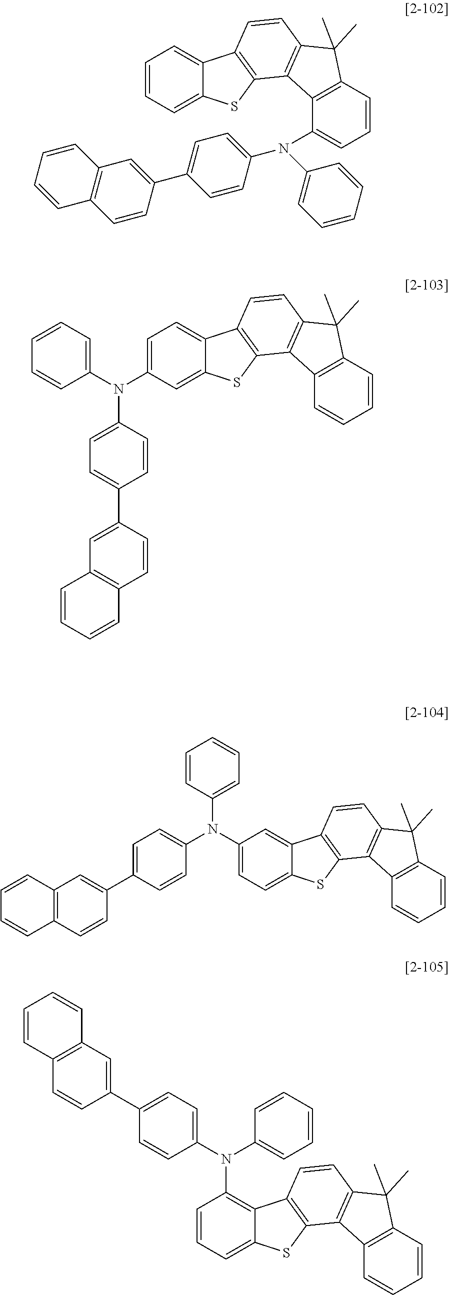 [SMILES: CC1(C)c2cccc(N(c3ccccc3)c3ccc(-c4ccc5ccccc5c4)cc3)c2-c2c1ccc1c2sc2ccccc21 ; CC1(C)c2ccccc2-c2c1ccc1c2sc2cc(N(c3ccccc3)c3ccc(-c4ccc5ccccc5c4)cc3)ccc21 ; CC1(C)c2ccccc2-c2c1ccc1c2sc2ccc(N(c3ccccc3)c3ccc(-c4ccc5ccccc5c4)cc3)cc21 ; CC1(C)c2ccccc2-c2c1ccc1c2sc2cccc(N(c3ccccc3)c3ccc(-c4ccc5ccccc5c4)cc3)c21]

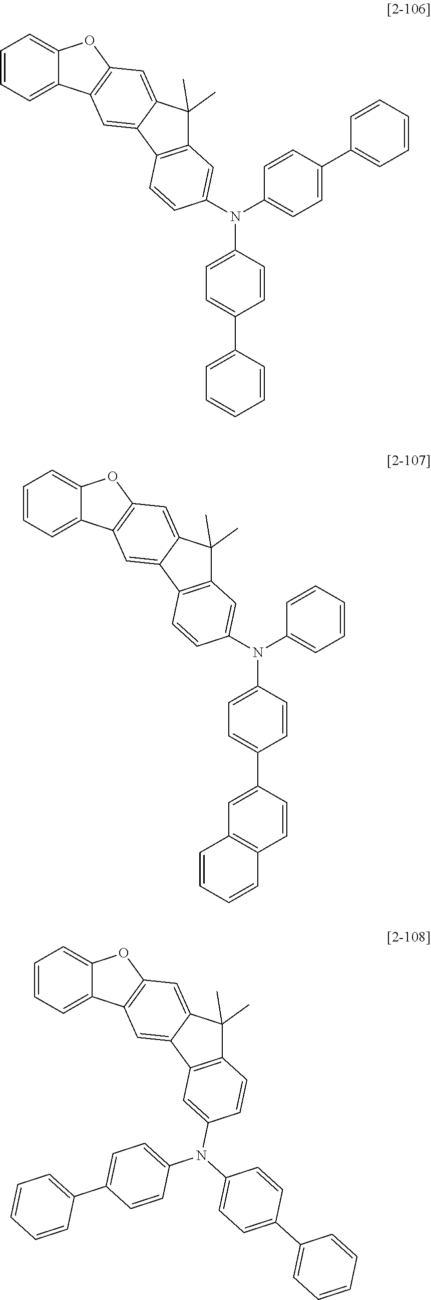 [SMILES: CC1(C)c2cc(N(c3ccc(-c4ccccc4)cc3)c3ccc(-c4ccccc4)cc3)ccc2-c2cc3c(cc21)oc1ccccc13 ; CC1(C)c2cc(N(c3ccccc3)c3ccc(-c4ccc5ccccc5c4)cc3)ccc2-c2cc3c(cc21)oc1ccccc13 ; CC1(C)c2ccc(N(c3ccc(-c4ccccc4)cc3)c3ccc(-c4ccccc4)cc3)cc2-c2cc3c(cc21)oc1ccccc13]

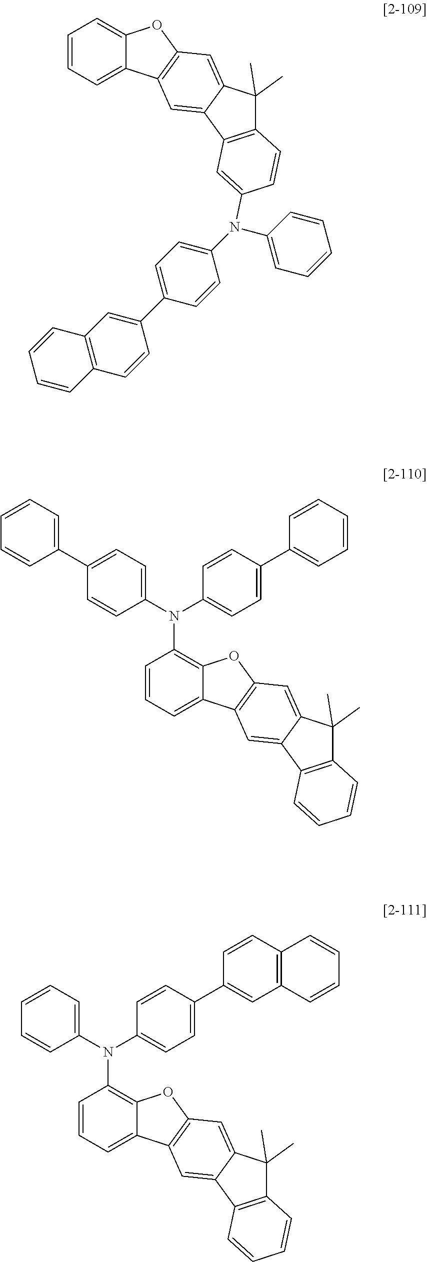 [SMILES: CC1(C)c2ccc(N(c3ccccc3)c3ccc(-c4ccc5ccccc5c4)cc3)cc2-c2cc3c(cc21)oc1ccccc13 ; CC1(C)c2ccccc2-c2cc3c(cc21)oc1c(N(c2ccc(-c4ccccc4)cc2)c2ccc(-c4ccccc4)cc2)cccc13 ; CC1(C)c2ccccc2-c2cc3c(cc21)oc1c(N(c2ccccc2)c2ccc(-c4ccc5ccccc5c4)cc2)cccc13]

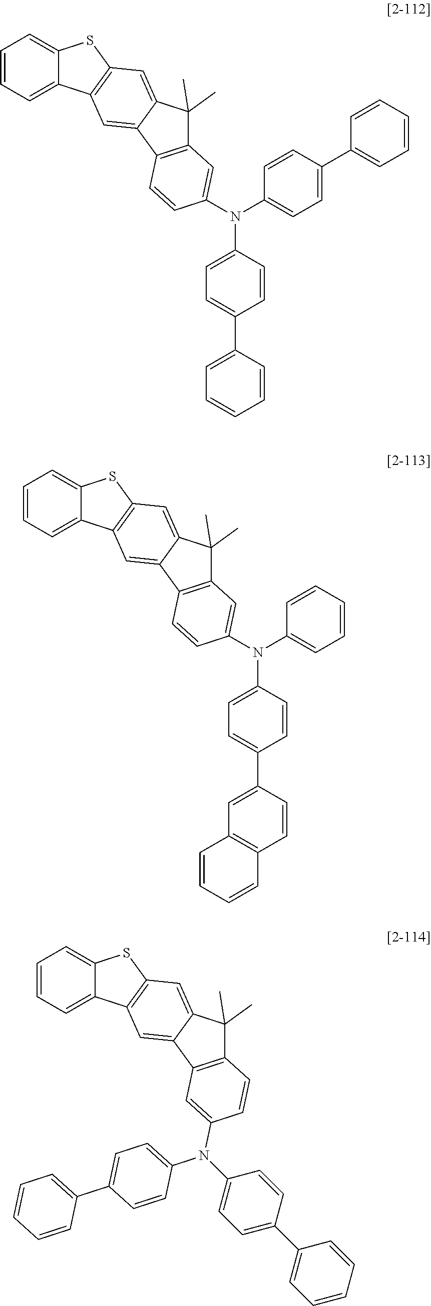 [SMILES: CC1(C)c2cc(N(c3ccc(-c4ccccc4)cc3)c3ccc(-c4ccccc4)cc3)ccc2-c2cc3c(cc21)sc1ccccc13 ; CC1(C)c2cc(N(c3ccccc3)c3ccc(-c4ccc5ccccc5c4)cc3)ccc2-c2cc3c(cc21)sc1ccccc13 ; CC1(C)c2ccc(N(c3ccc(-c4ccccc4)cc3)c3ccc(-c4ccccc4)cc3)cc2-c2cc3c(cc21)sc1ccccc13]

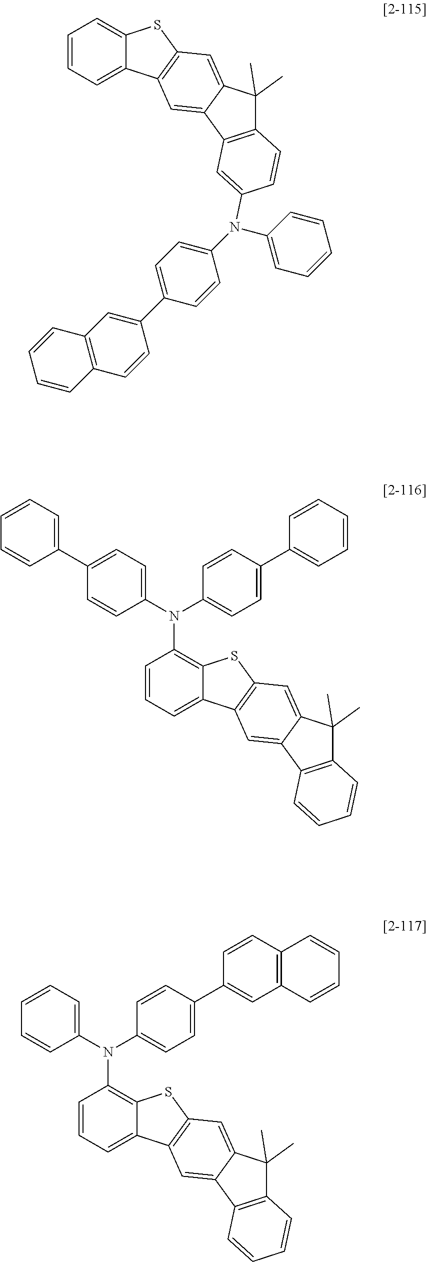 [SMILES: CC1(C)c2ccc(N(c3ccccc3)c3ccc(-c4ccc5ccccc5c4)cc3)cc2-c2cc3c(cc21)sc1ccccc13 ; CC1(C)c2ccccc2-c2cc3c(cc21)sc1c(N(c2ccc(-c4ccccc4)cc2)c2ccc(-c4ccccc4)cc2)cccc13 ; CC1(C)c2ccccc2-c2cc3c(cc21)sc1c(N(c2ccccc2)c2ccc(-c4ccc5ccccc5c4)cc2)cccc13]

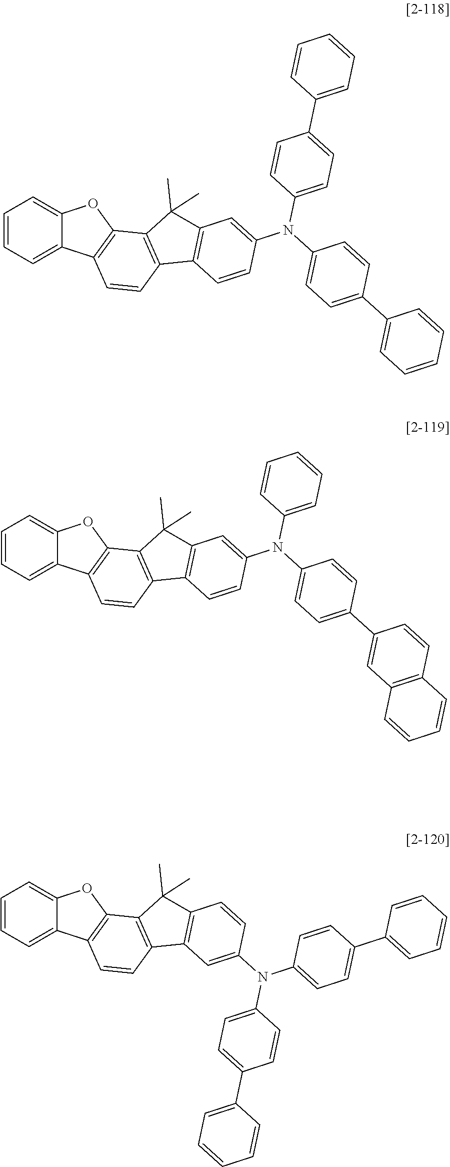 [SMILES: CC1(C)c2cc(N(c3ccc(-c4ccccc4)cc3)c3ccc(-c4ccccc4)cc3)ccc2-c2ccc3c(oc4ccccc43)c21 ; CC1(C)c2cc(N(c3ccccc3)c3ccc(-c4ccc5ccccc5c4)cc3)ccc2-c2ccc3c(oc4ccccc43)c21 ; CC1(C)c2ccc(N(c3ccc(-c4ccccc4)cc3)c3ccc(-c4ccccc4)cc3)cc2-c2ccc3c(oc4ccccc43)c21]

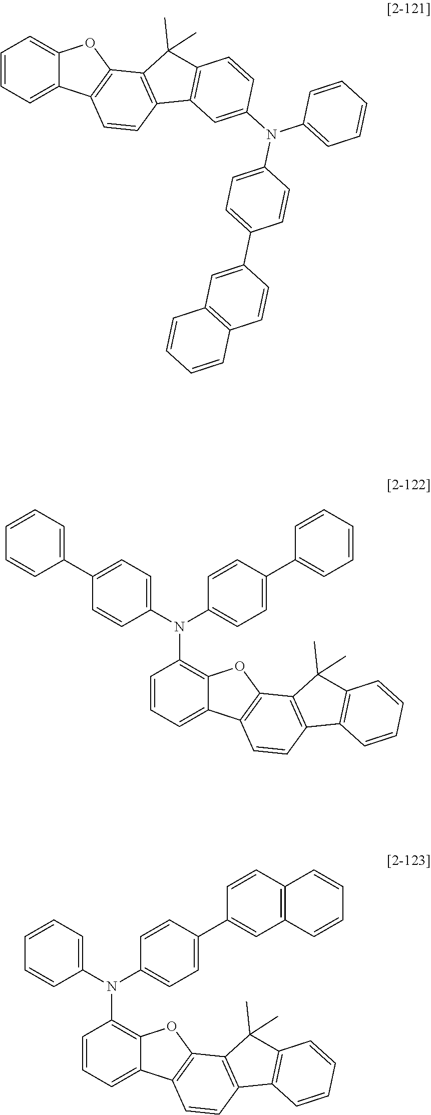 [SMILES: CC1(C)c2ccc(N(c3ccccc3)c3ccc(-c4ccc5ccccc5c4)cc3)cc2-c2ccc3c(oc4ccccc43)c21 ; CC1(C)c2ccccc2-c2ccc3c(oc4c(N(c5ccc(-c6ccccc6)cc5)c5ccc(-c6ccccc6)cc5)cccc43)c21 ; CC1(C)c2ccccc2-c2ccc3c(oc4c(N(c5ccccc5)c5ccc(-c6ccc7ccccc7c6)cc5)cccc43)c21]

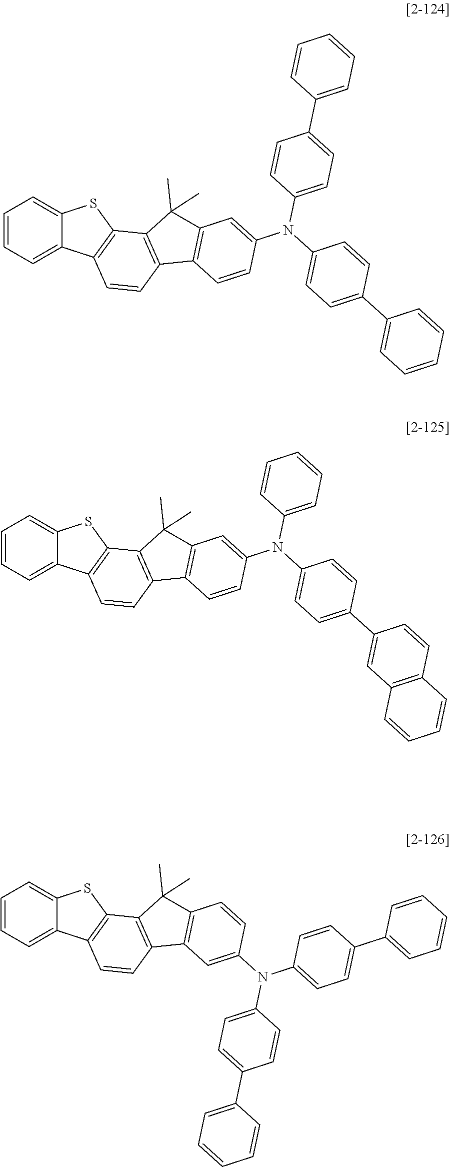 [SMILES: CC1(C)c2cc(N(c3ccc(-c4ccccc4)cc3)c3ccc(-c4ccccc4)cc3)ccc2-c2ccc3c(sc4ccccc43)c21 ; CC1(C)c2cc(N(c3ccccc3)c3ccc(-c4ccc5ccccc5c4)cc3)ccc2-c2ccc3c(sc4ccccc43)c21 ; CC1(C)c2ccc(N(c3ccc(-c4ccccc4)cc3)c3ccc(-c4ccccc4)cc3)cc2-c2ccc3c(sc4ccccc43)c21]

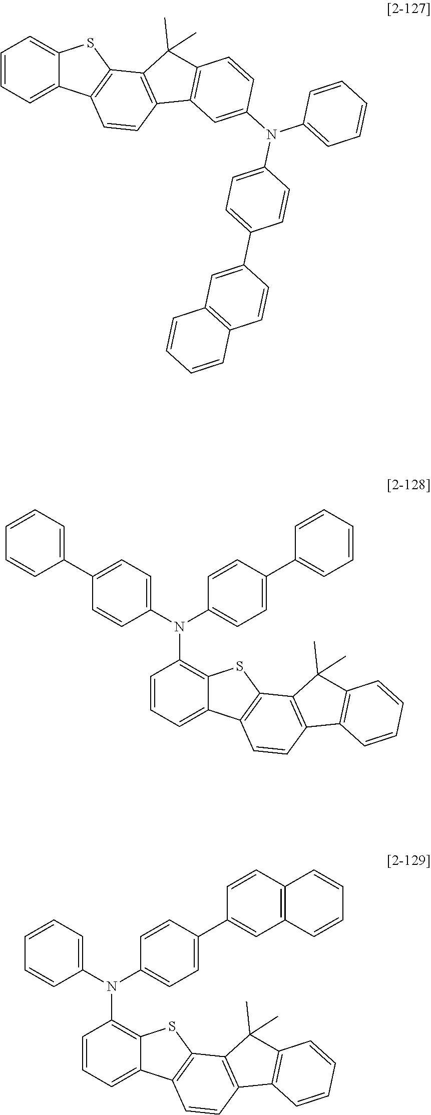 [SMILES: CC1(C)c2ccc(N(c3ccccc3)c3ccc(-c4ccc5ccccc5c4)cc3)cc2-c2ccc3c(sc4ccccc43)c21 ; CC1(C)c2ccccc2-c2ccc3c(sc4c(N(c5ccc(-c6ccccc6)cc5)c5ccc(-c6ccccc6)cc5)cccc43)c21 ; CC1(C)c2ccccc2-c2ccc3c(sc4c(N(c5ccccc5)c5ccc(-c6ccc7ccccc7c6)cc5)cccc43)c21]

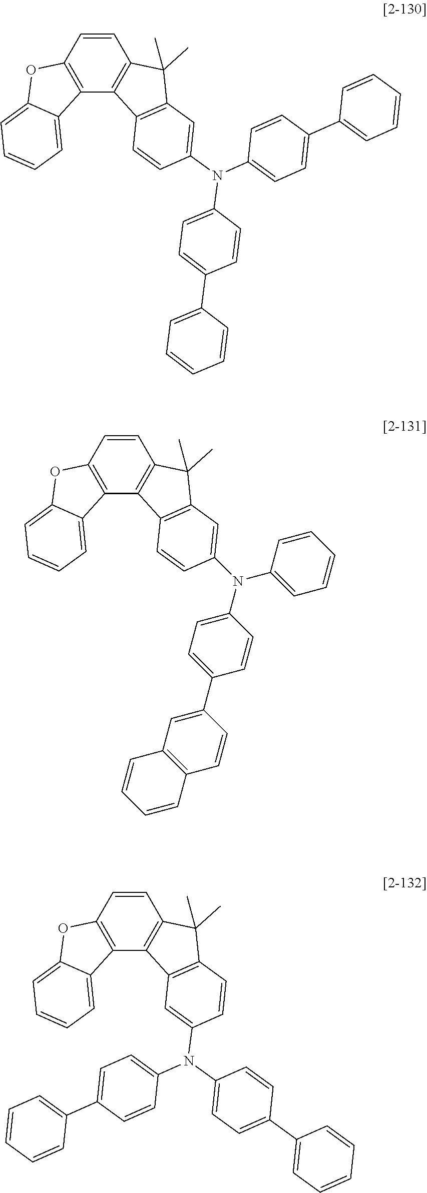 [SMILES: CC1(C)c2cc(N(c3ccc(-c4ccccc4)cc3)c3ccc(-c4ccccc4)cc3)ccc2-c2c1ccc1oc3ccccc3c21 ; CC1(C)c2cc(N(c3ccccc3)c3ccc(-c4ccc5ccccc5c4)cc3)ccc2-c2c1ccc1oc3ccccc3c21 ; CC1(C)c2ccc(N(c3ccc(-c4ccccc4)cc3)c3ccc(-c4ccccc4)cc3)cc2-c2c1ccc1oc3ccccc3c21]

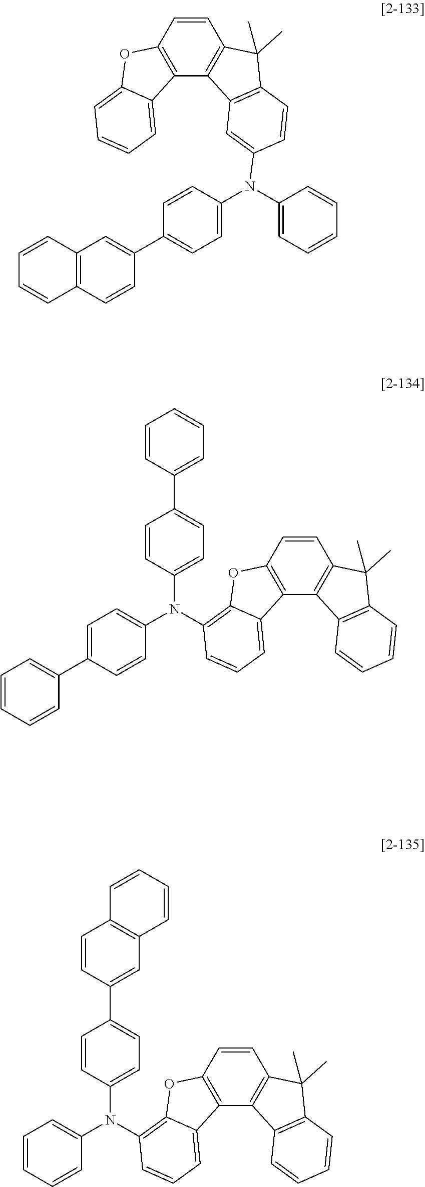 [SMILES: CC1(C)c2ccc(N(c3ccccc3)c3ccc(-c4ccc5ccccc5c4)cc3)cc2-c2c1ccc1oc3ccccc3c21 ; CC1(C)c2ccccc2-c2c1ccc1oc3c(N(c4ccc(-c5ccccc5)cc4)c4ccc(-c5ccccc5)cc4)cccc3c21 ; CC1(C)c2ccccc2-c2c1ccc1oc3c(N(c4ccccc4)c4ccc(-c5ccc6ccccc6c5)cc4)cccc3c21]

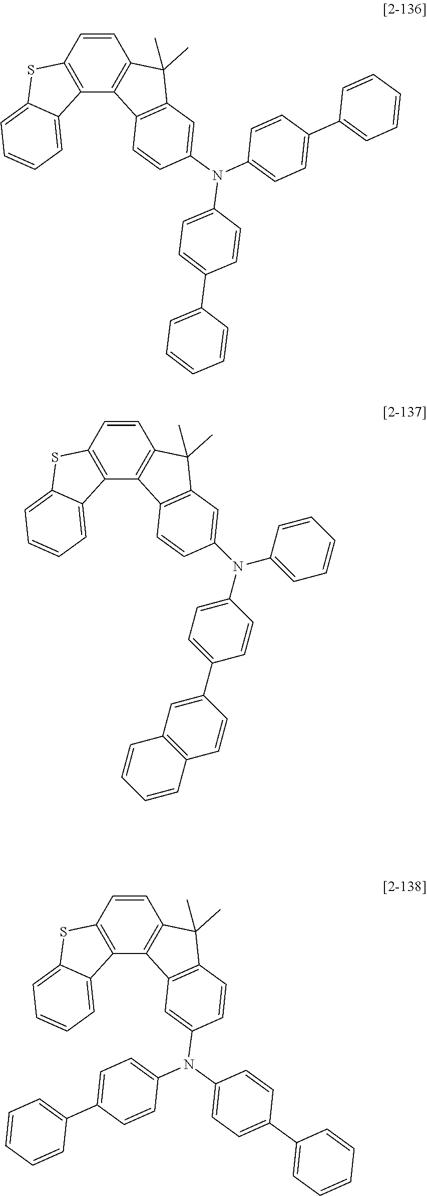 [SMILES: CC1(C)c2cc(N(c3ccc(-c4ccccc4)cc3)c3ccc(-c4ccccc4)cc3)ccc2-c2c1ccc1sc3ccccc3c21 ; CC1(C)c2cc(N(c3ccccc3)c3ccc(-c4ccc5ccccc5c4)cc3)ccc2-c2c1ccc1sc3ccccc3c21 ; CC1(C)c2ccc(N(c3ccc(-c4ccccc4)cc3)c3ccc(-c4ccccc4)cc3)cc2-c2c1ccc1sc3ccccc3c21]

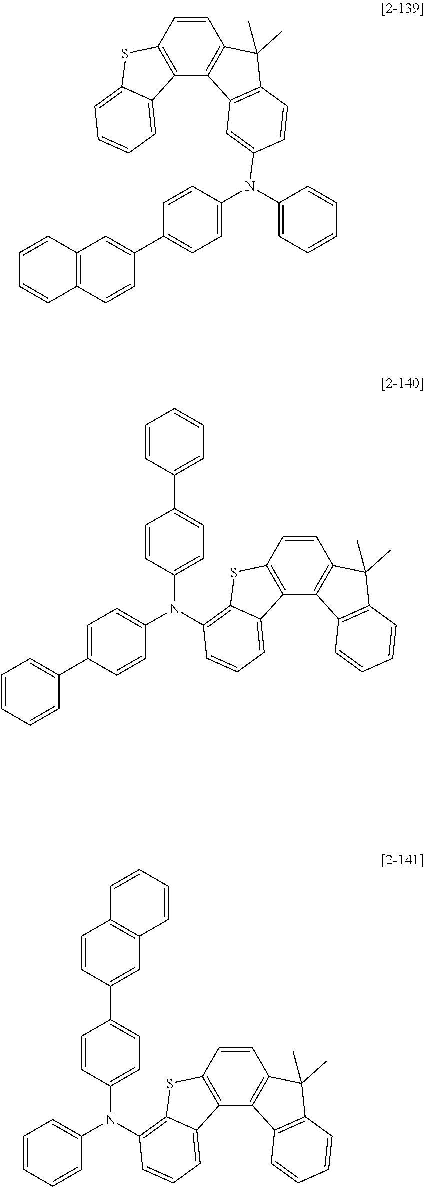 [SMILES: CC1(C)c2ccc(N(c3ccccc3)c3ccc(-c4ccc5ccccc5c4)cc3)cc2-c2c1ccc1sc3ccccc3c21 ; CC1(C)c2ccccc2-c2c1ccc1sc3c(N(c4ccc(-c5ccccc5)cc4)c4ccc(-c5ccccc5)cc4)cccc3c21 ; CC1(C)c2ccccc2-c2c1ccc1sc3c(N(c4ccccc4)c4ccc(-c5ccc6ccccc6c5)cc4)cccc3c21]

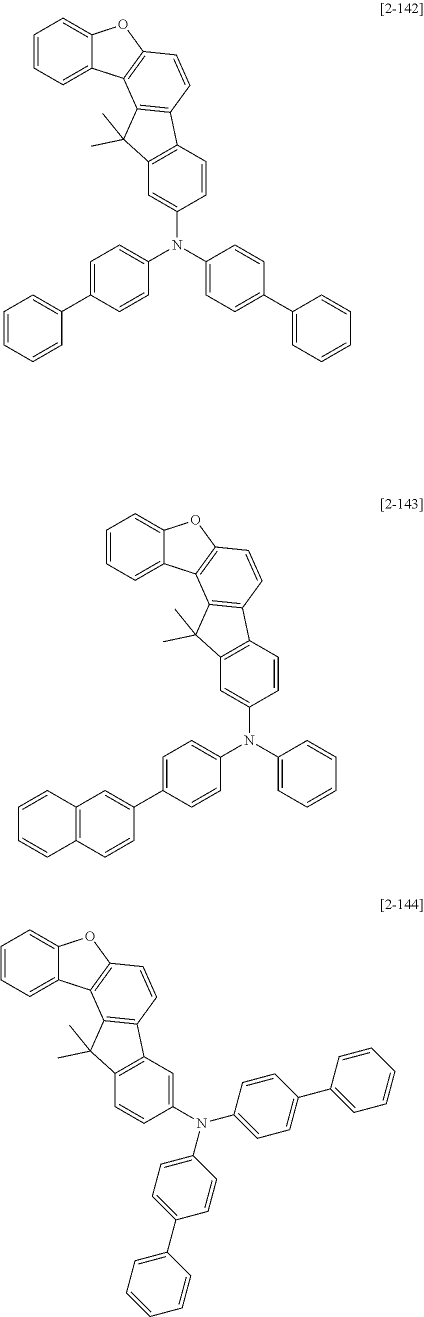 [SMILES: CC1(C)c2cc(N(c3ccc(-c4ccccc4)cc3)c3ccc(-c4ccccc4)cc3)ccc2-c2ccc3oc4ccccc4c3c21 ; CC1(C)c2cc(N(c3ccccc3)c3ccc(-c4ccc5ccccc5c4)cc3)ccc2-c2ccc3oc4ccccc4c3c21 ; CC1(C)c2ccc(N(c3ccc(-c4ccccc4)cc3)c3ccc(-c4ccccc4)cc3)cc2-c2ccc3oc4ccccc4c3c21]

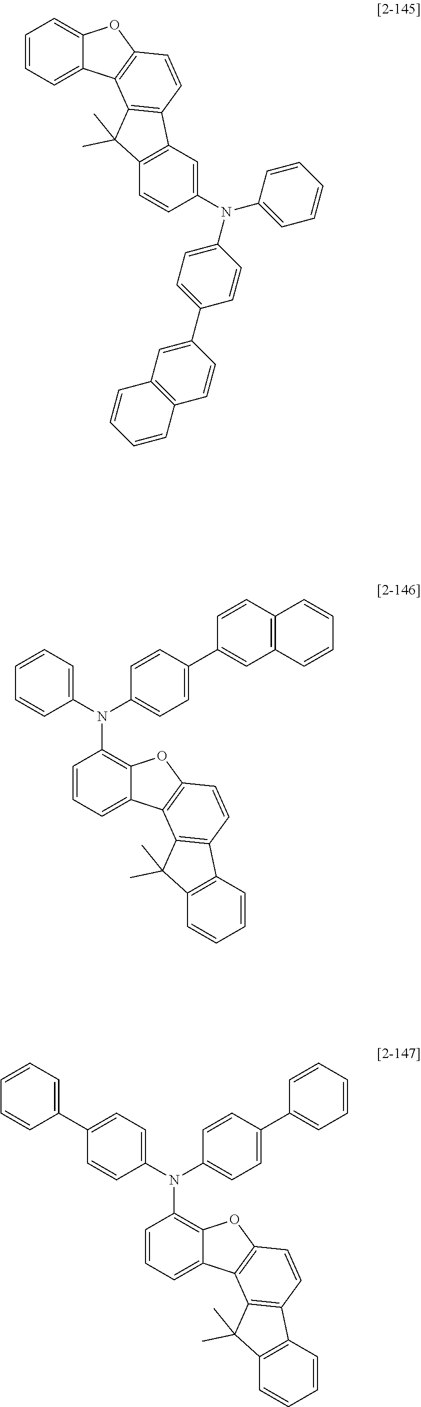 [SMILES: CC1(C)c2ccc(N(c3ccccc3)c3ccc(-c4ccc5ccccc5c4)cc3)cc2-c2ccc3oc4ccccc4c3c21 ; CC1(C)c2ccccc2-c2ccc3oc4c(N(c5ccccc5)c5ccc(-c6ccc7ccccc7c6)cc5)cccc4c3c21 ; CC1(C)c2ccccc2-c2ccc3oc4c(N(c5ccc(-c6ccccc6)cc5)c5ccc(-c6ccccc6)cc5)cccc4c3c21]

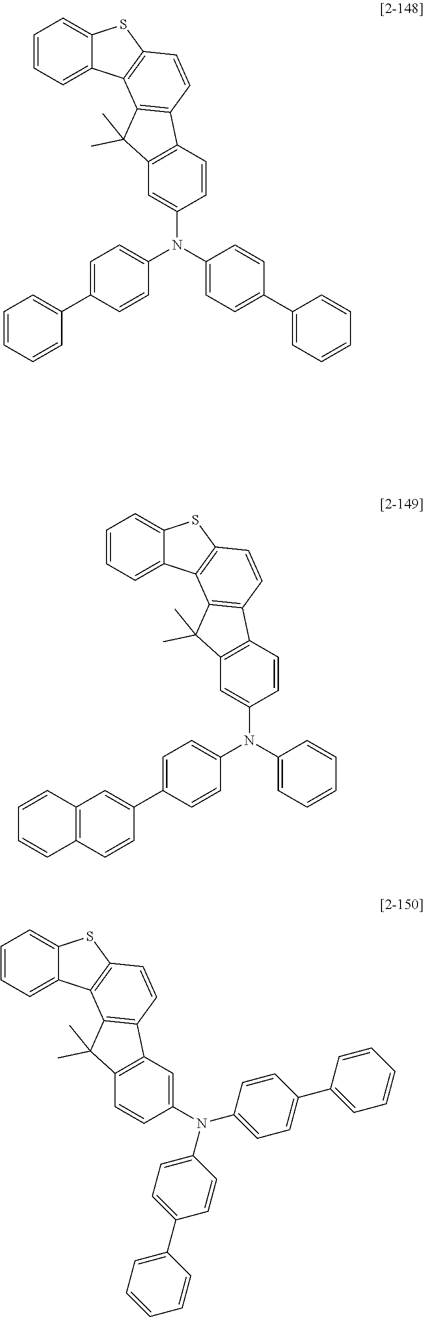 [SMILES: CC1(C)c2cc(N(c3ccc(-c4ccccc4)cc3)c3ccc(-c4ccccc4)cc3)ccc2-c2ccc3sc4ccccc4c3c21 ; CC1(C)c2cc(N(c3ccccc3)c3ccc(-c4ccc5ccccc5c4)cc3)ccc2-c2ccc3sc4ccccc4c3c21 ; CC1(C)c2ccc(N(c3ccc(-c4ccccc4)cc3)c3ccc(-c4ccccc4)cc3)cc2-c2ccc3sc4ccccc4c3c21]

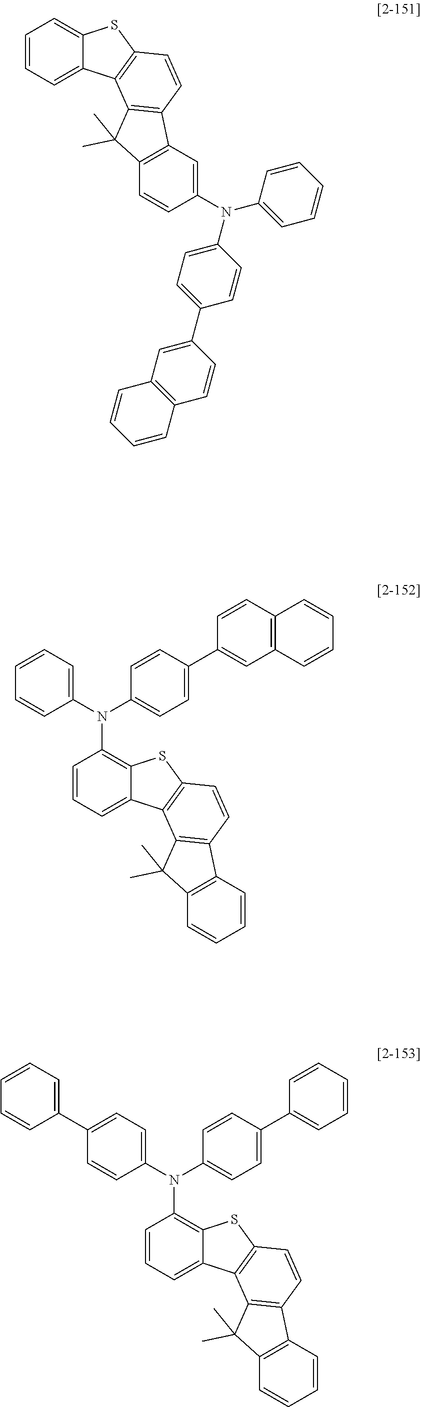 [SMILES: CC1(C)c2ccc(N(c3ccccc3)c3ccc(-c4ccc5ccccc5c4)cc3)cc2-c2ccc3sc4ccccc4c3c21 ; CC1(C)c2ccccc2-c2ccc3sc4c(N(c5ccccc5)c5ccc(-c6ccc7ccccc7c6)cc5)cccc4c3c21 ; CC1(C)c2ccccc2-c2ccc3sc4c(N(c5ccc(-c6ccccc6)cc5)c5ccc(-c6ccccc6)cc5)cccc4c3c21]

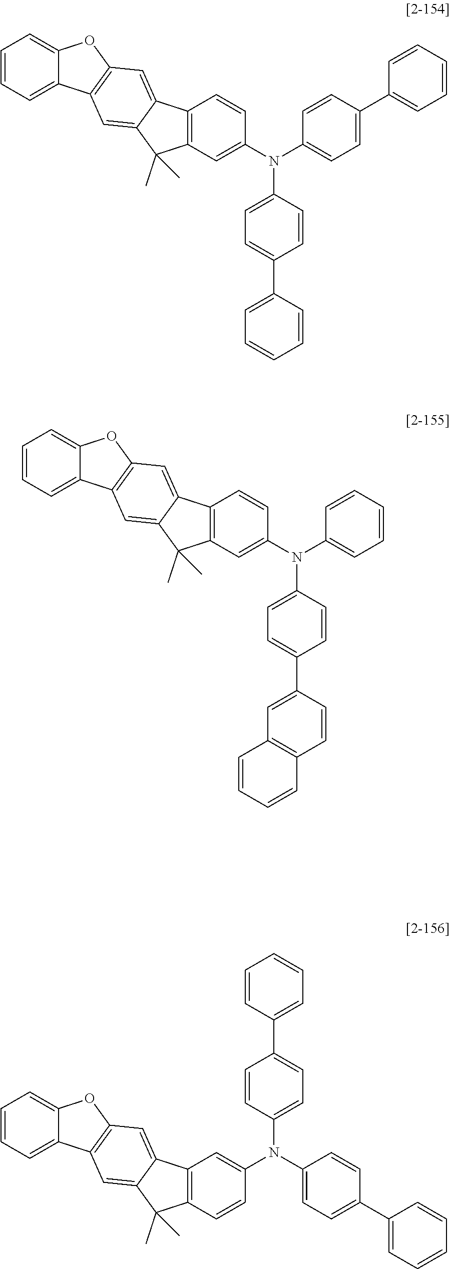 [SMILES: CC1(C)c2cc(N(c3ccc(-c4ccccc4)cc3)c3ccc(-c4ccccc4)cc3)ccc2-c2cc3oc4ccccc4c3cc21 ; CC1(C)c2cc(N(c3ccccc3)c3ccc(-c4ccc5ccccc5c4)cc3)ccc2-c2cc3oc4ccccc4c3cc21 ; CC1(C)c2ccc(N(c3ccc(-c4ccccc4)cc3)c3ccc(-c4ccccc4)cc3)cc2-c2cc3oc4ccccc4c3cc21]

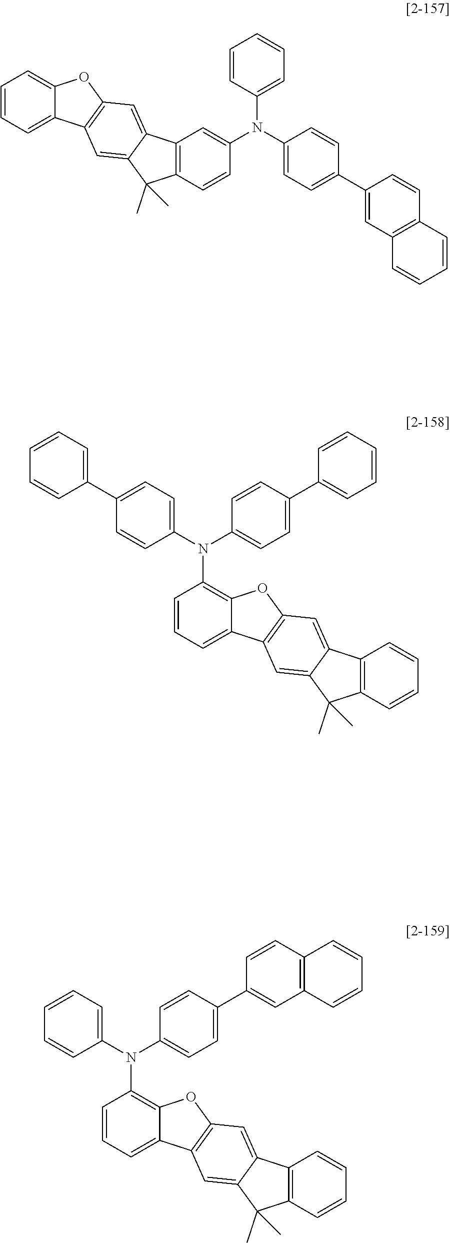 [SMILES: CC1(C)c2ccc(N(c3ccccc3)c3ccc(-c4ccc5ccccc5c4)cc3)cc2-c2cc3oc4ccccc4c3cc21 ; CC1(C)c2ccccc2-c2cc3oc4c(N(c5ccc(-c6ccccc6)cc5)c5ccc(-c6ccccc6)cc5)cccc4c3cc21 ; CC1(C)c2ccccc2-c2cc3oc4c(N(c5ccccc5)c5ccc(-c6ccc7ccccc7c6)cc5)cccc4c3cc21]

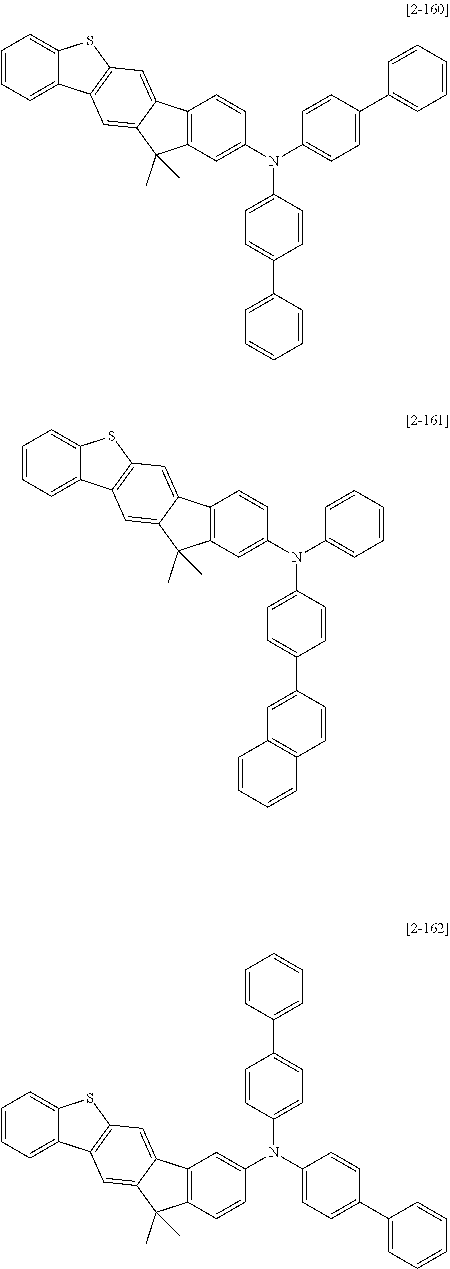 [SMILES: CC1(C)c2cc(N(c3ccc(-c4ccccc4)cc3)c3ccc(-c4ccccc4)cc3)ccc2-c2cc3sc4ccccc4c3cc21 ; CC1(C)c2cc(N(c3ccccc3)c3ccc(-c4ccc5ccccc5c4)cc3)ccc2-c2cc3sc4ccccc4c3cc21 ; CC1(C)c2ccc(N(c3ccc(-c4ccccc4)cc3)c3ccc(-c4ccccc4)cc3)cc2-c2cc3sc4ccccc4c3cc21]

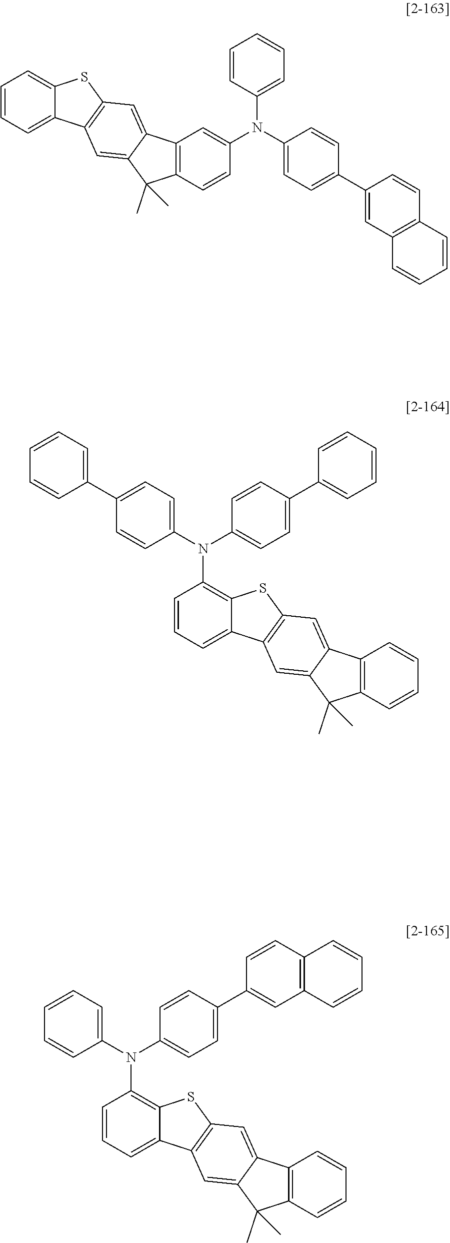 [SMILES: CC1(C)c2ccc(N(c3ccccc3)c3ccc(-c4ccc5ccccc5c4)cc3)cc2-c2cc3sc4ccccc4c3cc21 ; CC1(C)c2ccccc2-c2cc3sc4c(N(c5ccc(-c6ccccc6)cc5)c5ccc(-c6ccccc6)cc5)cccc4c3cc21 ; CC1(C)c2ccccc2-c2cc3sc4c(N(c5ccccc5)c5ccc(-c6ccc7ccccc7c6)cc5)cccc4c3cc21]

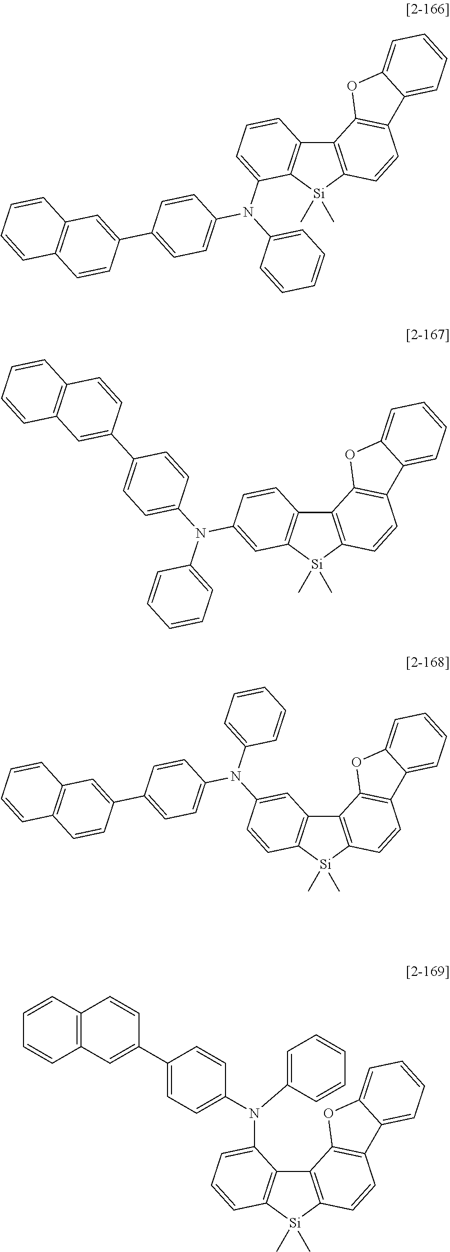 [SMILES: C[Si]1(C)c2ccc3c(oc4ccccc43)c2-c2cccc(N(c3ccccc3)c3ccc(-c4ccc5ccccc5c4)cc3)c21 ; C[Si]1(C)c2cc(N(c3ccccc3)c3ccc(-c4ccc5ccccc5c4)cc3)ccc2-c2c1ccc1c2oc2ccccc21 ; C[Si]1(C)c2ccc(N(c3ccccc3)c3ccc(-c4ccc5ccccc5c4)cc3)cc2-c2c1ccc1c2oc2ccccc21 ; C[Si]1(C)c2cccc(N(c3ccccc3)c3ccc(-c4ccc5ccccc5c4)cc3)c2-c2c1ccc1c2oc2ccccc21]

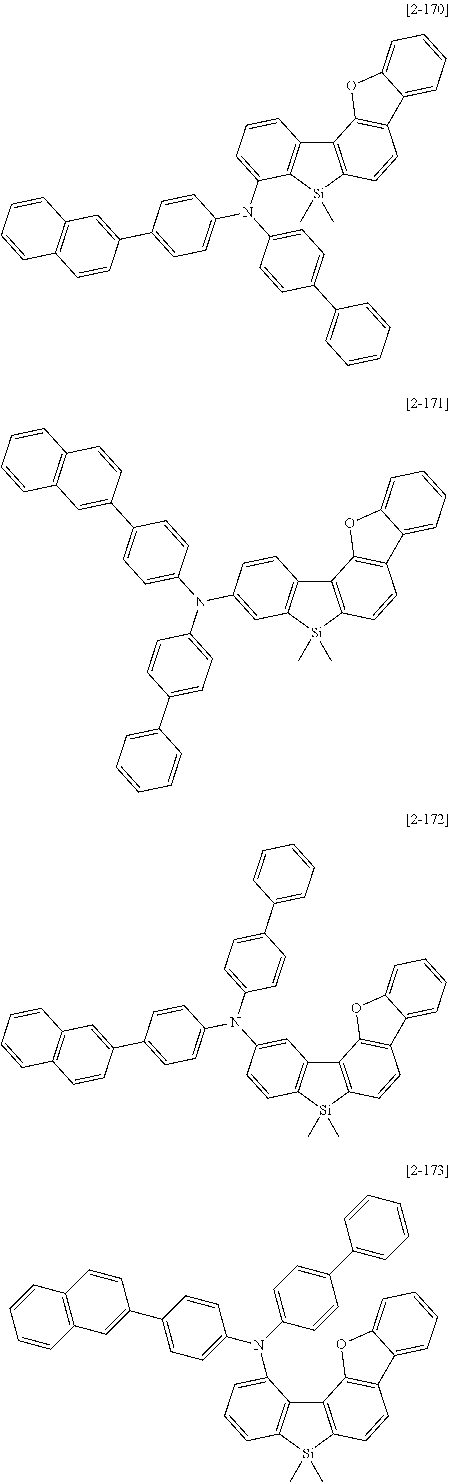 [SMILES: C[Si]1(C)c2ccc3c(oc4ccccc43)c2-c2cccc(N(c3ccc(-c4ccccc4)cc3)c3ccc(-c4ccc5ccccc5c4)cc3)c21 ; C[Si]1(C)c2cc(N(c3ccc(-c4ccccc4)cc3)c3ccc(-c4ccc5ccccc5c4)cc3)ccc2-c2c1ccc1c2oc2ccccc21 ; C[Si]1(C)c2ccc(N(c3ccc(-c4ccccc4)cc3)c3ccc(-c4ccc5ccccc5c4)cc3)cc2-c2c1ccc1c2oc2ccccc21 ; C[Si]1(C)c2cccc(N(c3ccc(-c4ccccc4)cc3)c3ccc(-c4ccc5ccccc5c4)cc3)c2-c2c1ccc1c2oc2ccccc21]

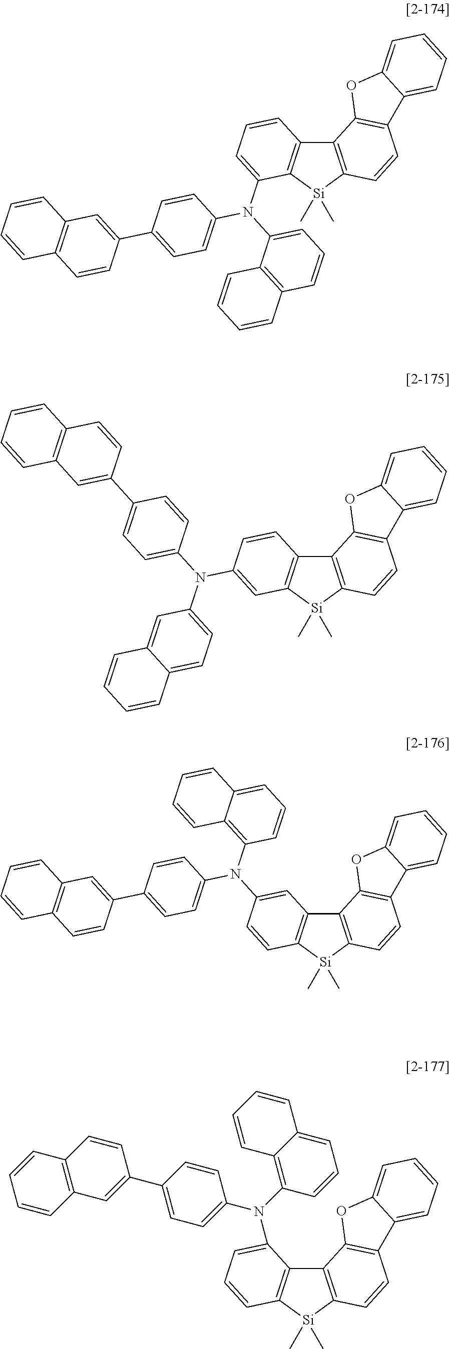 [SMILES: C[Si]1(C)c2ccc3c(oc4ccccc43)c2-c2cccc(N(c3ccc(-c4ccc5ccccc5c4)cc3)c3cccc4ccccc34)c21 ; C[Si]1(C)c2cc(N(c3ccc(-c4ccc5ccccc5c4)cc3)c3ccc4ccccc4c3)ccc2-c2c1ccc1c2oc2ccccc21 ; C[Si]1(C)c2ccc(N(c3ccc(-c4ccc5ccccc5c4)cc3)c3cccc4ccccc34)cc2-c2c1ccc1c2oc2ccccc21 ; C[Si]1(C)c2cccc(N(c3ccc(-c4ccc5ccccc5c4)cc3)c3cccc4ccccc34)c2-c2c1ccc1c2oc2ccccc21]

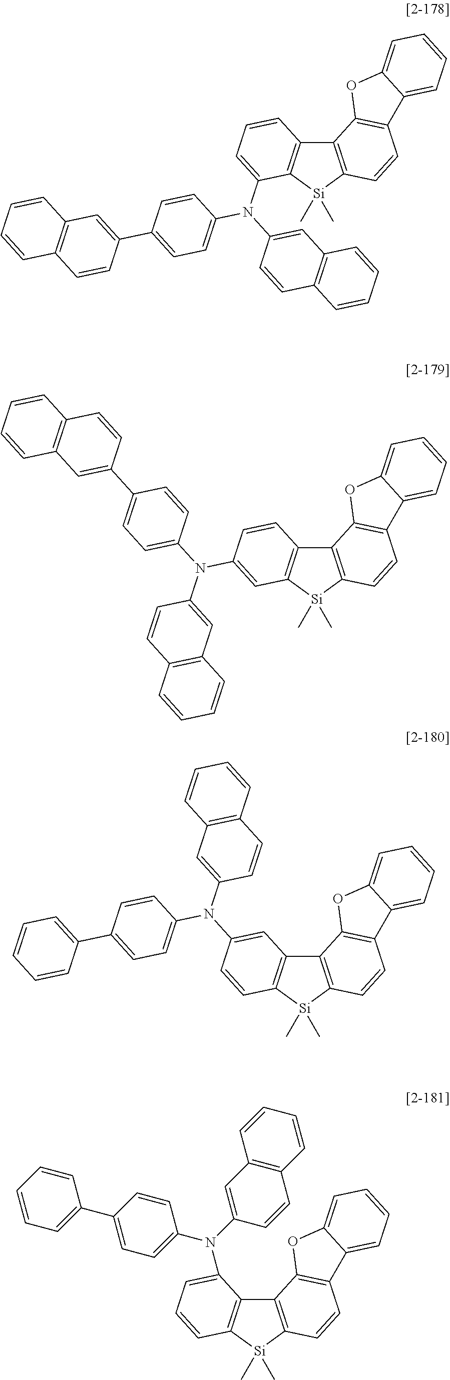 [SMILES: C[Si]1(C)c2ccc3c(oc4ccccc43)c2-c2cccc(N(c3ccc(-c4ccc5ccccc5c4)cc3)c3ccc4ccccc4c3)c21 ; C[Si]1(C)c2cc(N(c3ccc(-c4ccc5ccccc5c4)cc3)c3ccc4ccccc4c3)ccc2-c2c1ccc1c2oc2ccccc21 ; C[Si]1(C)c2ccc(N(c3ccc(-c4ccccc4)cc3)c3ccc4ccccc4c3)cc2-c2c1ccc1c2oc2ccccc21 ; C[Si]1(C)c2cccc(N(c3ccc(-c4ccccc4)cc3)c3ccc4ccccc4c3)c2-c2c1ccc1c2oc2ccccc21]

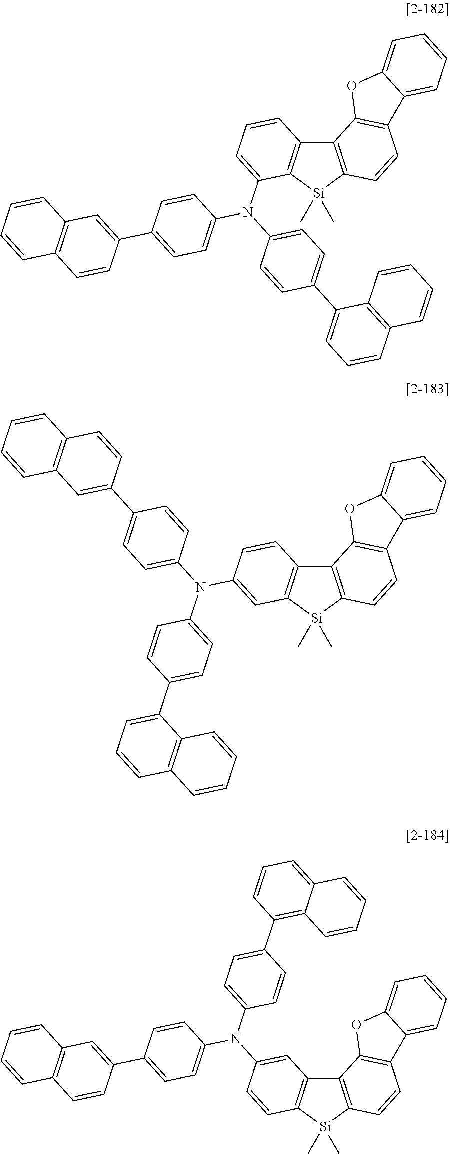 [SMILES: C[Si]1(C)c2ccc3c(oc4ccccc43)c2-c2cccc(N(c3ccc(-c4ccc5ccccc5c4)cc3)c3ccc(-c4cccc5ccccc45)cc3)c21 ; C[Si]1(C)c2cc(N(c3ccc(-c4ccc5ccccc5c4)cc3)c3ccc(-c4cccc5ccccc45)cc3)ccc2-c2c1ccc1c2oc2ccccc21 ; C[Si]1(C)c2ccc(N(c3ccc(-c4ccc5ccccc5c4)cc3)c3ccc(-c4cccc5ccccc45)cc3)cc2-c2c1ccc1c2oc2ccccc21]

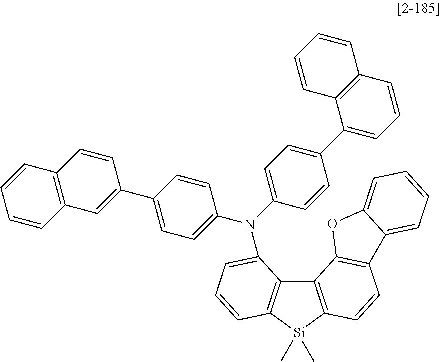 [SMILES: C[Si]1(C)c2cccc(N(c3ccc(-c4ccc5ccccc5c4)cc3)c3ccc(-c4cccc5ccccc45)cc3)c2-c2c1ccc1c2oc2ccccc21]

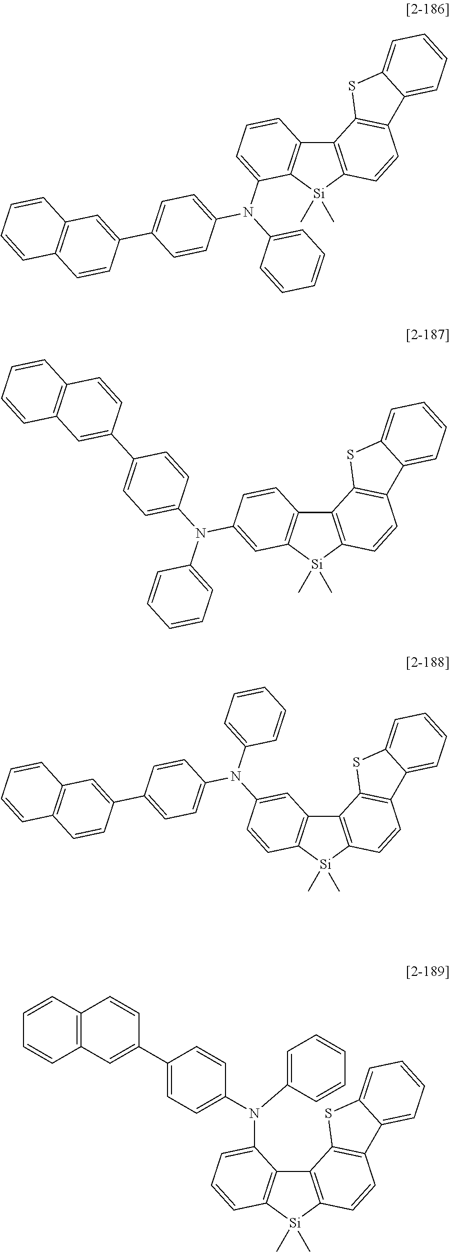 [SMILES: C[Si]1(C)c2ccc3c(sc4ccccc43)c2-c2cccc(N(c3ccccc3)c3ccc(-c4ccc5ccccc5c4)cc3)c21 ; C[Si]1(C)c2cc(N(c3ccccc3)c3ccc(-c4ccc5ccccc5c4)cc3)ccc2-c2c1ccc1c2sc2ccccc21 ; C[Si]1(C)c2ccc(N(c3ccccc3)c3ccc(-c4ccc5ccccc5c4)cc3)cc2-c2c1ccc1c2sc2ccccc21 ; C[Si]1(C)c2cccc(N(c3ccccc3)c3ccc(-c4ccc5ccccc5c4)cc3)c2-c2c1ccc1c2sc2ccccc21]

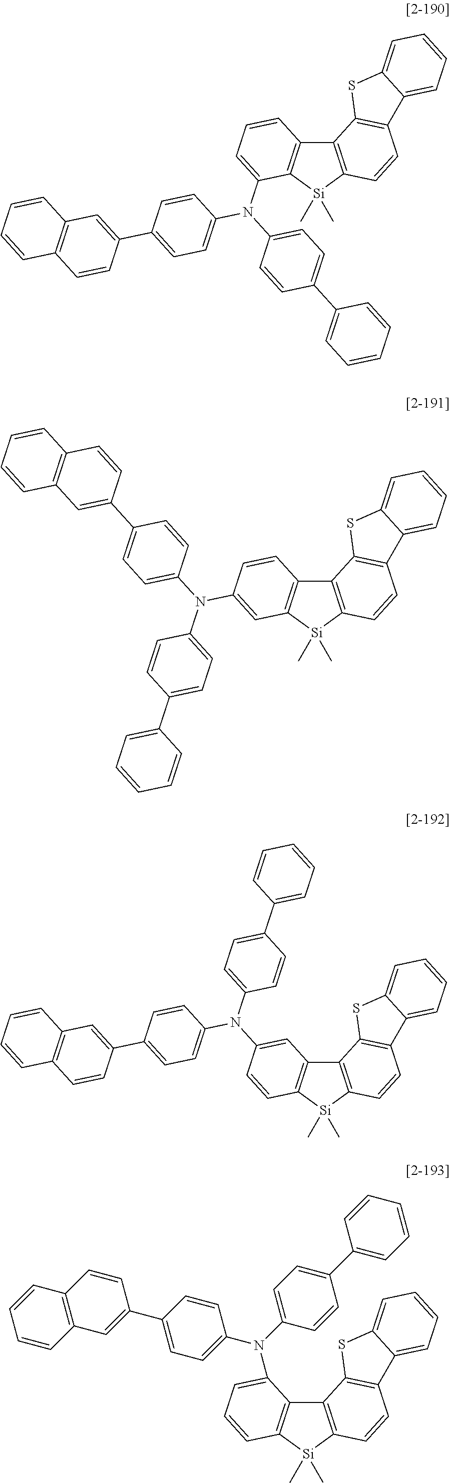 [SMILES: C[Si]1(C)c2ccc3c(sc4ccccc43)c2-c2cccc(N(c3ccc(-c4ccccc4)cc3)c3ccc(-c4ccc5ccccc5c4)cc3)c21 ; C[Si]1(C)c2cc(N(c3ccc(-c4ccccc4)cc3)c3ccc(-c4ccc5ccccc5c4)cc3)ccc2-c2c1ccc1c2sc2ccccc21 ; C[Si]1(C)c2ccc(N(c3ccc(-c4ccccc4)cc3)c3ccc(-c4ccc5ccccc5c4)cc3)cc2-c2c1ccc1c2sc2ccccc21 ; C[Si]1(C)c2cccc(N(c3ccc(-c4ccccc4)cc3)c3ccc(-c4ccc5ccccc5c4)cc3)c2-c2c1ccc1c2sc2ccccc21]

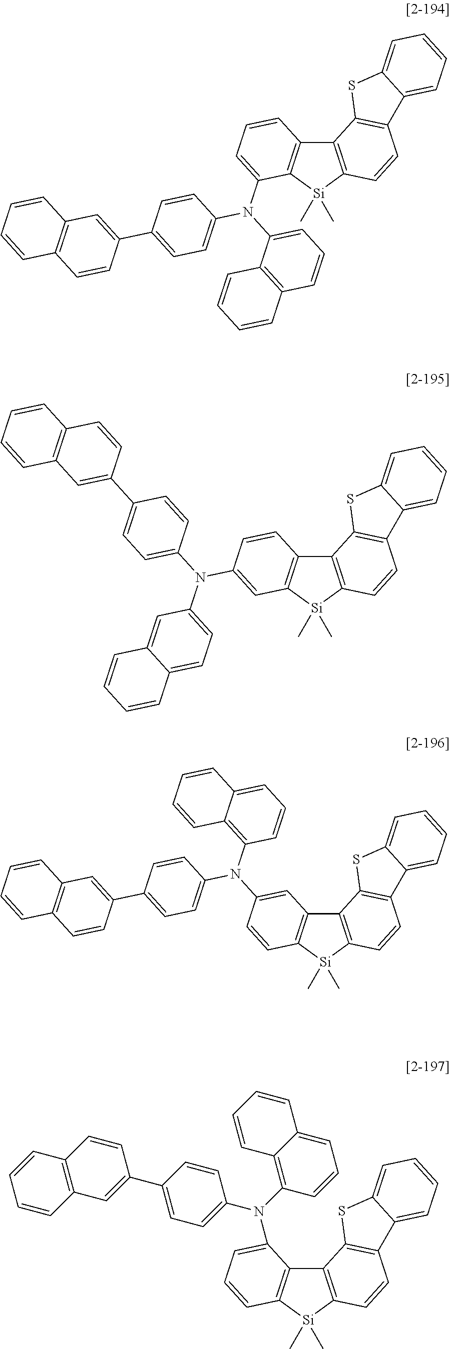 [SMILES: C[Si]1(C)c2ccc3c(sc4ccccc43)c2-c2cccc(N(c3ccc(-c4ccc5ccccc5c4)cc3)c3cccc4ccccc34)c21 ; C[Si]1(C)c2cc(N(c3ccc(-c4ccc5ccccc5c4)cc3)c3ccc4ccccc4c3)ccc2-c2c1ccc1c2sc2ccccc21 ; C[Si]1(C)c2ccc(N(c3ccc(-c4ccc5ccccc5c4)cc3)c3cccc4ccccc34)cc2-c2c1ccc1c2sc2ccccc21 ; C[Si]1(C)c2cccc(N(c3ccc(-c4ccc5ccccc5c4)cc3)c3cccc4ccccc34)c2-c2c1ccc1c2sc2ccccc21]

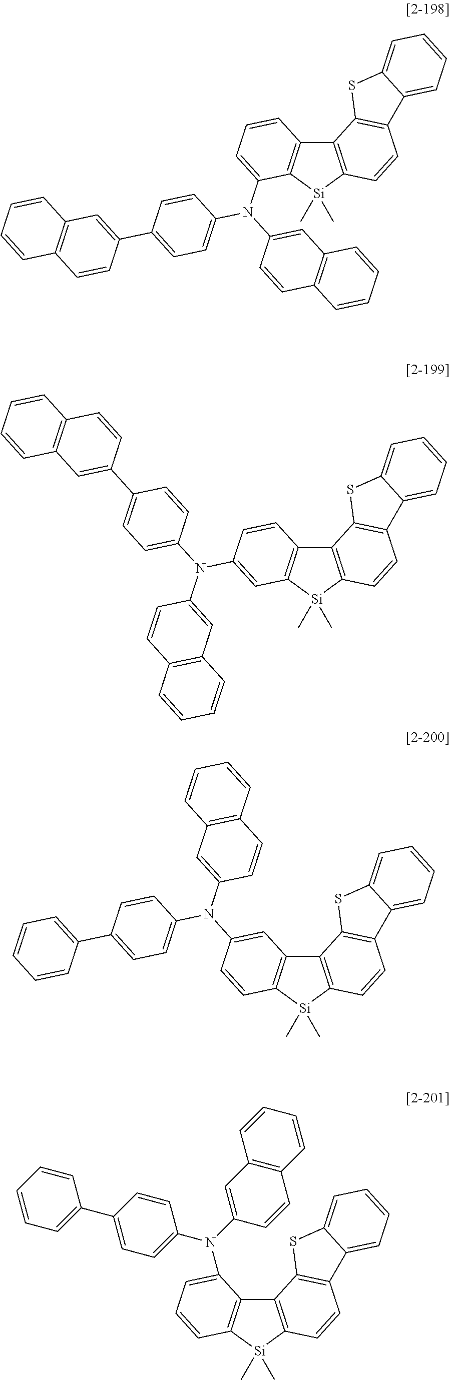 [SMILES: C[Si]1(C)c2ccc3c(sc4ccccc43)c2-c2cccc(N(c3ccc(-c4ccc5ccccc5c4)cc3)c3ccc4ccccc4c3)c21 ; C[Si]1(C)c2cc(N(c3ccc(-c4ccc5ccccc5c4)cc3)c3ccc4ccccc4c3)ccc2-c2c1ccc1c2sc2ccccc21 ; C[Si]1(C)c2ccc(N(c3ccc(-c4ccccc4)cc3)c3ccc4ccccc4c3)cc2-c2c1ccc1c2sc2ccccc21 ; C[Si]1(C)c2cccc(N(c3ccc(-c4ccccc4)cc3)c3ccc4ccccc4c3)c2-c2c1ccc1c2sc2ccccc21]

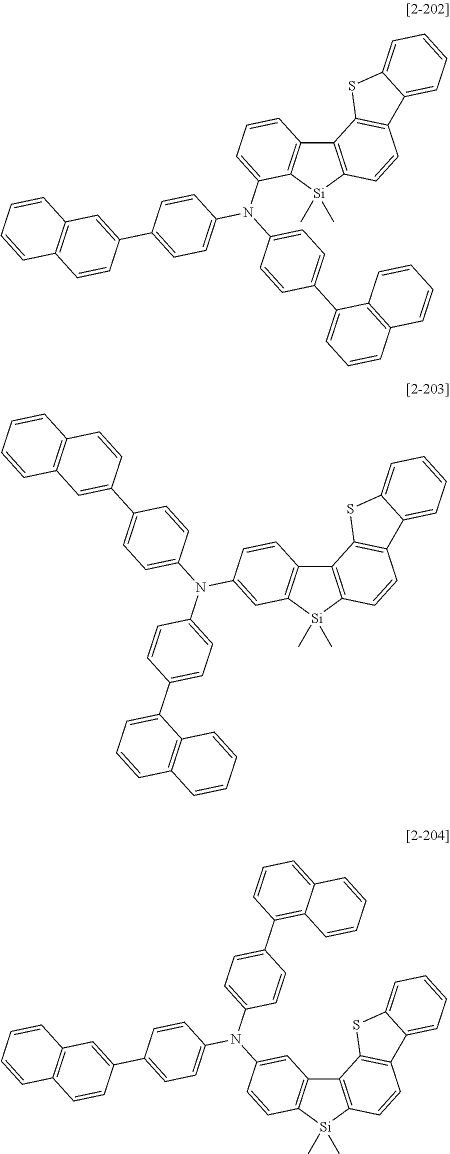 [SMILES: C[Si]1(C)c2ccc3c(sc4ccccc43)c2-c2cccc(N(c3ccc(-c4ccc5ccccc5c4)cc3)c3ccc(-c4cccc5ccccc45)cc3)c21 ; C[Si]1(C)c2cc(N(c3ccc(-c4ccc5ccccc5c4)cc3)c3ccc(-c4cccc5ccccc45)cc3)ccc2-c2c1ccc1c2sc2ccccc21 ; C[Si]1(C)c2ccc(N(c3ccc(-c4ccc5ccccc5c4)cc3)c3ccc(-c4cccc5ccccc45)cc3)cc2-c2c1ccc1c2sc2ccccc21]

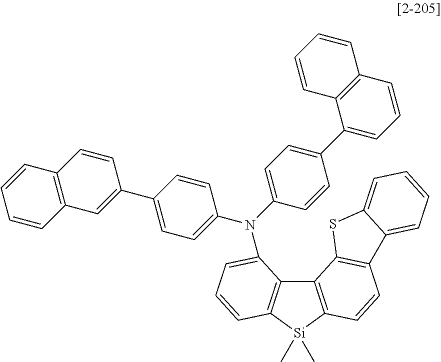 [SMILES: C[Si]1(C)c2cccc(N(c3ccc(-c4ccc5ccccc5c4)cc3)c3ccc(-c4cccc5ccccc45)cc3)c2-c2c1ccc1c2sc2ccccc21]

The first compound and the second compound may be included, for example, in a weight ratio of about 1:99 to about 99:1. Within the range, a desirable weight ratio may be adjusted using an electron transport capability of the first compound and a hole transport capability of the second compound to realize bipolar characteristics and thus to improve efficiency and life-span. Within the range, they may be for example included in a weight ratio of about 10:90 to about 90:10, about 20:80 to about 80:20, for example, about 20:80 to about 70:30, about 20:80 to about 60:40, and about 30:70 to about 60:40. As a specific example, they may be included in a weight ratio of about 40:60, about 50:50, or about 60:40.

One or more compounds may be further included in addition to the aforementioned first compound and second compound.

The aforementioned compound for the organic optoelectronic device or composition for an organic optoelectronic device may be a composition further including a dopant.

The dopant may be, for example, a phosphorescent dopant, for example, a red, green, or blue phosphorescent dopant, for example, a red or green phosphorescent dopant.

The dopant is a material mixed with the compound or composition for the organic optoelectronic device in small amount to cause light emission and may be generally a material such as a metal complex that emits light by multiple excitation into a triplet or more. The dopant may be, for example, an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example, a compound represented by Chemical Formula Z.

L⁷MX⁴ [Chemical Formula Z]

In Chemical Formula Z,

M is a metal, and, L⁷ and X⁴ are the same as or different from each other, and are ligands forming a complex compound with M,

the M may be, for example, Ir, Pt, Os, Ti, Zr, Hf, Eu, Tb, Tm, Fe, Co, Ni, Ru, Rh, Pd, or a combination thereof, and

L⁷ and X⁴ may be, for example, a bidentate ligand.

Examples of the ligands represented by L⁷ and X⁴ may be selected from the chemical formulas of Group A.

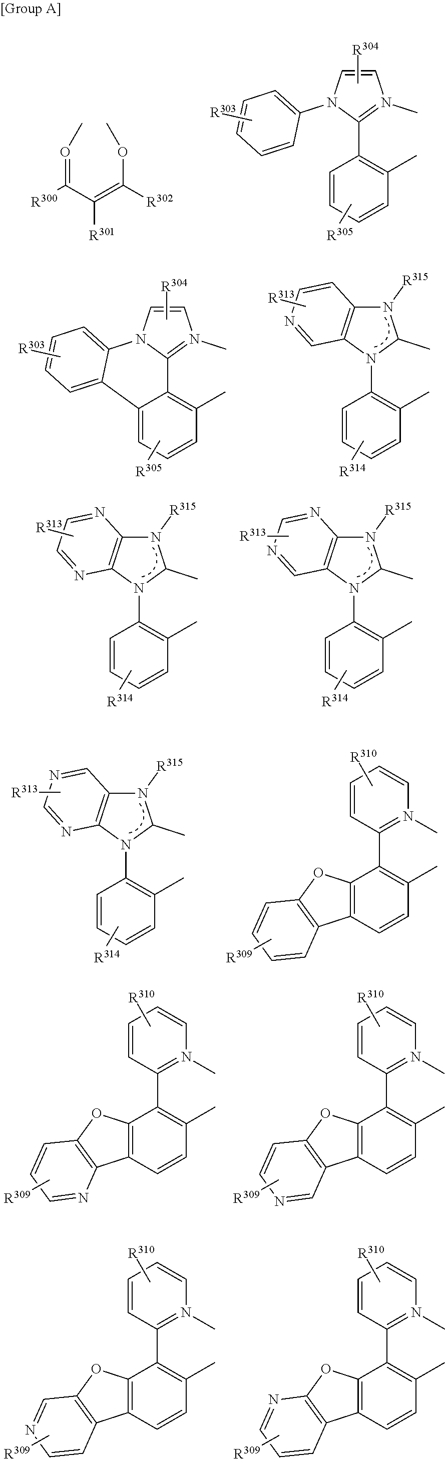

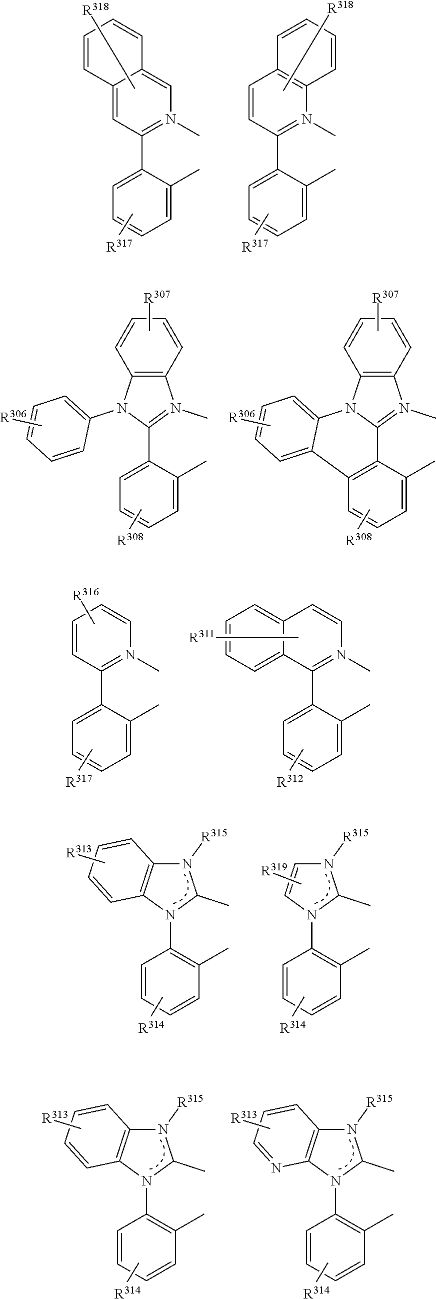

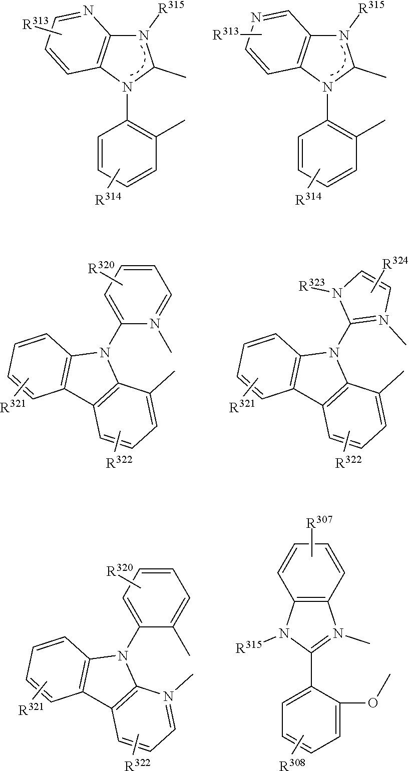

In Group A,

R³⁰⁰ to R³⁰² are each independently hydrogen, deuterium, a C1 to C30 alkyl group that is substituted or unsubstituted with a halogen, a C6 to C30 aryl group that is substituted or unsubstituted with a C1 to C30 alkyl, or a halogen, and

R³⁰³ to R³²⁴ are each independently hydrogen, deuterium, halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SFs, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

As an example, the composition may include a dopant represented by Chemical Formula V.

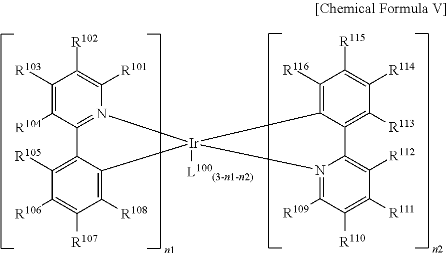

In Chemical Formula V,

R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,

R¹³² to R¹³⁴ are each independently a C1 to C6 alkyl group,

at least one of R¹⁰¹ to R¹¹⁶ is a functional group represented by Chemical Formula V-1,

L¹⁰⁰ is a bidentate ligand of a monovalent anion, and is a ligand that coordinates to iridium through a lone pair of electrons of carbon or heteroatom,

n1 and n2 are each independently any one of integers of 0 to 3, and

n1+n2 is any one of integers of 1 to 3.

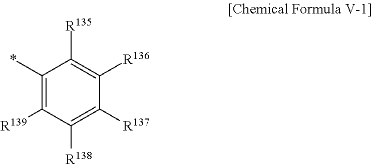

In Chemical Formula V-1,

R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and

* means a portion linked to a carbon atom.

In an example embodiment, a dopant represented by Chemical Formula z-1 may be included in the composition.

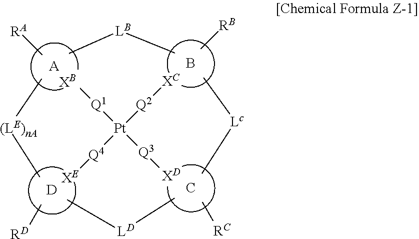

In Chemical Formula Z-1,

rings A, B, C, and D each independently represent a 5-membered or 6-membered carbocyclic or heterocyclic ring;

R^{A}, R^{B}, R^{C}, and R^{D} each independently represent mono-, di-, tri-, or tetra-substitution, or may be unsubstituted or omitted;

L^{B}, L^{C}, and L^{D} are each independent selected from a direct bond, BR, NR, PR, O, S, Se, C═O, S═O, SO₂, CRR′, SiRR′, GeRR′, and a combination thereof,

when nA is 1, L^{E} is selected from a direct bond, BR, NR, PR, O, S, Se, C═O, S═O, SO₂, CRR′, SiRR′, GeRR′, and a combination thereof,

when nA is 0, L^{E} does not exist; and

R^{A}, R^{B}, R^{C}, R^{D}, R, and R′ are each independently selected from hydrogen, deuterium, a halogen, an alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, and a combination thereof, any adjacent R^{A}, R^{B}, R^{C}, R^{D}, R, and R′ are optionally linked to each other to provide a ring;

X^{B}, X^{C}, X^{D}, and X^{E} are each independently selected from carbon and nitrogen; and

Q¹, Q², Q³, and Q⁴ each represent oxygen or a direct bond.

The dopant according to an example embodiment may be a platinum complex, and may be, for example, represented by Chemical Formula VI.

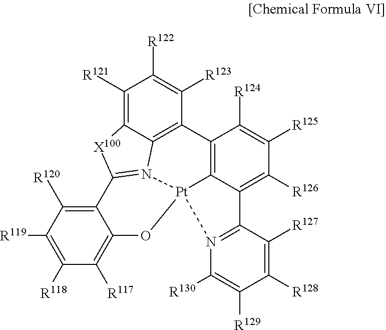

In Chemical Formula VI,

X¹⁰⁰ is selected from O, S, and NR¹³¹,

R¹¹⁷ to R¹³¹ are each independently hydrogen, deuterium, a substituted or unsubstituted C1 to C10 alkyl group, a substituted or unsubstituted C6 to C20 aryl group, or —SiR¹³²R¹³³R¹³⁴,

R¹³² to R¹³⁴ are each independently a C1 to C6 alkyl group, and

at least one of R¹¹⁷ to R¹³¹ is —SiR¹³²R¹³³R¹³⁴ or a tert-butyl group.

Hereinafter, an organic optoelectronic device including the aforementioned compound for the organic optoelectronic device or composition for the organic optoelectronic device is described.

The organic optoelectronic device may be any device to convert electrical energy into photoenergy and vice versa, and may be, for example an organic photoelectric device, an organic light emitting diode, an organic solar cell, and an organic photo-conductor drum.

Herein, an organic light emitting diode as one example of an organic optoelectronic device is described referring to the drawing.

The FIGURE is a cross-sectional view illustrating an organic light emitting diode according to an example embodiment.

Referring to the FIGURE, an organic light emitting diode 100 according to an example embodiment includes an anode 120 and a cathode 110 facing each other and an organic layer 105 between the anode 120 and cathode 110.

The anode 120 may be made of a conductor having a large work function to help hole injection, and may be for example a metal, a metal oxide and/or a conductive polymer. The anode 120 may be, for example, a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline.

The cathode 110 may be made of a conductor having a small work function to help electron injection, and may be for example a metal, a metal oxide and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, and BaF₂/Ca.

The organic layer 105 may include the aforementioned compound for the organic optoelectronic device or composition for the organic optoelectronic device.

The organic layer 105 may include a light emitting layer 130 and the light emitting layer 130 may include the aforementioned compound for the organic optoelectronic device or composition for the organic optoelectronic device.

The composition for the organic optoelectronic device further including the dopant may be, for example, a red light emitting composition.

The light emitting layer 130 may include, for example, the aforementioned first compound and second compound as phosphorescent hosts, respectively.

The organic layer may further include a charge transport region in addition to the light emitting layer.

The charge transport region may be for example a hole transport region 140.

The hole transport region 140 may further increase hole injection and/or hole mobility between the anode 120 and the light emitting layer 130 and block electrons. Specifically, the hole transport region 140 may include a hole transport layer between the anode 120 and the light emitting layer 130, and a hole transport auxiliary layer between the light emitting layer 130 and the hole transport layer.

At least one of the compounds of Group B may be included in at least one of the hole transport layer and the hole transport auxiliary layer.

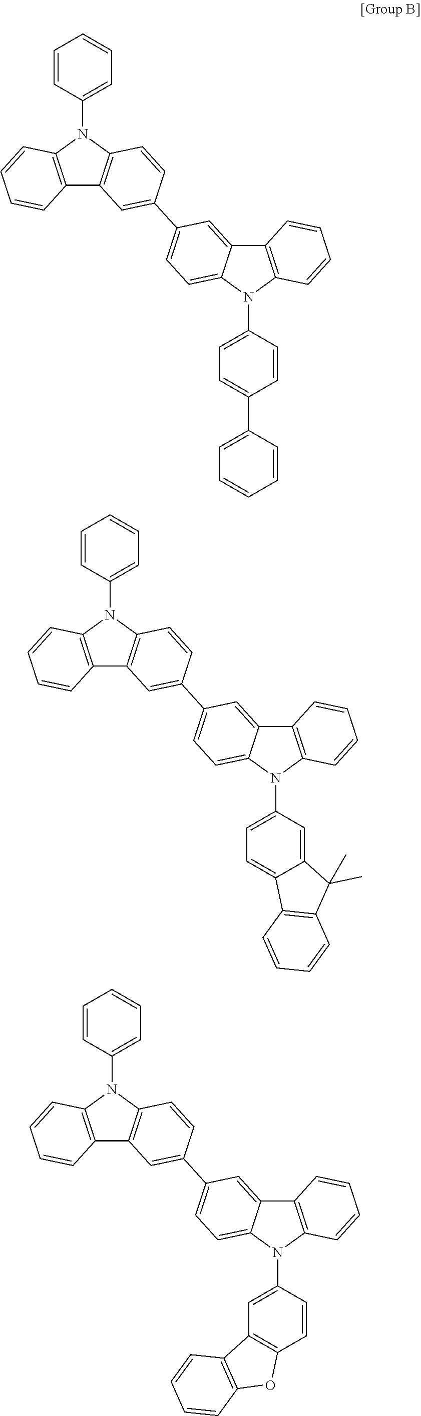 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21 ; c1ccc(-n2c3ccccc3c3cc(-c4ccc5c(c4)c4ccccc4n5-c4ccc5oc6ccccc6c5c4)ccc32)cc1]

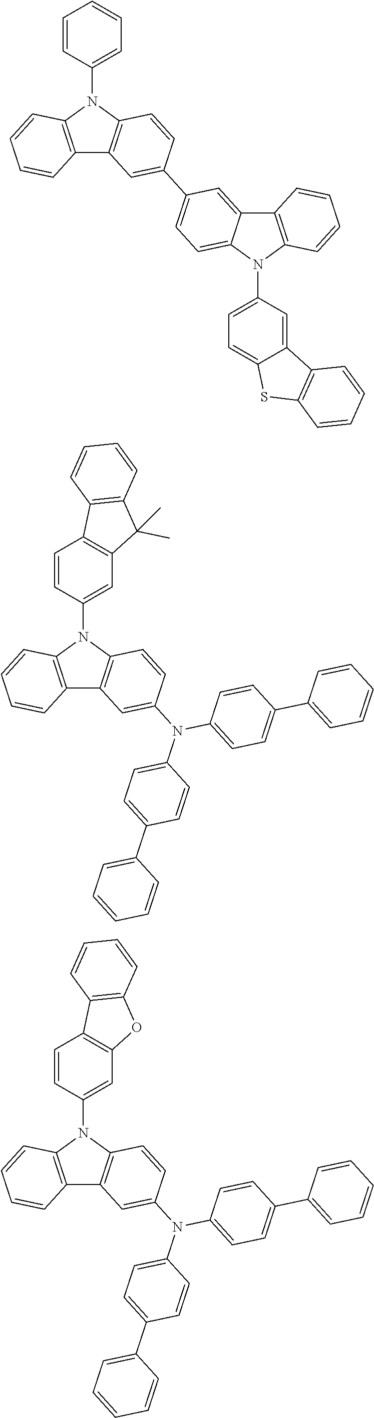

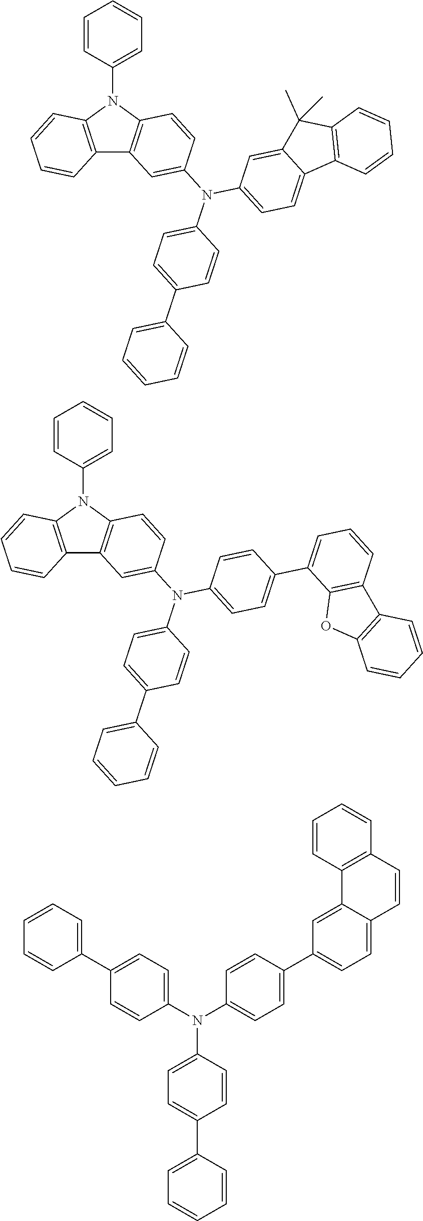 [SMILES: CC1(C)c2ccccc2-c2ccc(N(c3ccc(-c4ccccc4)cc3)c3ccc4c(c3)c3ccccc3n4-c3ccccc3)cc21 ; c1ccc(-c2ccc(N(c3ccc(-c4cccc5c4oc4ccccc45)cc3)c3ccc4c(c3)c3ccccc3n4-c3ccccc3)cc2)cc1 ; c1ccc(-c2ccc(N(c3ccc(-c4ccccc4)cc3)c3ccc(-c4ccc5ccc6ccccc6c5c4)cc3)cc2)cc1]

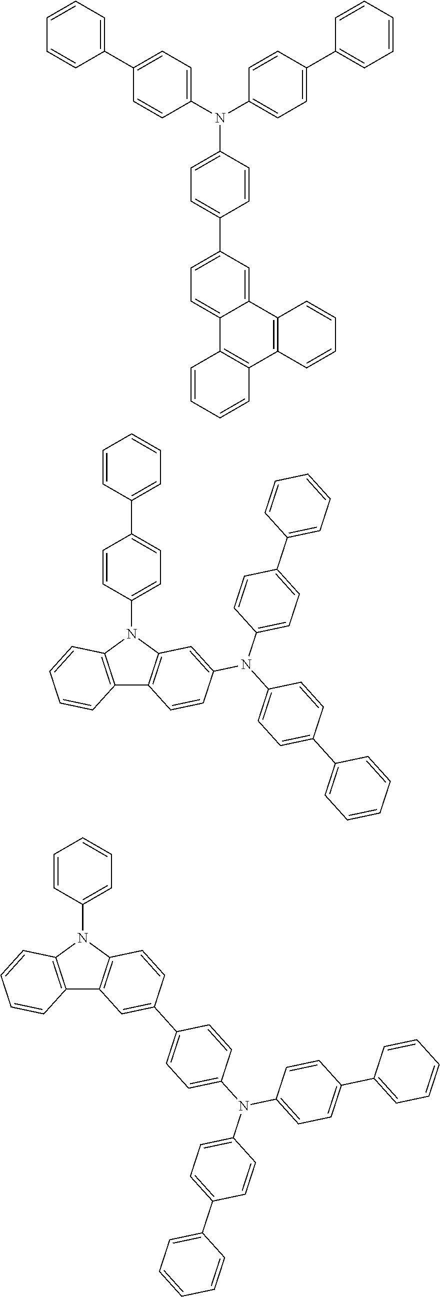 [SMILES: c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1 ; c1ccc(-c2ccc(N(c3ccc(-c4ccccc4)cc3)c3ccc(-c4ccc5c(c4)c4ccccc4n5-c4ccccc4)cc3)cc2)cc1]

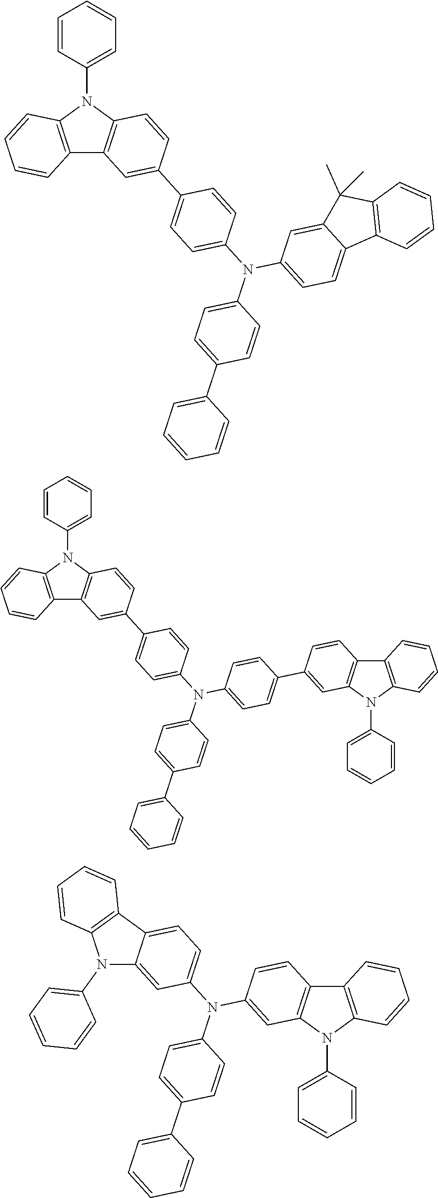

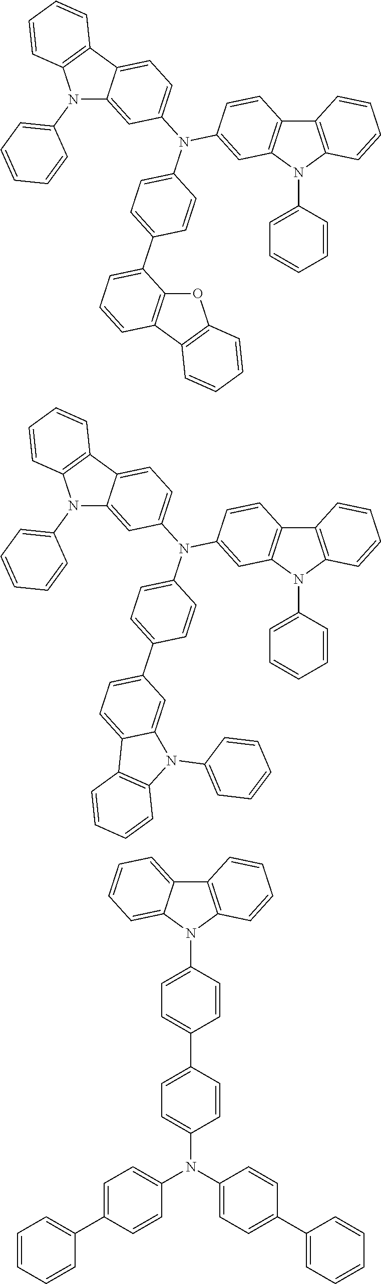

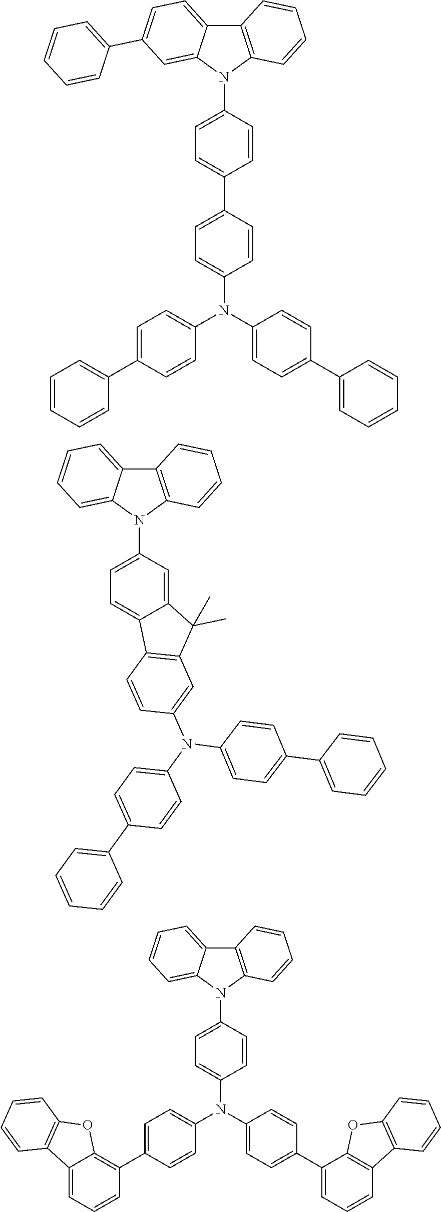

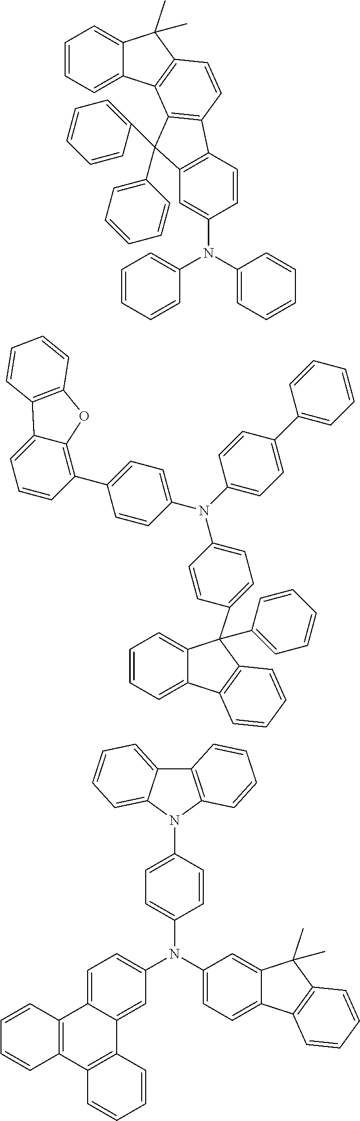

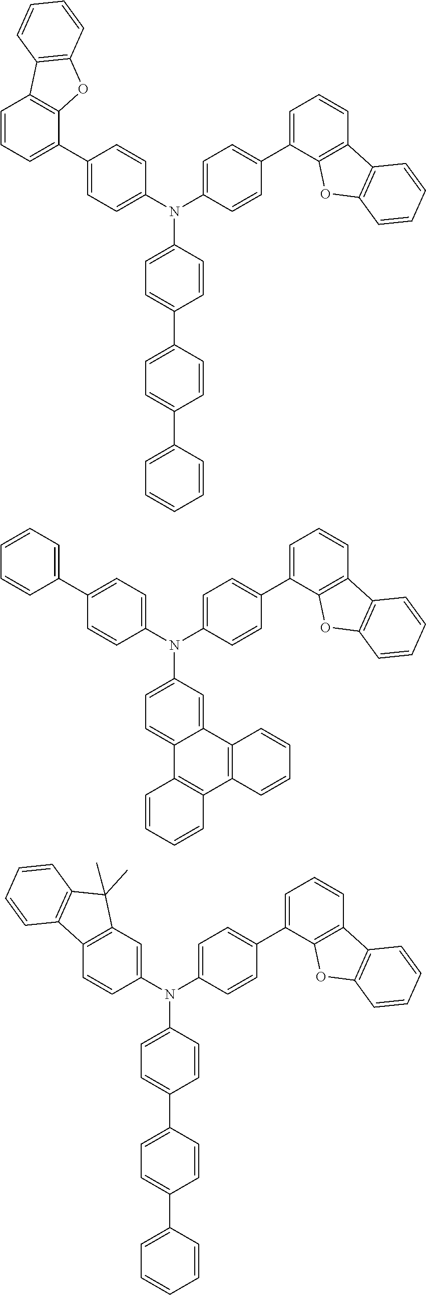

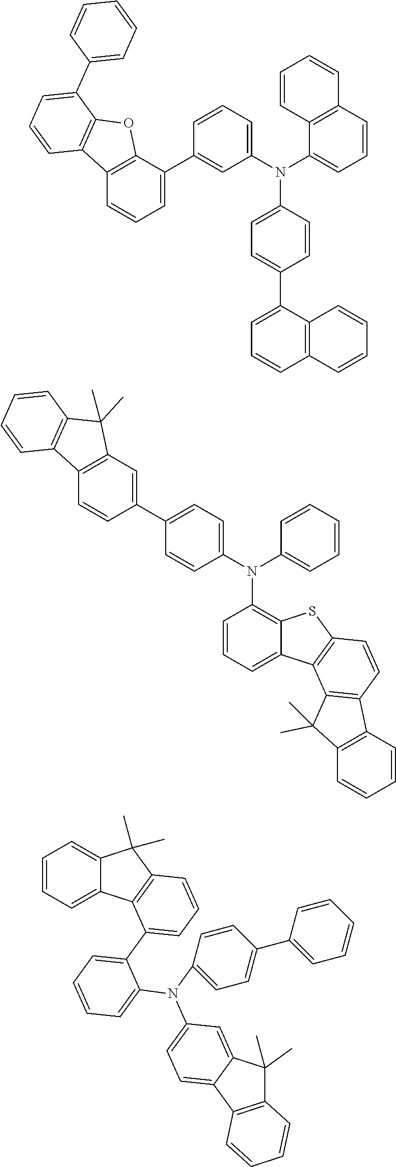 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

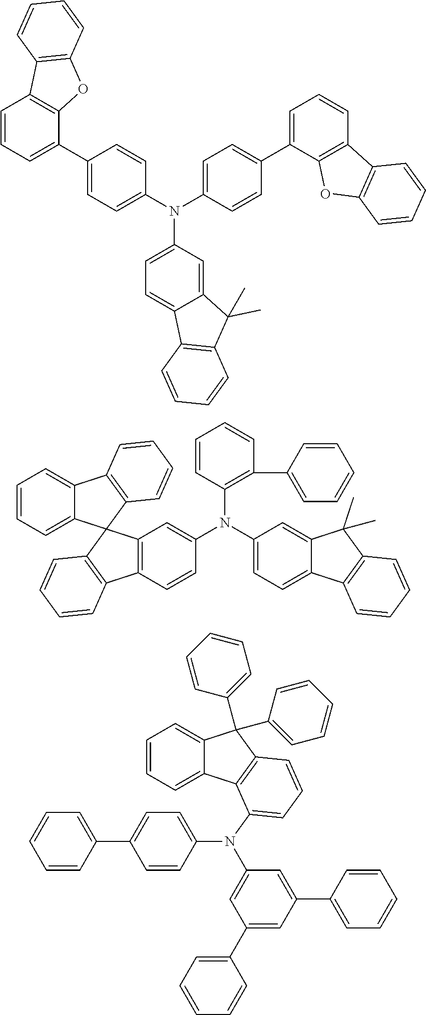

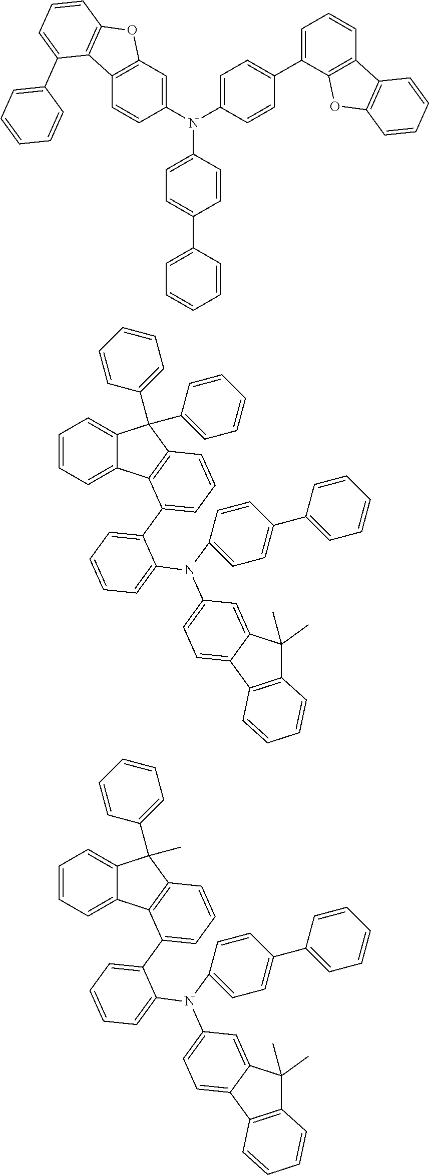

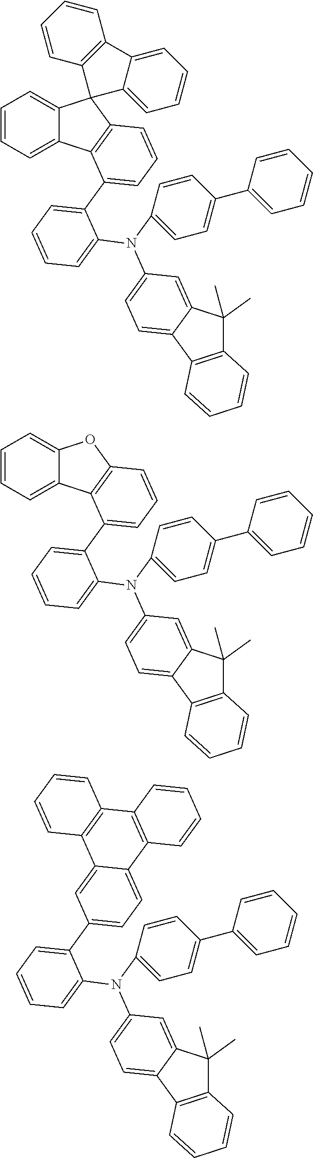

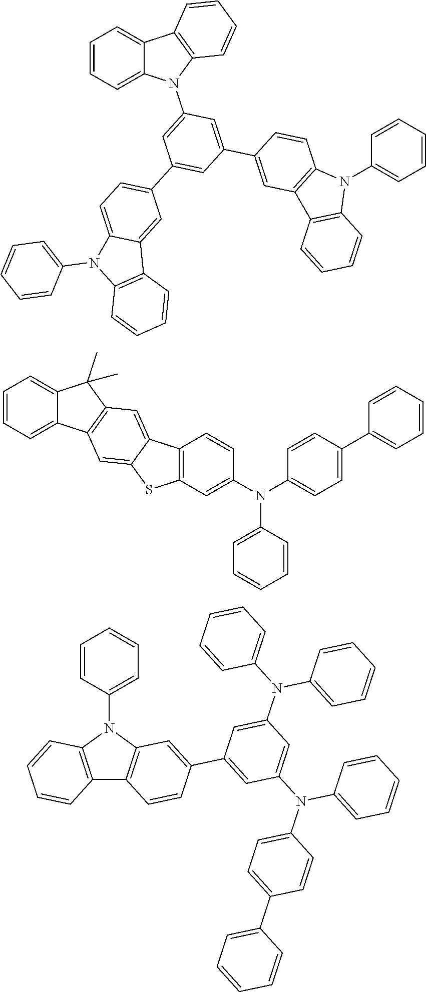

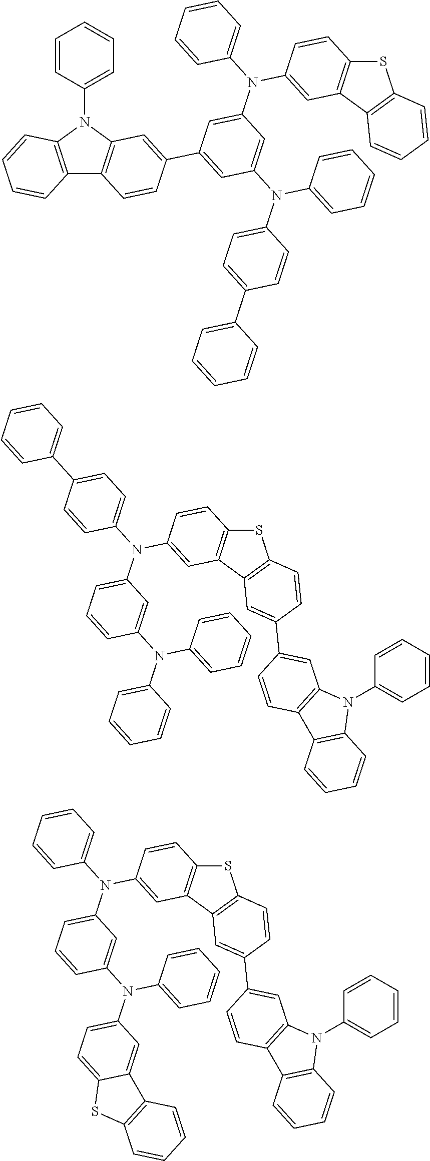 [SMILES: c1ccc(-c2ccc(N(c3ccccc3)c3cc(-c4ccc5c6ccccc6n(-c6ccccc6)c5c4)cc(N(c4ccccc4)c4ccc5sc6ccccc6c5c4)c3)cc2)cc1 ; c1ccc(-c2ccc(N(c3cccc(N(c4ccccc4)c4ccccc4)c3)c3ccc4sc5ccc(-c6ccc7c8ccccc8n(-c8ccccc8)c7c6)cc5c4c3)cc2)cc1 ; c1ccc(N(c2cccc(N(c3ccccc3)c3ccc4sc5ccc(-c6ccc7c8ccccc8n(-c8ccccc8)c7c6)cc5c4c3)c2)c2ccc3sc4ccccc4c3c2)cc1]

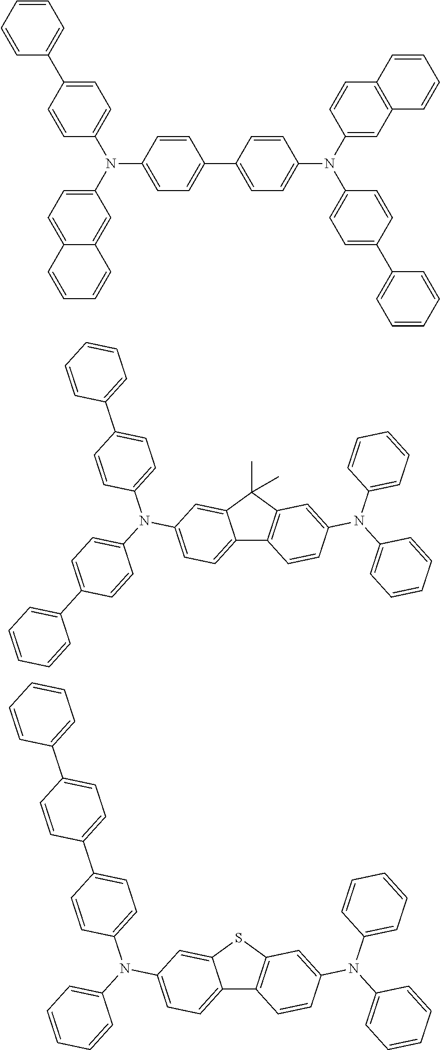

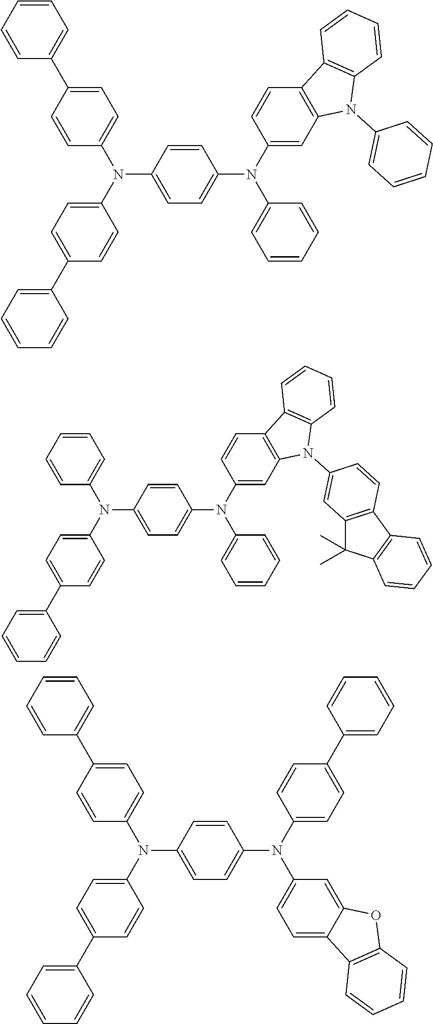

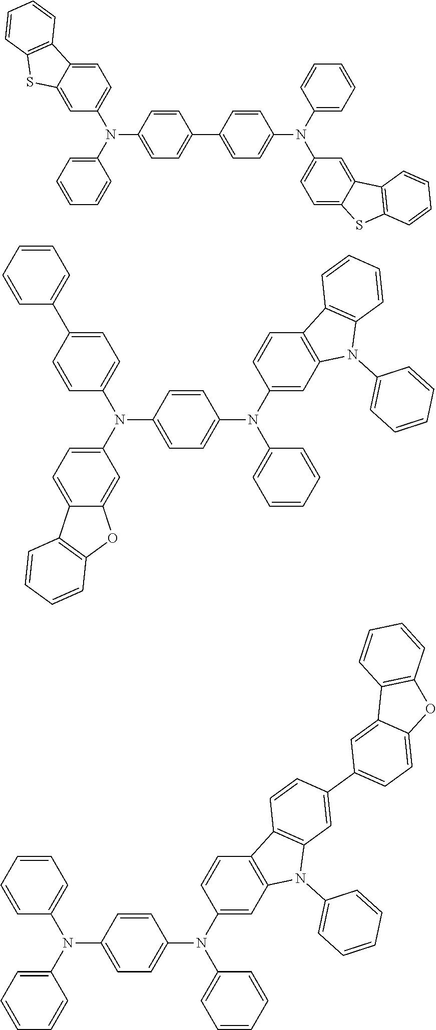

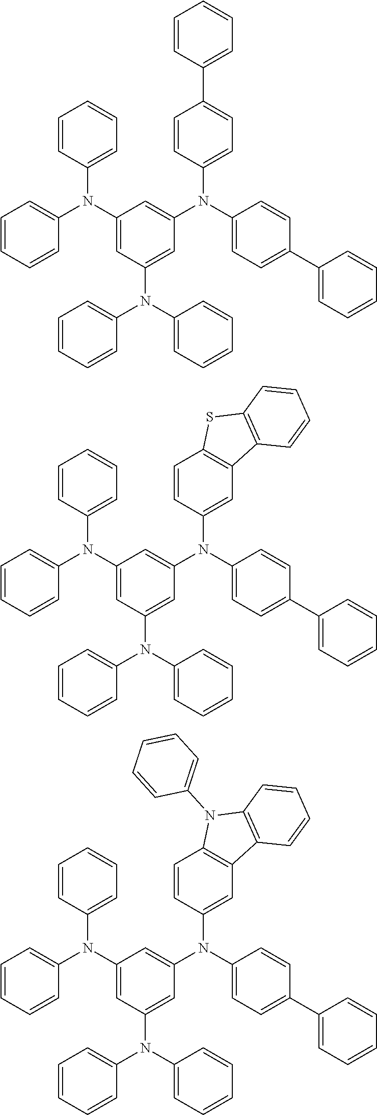

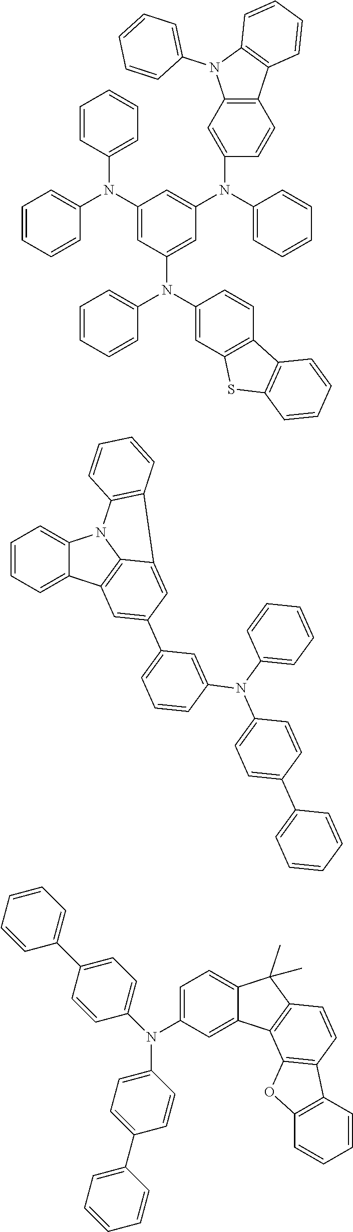 [SMILES: c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1 ; c1ccc(-c2ccc(N(c3ccccc3)c3cccc(-c4cc5c6ccccc6n6c7ccccc7c(c4)c56)c3)cc2)cc1 ; CC1(C)c2ccc(N(c3ccc(-c4ccccc4)cc3)c3ccc(-c4ccccc4)cc3)cc2-c2c1ccc1c2oc2ccccc21]

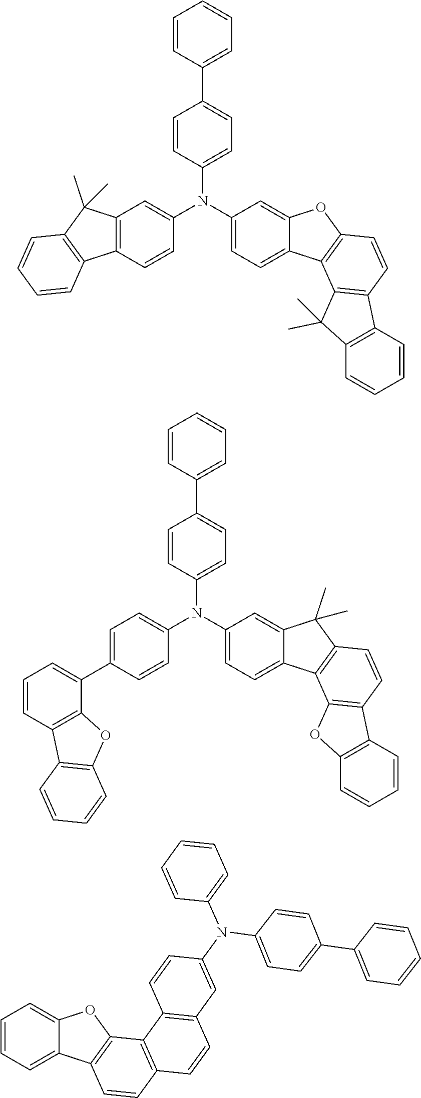 [SMILES: CC1(C)c2ccccc2-c2ccc(N(c3ccc(-c4ccccc4)cc3)c3ccc4c(c3)oc3ccc5c(c34)C(C)(C)c3ccccc3-5)cc21 ; CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1]

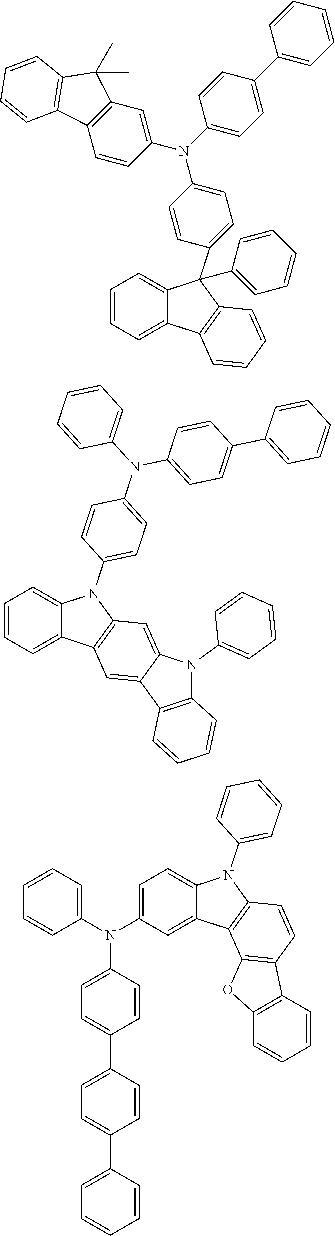

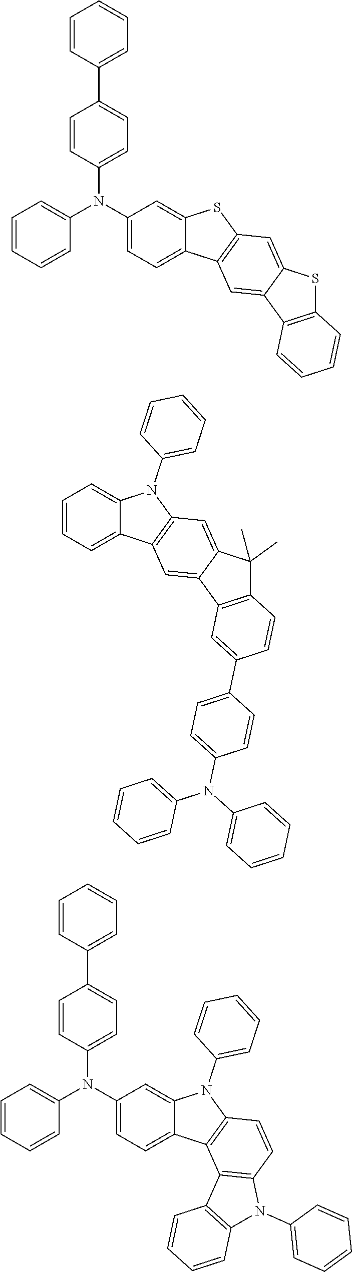 [SMILES: c1ccc(-c2ccc(N(c3ccccc3)c3ccc4c(c3)sc3cc5sc6ccccc6c5cc34)cc2)cc1 ; CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1]

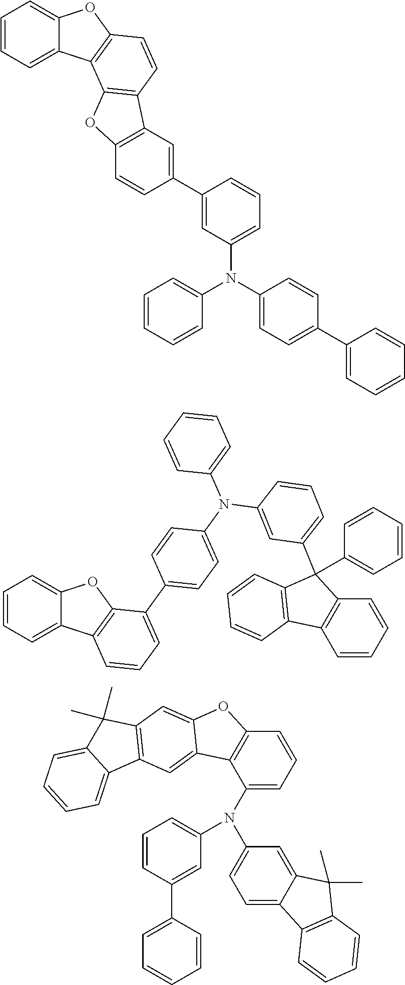

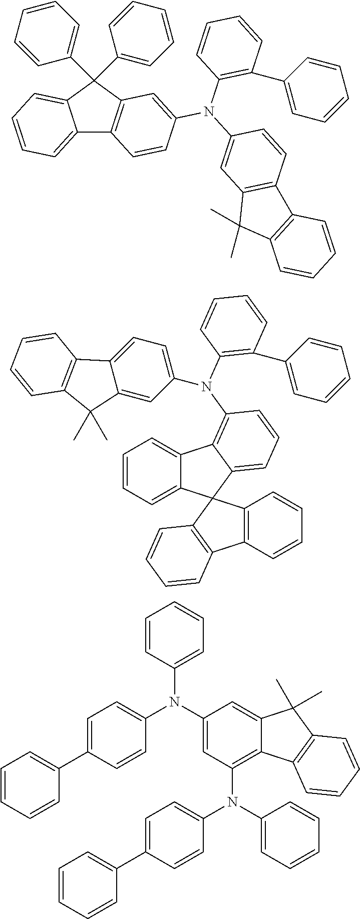

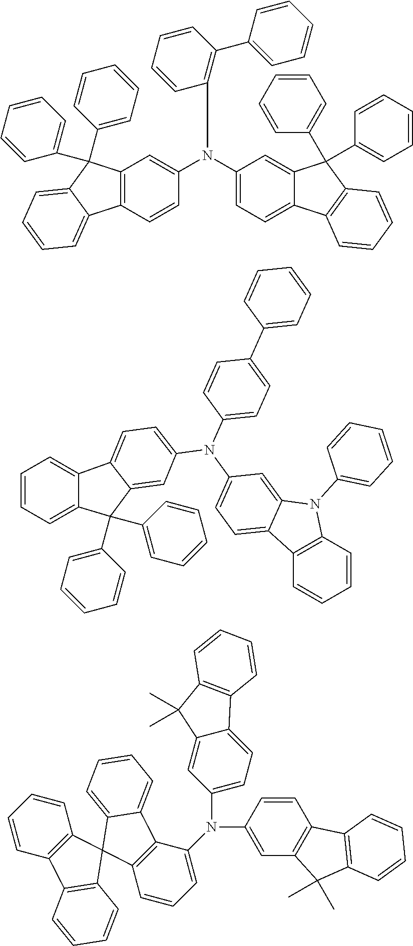 [SMILES: c1ccc(-c2ccccc2N(c2ccc3c(c2)C(c2ccccc2)(c2ccccc2)c2ccccc2-3)c2ccc3c(c2)C(c2ccccc2)(c2ccccc2)c2ccccc2-3)cc1 ; c1ccc(-c2ccc(N(c3ccc4c(c3)C(c3ccccc3)(c3ccccc3)c3ccccc3-4)c3ccc4c5ccccc5n(-c5ccccc5)c4c3)cc2)cc1 ; CC1(C)c2ccccc2-c2ccc(N(c3ccc4c(c3)C(C)(C)c3ccccc3-4)c3cccc4c3-c3ccccc3C43c4ccccc4-c4ccccc43)cc21]

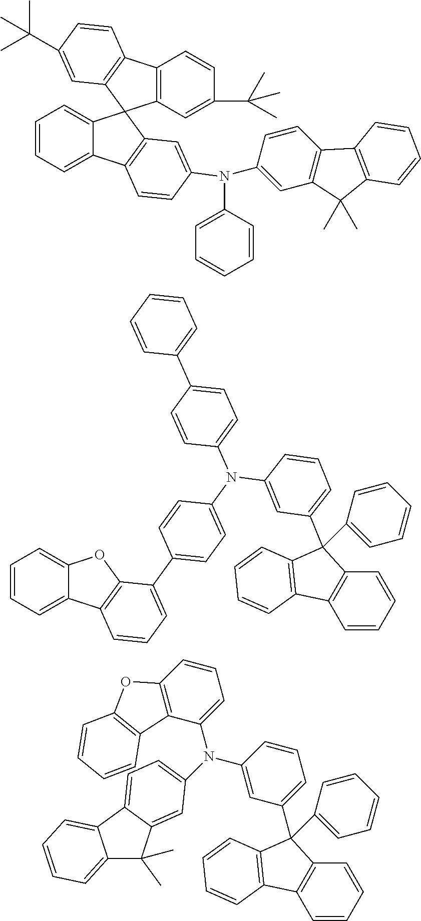

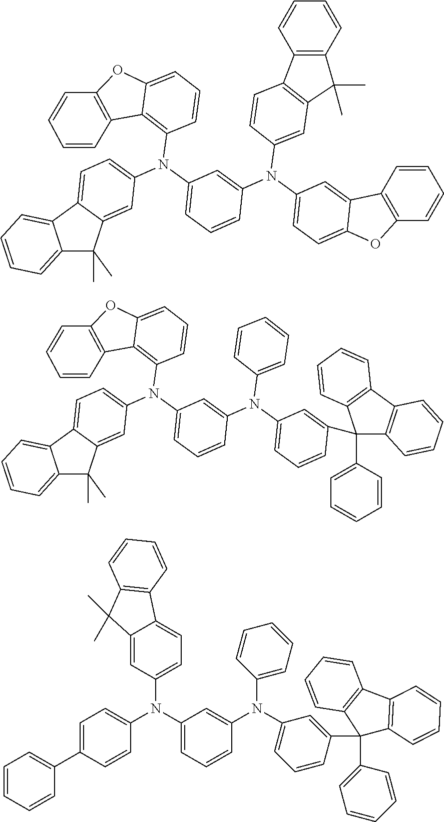

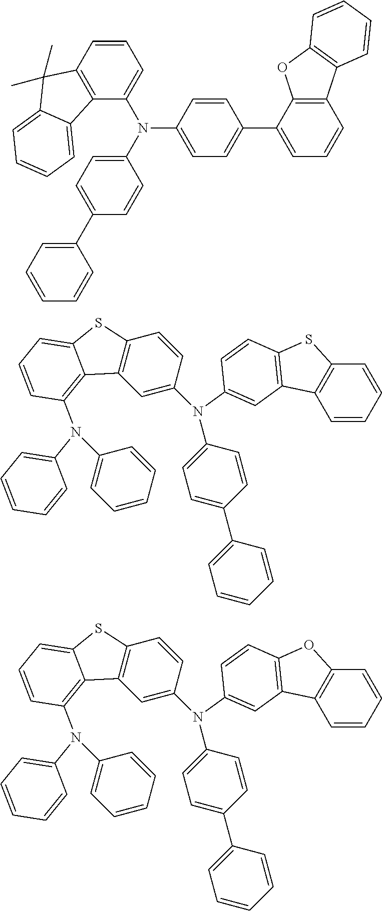

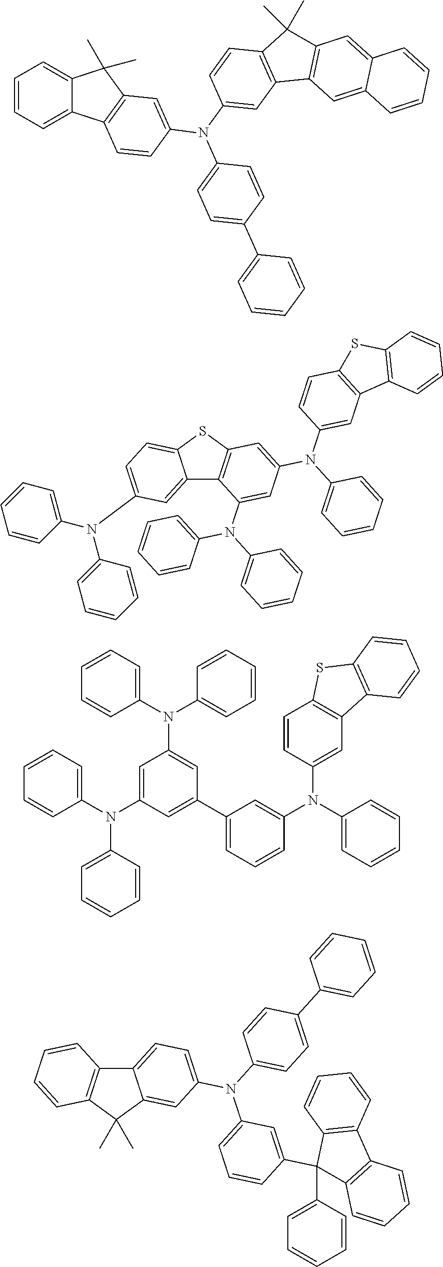

In the hole transport region 140, compounds disclosed in U.S. Pat. No. 5,061,569, JP 1993-009471 A, WO 1995-009147 A1, JP 1995-126615 A, JP 1998-095973 A, and the like and compounds similar thereto may be used in addition to the aforementioned compound.

In addition, the charge transport region may be for example an electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer.

At least one of the compounds of Group C may be included in at least one of the electron transport layer and the electron transport auxiliary layer.

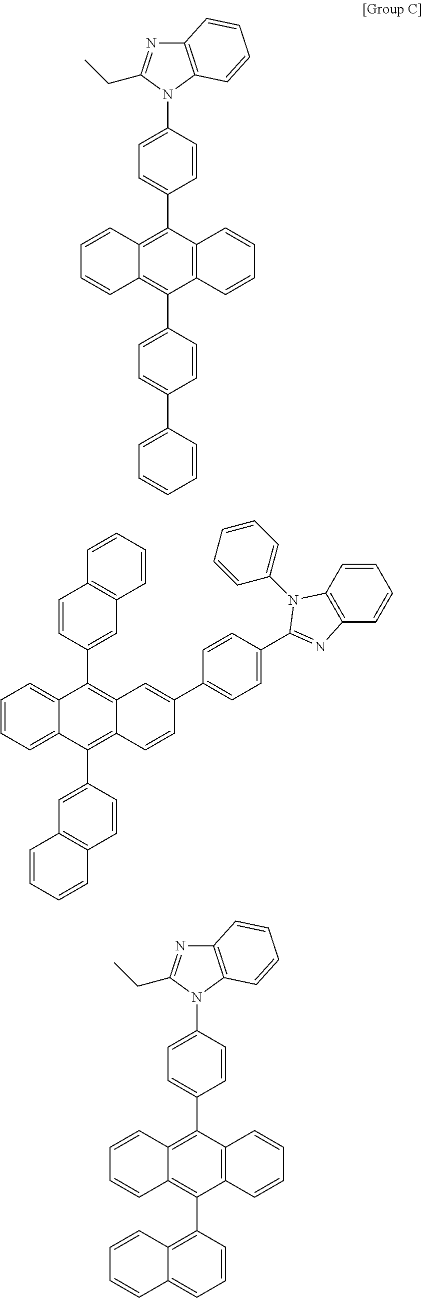 [SMILES: CCc1nc2ccccc2n1-c1ccc(-c2c3ccccc3c(-c3ccc(-c4ccccc4)cc3)c3ccccc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CCc1nc2ccccc2n1-c1ccc(-c2c3ccccc3c(-c3cccc4ccccc34)c3ccccc23)cc1]

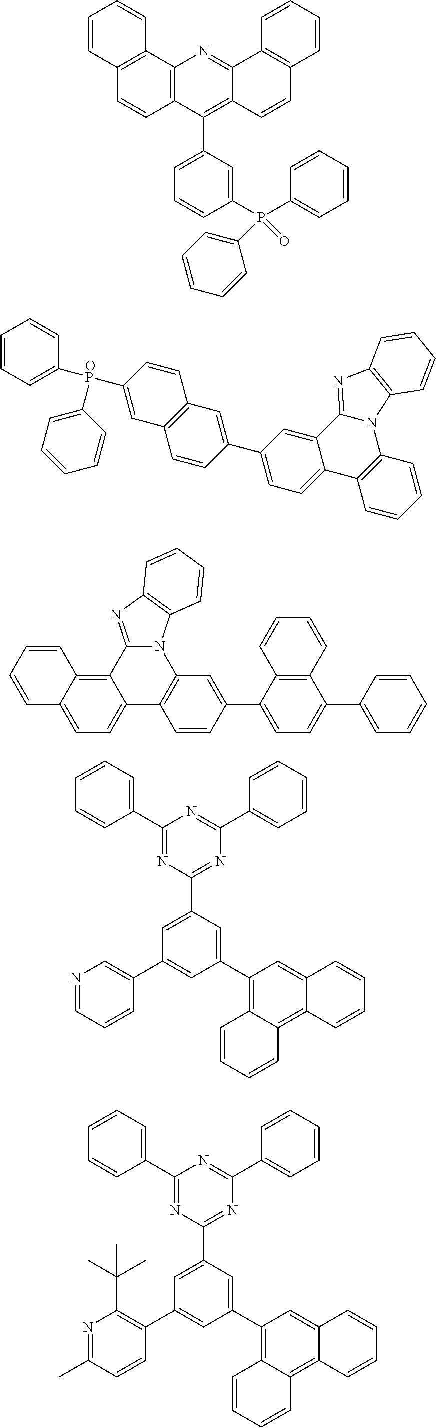

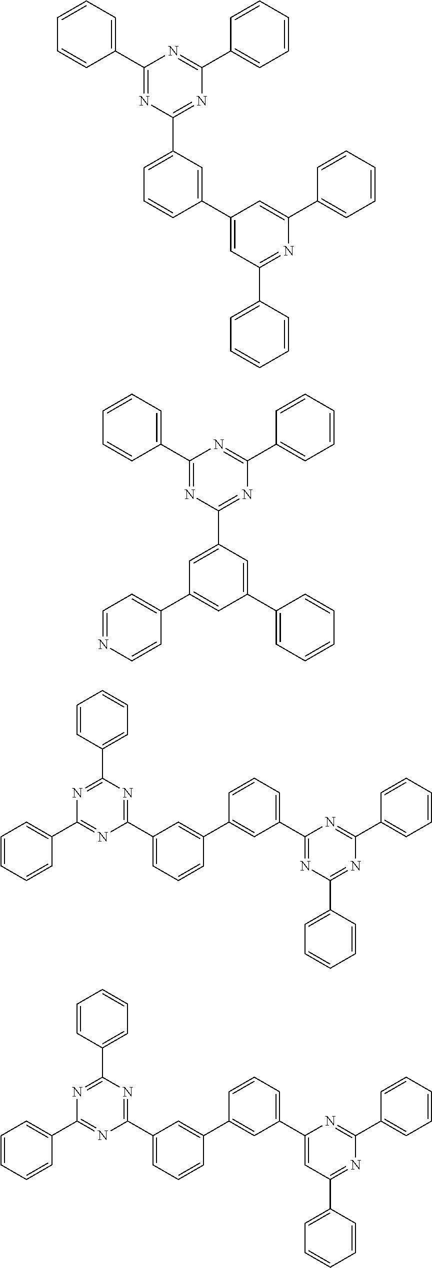 [SMILES: c1ccc(-c2cc(-c3cccc(-c4nc(-c5ccccc5)nc(-c5ccccc5)n4)c3)cc(-c3ccccc3)n2)cc1 ; c1ccc(-c2cc(-c3ccncc3)cc(-c3nc(-c4ccccc4)nc(-c4ccccc4)n3)c2)cc1 ; c1ccc(-c2nc(-c3ccccc3)nc(-c3cccc(-c4cccc(-c5nc(-c6ccccc6)nc(-c6ccccc6)n5)c4)c3)n2)cc1 ; c1ccc(-c2cc(-c3cccc(-c4cccc(-c5nc(-c6ccccc6)nc(-c6ccccc6)n5)c4)c3)nc(-c3ccccc3)n2)cc1]

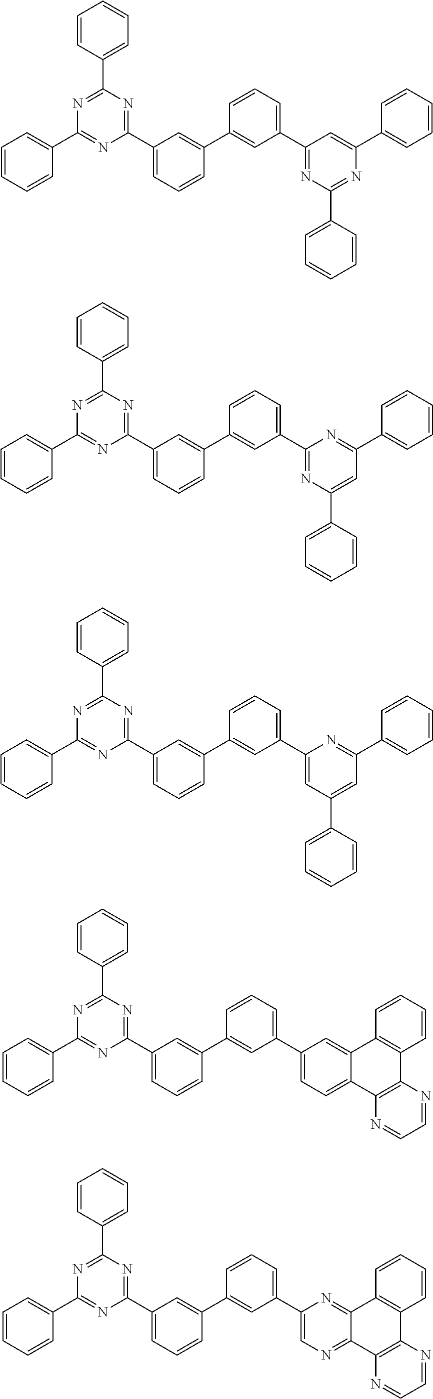 [SMILES: c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c(c5)c5ccccc5c5nccnc65)c4)c3)n2)cc1 ; c1ccc(-c2nc(-c3ccccc3)nc(-c3cccc(-c4cccc(-c5cnc6c(n5)c5ccccc5c5nccnc56)c4)c3)n2)cc1]

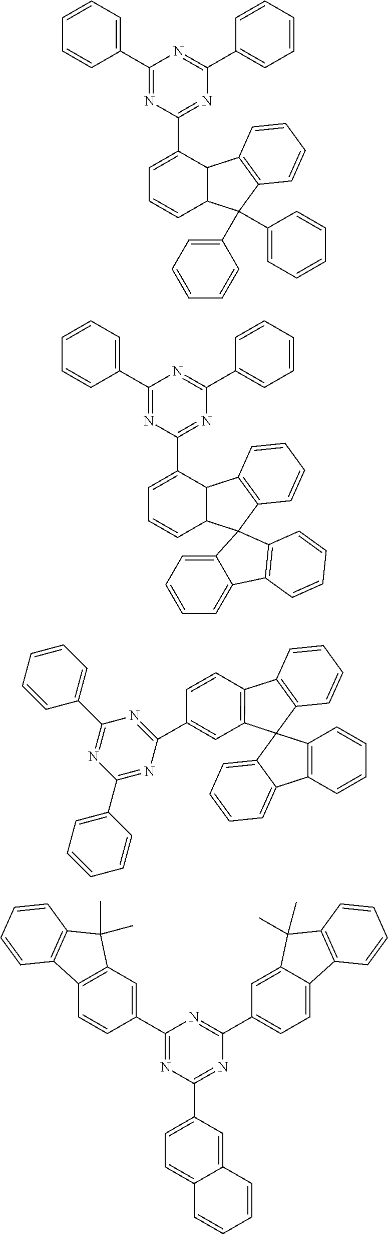

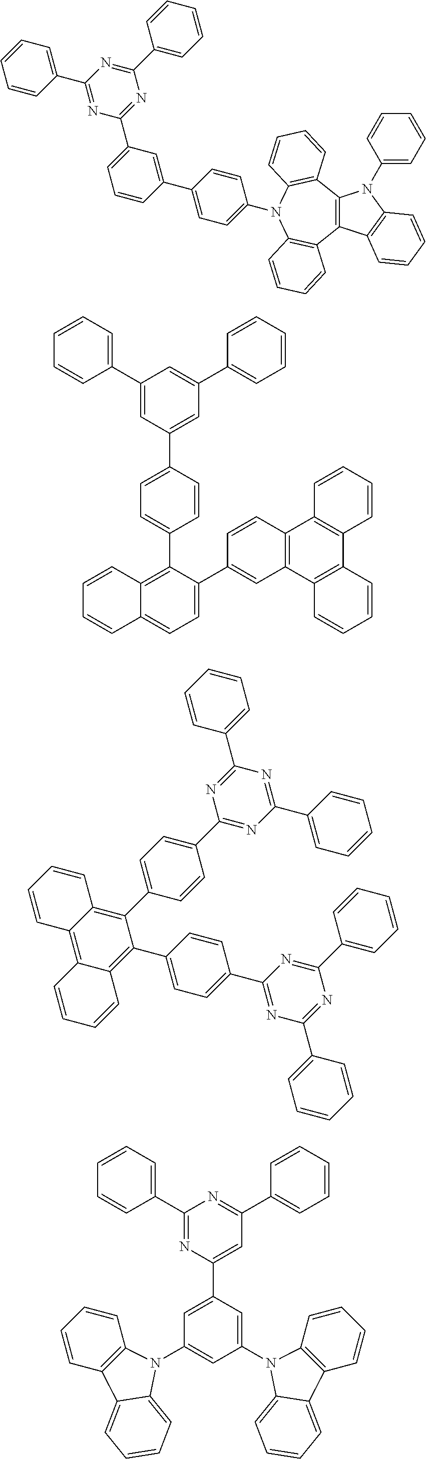

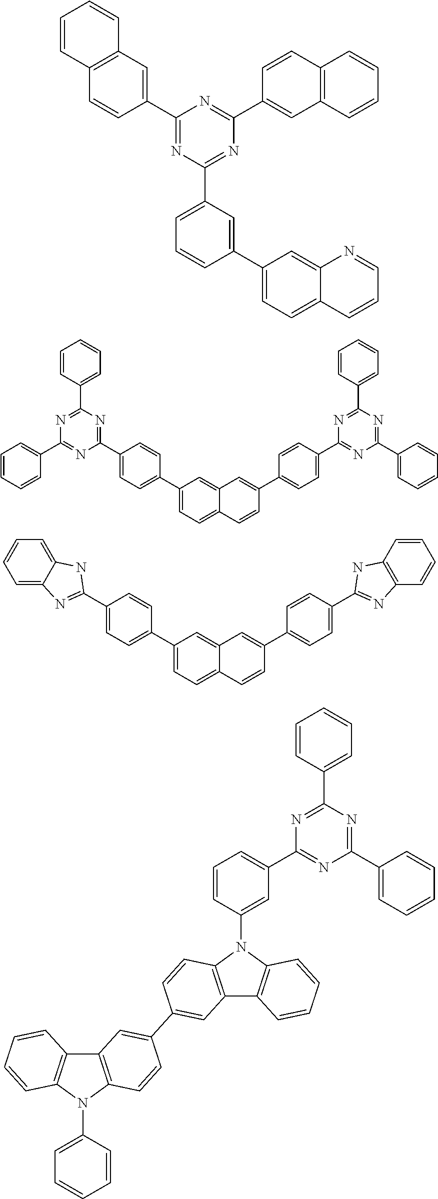

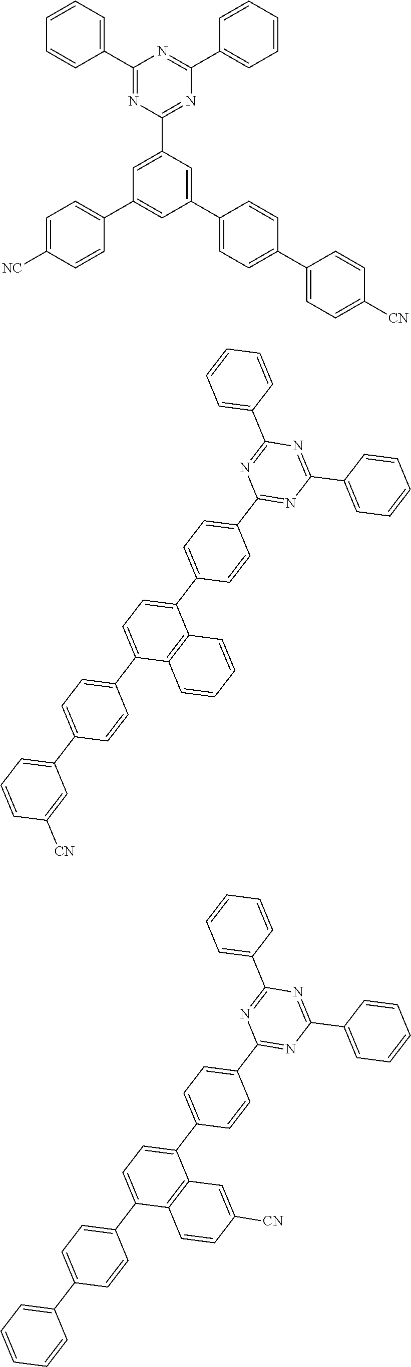 [SMILES: N#Cc1ccc(-c2ccc(-c3cc(-c4ccc(C#N)cc4)cc(-c4nc(-c5ccccc5)nc(-c5ccccc5)n4)c3)cc2)cc1 ; N#Cc1cccc(-c2ccc(-c3ccc(-c4ccc(-c5nc(-c6ccccc6)nc(-c6ccccc6)n5)cc4)c4ccccc34)cc2)c1 ; N#Cc1ccc2c(-c3ccc(-c4ccccc4)cc3)ccc(-c3ccc(-c4nc(-c5ccccc5)nc(-c5ccccc5)n4)cc3)c2c1]

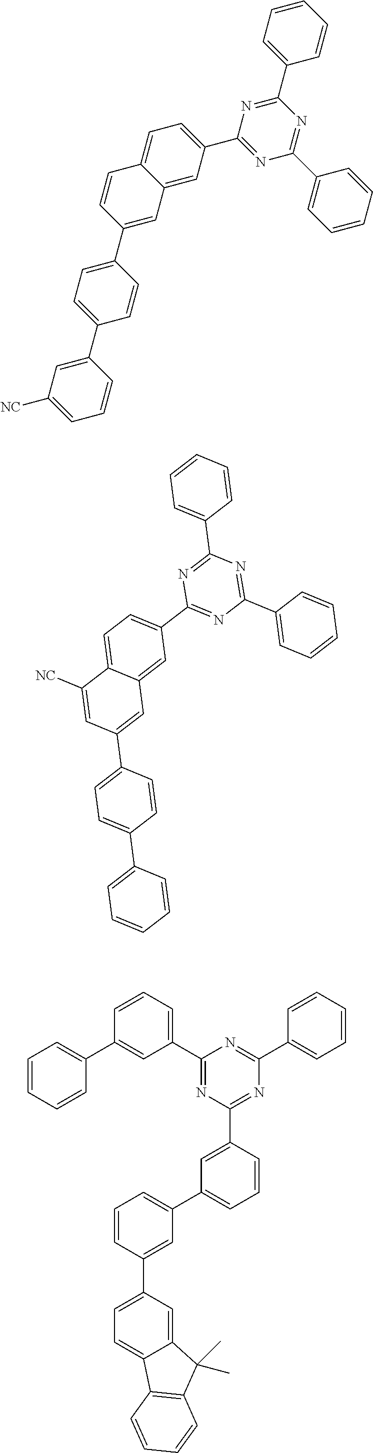 [SMILES: N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12 ; CC1(C)c2ccccc2-c2ccc(-c3cccc(-c4cccc(-c5nc(-c6ccccc6)nc(-c6cccc(-c7ccccc7)c6)n5)c4)c3)cc21]

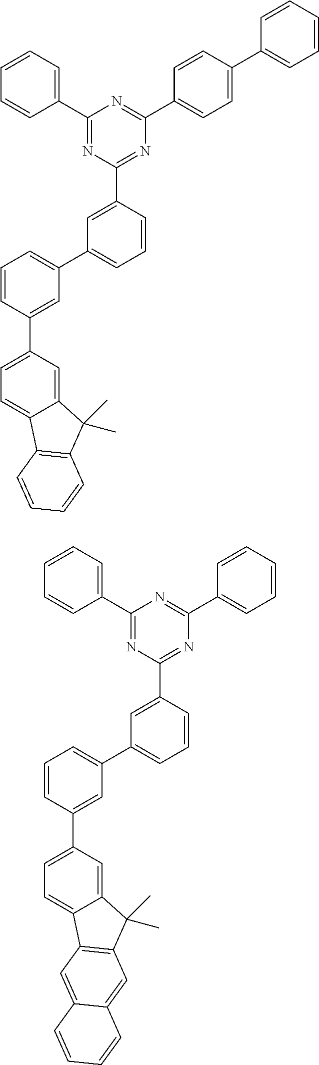

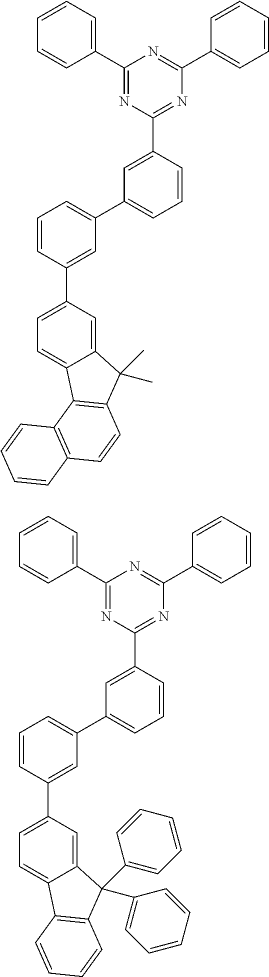

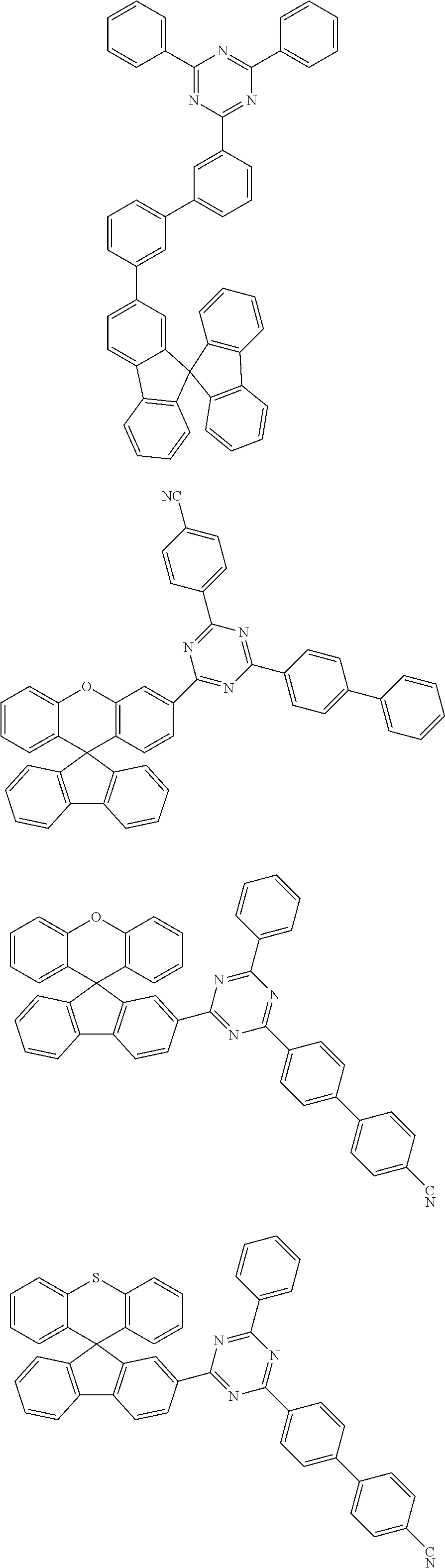

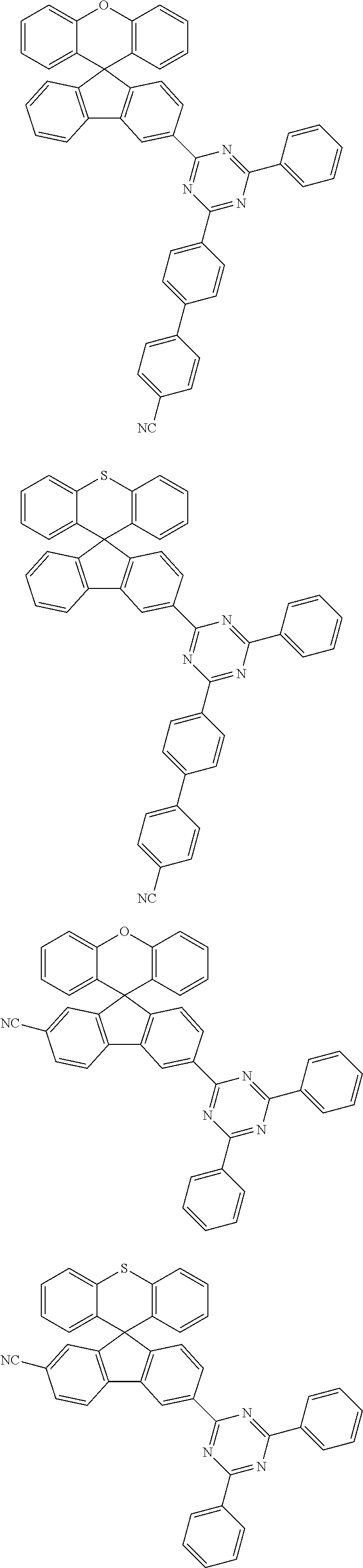

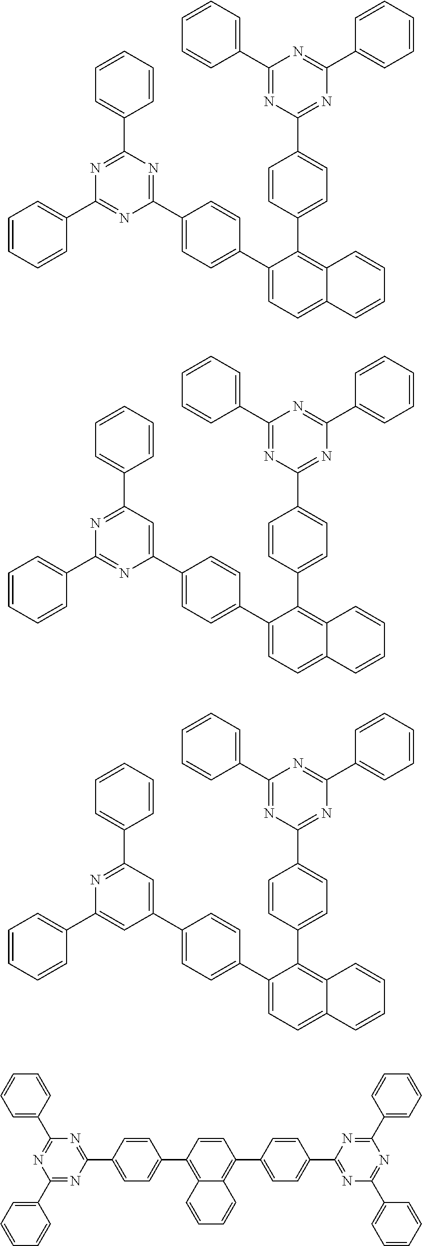 [SMILES: c1ccc(-c2nc(-c3ccccc3)nc(-c3ccc(-c4ccc5ccccc5c4-c4ccc(-c5nc(-c6ccccc6)nc(-c6ccccc6)n5)cc4)cc3)n2)cc1 ; c1ccc(-c2cc(-c3ccc(-c4ccc5ccccc5c4-c4ccc(-c5nc(-c6ccccc6)nc(-c6ccccc6)n5)cc4)cc3)nc(-c3ccccc3)n2)cc1 ; c1ccc(-c2cc(-c3ccc(-c4ccc5ccccc5c4-c4ccc(-c5nc(-c6ccccc6)nc(-c6ccccc6)n5)cc4)cc3)cc(-c3ccccc3)n2)cc1 ; c1ccc(-c2nc(-c3ccccc3)nc(-c3ccc(-c4ccc(-c5ccc(-c6nc(-c7ccccc7)nc(-c7ccccc7)n6)cc5)c5ccccc45)cc3)n2)cc1]

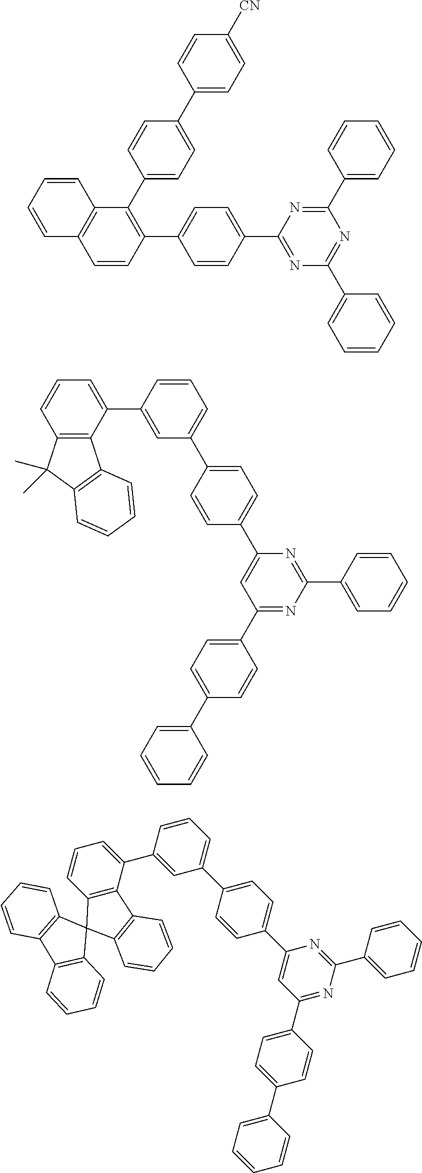

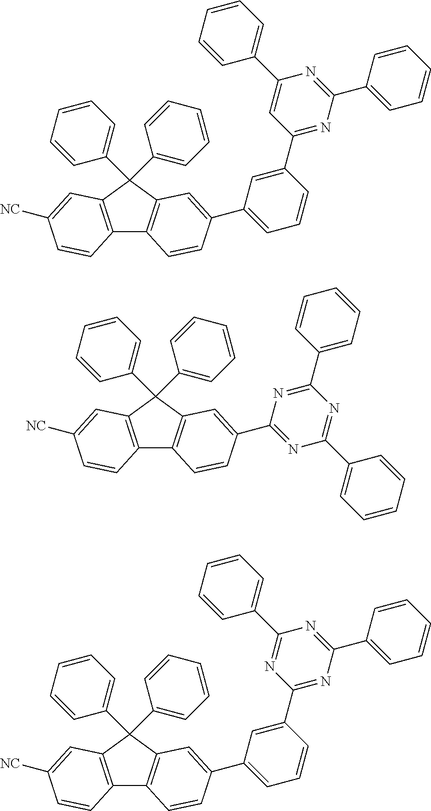 [SMILES: N#Cc1ccc2c(c1)C(c1ccccc1)(c1ccccc1)c1cc(-c3cccc(-c4cc(-c5ccccc5)nc(-c5ccccc5)n4)c3)ccc1-2 ; N#Cc1ccc2c(c1)C(c1ccccc1)(c1ccccc1)c1cc(-c3nc(-c4ccccc4)nc(-c4ccccc4)n3)ccc1-2 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

An example embodiment may be an organic light emitting diode including a light emitting layer as an organic layer.

Another example embodiment may provide an organic light emitting diode including a light emitting layer and a hole transport region as an organic layer.

Another example embodiment may provide an organic light emitting diode including a light emitting layer and an electron transport region as an organic layer.

Referring to the FIGURE, the organic light emitting diode according to an example embodiment may include a hole transport region 140 and an electron transport region 150 in addition to the light emitting layer 130 as the organic layer 105.

On the other hand, the organic light emitting diode may further include an electron injection layer (not shown), a hole injection layer (not shown), etc. in addition to the light emitting layer as the aforementioned organic layer.

The organic light emitting diode 100 may be produced by forming an anode or a cathode on a substrate, forming an organic layer using a dry film formation method such as a vacuum deposition method (evaporation), sputtering, plasma plating, and ion plating, and forming a cathode or an anode thereon.

The organic light emitting diode may be applied to an organic light emitting display device.

The following Examples and Comparative Examples are provided in order to highlight characteristics of one or more embodiments, but it will be understood that the Examples and Comparative Examples are not to be construed as limiting the scope of the embodiments, nor are the Comparative Examples to be construed as being outside the scope of the embodiments. Further, it will be understood that the embodiments are not limited to the particular details described in the Examples and Comparative Examples.

Hereinafter, starting materials and reactants used in examples and synthesis examples were purchased from Sigma-Aldrich Co. Ltd., TCI Inc., Tokyo Chemical Industry, or P&H tech as far as there in no particular comment, or were synthesized by known methods.

(Preparation of Compound for Organic Optoelectronic Device)

The compound presented as a more specific example of the compound was synthesized through the following steps.

### Synthesis Example 1: Synthesis of Intermediate A

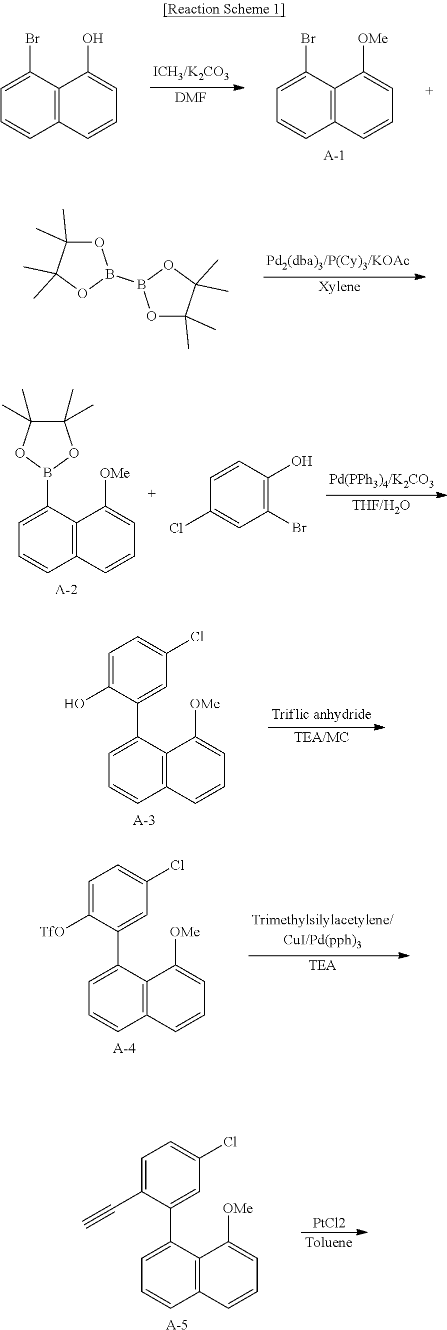

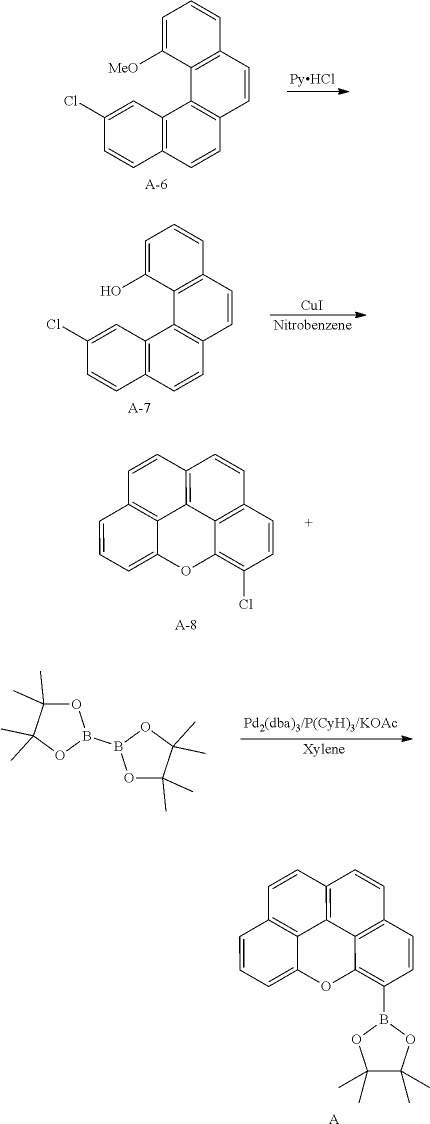

1st Step: Synthesis of Intermediate A-1

In a round-bottomed flask, 40.0 g (179.32 mmol) of 8-bromonaphthalen-1-ol, 50.9 g (358.63 mmol) of iodomethane, and 49.57 g (358.63 mmol) of K₂CO₃ were dissolved in 500 ml of DMF and then, stirred at 60° C. for 5 hours. When a reaction was completed, the resultant was concentrated and extracted with methylene chloride, and an organic layer therefrom was silica gel-columned, obtaining 35.0 g (yield 82%) of Intermediate A-1.

2nd Step: Synthesis of Intermediate A-2

35.0 g (147.62 mmol) of Intermediate A-1, 41.23 g (162.38 mmol) of bis(pinacolato)diboron, 8.11 g (8.86 mmol) of Pd₂(dba)₃, 9.94 g (35.43 mmol) of P(Cy)₃, and 43.37 g (442.85 mmol) of KOAc were dissolved in 200 ml of xylene and then, stirred under reflux for 12 hours. When a reaction was completed, after removing the reaction solvent with a rotary evaporator and conducting an extraction with methylene chloride, an organic layer therefrom was columned with hexane:ethyl acetate (EA)=4:1 (v/v), obtaining 38.5 g (yield 92%) of Intermediate A-2.

3rd Step: Synthesis of Intermediate A-3

In a round-bottomed flask, 38.5 g (135.21 mmol) of Intermediate A-2, 25.50 g (122.92 mmol) of 2-bromo-4-chlorophenol, 4.26 g (3.69 mmol) of Pd(PPh₃)₄, and 33.98 g (245.84 mmol) of K₂CO₃ were dissolved in 600 mL of THE and 300 mL of distilled water and then, heated to reflux under a nitrogen atmosphere. After 12 hours, the reaction solution was cooled, and after removing an aqueous layer, an organic layer therefrom was dried under a reduced pressure. The obtained solid was washed with water and methanol and recrystallized with 200 mL of toluene, obtaining 27.0 g (yield 78%) of Intermediate A-3.

4th Step: Synthesis of Intermediate A-4

In a round-bottomed flask, 27 g (94.82 mmol) of Intermediate A-3 and 26.75 g (94.82 mmol) of triflic anhydride were dissolved in (methylene chloride) MC and then, stirred at 0° C. for 30 minutes, and 19.72 g (142.24 mmol) of triethylamine was slowly added dropwise thereto and slowly heated to room temperature and additionally stirred for 3 hours. When a reaction was completed, after decreasing the temperature again to 0° C. and slowly adding ice was added thereto to quench acid, an organic layer extracted therefrom with methylene chloride was silica gel-columned, obtaining 33.0 g (yield 83%) of Intermediate A-4.

5th Step: Synthesis of Intermediate A-5

33 g (79.17 mmol) of Intermediate A-4, 15.55 g (158.35 mmol) of trimethylsilylacetylene, 18.09 g (95.01 mmol) of CuI, and 4.57 g (3.96 mmol) of Pd(PPh₃)₄ were dissolved in 320 ml of triethylamine and then, stirred under reflux at 70° C. for 6 hours. When a reaction was completed, the reaction solution was cooled to 0° C. and neutralized with iN HCl. Subsequently, a resulting material therefrom was three times extracted with ethyl acetate, dried with anhydrous magnesium sulfate, and distilled under a reduced pressure, and an organic layer therefrom was silica gel-columned, obtaining 19.0 g (yield 82%) of Intermediate A-5.

6th Step: Synthesis of Intermediate A-6

19.0 g (64.9 mmol) of Intermediate A-5 and 0.86 g (3.24 mmol) of PtCl₂ were dissolved in 300 ml of toluene and then, stirred under reflux at 80° C. for 24 hours. After completing a reaction with water, extraction was three times conducted by using ethyl acetate. The separated organic layer was dried with anhydrous magnesium sulfate, distilled under a reduced pressure, and silica gel-columned, obtaining 17 g (yield 89%) of Intermediate A-6.

7th Step: Synthesis of Intermediate A-7

17 g (58.07 mmol) of Intermediate A-6 and 67 g (580.68 mmol) of pyridinium chloride were stirred under reflux at 200° C. for 12 hours. When a reaction was completed, the resultant was cooled to 80° C. or less and neutralized by adding water thereto. The obtained resultant was three times extracted with ethyl acetate, dried with anhydrous magnesium sulfate, and distilled under a reduced pressure, and an organic layer obtained therefrom was silica gel-columned, obtaining 14.0 g (yield 85%) of Intermediate A-7. LC/MS calculated for: C18H11ClO Exact Mass: 278.05 found for 278.64 [M+H]

8th Step: Synthesis of Intermediate A-8

14.0 g (50.23 mmol) of Intermediate A-7 was dissolved in 300 ml of nitrobenzene, and 9.57 g (50.23 mmol) of CuI was slowly added thereto in a dropwise fashion and then, stirred under reflux at 180° C. for 50 hours. When a reaction was completed, after slowly decreasing the reaction temperature to room temperature and then, distilling and removing the nitrobenzene under a reduced pressure, an organic layer therefrom was silica gel-columned, obtaining 10.0 g (yield 72%) of Intermediate A-8. LC/MS calculated for: C18H9ClO Exact Mass: 276.03 found for 276.61 [M+H]

9th Step: Synthesis of Intermediate A

10.0 g (36.23 mmol) of Intermediate A-8, 11.04 g (43.47 mmol) of bis(pinacolato)diboron, 1.99 g (2.17 mmol) of Pd₂(dba)₃, 2.44 g (8.69 mmol) of P(Cy)₃, and 10.67 g (108.68 mmol) of KOAc were dissolved in 100 ml of xylene and then, stirred under reflux for 12 hours. When a reaction was completed, after removing the reaction solvent by using a rotary evaporator, an organic layer extracted therefrom with methylene chloride was columned with hexane:EA=4:1 (v/v), obtaining 12.3 g (yield 92%) of Intermediate A. LC/MS calculated for: C24H21BO3 Exact Mass 368.16 found for 368.54 [M+H]

### Synthesis Example 2: Synthesis of Compound 1

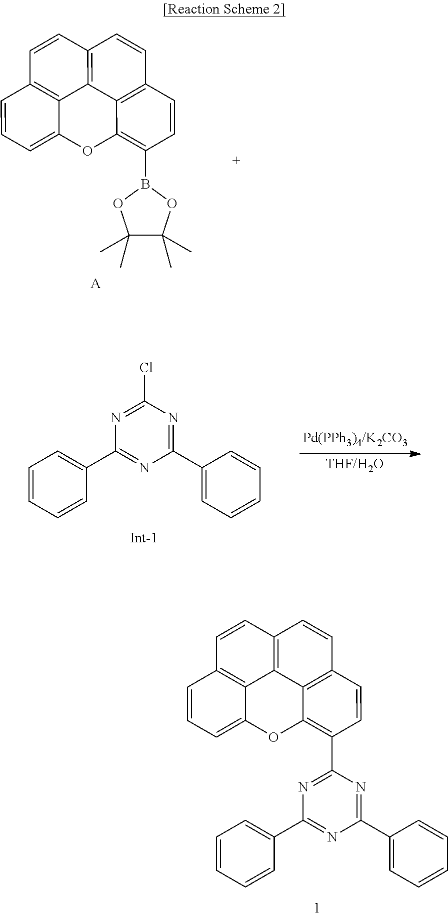

In a round-bottomed flask, 12.0 g (32.87 mmol) of Intermediate A, 8.0 g (29.88 mmol) of 2-chloro-4,6-diphenyl-1,3,5-triazine (Int-1), 1.04 g (0.9 mmol) of Pd(PPh₃)₄, and 8.26 g (59.76 mmol) of K₂CO₃ were dissolved in 150 mL of THF and 75 mL of distilled water and then, heated to reflux under a nitrogen atmosphere. 12 hours later, after cooling the reaction solution and removing an aqueous layer, an organic layer therefrom was dried under a reduced pressure. The obtained solid was washed with water and methanol and twice recrystallized with 200 mL of toluene, obtaining 11.0 g (yield 78%) of Compound 1. LC/MS calculated for: C33H19N3O Exact Mass 473.15 found for 473.54 [M+H]

### Synthesis Example 3: Synthesis of Intermediate B

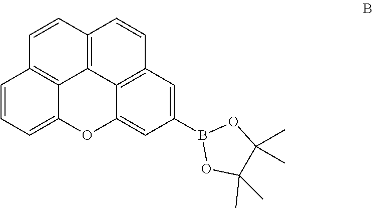 [SMILES: CC1(C)OB(c2cc3c4c(ccc5ccc6cccc(c6c54)O3)c2)OC1(C)C]

Intermediate B was synthesized in the same manner as in Synthesis Example 1 except that 2-bromo-5-chlorophenol was used instead of the 2-bromo-4-chlorophenol in step 3 of Synthesis Example 1. C24H21BO3 Exact Mass 368.16 found for 368.24 [M+H]

### Synthesis Examples 4 to 9

Each compound shown in Table 1 was synthesized in the same manner as in Synthesis Example 2 except that intermediate A or Intermediate B and a compound of Int B instead of Int-2 was used instead of the 2-bromo-4-chlorophenol in Synthesis Example 2.

TABLE 1

| Synthesis | | | Final | Amount | |
|---|---|---|---|---|---|
| Examples | Int A | Int B | product | (yield) | Property data of final product |
| Synthesis | Inter- | Int-2 | 3 | 11.0 g, | LC/MS calculated for: |
| Example 4 | mediate | | | 72% | C43H25N3O Exact Mass: |
| | A | | | | 599.2 found for 599.5 [M + H] |
| Synthesis | Inter- | Int-3 | 4 | 12.0 g, | LC/MS calculated for: |
| Example 5 | mediate | | | 76% | C39H21N3O2 Exact Mass: |
| | A | | | | 563.16 found for 563.7 [M + H] |
| Synthesis | Inter- | Int-4 | 5 | 11.5 g, | LC/MS calculated for: |
| Example 6 | mediate | | | 76% | C43H25N3O Exact Mass: |
| | A | | | | 599.20 found for 599.62 [M + H] |
| Synthesis | Inter- | Int-5 | 6 | 10.8 g, | LC/MS calculated for: |
| Example 7 | mediate | | | 74% | C47H27N3O Exact Mass: |
| | A | | | | 649.22 found for 649.63 [M + H] |
| Synthesis | Inter- | Int-1 | 7 | 10.4 g, | LC/MS calculated for: |
| Example 8 | mediate | | | 73% | C33H19N3O Exact Mass: |
| | B | | | | 473.15 found for [M + H] 473.41 |
| Synthesis | Inter- | Int-6 | 8 | 9.8 g, | LC/MS calculated for: |
| Example 9 | mediate | | | 73% | C44H26N2O Exact Mass: |
| | A | | | | 598.2 found for 598.8 [M + H] |

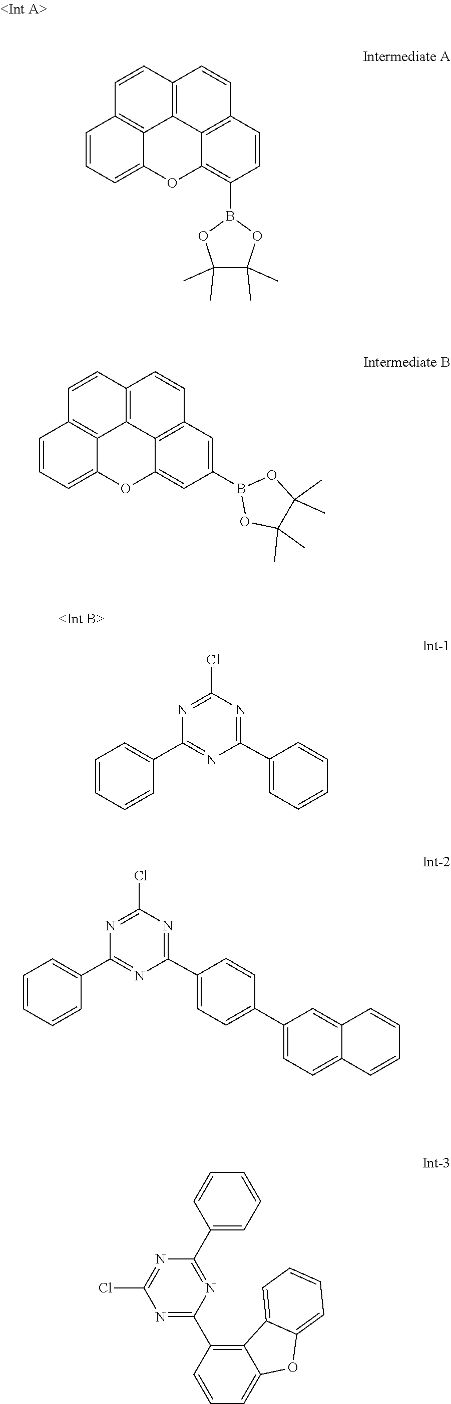 [SMILES: CC1(C)OB(c2ccc3ccc4ccc5cccc6c5c4c3c2O6)OC1(C)C ; CC1(C)OB(c2cc3c4c(ccc5ccc6cccc(c6c54)O3)c2)OC1(C)C ; Clc1nc(-c2ccccc2)nc(-c2ccccc2)n1 ; Clc1nc(-c2ccccc2)nc(-c2ccc(-c3ccc4ccccc4c3)cc2)n1 ; Clc1nc(-c2ccccc2)nc(-c2cccc3oc4ccccc4c23)n1]

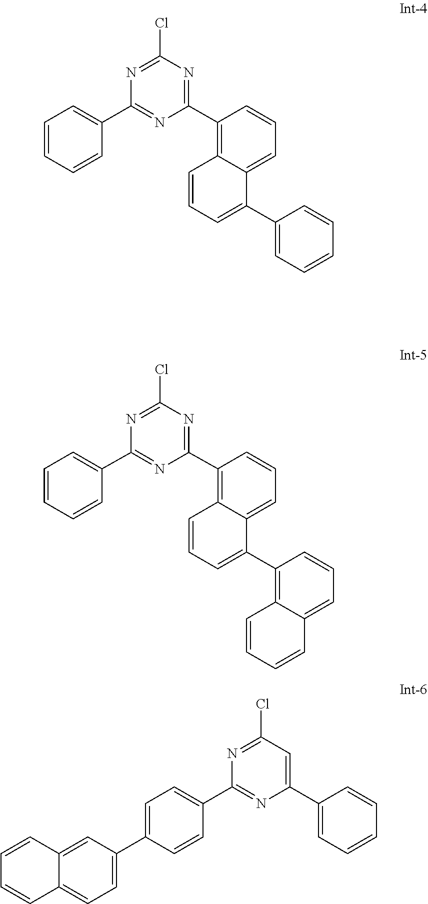

Comparative Synthesis Example 1: Synthesis of Compound R1

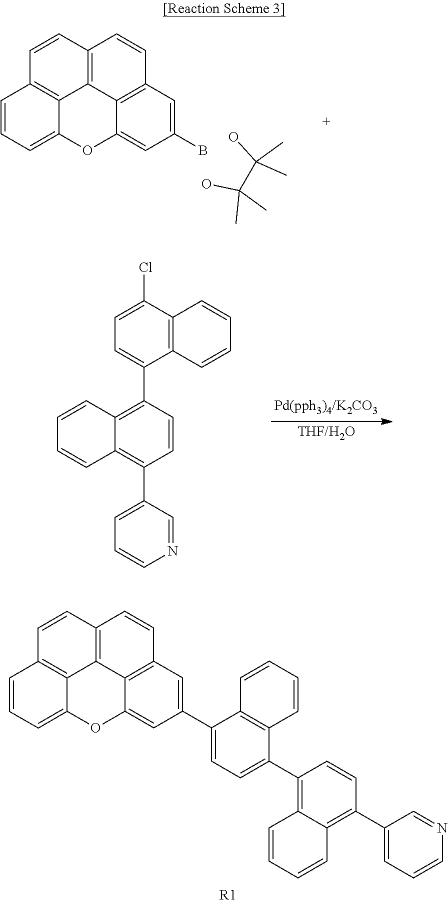

In a round-bottomed flask, 10.0 g (27.15 mmol) of Intermediate B, 9.0 g (24.68 mmol) of 3-(4′-chloro-[1,1′-binaphthalen]-4-yl)pyridine, 0.86 g (0.74 mmol) of Pd(PPh₃)₄, and 6.82 g (49.36 mmol) of K₂CO₃ were dissolved in 150 mL of THF and 75 mL of distilled water and then, heated to reflux under a nitrogen atmosphere. After 12 hours, the reaction solution was cooled, and an organic layer obtained after removing an aqueous layer was dried under a reduced pressure. The solid was washed with water and methanol and twice recrystallized with 200 mL of toluene, obtaining 10.4 g (yield 74%) of Compound R1. LC/MS calculated for: C43H25NO Exact Mass 571.13 found for 571.78 [M+H]

### Comparative Synthesis Example 2: Synthesis of Compound R2

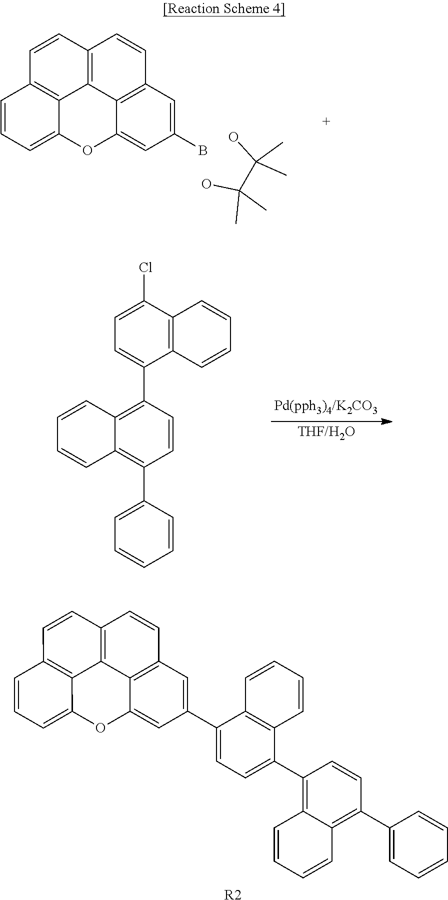

In a round-bottomed flask, 10.0 g (27.15 mmol) of Intermediate B, 9.03 g (24.68 mmol) of 4-chloro-4′-phenyl-1,1′-binaphthalene, 0.86 g (0.74 mmol) of Pd(PPh₃)₄, and 6.82 g (49.36 mmol) of K₂CO₃ were dissolved in 150 mL of THF and 75 mL of distilled water and then, heated to reflux under a nitrogen atmosphere. After 12 hours, the reaction solution was cooled, and after removing an aqueous layer, an organic layer therefrom was dried under a reduced pressure. The obtained solid was washed with water and methanol and twice recrystallized with 200 mL of toluene, obtaining 10.0 g (yield 71%) of Compound R2. LC/MS calculated for: C44H260 Exact Mass 570.20 found for 570.61 [M+H]

### Synthesis Example 10: Synthesis of Compound A-84

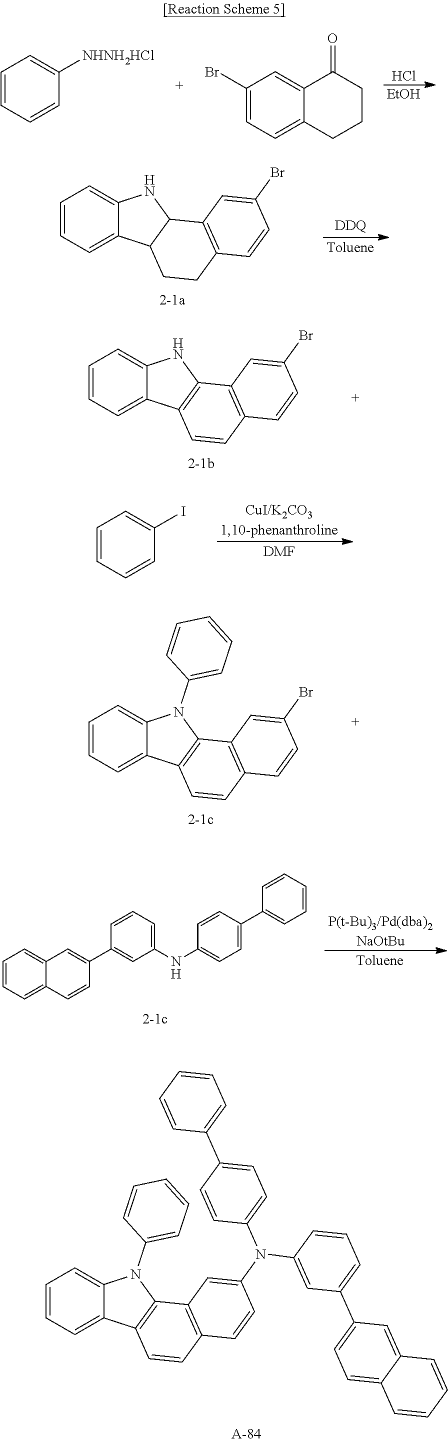

1st Step: Synthesis of Intermediate 2-1a

Phenylhydrazinehydrochloride (70.0 g, 484.1 mmol) and 7-bromo-3,4-dihydro-2H-naphthalen-1-one (108.9 g, 484.1 mmol) were put in a round-bottomed flask and dissolved in ethanol (1200 ml). Subsequently, 60 mL of hydrochloric acid was slowly added thereto in a dropwise fashion at room temperature and then, stirred at 90° C. for 12 hours. When a reaction was completed, after removing the solvent under a reduced pressure, an excessive amount of EA was used for extraction. After removing an organic solvent under a reduced pressure, the residue was stirred in a small amount of methanol and then, filtered, obtaining 95.2 g (yield 66%) of Intermediate 2-1a.

2nd Step: Synthesis of Intermediate 2-1b

Intermediate 2-1a (95.2 g, 319.3 mmol) and 2,3-dichloro-5,6-dicyano-1,4-benzoquinone (108.7 g, 478.9 mmol) were put in a round-bottomed flask and dissolved in 600 ml of toluene. The solution was stirred at 80° C. for 12 hours. When a reaction was completed, after removing the reaction solvent, the residue was treated through column chromatography, obtaining 41.3 g (yield 44%) of Intermediate 2-1b.

3rd Step: Synthesis of Intermediate 2-1c

Intermediate 2-1b (41.3 g, 139.0 mmol), iodobenzene (199.2 g, 976.0 mmol), CuI (5.31 g, 28.0 mmol), K₂CO₃ (28.9 g, 209.0 mmol), and 1,10-phenanthroline (5.03 g, 28.0 mmol) were put in a round-bottomed flask and dissolved in 500 ml of DMF. The solution was stirred at 180° C. for 12 hours. When a reaction was completed, after removing the reaction solvent under a reduced pressure, the residue was dissolved in dichloromethane and silica gel-filtered. A product therefrom was concentrated with dichloromethane and recrystallized with hexane, obtaining 39.0 g (yield 75%) of Intermediate 2-1c.

4th Step: Synthesis of Compound A-84

5.0 g (13.46 mmol) of Intermediate 2-1c, 4.41 g (13.46 mmol) of amine intermediate 2-1d, 1.94 g (20.19 mmol) of sodium t-butoxide, and 0.54 g (1.35 mmol) of tri-tert-butylphosphine were dissolved in 100 ml of toluene, and 0.37 g (0.4 mmol) of Pd(dba)₂ was added thereto and then, stirred under reflux for 12 hours under a nitrogen atmosphere. When a reaction was completed, the resultant was extracted with toluene and distilled water, and an organic layer therefrom was dried with anhydrous magnesium sulfate and filtered, and a filtrate therefrom was concentrated under a reduced pressure. A product therefrom was purified through silica gel column chromatography with normal hexane/dichloromethane (2:1 in a volume ratio), obtaining 6.4 g (yield 82.0%) of Compound A-84.

(Manufacture of Organic Light Emitting Diode)

### Example 1

A glass substrate coated with a thin film of indium tin oxide (ITO) to a thickness of 1,500 Å was washed with distilled water. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This obtained ITO transparent electrode was used as an anode, Compound A was vacuum-deposited on the ITO substrate to form a 700 Å-thick hole injection layer, and Compound B was deposited to a thickness of 50 Å on the injection layer and then Compound C was deposited to a thickness of 1,020 Å to form a hole transport layer. On the hole transport layer, 400 Å-thick light emitting layer was formed by using Compound 1 of Synthesis Example 1 as a host and doping 2 wt % of [Ir(piq)₂acac] as a dopant by vacuum-deposition. Subsequently, on the light emitting layer, Compound D and LiQ were simultaneously vacuum-deposited in a weight ratio of 1:1 to form a 300 Å-thick electron transport layer and on the electron transport layer, LiQ and Al were sequentially vacuum-deposited to be 15 Å-thick and 1200 Å-thick, manufacturing an organic light emitting diode.

The organic light emitting diode has a structure having a five-layered organic thin film layer, specifically as follows:

ITO/Compound A (700 Å)/Compound B (50 Å)/Compound C (1,020 Å)/EML [Compound 1: [Ir(piq)₂acac]=98:2 (wt %/wt %)] (400 Å)/Compound D: LiQ (300 Å)/LiQ (15 Å)/Al (1,200 Å).

Compound A: N4,N4′-diphenyl-N4,N4′-bis(9-phenyl-9H-carbazol-3-yl)biphenyl-4,4′-diamine

Compound B: 1,4,5,8,9,11-hexaazatriphenylene-hexacarbonitrile(HAT-CN),

Compound C: N-(biphenyl-4-yl)-9,9-dimethyl-N-(4-(9-phenyl-9H-carbazol-3-yl)phenyl)-9H-fluoren-2-amine

Compound D: 8-(4-(4,6-di(naphthalen-2-yl)-1,3,5-triazin-2-yl)phenyl)quinoline

### Examples 2 to 5 and Comparative Examples 1 and 2

Diodes of Examples 2 to 5 and Comparative Examples 1 and 2 were manufactured in the same manner as in Example 1, except that the host was changed as shown in Table 2.

### Examples 6 to 12 and Comparative Examples 3 and 4

Diodes of Examples 6 to 12 and Comparative Examples 3 and 4 in the same manner as in Example 1 except that the host was changed as shown in Table 3, and the first host and the second host were mixed in a weight ratio of 5:5.

Evaluation

The luminous efficiency and life-span characteristics of the organic light emitting diodes according to Examples 1 to 12 and Comparative Examples 1 to 4 were evaluated. Specific measurement methods are as follows, and the results are shown in Tables 2 and 3.

(1) Measurement of Current Density Change Depending on Voltage Change

The obtained organic light emitting diodes were measured regarding a current value flowing in the unit device, while increasing the voltage from 0 V to 10 V using a current-voltage meter (Keithley 2400), and the measured current value is divided by area to provide the results.

(2) Measurement of Luminance Change Depending on Voltage Change

Luminance was measured by using a luminance meter (Minolta Cs-1000A), while the voltage of the organic light emitting diodes was increased from 0 V to 10 V.

(3) Measurement of Luminous Efficiency

The luminous efficiency (cd/A) of the same current density (10 mA/cm²) was calculated using the luminance and current density from the items (1) and (2) and voltages.

Relative values based on the luminous efficiency of Comparative Examples 1 and 3 were calculated and shown in Tables 2 and 3.

(4) Measurement of Life-Span

T95 life-spans of the organic light emitting diodes according to Examples 1 to 12, and Comparative Examples 1 to Comparative Example 4 were measured as a time when their luminance decreased down to 95% relative to the initial luminance after emitting light with 6000 cd/m² as the initial luminance (cd/m²) and measuring their luminance decrease depending on a time with a Polanonix life-span measurement system.

The relative values based on the T95 life-spans of Comparative Examples 1 and 3 were calculated and shown in Tables 2 and 3.

TABLE 2

| | Single host | T95 life-span (%) | Efficiency (%) |
|---|---|---|---|
| Example 1 | 1 | 180 | 200 |
| Example 2 | 3 | 200 | 220 |
| Example 3 | 4 | 220 | 210 |
| Example 4 | 7 | 160 | 180 |
| Example 5 | 8 | 165 | 150 |
| Comparative Example 1 | R1 | 100 | 100 |
| Comparative Example 2 | R2 | 90 | 85 |

TABLE 3

| | Host | | T95 life- | Efficiency |
|---|---|---|---|---|
| | First host | Second host | span (%) | (%) |
| Example 6 | 1 | A-84 | 180 | 200 |
| Example 7 | 3 | A-84 | 200 | 220 |
| Example 8 | 4 | A-84 | 220 | 210 |
| Example 9 | 5 | A-84 | 200 | 210 |
| Example 10 | 6 | A-84 | 230 | 160 |
| Example 11 | 7 | A-84 | 160 | 180 |
| Example 12 | 8 | A-84 | 165 | 150 |
| Comparative Example 3 | R1 | A-84 | 100 | 100 |
| Comparative Example 4 | R2 | A-84 | 92 | 87 |

Referring to Tables 2 and 3, when the compound according to an example embodiment is used as a single host and a host in combination with a second host, it can be confirmed that the efficiency and life-span are significantly improved compared with those using the comparative compounds.

As described above, an example embodiment may provides a compound for an organic optoelectronic device capable of realizing high-efficiency and long life-span organic optoelectronic device. Another example embodiment may provide a composition for an organic optoelectronic device including the compound. Another example embodiment may provide an organic optoelectronic device including the compound. Another example embodiment may provide a display device including the organic optoelectronic device.

A high-efficiency and long life-span organic optoelectronic device may be realized.

Example embodiments have been disclosed herein, and although specific terms are employed, they are used and are to be interpreted in a generic and descriptive sense only and not for purpose of limitation. In some instances, as would be apparent to one of ordinary skill in the art as of the filing of the present application, features, characteristics, and/or elements described in connection with a particular embodiment may be used singly or in combination with features, characteristics, and/or elements described in connection with other embodiments unless otherwise specifically indicated. Accordingly, it will be understood by those of skill in the art that various changes in form and details may be made without departing from the spirit and scope as set forth in the following claims.

### DESCRIPTION OF SYMBOLS

100: organic light emitting diode
105: organic layer
110: cathode
120: anode
130: light emitting layer
140: hole transport region
150: electron transport region

What is claimed is:

1. A compound for an organic optoelectronic device, the compound being represented by Chemical Formula 1: 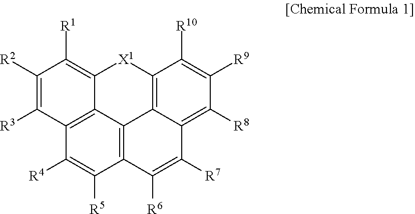
wherein, in Chemical Formula 1,
X¹ is O or S,
R¹ to R¹⁰ are each independently hydrogen, deuterium, a halogen, a hydroxy group, a cyano group, a nitro group, a substituted or unsubstituted C1 to C30 alkyl group, a substituted or unsubstituted C6 to C30 aryl group, or a substituted or unsubstituted C2 to C30 heterocyclic group, and
at least one of R¹ to R⁵ is represented by Chemical Formula a, 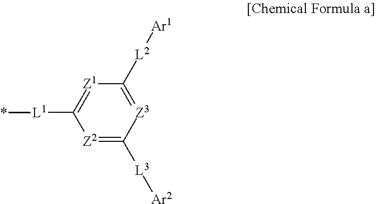
wherein, in Chemical Formula a,
Z¹ to Z³ are each independently N or CR^{a},
at least two of Z¹ to Z³ are N,
L¹ to L³ are each independently a single bond or a substituted or unsubstituted C6 to C30 arylene group,
Ar¹ and Ar² are each independently a substituted or unsubstituted C6 to C30 aryl group or a substituted or unsubstituted C2 to C30 heterocyclic group, and
R^{a} is hydrogen, deuterium, a halogen, a hydroxy group, a cyano group, a nitro group, a substituted or unsubstituted C1 to C20 alkyl group, or a substituted or unsubstituted C6 to C20 aryl group.

2. The compound as claimed in claim 1, wherein Chemical Formula 1 is represented by any one of Chemical Formula 1-1 to Chemical Formula 1-5: 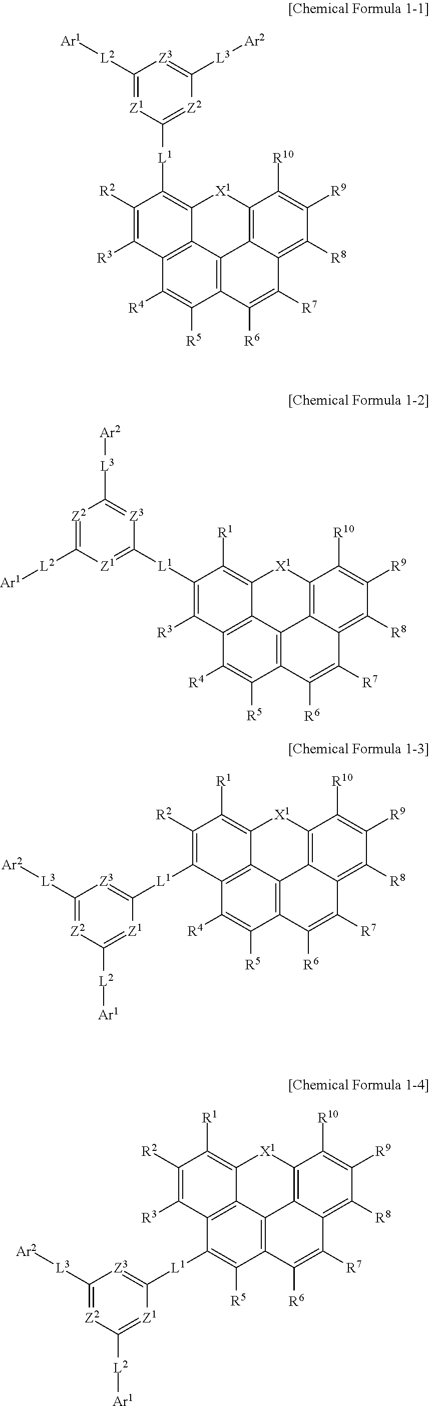 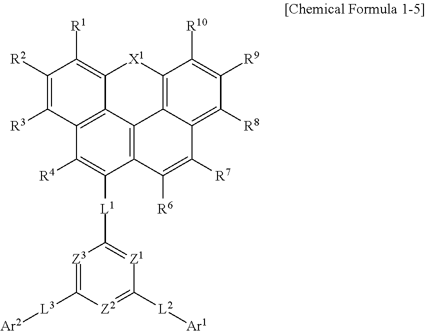
wherein, in Chemical Formula 1-1 to Chemical Formula 1-5,
the definitions of X¹, R¹ to R¹⁰, L¹ to L³, Z¹ to Z³, Ar¹, and Ar² are the same as for Chemical Formula 1 and Chemical Formula a.

3. The compound as claimed in claim 1, wherein L¹ to L³ of Chemical Formula a are each independently a single bond, a substituted or unsubstituted phenylene group, a substituted or unsubstituted biphenylene group, a substituted or unsubstituted naphthylene group, or a substituted or unsubstituted phenanthrenylene group.

4. The compound as claimed in claim 1, wherein Ar¹ and Ar² of Chemical Formula a are each independently a substituted or unsubstituted phenyl group, a substituted or unsubstituted biphenyl group, a substituted or unsubstituted naphthyl group, a substituted or unsubstituted phenanthrenyl group, a substituted or unsubstituted fluorenyl group, a substituted or unsubstituted chrysenyl group, a substituted or unsubstituted carbazolyl group, a substituted or unsubstituted dibenzofuranyl group, a substituted or unsubstituted dibenzothiophenyl group, a substituted or unsubstituted dibenzosilolyl group, a substituted or unsubstituted benzonaphthofuranyl group, a substituted or unsubstituted benzonaphthothiophenyl group, a substituted or unsubstituted dinaphthofuranyl group, a substituted or unsubstituted dinaphthothiophenyl group, a substituted or unsubstituted benzophenanthrofuranyl group, or a substituted or unsubstituted benzophenanthrothiophenyl group.

5. The compound as claimed in claim 1, wherein *-L²-Ar¹ and *-L³-Ar² of Chemical Formula a are each independently selected from the substituents of Group I: 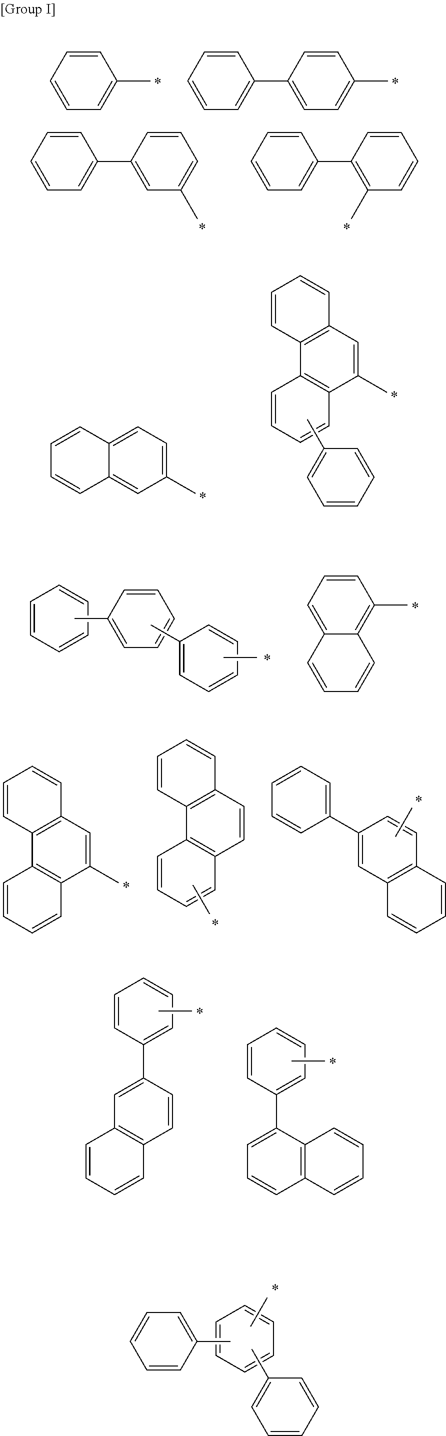 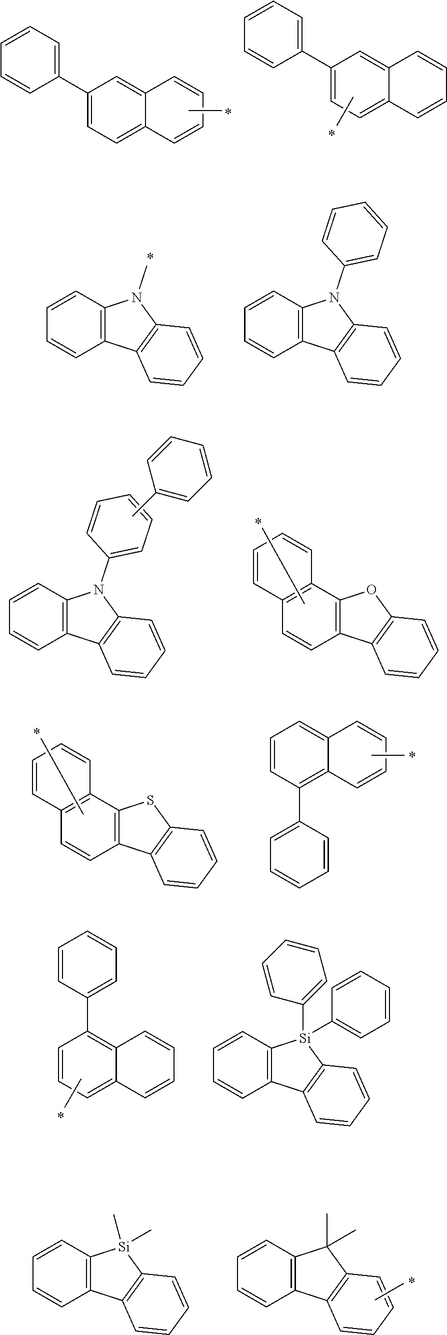 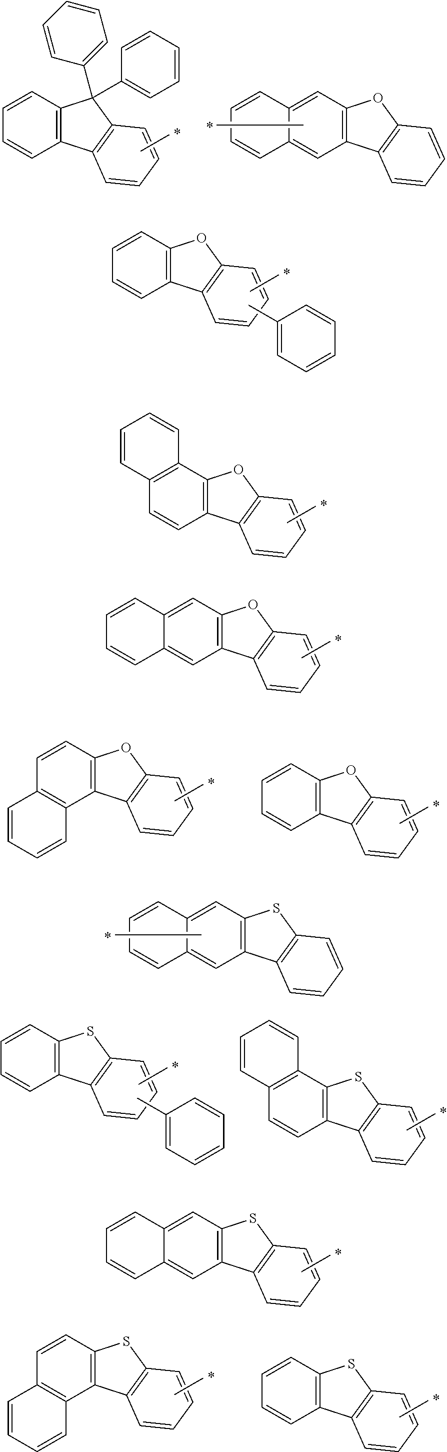 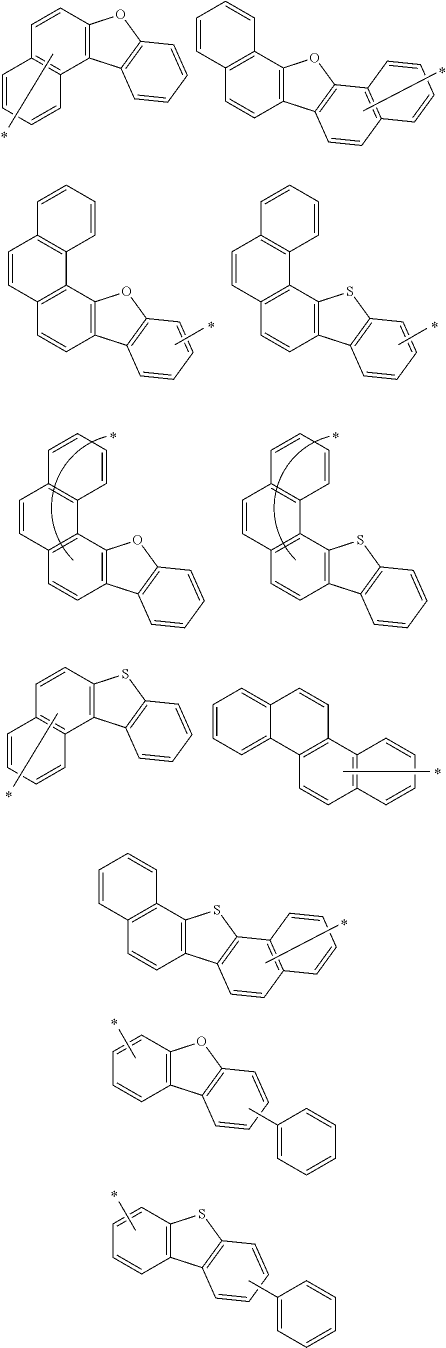 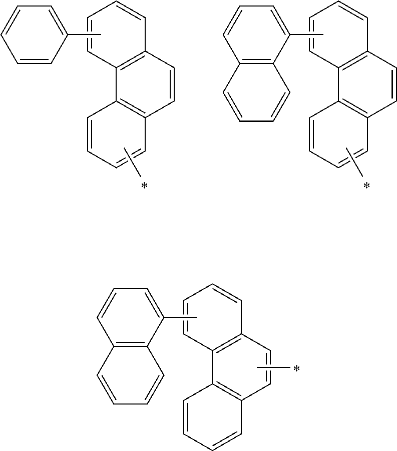
wherein, in Group I, * is a linking point.

6. The compound as claimed in claim 1, wherein the compound is selected from compounds of Group 1:

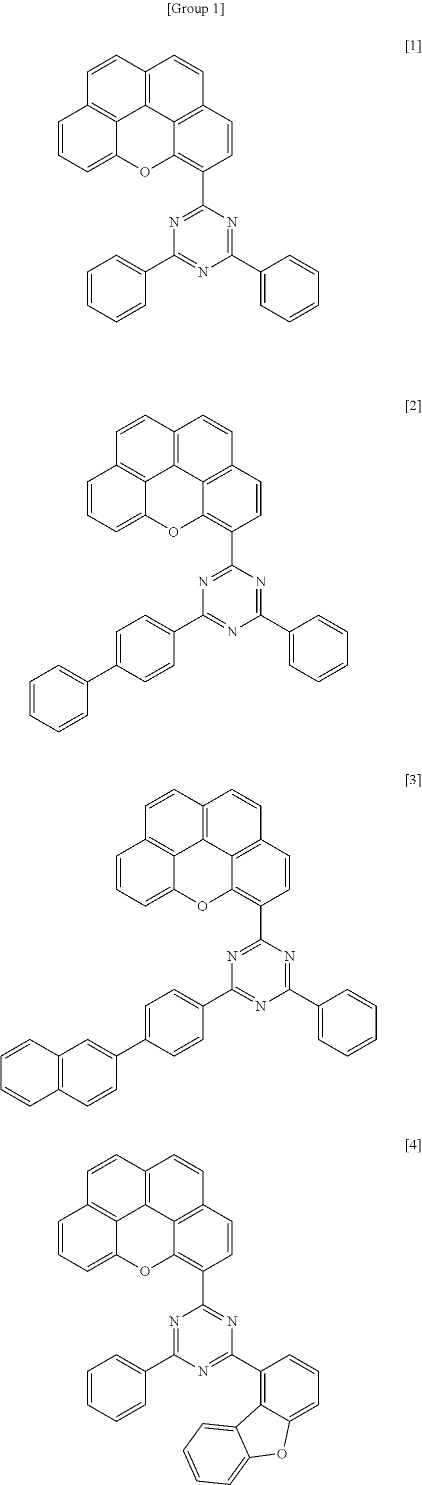 [SMILES: c1ccc(-c2nc(-c3ccccc3)nc(-c3ccc4ccc5ccc6cccc7c6c5c4c3O7)n2)cc1 ; c1ccc(-c2ccc(-c3nc(-c4ccccc4)nc(-c4ccc5ccc6ccc7cccc8c7c6c5c4O8)n3)cc2)cc1 ; c1ccc(-c2nc(-c3ccc(-c4ccc5ccccc5c4)cc3)nc(-c3ccc4ccc5ccc6cccc7c6c5c4c3O7)n2)cc1 ; c1ccc(-c2nc(-c3ccc4ccc5ccc6cccc7c6c5c4c3O7)nc(-c3cccc4oc5ccccc5c34)n2)cc1]

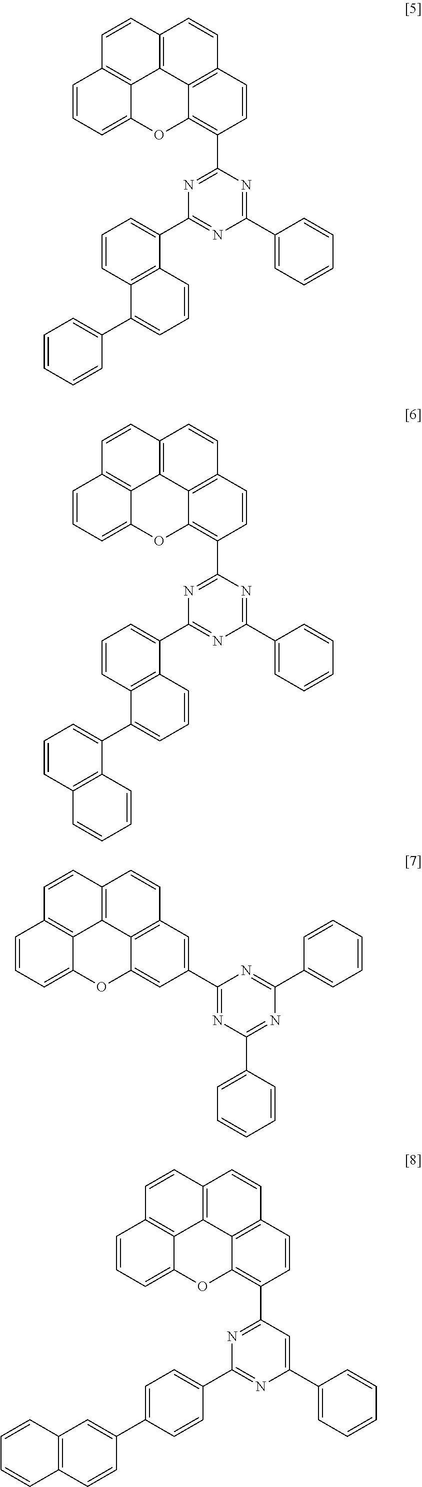

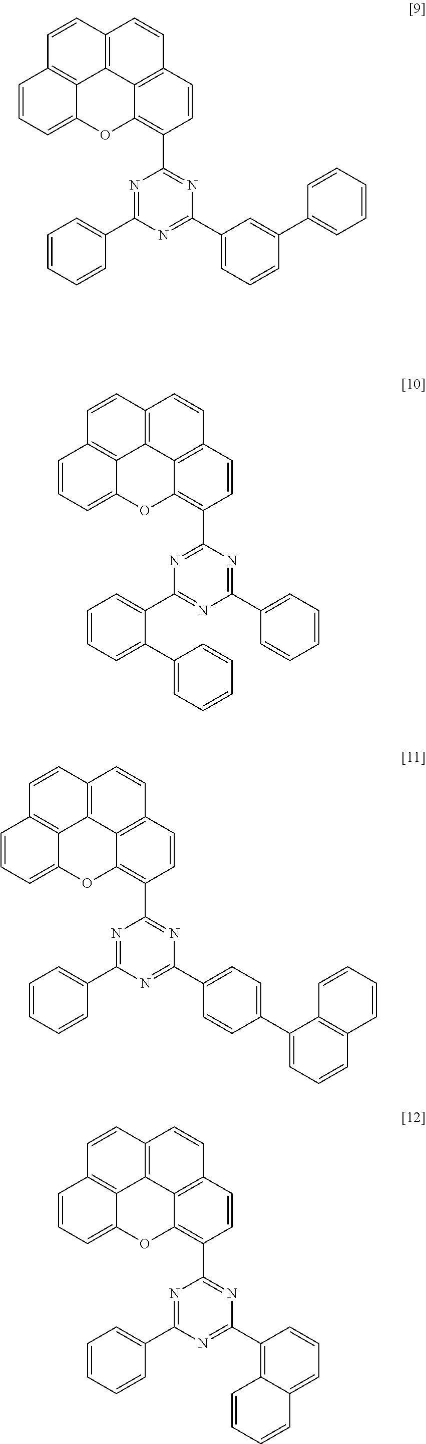 [SMILES: c1ccc(-c2cccc(-c3nc(-c4ccccc4)nc(-c4ccc5ccc6ccc7cccc8c7c6c5c4O8)n3)c2)cc1 ; c1ccc(-c2nc(-c3ccccc3-c3ccccc3)nc(-c3ccc4ccc5ccc6cccc7c6c5c4c3O7)n2)cc1 ; c1ccc(-c2nc(-c3ccc(-c4cccc5ccccc45)cc3)nc(-c3ccc4ccc5ccc6cccc7c6c5c4c3O7)n2)cc1 ; c1ccc(-c2nc(-c3ccc4ccc5ccc6cccc7c6c5c4c3O7)nc(-c3cccc4ccccc34)n2)cc1]

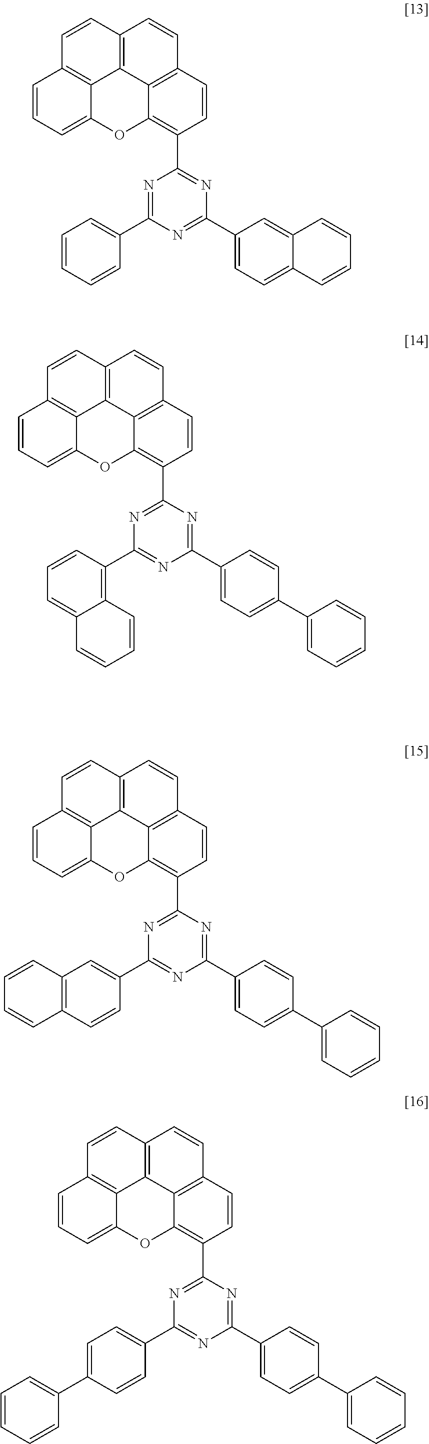 [SMILES: c1ccc(-c2nc(-c3ccc4ccccc4c3)nc(-c3ccc4ccc5ccc6cccc7c6c5c4c3O7)n2)cc1 ; c1ccc(-c2ccc(-c3nc(-c4ccc5ccc6ccc7cccc8c7c6c5c4O8)nc(-c4cccc5ccccc45)n3)cc2)cc1 ; c1ccc(-c2ccc(-c3nc(-c4ccc5ccccc5c4)nc(-c4ccc5ccc6ccc7cccc8c7c6c5c4O8)n3)cc2)cc1 ; c1ccc(-c2ccc(-c3nc(-c4ccc(-c5ccccc5)cc4)nc(-c4ccc5ccc6ccc7cccc8c7c6c5c4O8)n3)cc2)cc1]

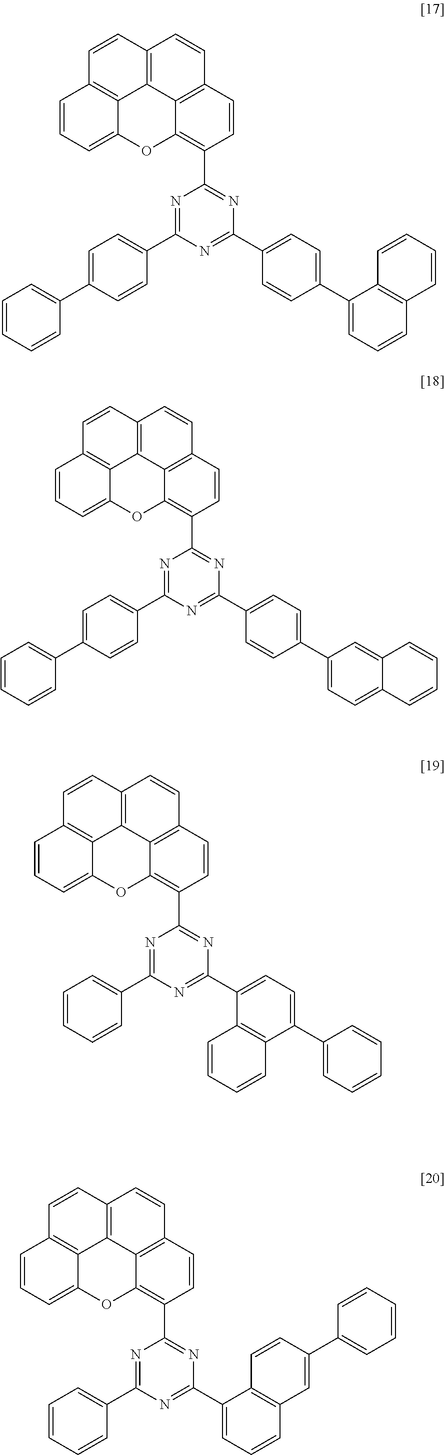 [SMILES: c1ccc(-c2ccc(-c3nc(-c4ccc(-c5cccc6ccccc56)cc4)nc(-c4ccc5ccc6ccc7cccc8c7c6c5c4O8)n3)cc2)cc1 ; c1ccc(-c2ccc(-c3nc(-c4ccc(-c5ccc6ccccc6c5)cc4)nc(-c4ccc5ccc6ccc7cccc8c7c6c5c4O8)n3)cc2)cc1 ; c1ccc(-c2nc(-c3ccc4ccc5ccc6cccc7c6c5c4c3O7)nc(-c3ccc(-c4ccccc4)c4ccccc34)n2)cc1 ; c1ccc(-c2ccc3c(-c4nc(-c5ccccc5)nc(-c5ccc6ccc7ccc8cccc9c8c7c6c5O9)n4)cccc3c2)cc1]

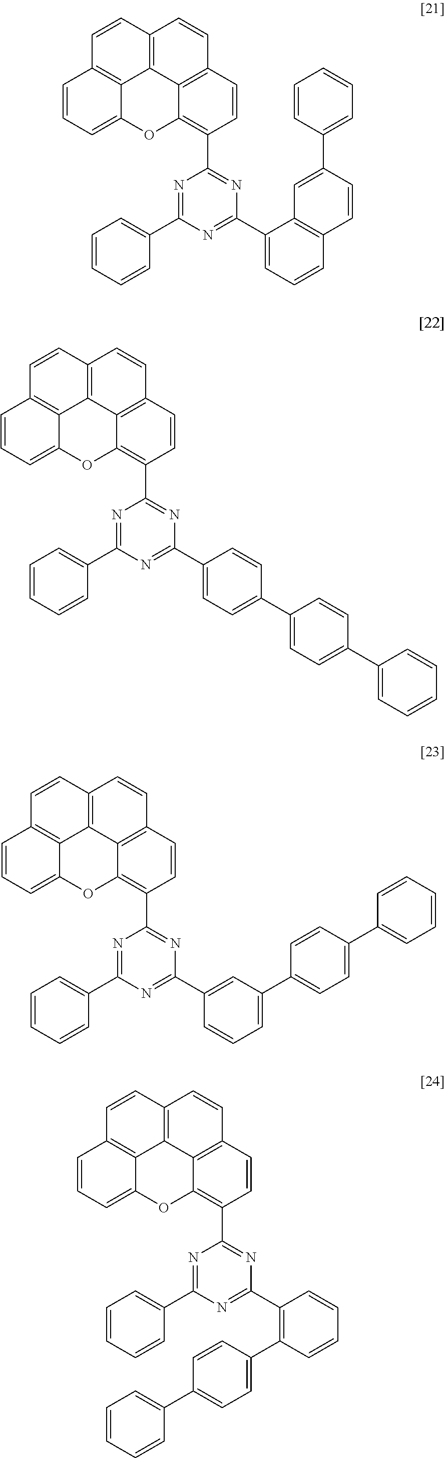 [SMILES: c1ccc(-c2ccc3cccc(-c4nc(-c5ccccc5)nc(-c5ccc6ccc7ccc8cccc9c8c7c6c5O9)n4)c3c2)cc1 ; c1ccc(-c2ccc(-c3ccc(-c4nc(-c5ccccc5)nc(-c5ccc6ccc7ccc8cccc9c8c7c6c5O9)n4)cc3)cc2)cc1 ; c1ccc(-c2ccc(-c3cccc(-c4nc(-c5ccccc5)nc(-c5ccc6ccc7ccc8cccc9c8c7c6c5O9)n4)c3)cc2)cc1 ; c1ccc(-c2ccc(-c3ccccc3-c3nc(-c4ccccc4)nc(-c4ccc5ccc6ccc7cccc8c7c6c5c4O8)n3)cc2)cc1]

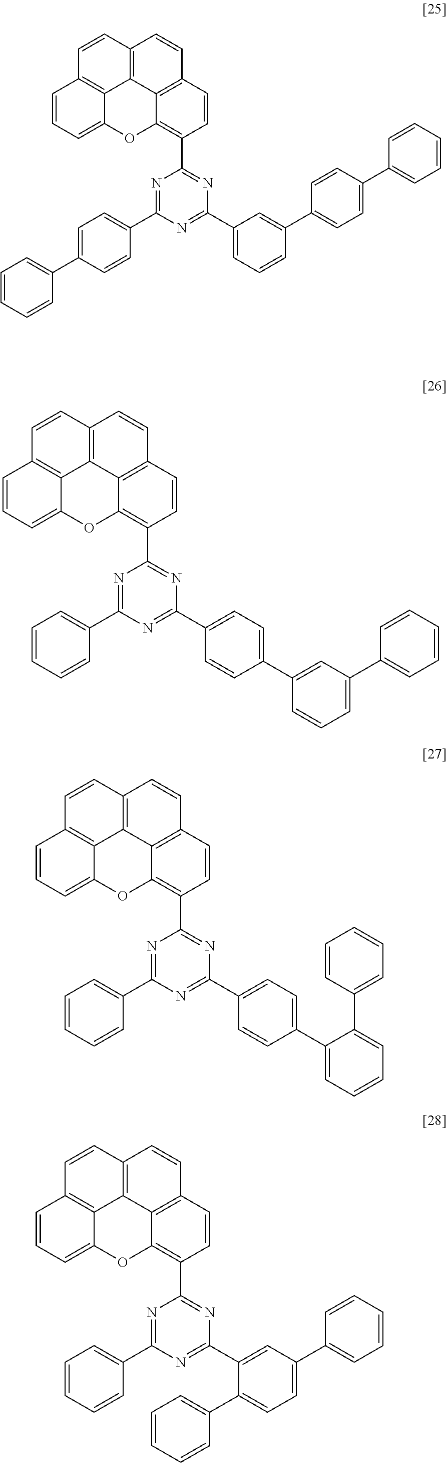 [SMILES: c1ccc(-c2ccc(-c3cccc(-c4nc(-c5ccc(-c6ccccc6)cc5)nc(-c5ccc6ccc7ccc8cccc9c8c7c6c5O9)n4)c3)cc2)cc1 ; c1ccc(-c2cccc(-c3ccc(-c4nc(-c5ccccc5)nc(-c5ccc6ccc7ccc8cccc9c8c7c6c5O9)n4)cc3)c2)cc1 ; c1ccc(-c2nc(-c3ccc(-c4ccccc4-c4ccccc4)cc3)nc(-c3ccc4ccc5ccc6cccc7c6c5c4c3O7)n2)cc1 ; c1ccc(-c2ccc(-c3ccccc3)c(-c3nc(-c4ccccc4)nc(-c4ccc5ccc6ccc7cccc8c7c6c5c4O8)n3)c2)cc1]

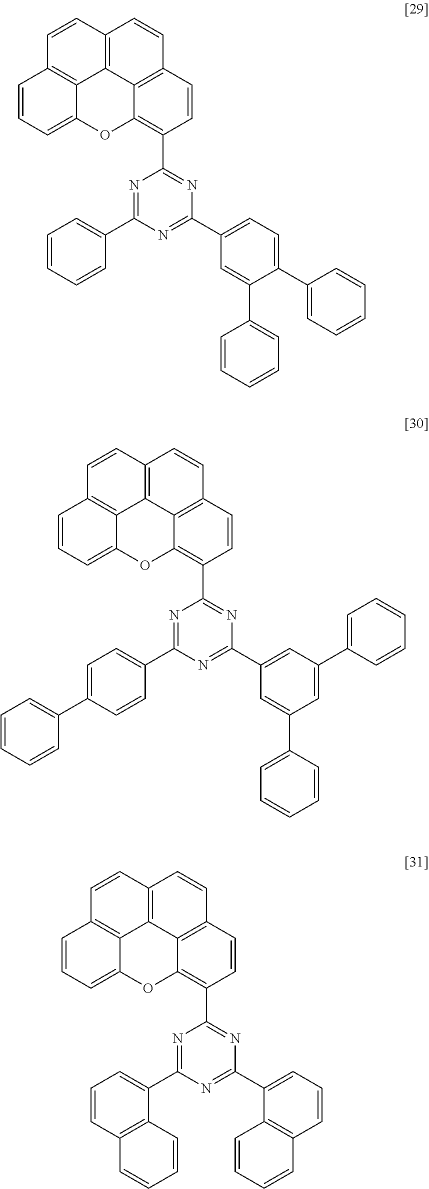 [SMILES: c1ccc(-c2nc(-c3ccc(-c4ccccc4)c(-c4ccccc4)c3)nc(-c3ccc4ccc5ccc6cccc7c6c5c4c3O7)n2)cc1 ; c1ccc(-c2ccc(-c3nc(-c4cc(-c5ccccc5)cc(-c5ccccc5)c4)nc(-c4ccc5ccc6ccc7cccc8c7c6c5c4O8)n3)cc2)cc1 ; c1ccc2c(-c3nc(-c4ccc5ccc6ccc7cccc8c7c6c5c4O8)nc(-c4cccc5ccccc45)n3)cccc2c1]

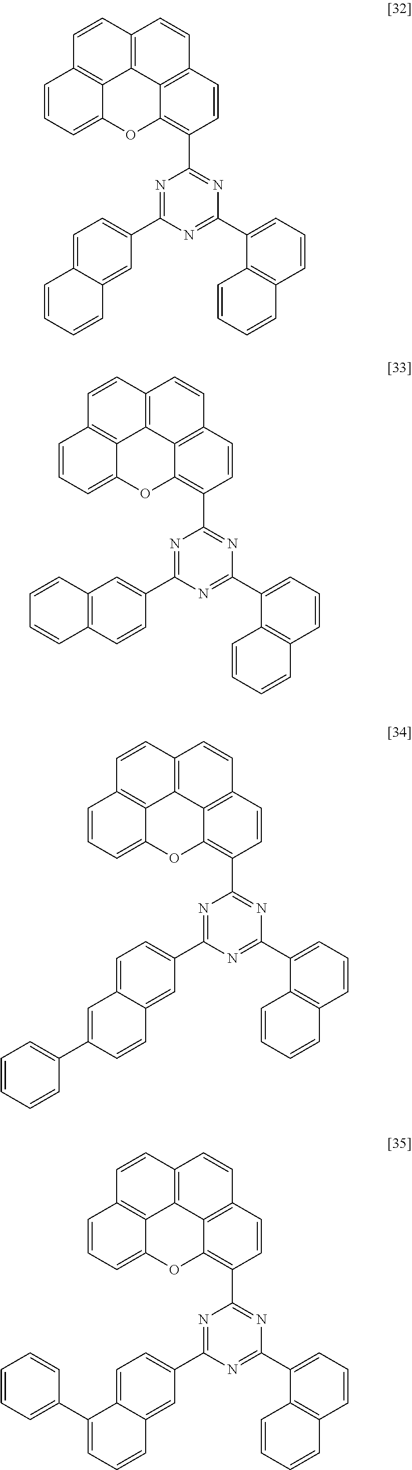 [SMILES: c1ccc2cc(-c3nc(-c4ccc5ccc6ccc7cccc8c7c6c5c4O8)nc(-c4cccc5ccccc45)n3)ccc2c1 ; c1ccc2cc(-c3nc(-c4ccc5ccc6ccc7cccc8c7c6c5c4O8)nc(-c4cccc5ccccc45)n3)ccc2c1 ; c1ccc(-c2ccc3cc(-c4nc(-c5ccc6ccc7ccc8cccc9c8c7c6c5O9)nc(-c5cccc6ccccc56)n4)ccc3c2)cc1 ; c1ccc(-c2cccc3cc(-c4nc(-c5ccc6ccc7ccc8cccc9c8c7c6c5O9)nc(-c5cccc6ccccc56)n4)ccc23)cc1]

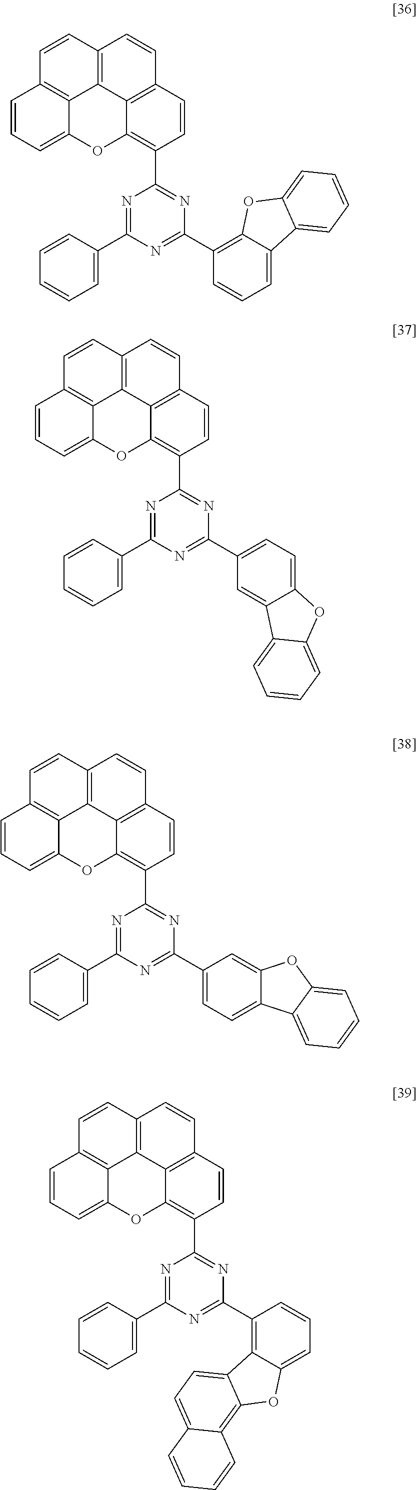

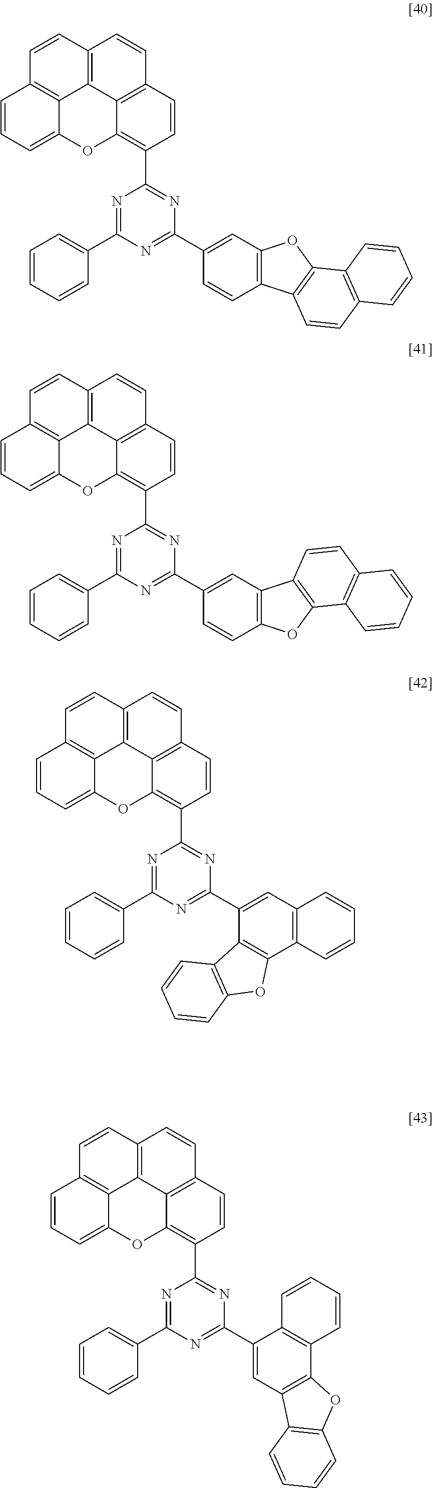 [SMILES: c1ccc(-c2nc(-c3ccc4c(c3)oc3c5ccccc5ccc43)nc(-c3ccc4ccc5ccc6cccc7c6c5c4c3O7)n2)cc1 ; c1ccc(-c2nc(-c3ccc4oc5c6ccccc6ccc5c4c3)nc(-c3ccc4ccc5ccc6cccc7c6c5c4c3O7)n2)cc1 ; c1ccc(-c2nc(-c3ccc4ccc5ccc6cccc7c6c5c4c3O7)nc(-c3cc4ccccc4c4oc5ccccc5c34)n2)cc1 ; c1ccc(-c2nc(-c3ccc4ccc5ccc6cccc7c6c5c4c3O7)nc(-c3cc4c5ccccc5oc4c4ccccc34)n2)cc1]

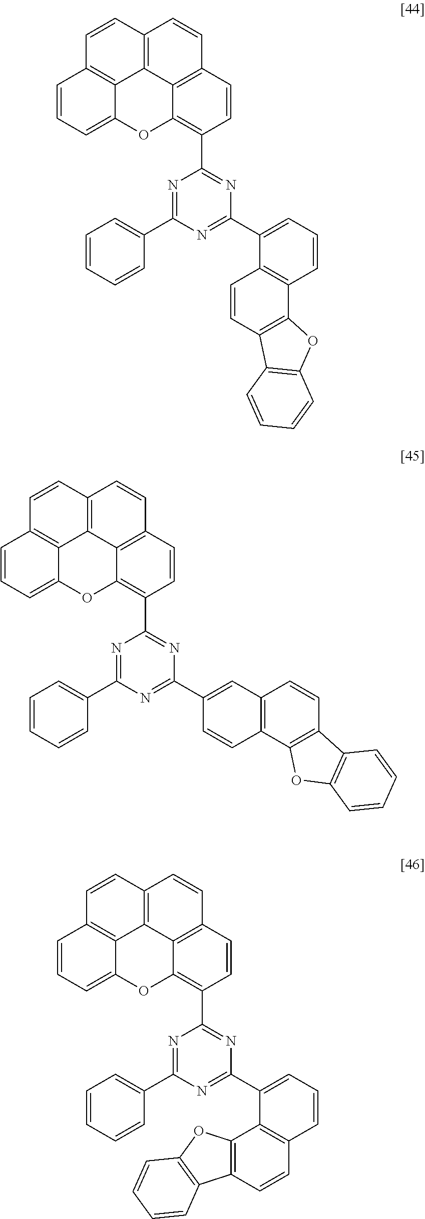 [SMILES: c1ccc(-c2nc(-c3ccc4ccc5ccc6cccc7c6c5c4c3O7)nc(-c3cccc4c3ccc3c5ccccc5oc43)n2)cc1 ; c1ccc(-c2nc(-c3ccc4c(ccc5c6ccccc6oc45)c3)nc(-c3ccc4ccc5ccc6cccc7c6c5c4c3O7)n2)cc1 ; c1ccc(-c2nc(-c3ccc4ccc5ccc6cccc7c6c5c4c3O7)nc(-c3cccc4ccc5c6ccccc6oc5c34)n2)cc1]

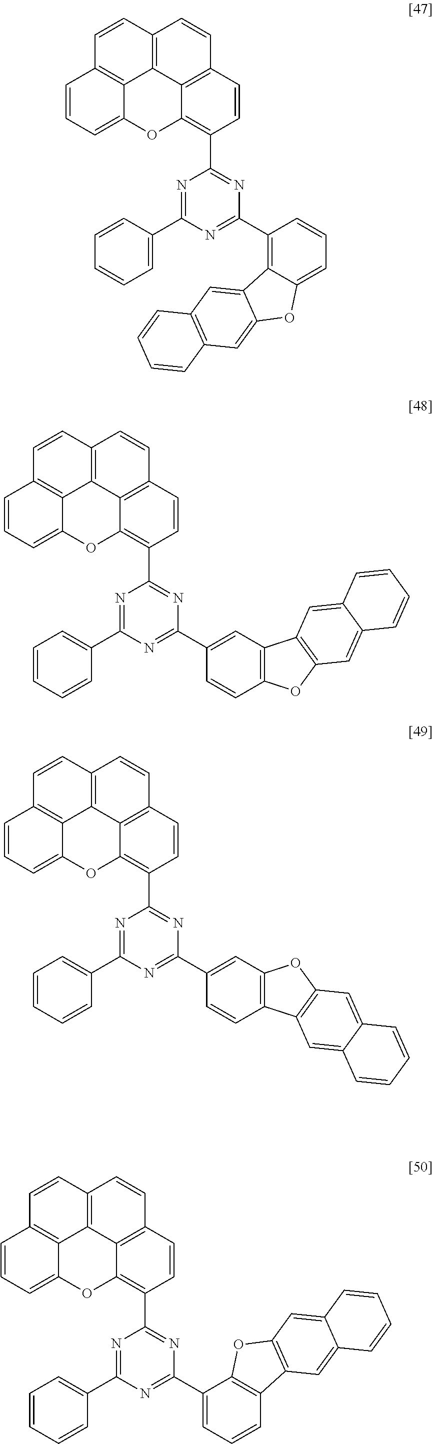 [SMILES: c1ccc(-c2nc(-c3ccc4ccc5ccc6cccc7c6c5c4c3O7)nc(-c3cccc4oc5cc6ccccc6cc5c34)n2)cc1 ; c1ccc(-c2nc(-c3ccc4oc5cc6ccccc6cc5c4c3)nc(-c3ccc4ccc5ccc6cccc7c6c5c4c3O7)n2)cc1 ; c1ccc(-c2nc(-c3ccc4c(c3)oc3cc5ccccc5cc34)nc(-c3ccc4ccc5ccc6cccc7c6c5c4c3O7)n2)cc1 ; c1ccc(-c2nc(-c3ccc4ccc5ccc6cccc7c6c5c4c3O7)nc(-c3cccc4c3oc3cc5ccccc5cc34)n2)cc1]

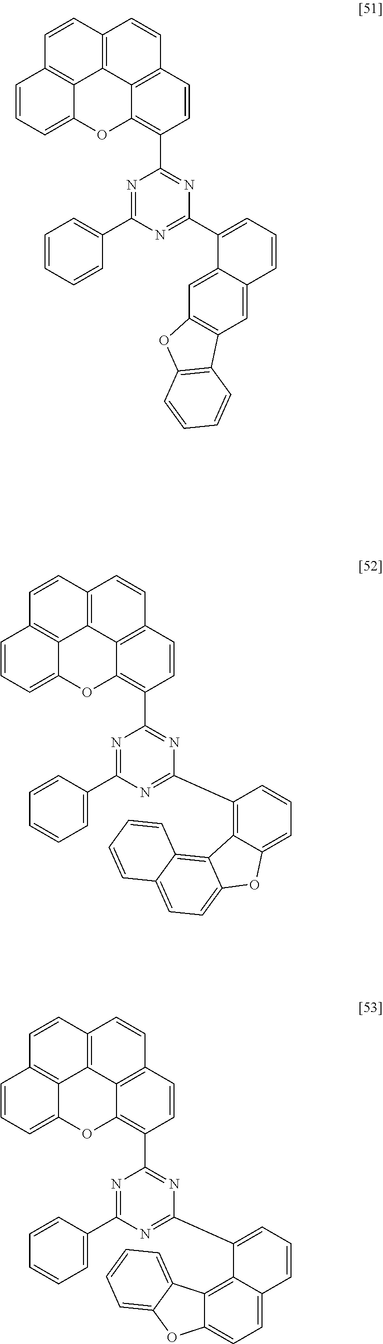 [SMILES: c1ccc(-c2nc(-c3ccc4ccc5ccc6cccc7c6c5c4c3O7)nc(-c3cccc4cc5c(cc34)oc3ccccc35)n2)cc1 ; c1ccc(-c2nc(-c3ccc4ccc5ccc6cccc7c6c5c4c3O7)nc(-c3cccc4oc5ccc6ccccc6c5c34)n2)cc1 ; c1ccc(-c2nc(-c3ccc4ccc5ccc6cccc7c6c5c4c3O7)nc(-c3cccc4ccc5oc6ccccc6c5c34)n2)cc1]

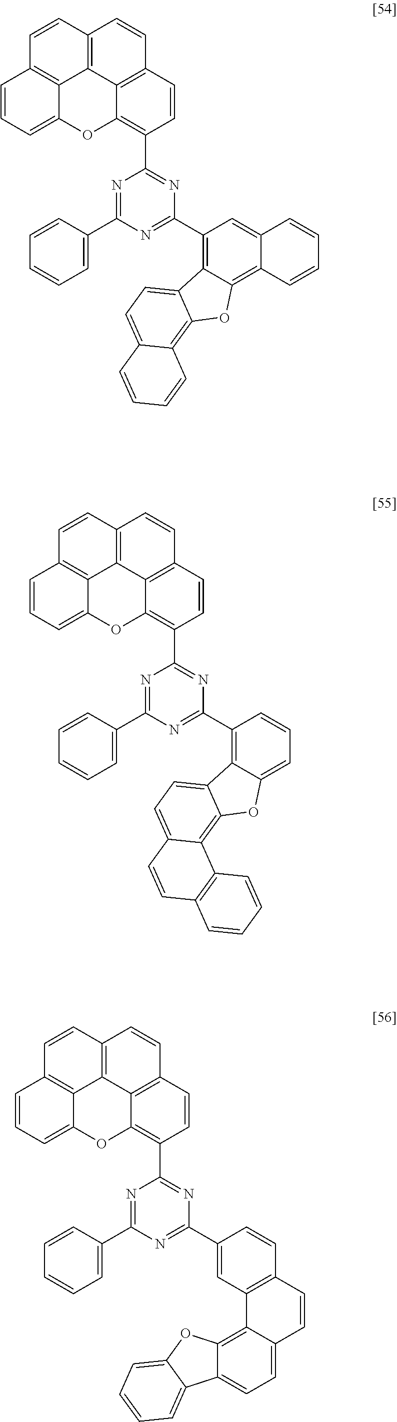 [SMILES: c1ccc(-c2nc(-c3ccc4ccc5ccc6cccc7c6c5c4c3O7)nc(-c3cc4ccccc4c4oc5c6ccccc6ccc5c34)n2)cc1 ; c1ccc(-c2nc(-c3ccc4ccc5ccc6cccc7c6c5c4c3O7)nc(-c3cccc4oc5c(ccc6ccc7ccccc7c65)c34)n2)cc1 ; c1ccc(-c2nc(-c3ccc4ccc5ccc6c7ccccc7oc6c5c4c3)nc(-c3ccc4ccc5ccc6cccc7c6c5c4c3O7)n2)cc1]

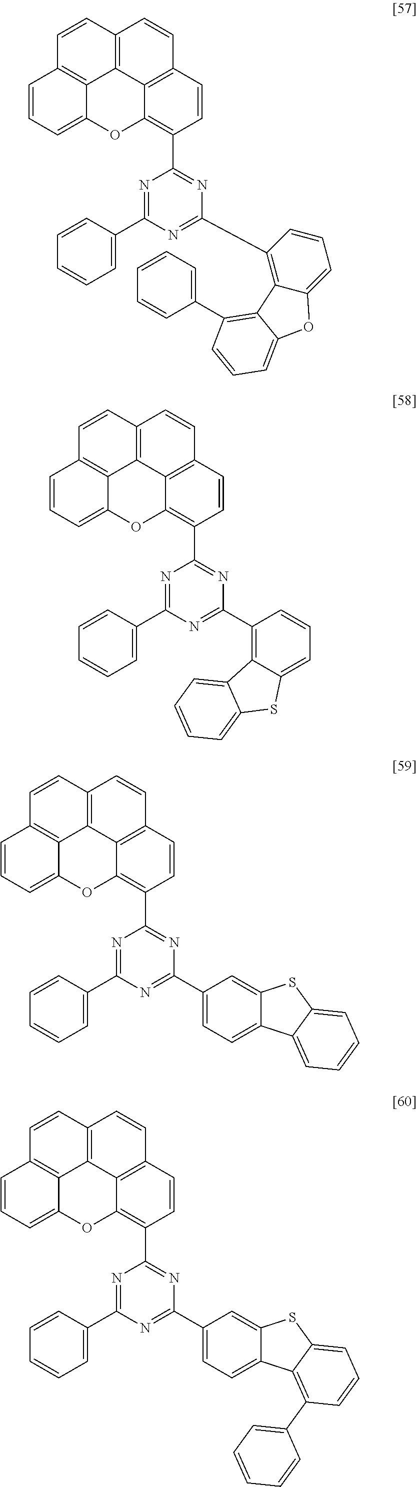 [SMILES: c1ccc(-c2nc(-c3ccc4ccc5ccc6cccc7c6c5c4c3O7)nc(-c3cccc4oc5cccc(-c6ccccc6)c5c34)n2)cc1 ; c1ccc(-c2nc(-c3ccc4ccc5ccc6cccc7c6c5c4c3O7)nc(-c3cccc4sc5ccccc5c34)n2)cc1 ; c1ccc(-c2nc(-c3ccc4c(c3)sc3ccccc34)nc(-c3ccc4ccc5ccc6cccc7c6c5c4c3O7)n2)cc1 ; c1ccc(-c2nc(-c3ccc4c(c3)sc3cccc(-c5ccccc5)c34)nc(-c3ccc4ccc5ccc6cccc7c6c5c4c3O7)n2)cc1]

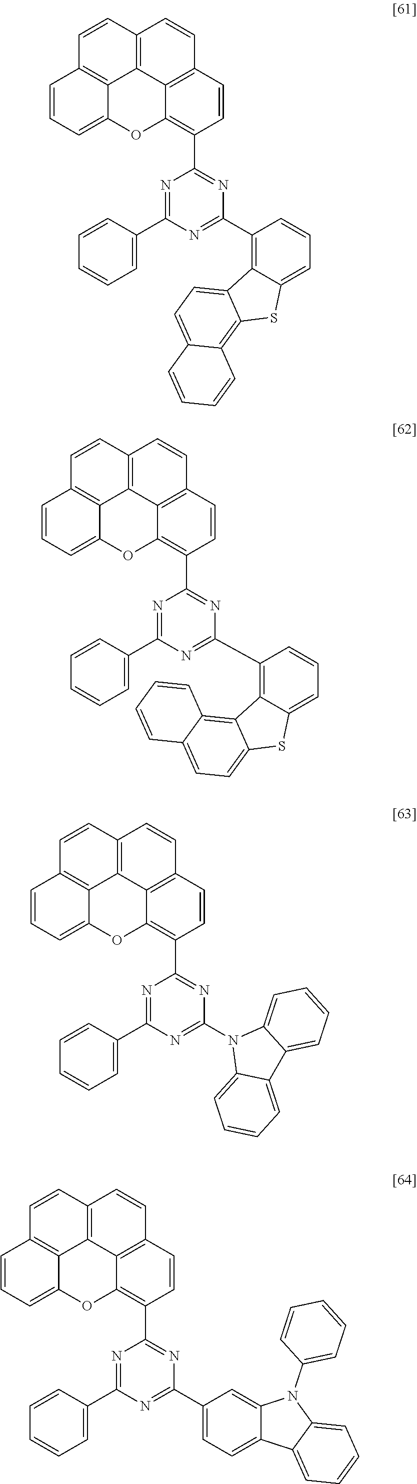

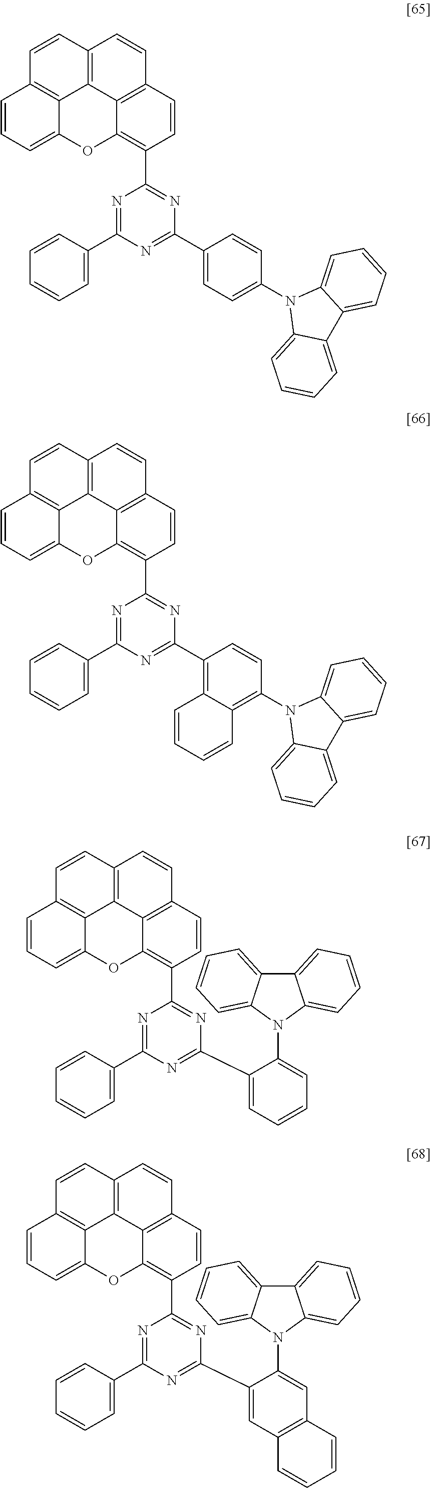

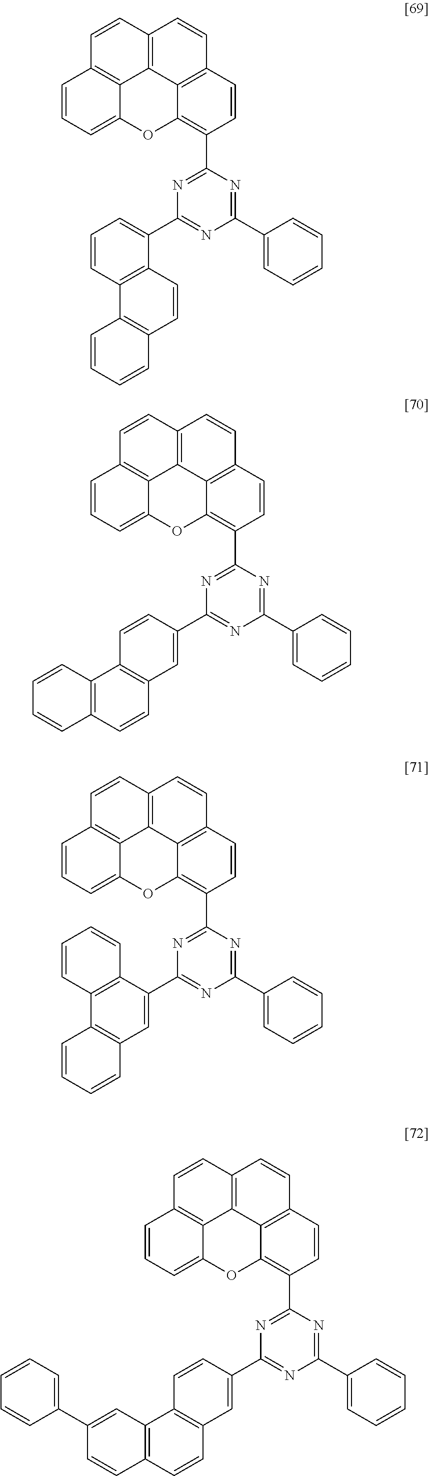

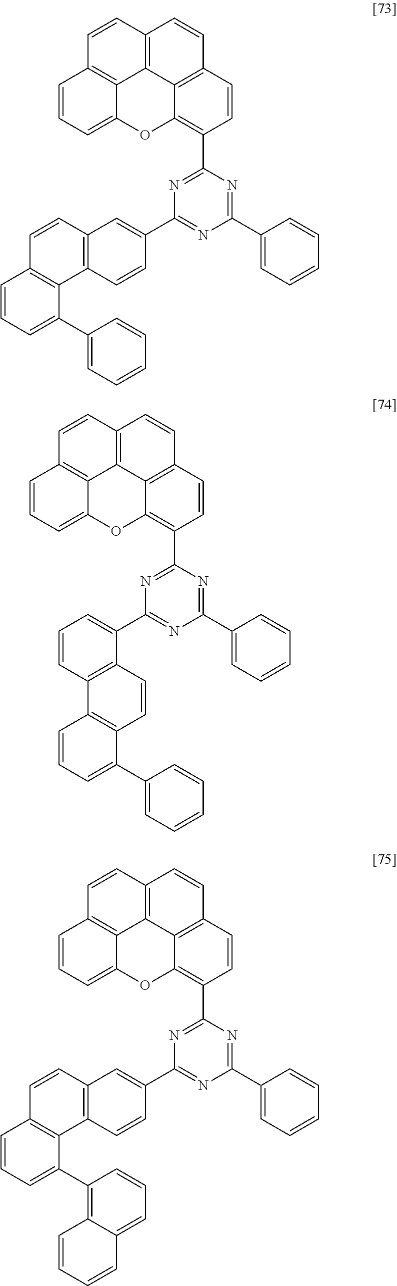

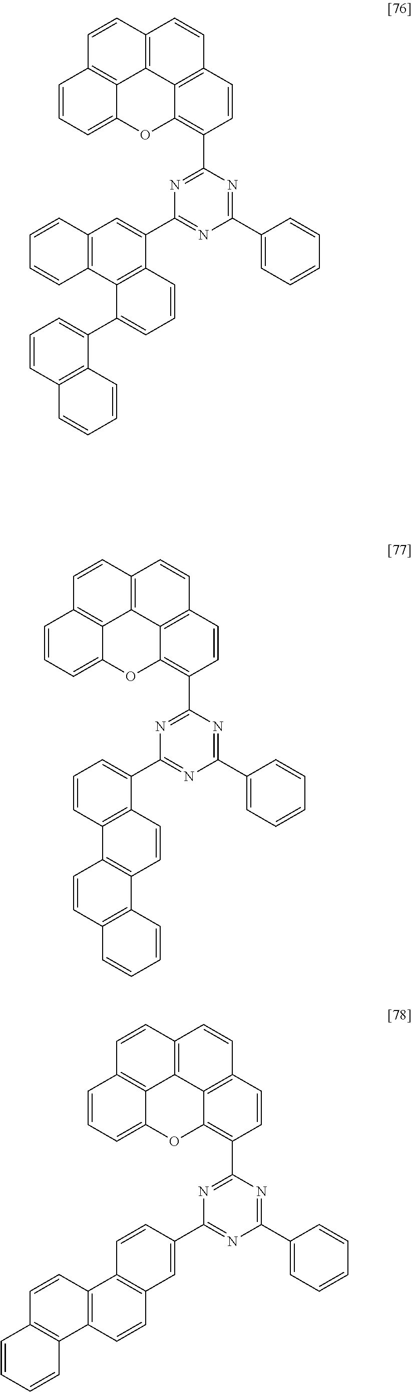 [SMILES: c1ccc(-c2nc(-c3ccc4ccc5ccc6cccc7c6c5c4c3O7)nc(-c3cc4ccccc4c4c(-c5cccc6ccccc56)cccc34)n2)cc1 ; c1ccc(-c2nc(-c3ccc4ccc5ccc6cccc7c6c5c4c3O7)nc(-c3cccc4c3ccc3c5ccccc5ccc43)n2)cc1 ; c1ccc(-c2nc(-c3ccc4c(ccc5c6ccccc6ccc45)c3)nc(-c3ccc4ccc5ccc6cccc7c6c5c4c3O7)n2)cc1]

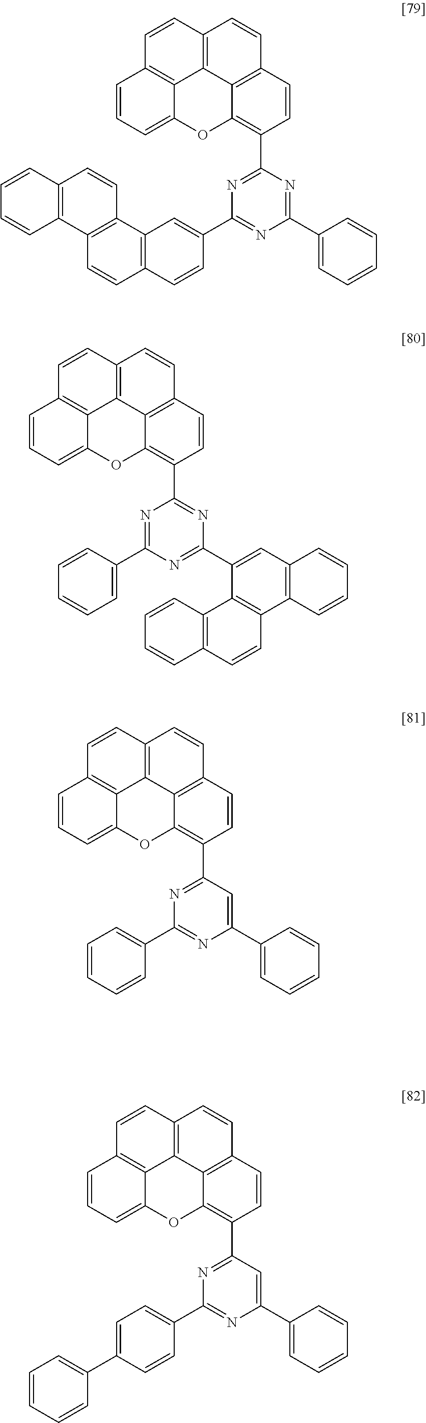 [SMILES: c1ccc(-c2nc(-c3ccc4ccc5c6ccccc6ccc5c4c3)nc(-c3ccc4ccc5ccc6cccc7c6c5c4c3O7)n2)cc1 ; c1ccc(-c2nc(-c3ccc4ccc5ccc6cccc7c6c5c4c3O7)nc(-c3cc4ccccc4c4ccc5ccccc5c34)n2)cc1 ; c1ccc(-c2cc(-c3ccc4ccc5ccc6cccc7c6c5c4c3O7)nc(-c3ccccc3)n2)cc1 ; c1ccc(-c2ccc(-c3nc(-c4ccccc4)cc(-c4ccc5ccc6ccc7cccc8c7c6c5c4O8)n3)cc2)cc1]

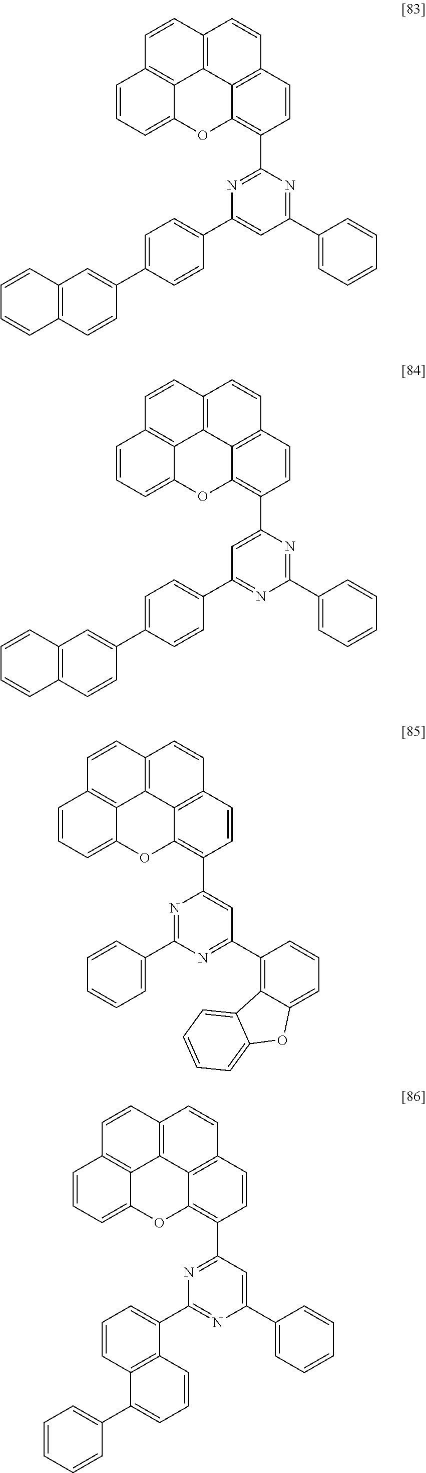

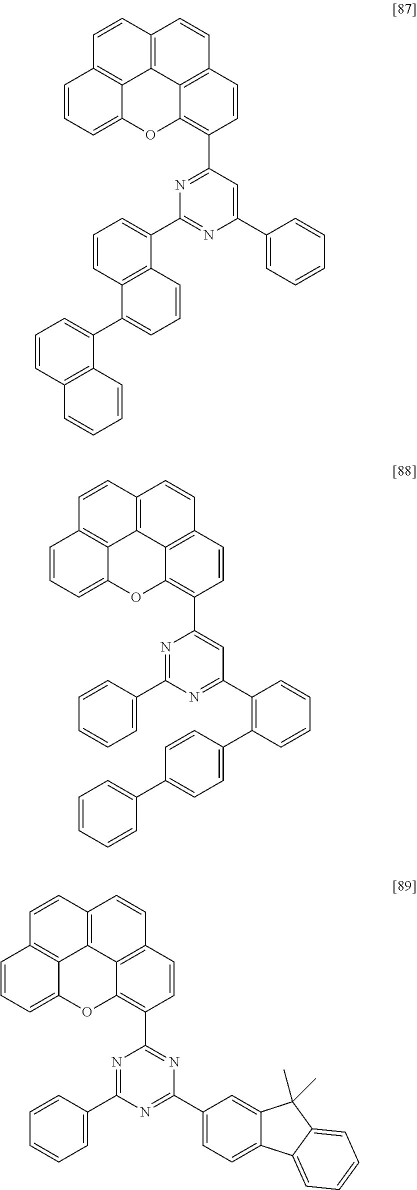 [SMILES: c1ccc(-c2cc(-c3ccc4ccc5ccc6cccc7c6c5c4c3O7)nc(-c3cccc4c(-c5cccc6ccccc56)cccc34)n2)cc1 ; c1ccc(-c2ccc(-c3ccccc3-c3cc(-c4ccc5ccc6ccc7cccc8c7c6c5c4O8)nc(-c4ccccc4)n3)cc2)cc1 ; CC1(C)c2ccccc2-c2ccc(-c3nc(-c4ccccc4)nc(-c4ccc5ccc6ccc7cccc8c7c6c5c4O8)n3)cc21]

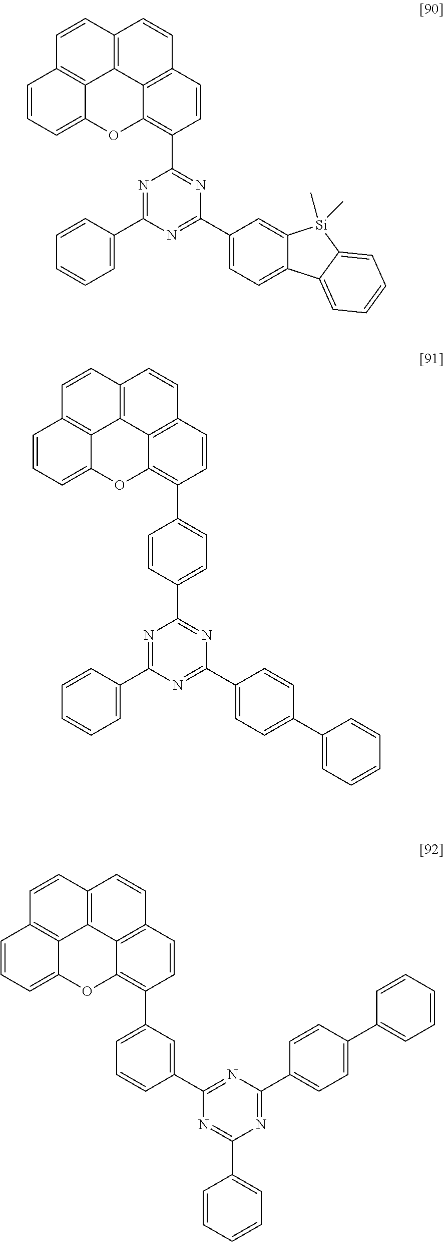 [SMILES: C[Si]1(C)c2ccccc2-c2ccc(-c3nc(-c4ccccc4)nc(-c4ccc5ccc6ccc7cccc8c7c6c5c4O8)n3)cc21 ; c1ccc(-c2ccc(-c3nc(-c4ccccc4)nc(-c4ccc(-c5ccc6ccc7ccc8cccc9c8c7c6c5O9)cc4)n3)cc2)cc1 ; c1ccc(-c2ccc(-c3nc(-c4ccccc4)nc(-c4cccc(-c5ccc6ccc7ccc8cccc9c8c7c6c5O9)c4)n3)cc2)cc1]

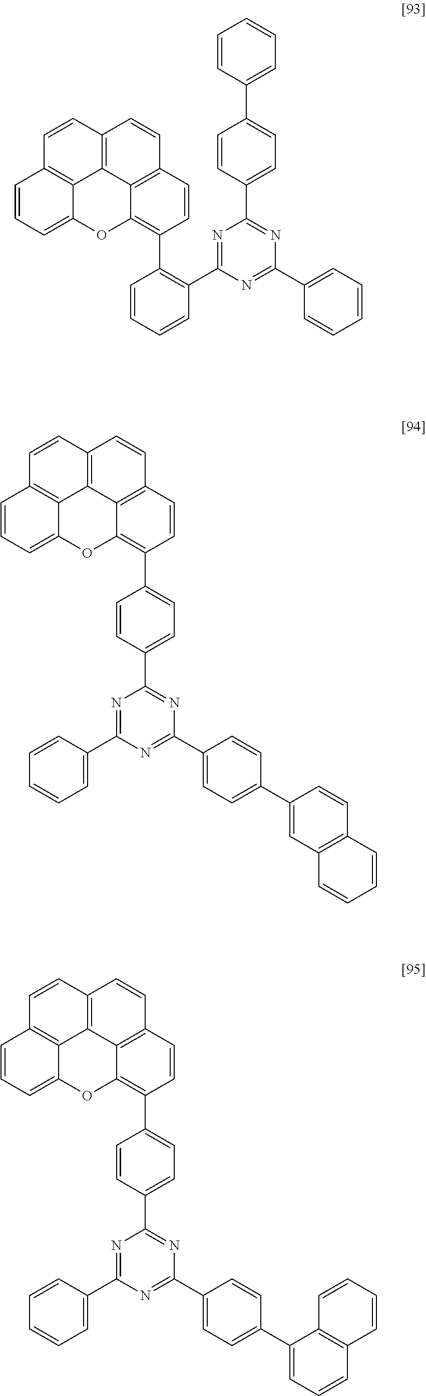 [SMILES: c1ccc(-c2ccc(-c3nc(-c4ccccc4)nc(-c4ccccc4-c4ccc5ccc6ccc7cccc8c7c6c5c4O8)n3)cc2)cc1 ; c1ccc(-c2nc(-c3ccc(-c4ccc5ccccc5c4)cc3)nc(-c3ccc(-c4ccc5ccc6ccc7cccc8c7c6c5c4O8)cc3)n2)cc1 ; c1ccc(-c2nc(-c3ccc(-c4ccc5ccc6ccc7cccc8c7c6c5c4O8)cc3)nc(-c3ccc(-c4cccc5ccccc45)cc3)n2)cc1]

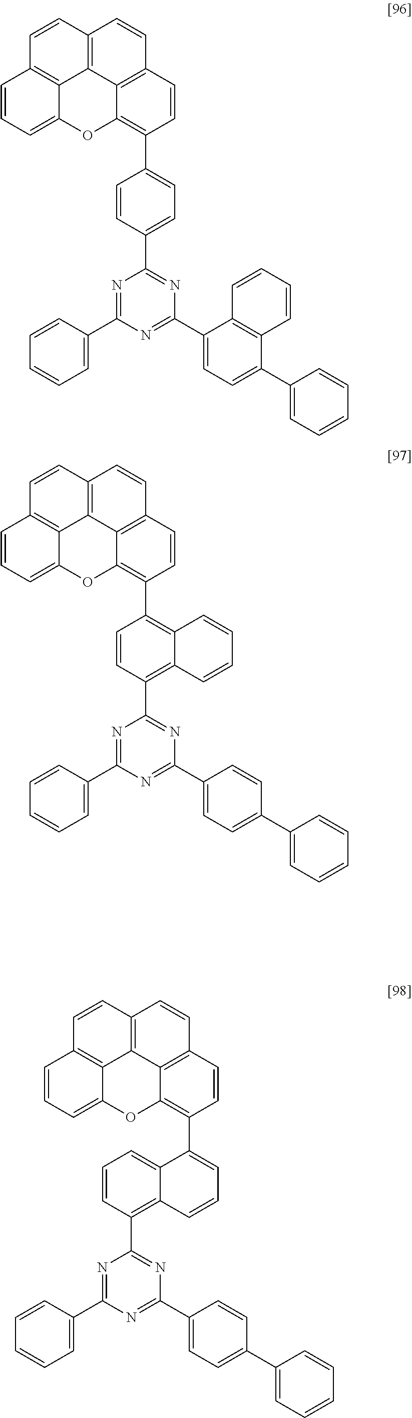 [SMILES: c1ccc(-c2nc(-c3ccc(-c4ccc5ccc6ccc7cccc8c7c6c5c4O8)cc3)nc(-c3ccc(-c4ccccc4)c4ccccc34)n2)cc1 ; c1ccc(-c2ccc(-c3nc(-c4ccccc4)nc(-c4ccc(-c5ccc6ccc7ccc8cccc9c8c7c6c5O9)c5ccccc45)n3)cc2)cc1 ; c1ccc(-c2ccc(-c3nc(-c4ccccc4)nc(-c4cccc5c(-c6ccc7ccc8ccc9cccc%10c9c8c7c6O%10)cccc45)n3)cc2)cc1]

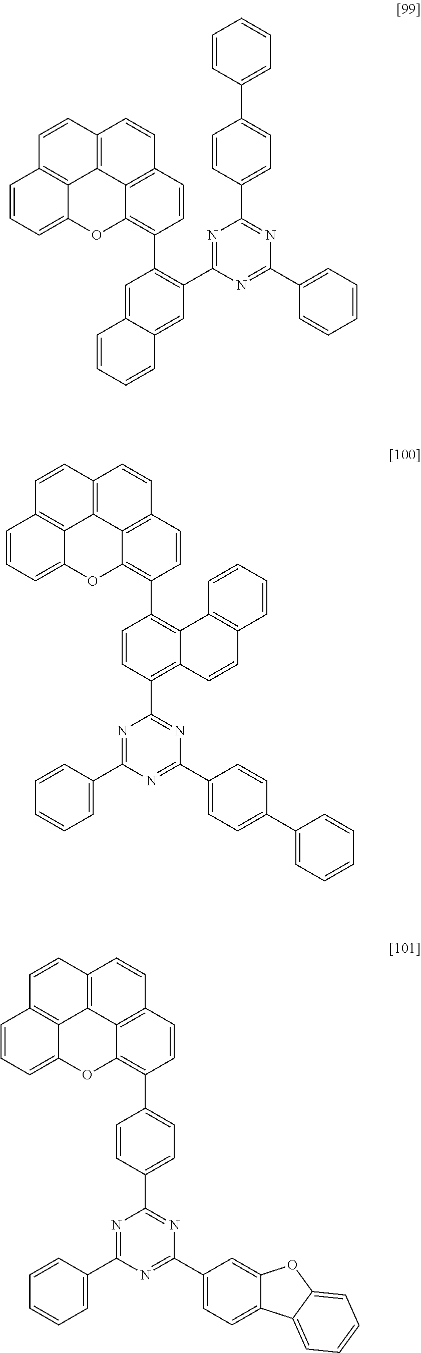 [SMILES: c1ccc(-c2ccc(-c3nc(-c4ccccc4)nc(-c4cc5ccccc5cc4-c4ccc5ccc6ccc7cccc8c7c6c5c4O8)n3)cc2)cc1 ; c1ccc(-c2ccc(-c3nc(-c4ccccc4)nc(-c4ccc(-c5ccc6ccc7ccc8cccc9c8c7c6c5O9)c5c4ccc4ccccc45)n3)cc2)cc1 ; c1ccc(-c2nc(-c3ccc(-c4ccc5ccc6ccc7cccc8c7c6c5c4O8)cc3)nc(-c3ccc4c(c3)oc3ccccc34)n2)cc1]

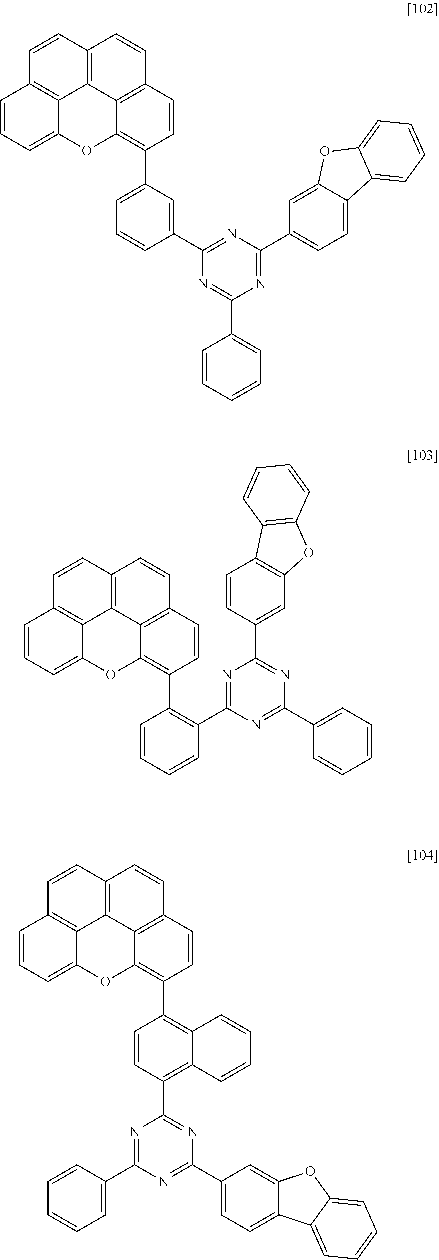 [SMILES: c1ccc(-c2nc(-c3cccc(-c4ccc5ccc6ccc7cccc8c7c6c5c4O8)c3)nc(-c3ccc4c(c3)oc3ccccc34)n2)cc1 ; c1ccc(-c2nc(-c3ccc4c(c3)oc3ccccc34)nc(-c3ccccc3-c3ccc4ccc5ccc6cccc7c6c5c4c3O7)n2)cc1 ; c1ccc(-c2nc(-c3ccc4c(c3)oc3ccccc34)nc(-c3ccc(-c4ccc5ccc6ccc7cccc8c7c6c5c4O8)c4ccccc34)n2)cc1]

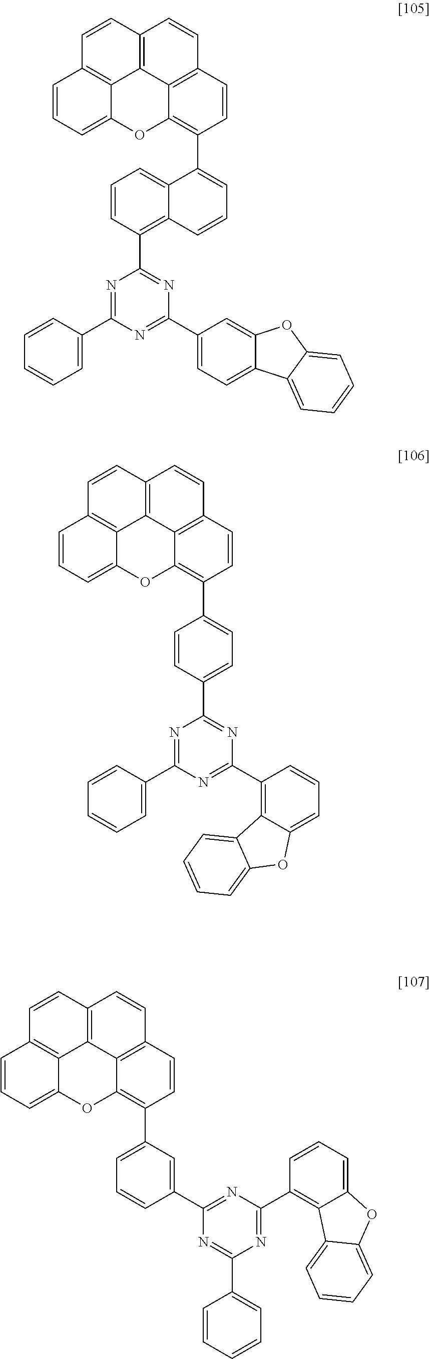 [SMILES: c1ccc(-c2nc(-c3ccc4c(c3)oc3ccccc34)nc(-c3cccc4c(-c5ccc6ccc7ccc8cccc9c8c7c6c5O9)cccc34)n2)cc1 ; c1ccc(-c2nc(-c3ccc(-c4ccc5ccc6ccc7cccc8c7c6c5c4O8)cc3)nc(-c3cccc4oc5ccccc5c34)n2)cc1 ; c1ccc(-c2nc(-c3cccc(-c4ccc5ccc6ccc7cccc8c7c6c5c4O8)c3)nc(-c3cccc4oc5ccccc5c34)n2)cc1]

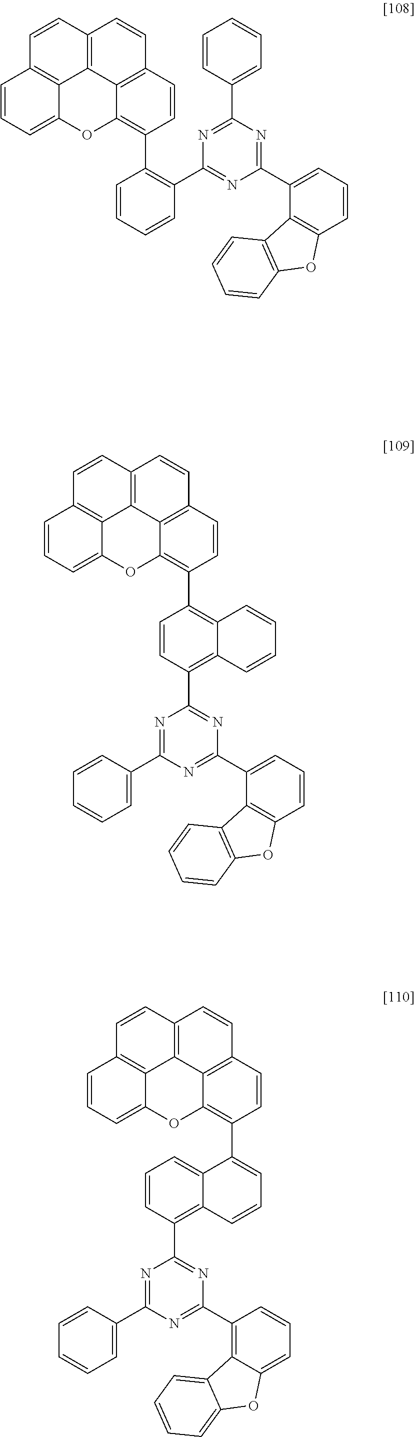 [SMILES: c1ccc(-c2nc(-c3ccccc3-c3ccc4ccc5ccc6cccc7c6c5c4c3O7)nc(-c3cccc4oc5ccccc5c34)n2)cc1 ; c1ccc(-c2nc(-c3ccc(-c4ccc5ccc6ccc7cccc8c7c6c5c4O8)c4ccccc34)nc(-c3cccc4oc5ccccc5c34)n2)cc1 ; c1ccc(-c2nc(-c3cccc4c(-c5ccc6ccc7ccc8cccc9c8c7c6c5O9)cccc34)nc(-c3cccc4oc5ccccc5c34)n2)cc1]

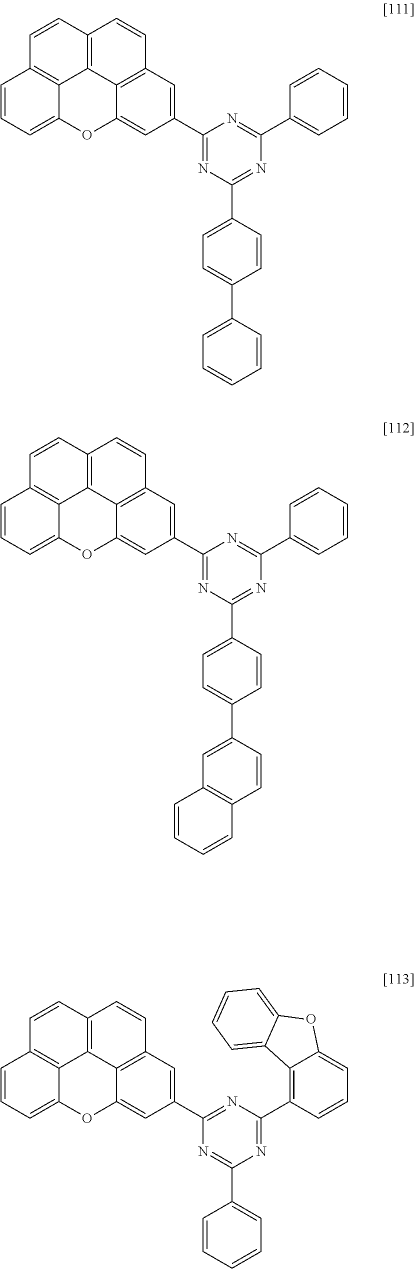 [SMILES: c1ccc(-c2ccc(-c3nc(-c4ccccc4)nc(-c4cc5c6c(ccc7ccc8cccc(c8c76)O5)c4)n3)cc2)cc1 ; c1ccc(-c2nc(-c3ccc(-c4ccc5ccccc5c4)cc3)nc(-c3cc4c5c(ccc6ccc7cccc(c7c65)O4)c3)n2)cc1 ; c1ccc(-c2nc(-c3cc4c5c(ccc6ccc7cccc(c7c65)O4)c3)nc(-c3cccc4oc5ccccc5c34)n2)cc1]

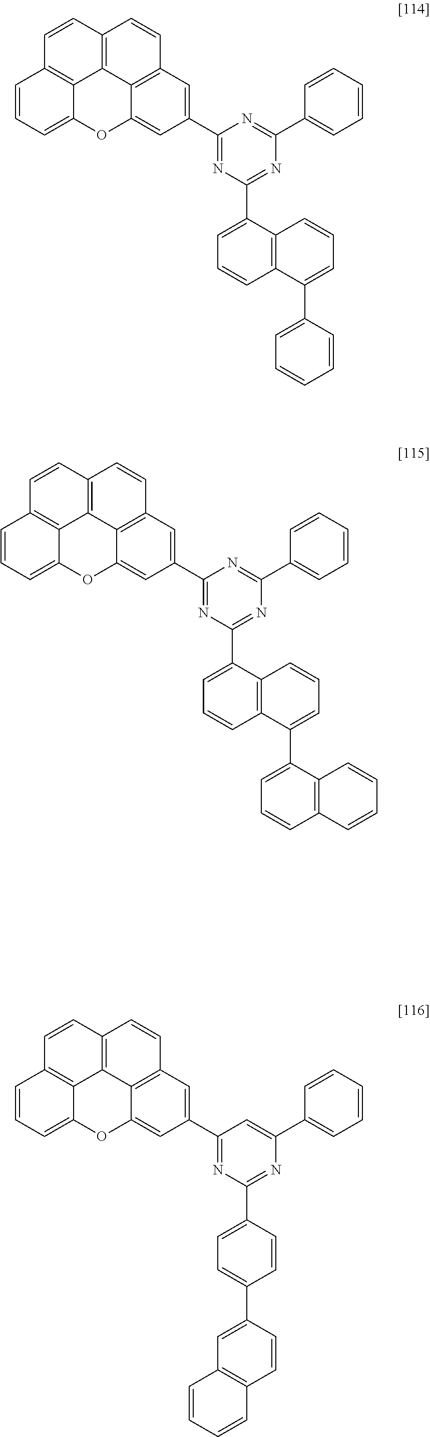 [SMILES: c1ccc(-c2nc(-c3cc4c5c(ccc6ccc7cccc(c7c65)O4)c3)nc(-c3cccc4c(-c5ccccc5)cccc34)n2)cc1 ; c1ccc(-c2nc(-c3cc4c5c(ccc6ccc7cccc(c7c65)O4)c3)nc(-c3cccc4c(-c5cccc6ccccc56)cccc34)n2)cc1 ; c1ccc(-c2cc(-c3cc4c5c(ccc6ccc7cccc(c7c65)O4)c3)nc(-c3ccc(-c4ccc5ccccc5c4)cc3)n2)cc1]

7. A composition for an organic optoelectronic device, the composition comprising
a first compound; and
a second compound, wherein:
the first compound is the compound for the organic optoelectronic device as claimed in claim 1, and
the second compound is a compound for an organic optoelectronic device represented by Chemical Formula 2: 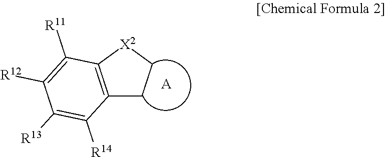
wherein, in Chemical Formula 2,
X² is O, S, NR^{b}, CR^{c}R^{d}, or SiR^{e}R^{f},
R^{b}, R^{c}, R^{d}, R^{e}, R^{f}, and R¹¹ to R¹⁴ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and
ring A is any one selected from the rings of Group II, 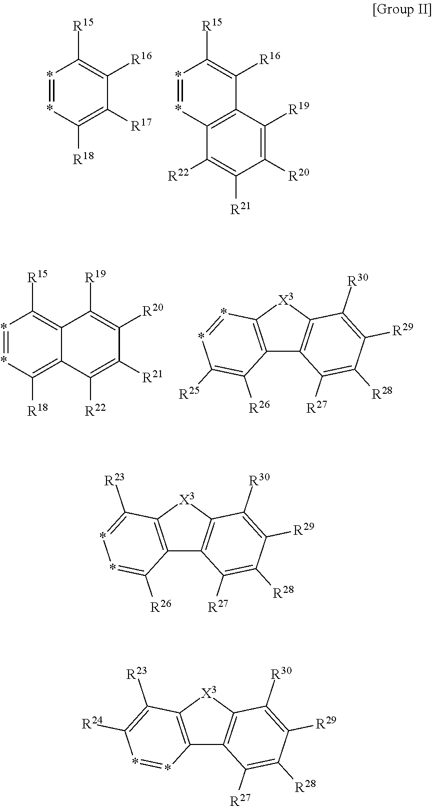
wherein, in Group II,
* is a linking point,
X³ is O, S, NR^{g}, CR^{h}Rⁱ, or SiR^{j}R^{k},
R^{g}, R^{h}, Rⁱ, R^{j}, R^{k}, and R¹⁵ to R³⁰ are each independently hydrogen, deuterium, a substituted or unsubstituted amine group, a substituted or unsubstituted C1 to C30 alkyl group, a substituted or unsubstituted C6 to C30 aryl group, or a substituted or unsubstituted C2 to C30 heterocyclic group, and
at least one of R¹¹ to R³¹ is a group represented by Chemical Formula b, 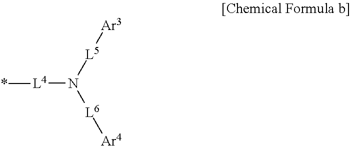
wherein, in Chemical Formula b,
L⁴ to L⁶ are each independently a single bond, or a substituted or unsubstituted C6 to C30 arylene group,
Ar³ and Ar⁴ are each independently a substituted or unsubstituted amine group, a substituted or unsubstituted C6 to C30 aryl group, or a substituted or unsubstituted C2 to C30 heterocyclic group, and
* is a linking point.

8. The composition as claimed in claim 7, wherein
Chemical Formula 2 is represented by any one of Chemical Formula 2-I to Chemical Formula 2-IX: 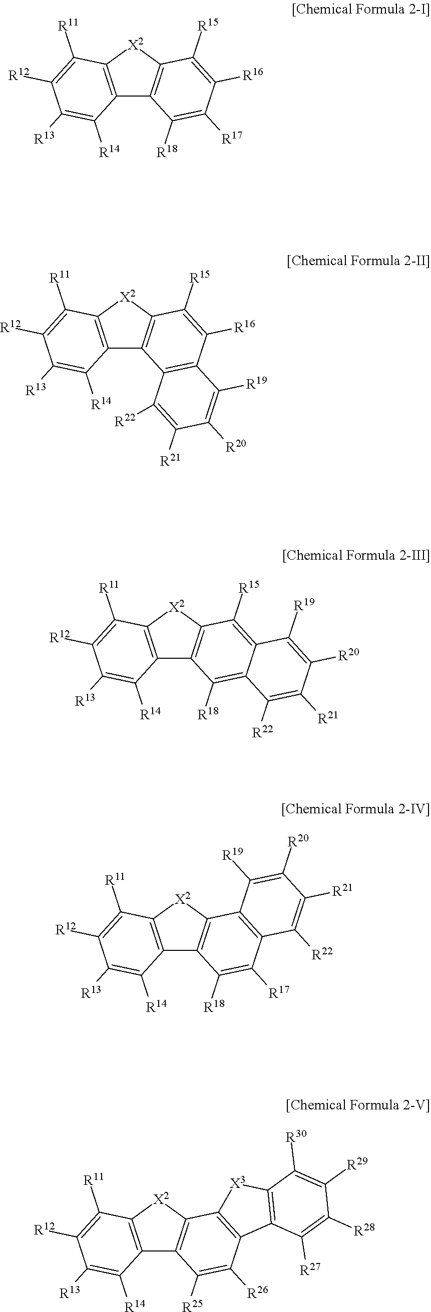 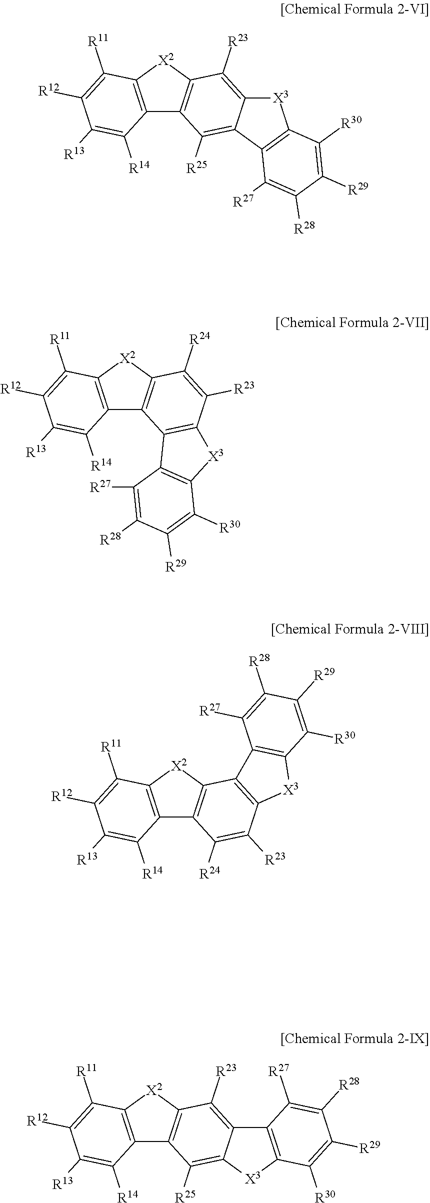
wherein, in Chemical Formula 2-I to Chemical Formula 2-IX,
the definitions of X², X³, and R¹¹ to R³⁰ are the same as for Chemical Formula 2.

9. The composition as claimed in claim 7, wherein the second compound is represented by any one of Chemical Formula 2-IA to Chemical Formula 2-IXA, Chemical Formula 2-TB to Chemical Formula 2-IXB, and Chemical Formula 2-IC to Chemical Formula 2-IIIC: 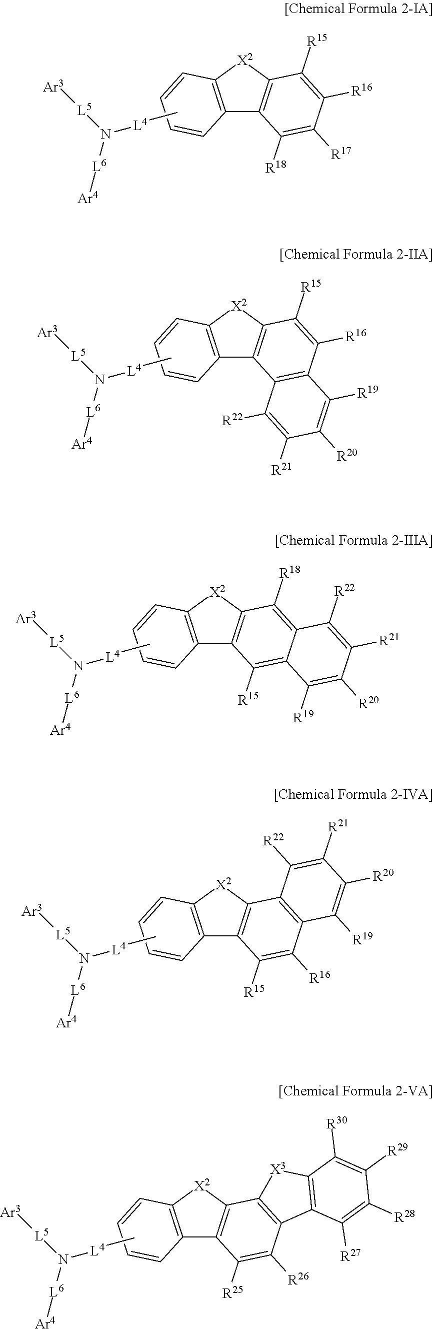 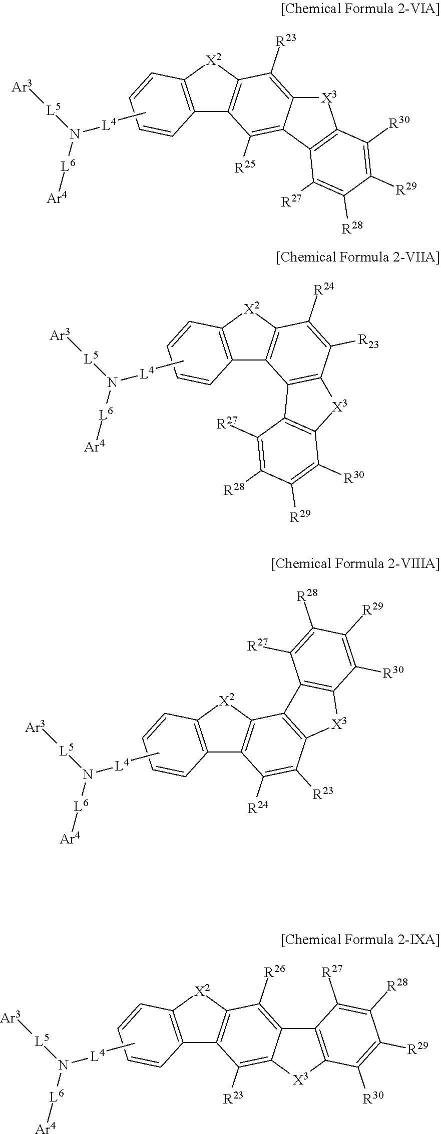 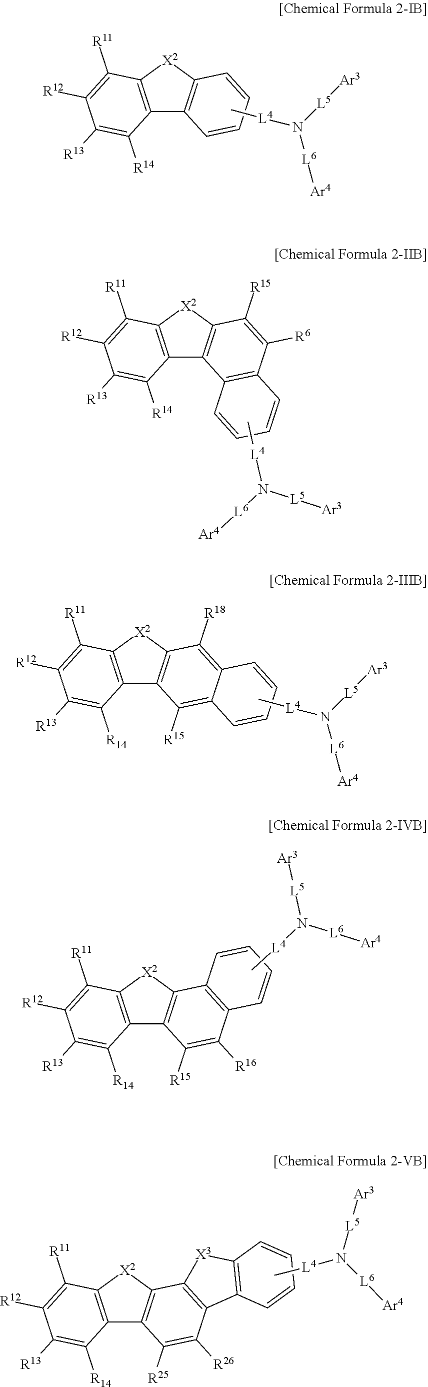 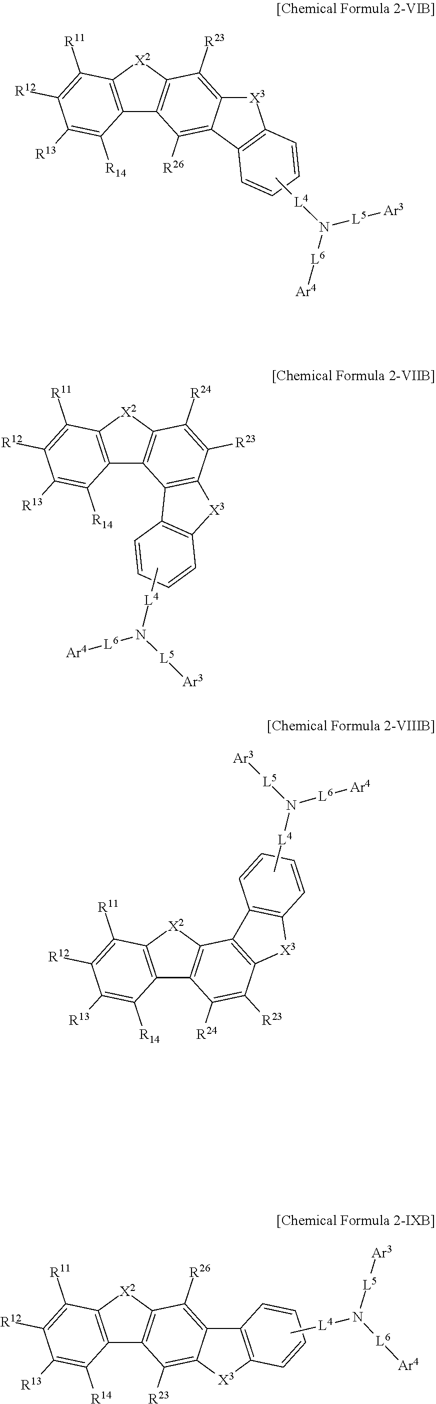 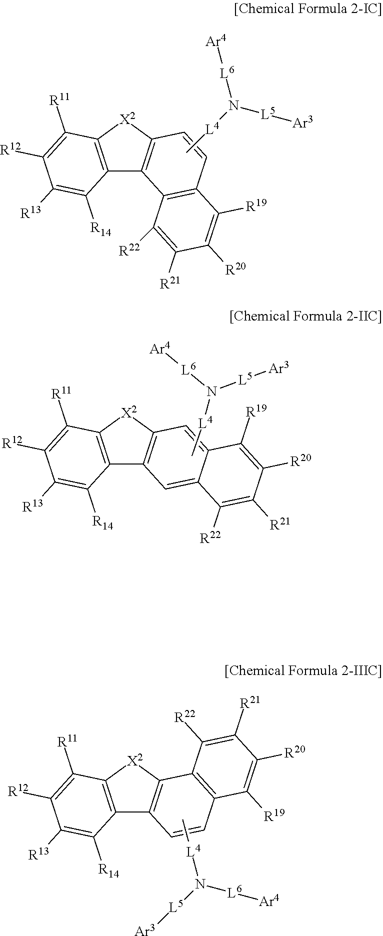
wherein, in Chemical Formula 2-IA to Chemical Formula 2-IXA, Chemical Formula 2-IB to Chemical Formula 2-IXB, and Chemical Formula 2-IC to Chemical Formula 2-IIIC,
X², X³, L⁴ to L, Ar³, and Ar⁴ are the same as for Chemical Formula 2, and
R¹¹ to R³⁰ are each independently hydrogen, deuterium, a substituted or unsubstituted C1 to C30 alkyl group, or a substituted or unsubstituted C6 to C30 aryl group.

10. The composition as claimed in claim 7, wherein the second compound is represented by any one of Chemical Formula 2-IVB-2 and Chemical Formula 2-VIIIB-2: 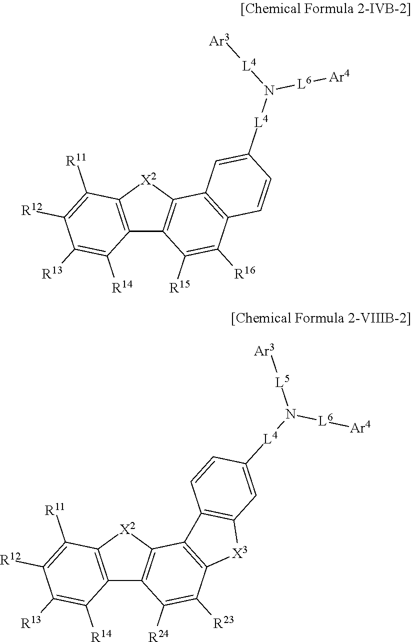
wherein, in Chemical Formula 2-IVB-2 and Chemical Formula 2-VIIIB-2,
L⁴ to L⁶ are each independently a single bond or a substituted or unsubstituted phenylene group,
Ar³ and Ar⁴ are each independently a substituted or unsubstituted phenyl group, a substituted or unsubstituted biphenyl group, or a substituted or unsubstituted naphthyl group,
X² is NW, O, or S,
X³ is CR^{h}Rⁱ or SiR^{j}R^{k},
R^{b}, R^{h}, Rⁱ, R^{j}, and R^{k} are each independently a substituted or unsubstituted C1 to C10 alkyl group, or a substituted or unsubstituted C6 to C20 aryl group, and
R¹¹ to R¹⁴, R¹⁵, R¹⁶, R²³, and R²⁴ are each independently hydrogen, deuterium, a substituted or unsubstituted C1 to C30 alkyl group or a substituted or unsubstituted C6 to C30 aryl group.

11. An organic optoelectronic device, comprising:
an anode and a cathode facing each other, and
at least one organic layer between the anode and the cathode,
wherein the at least one organic layer includes a light emitting layer that includes the compound as claimed in claim 1.

12. The organic optoelectronic device as claimed in claim 11, wherein the compound is included as a host of the light emitting layer.

13. A display device, comprising the organic optoelectronic device as claimed in claim 11.

14. An organic optoelectronic device, comprising:
an anode and a cathode facing each other, and
at least one organic layer between the anode and the cathode,
wherein the at least one organic layer includes a light emitting layer that includes the composition as claimed in claim 7.

15. The organic optoelectronic device as claimed in claim 14, wherein the composition is included as a host of the light emitting layer.

16. A display device, comprising the organic optoelectronic device as claimed in claim 14.
